SEQUENCE LISTING (21 sequences)

SEQ ID NO: 1 - protein, 7 aa, Artificial Sequence
  Synthetic
PKKKRKV

SEQ ID NO: 2 - protein, 16 aa, Artificial Sequence
  Synthetic
KRPAATKKAGQAKKKK

SEQ ID NO: 3 - protein, 9 aa, Artificial Sequence
  Synthetic
PAAKRVKLD

SEQ ID NO: 4 - protein, 11 aa, Artificial Sequence
  Synthetic
RQRRNELKRSP

SEQ ID NO: 5 - protein, 38 aa, Artificial Sequence
  Synthetic
NQSSNFGPMKGGNFGGRSSGPYGGGGQYFAKPRNQGGY

SEQ ID NO: 6 - protein, 42 aa, Artificial Sequence
  Synthetic
RMRIZFKNKGKDTAELRRRRVEVSVELRKAKKDEQILKRRNV

SEQ ID NO: 7 - protein, 8 aa, Artificial Sequence
  Synthetic
VSRKRPRP

SEQ ID NO: 8 - protein, 8 aa, Artificial Sequence
  Synthetic
PPKKARED

SEQ ID NO: 9 - protein, 8 aa, Artificial Sequence
  Synthetic
PQPKKKPL

SEQ ID NO: 10 - protein, 12 aa, Artificial Sequence
  Synthetic
SALIKKKKKMAP

SEQ ID NO: 11 - protein, 5 aa, Artificial Sequence
  Synthetic
DRLRR

SEQ ID NO: 12 - protein, 7 aa, Artificial Sequence
  Synthetic
PKQKKRK

SEQ ID NO: 13 - protein, 10 aa, Artificial Sequence
  Synthetic
RKLKKKIKKL

SEQ ID NO: 14 - protein, 10 aa, Artificial Sequence
  Synthetic
REKKKFLKRR

SEQ ID NO: 15 - protein, 20 aa, Artificial Sequence
  Synthetic
KRKGDEVDGVDEVAKKKSKK

SEQ ID NO: 16 - protein, 17 aa, Artificial Sequence
  Synthetic
RKCLQAGMNLEARKTKK

SEQ ID NO: 17 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 18 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 19 - DNA, 34 bp, Artificial Sequence
  Synthetic
ccctccaaggtttgtctgcagccattcgaggtgc

SEQ ID NO: 20 - DNA, 34 bp, Artificial Sequence
  Synthetic
ccctccaaggtttgtctgcagccattcgaggtgt

SEQ ID NO: 21 - DNA, 21 bp, Artificial Sequence
  Synthetic
tcgggcagcctgcatgatctt

(19) United States
(12) Patent Application Publication
(10) Pub. No.: US 2023/0000912 A1
(43) Pub. Date: Jan. 5, 2023

(54) GENETIC, DEVELOPMENTAL AND MICRO-ENVIRONMENTAL PROGRAMS IN IDH-MUTANT GLIOMAS, COMPOSITIONS OF MATTER AND METHODS OF USE THEREOF

(71) Applicants: THE BROAD INSTITUTE, INC., (US); MASSACHUSETTS INSTITUTE OF TECHNOLOGY, (US); THE GENERAL HOSPITAL CORPORATION, (US)

(72) Inventors: Bradley Bernstein, Boston, MA (US); Itay Tirosh, Cambridge, MA (US); Mario Suva, Boston, MA (US); Aviv Regev, Cambridge, MA (US); Orit Rozenblatt-Rosen, Cambridge, MA (US); Andrew Venteicher, Boston, MA (US)

(21) Appl. No.: 16/072,674

(22) PCT Filed: Jan. 25, 2017
(86) PCT No.: PCT/US2017/014995

§ 371 Date: Jul. 25, 2018

(51) Int. Cl. A61K 35/17; C12Q 1/6886; A61P 35/00; G01N 33/574; C07K 16/30

(52) U.S. Cl. CPC A61K 35/17; C12Q 1/6886; A61P 35/00; G01N 33/57407; C07K 16/30; C12Q 2600/106; C12Q 2600/112; C12Q 2600/118; C12Q 2600/136; C12Q 2600/158

(57) ABSTRACT

This invention relates generally to compositions and methods for identifying genes and gene networks that respond to, modulate, control or otherwise influence tumors and tissues, including cells and cell types of the tumors and tissues, and malignant, microenvironmental, or immunologic states of the tumor cells and tissues. The invention also relates to methods of diagnosing, prognosing and/or staging of tumors, tissues and cells, and provides compositions and methods of modulating expression of genes and gene networks of tumors, tissues and cells, as well as methods of identifying, designing and selecting appropriate treatment regimens.

### RELATED APPLICATIONS AND INCORPORATION BY REFERENCE

This application claims priority and benefit of U.S. provisional application Ser. No. 62/286,850, filed Jan. 25, 2016 and 62/437,558, filed Dec. 21, 2016.

Reference is made to International Patent Application Serial No. PCT/US16/40015, filed Jun. 29, 2016 and U.S. Provisional Application Ser. No. 62/186,227, filed Jun. 29, 2015. The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### STATEMENT REGARDING FEDERALLY SPONSORED RESEARCH

This invention was made with government support under grant numbers CA180922, CA14051 and CA165962 awarded by the National Institutes of Health. The government has certain rights in the invention.

### FIELD OF THE INVENTION

The present invention generally relates to the methods of identifying and using gene expression profiles representative of malignant, microenvironmental, or immunologic states of tumors, and use of such profiles for diagnosing, prognosing and/or staging of gliomas and designing and selecting appropriate treatment regimens.

### BACKGROUND OF THE INVENTION

Tumors are complex ecosystems defined by spatiotemporal interactions between heterogeneous cell types, including malignant, immune and stromal cells (1). Each tumor's cellular composition, as well as the interplay between these components, may exert critical roles in cancer development (2). However, the specific components, their salient biological functions, and the means by which they collectively define tumor behavior remain incompletely characterized.

Tumor cellular diversity poses both challenges and opportunities for cancer therapy. This is most clearly demonstrated by the remarkable but varied clinical efficacy achieved in malignant melanoma with targeted therapies and immunotherapies. First, immune checkpoint inhibitors produce substantial clinical responses in some patients with metastatic melanomas (3-7); however, the genomic and molecular determinants of response to these agents remain poorly understood. Although tumor neoantigens and PD-L1 expression clearly contribute (8-10), it is likely that other factors from subsets of malignant cells, the microenvironment, and tumor-infiltrating lymphocytes (TILs) also play essential roles (11). Second, melanomas that harbor the BRAFV600E mutation are commonly treated with RAF/MEK-inhibition prior to or following immune checkpoint inhibition. Although this regimen improves survival, virtually all patients eventually develop resistance to these drugs (12,13). Unfortunately, no targeted therapy currently exists for patients whose tumors lack BRAF mutations including NRAS mutant tumors, those with inactivating NF1 mutations, or rarer events (e.g., RAF fusions). Collectively, these factors highlight the need for a deeper understanding of melanoma composition and its impact on clinical course.

The next wave of therapeutic advances in cancer will likely be accelerated by emerging technologies that systematically assess the malignant, microenvironmental, and immunologic states most likely to inform treatment response and resistance. An ideal approach would assess salient cellular heterogeneity by quantifying variation in oncogenic signaling pathways, drug-resistant tumor cell subsets, and the spectrum of immune, stromal and other cell states that may inform immunotherapy response. Toward this end, emerging single-cell genomic approaches enable detailed evaluation of genetic and transcriptional features present in 100s-1000s of individual cells per tumor (14-16). In principle, this approach may provide a comprehensive means to identify all major cellular components simultaneously, determine their individual genomic and molecular states (15), and ascertain which of these features may predict or explain clinical responses to anticancer agents.

Intra-tumoral heterogeneity contributes to therapy failure and disease progression in cancer. Tumor cells vary in proliferation, sternness, invasion, apoptosis, chemoresistance and metabolism (72). Various factors may contribute to this heterogeneity. On the one hand, in the genetic model of cancer, distinct tumor subclones are generated by branched genetic evolution of cancer cells; on the other hand, it is also becoming increasingly clear that certain cancers display diversity due to features of normal tissue organization. From this perspective, non-genetic determinants, related to developmental pathways and epigenetic programs, such as those associated with the self-renewal of tissue stem cells and their differentiation into specialized cell types, contribute to tumor functional heterogeneity (73,74). In particular, in a hierarchical developmental model of cancer, cancer stem cells (CSC) have the unique capacity to self-renew and to generate non-tumorigenic differentiated cancer cells. This model is still controversial, but—if correct—has important practical implications for patient management (75,76). Pioneering studies in leukemias have indeed demonstrated that targeting stem cell programs or triggering cellular differentiation can override genetic alterations and yield clinical benefit (72,77).

Relating the genetic and non-genetic models of cancer heterogeneity, especially in solid human tumors, has been limited due to technical challenges. Analysis of human tumor genomes has shed light on the genetic model, but is typically performed in bulk and does not inform us on the concomitant functional states of cancer cells. Conversely, various markers have been used to isolate candidate CSCs across different human malignancies, and to demonstrate their capacity to propagate tumors in mouse xenograft experiments (72, 78-80). For example, in the field of human gliomas, candidate CSCs have been isolated in high-grade (WHO grades III-IV) lesions, using either combinations of cell surface markers such as CD133, SSEA-1, A2B5, CD44 and α-6 integrin or by in vitro selection and expansion of gliomaspheres in serum-free conditions (75, 76, 78, 80-83). However, these functional approaches have generated controversy, as they require in vitro or in vivo selection in animal models with results dependent on xenogeneic environments that are very different from the native human tumor milieu. In addition, these methods do not interrogate the relative contribution of genetic mutations to the observed phenotypes (which can limit reproducibility) and do not allow an unbiased analysis of cellular states in situ in human patients (72). It also remains largely unknown if candidate CSC-like cells described in human high-grade tumors are aberrantly generated during glioma progression by dedifferentiation of mature glial cells or if gliomas contain CSC-like cells early in their development—as grade II lesions—a question central for our understanding of the initial steps of gliomagenesis (84).

Tumor fitness, evolution and resistance to therapy are governed by genetic selection of cancer cells, by non-genetic programs related to developmental pathways and by influences of the tumor microenvironment (TME) (72). In recent years, seminal studies such as those of The Cancer Genome Atlas (TCGA) have charted the genetic landscape and the bulk expression states of thousands of tumors, identifying novel driver mutations and defining tumor transcriptional subtypes (112, 125). While the genetic state of tumors could be studied with high precision, due to the ability to distinguish malignant from germline genetic variation, bulk transcriptional profiles provided only limited insight into non-genetic determinants of cancer programs, TME influences and intra-tumoral heterogeneity. Single-cell RNA-seq analysis can help address those challenges (15, 85, 86, 126), but financial and logistic considerations, including the time required to accrue large cohorts of fresh tumor specimens, especially in rare entities, limit the ability to repeat a TCGA like effort at single-cell resolution. Thus, it is critical to cancer biology to develop a framework that allows the unbiased analysis of cellular programs at the single-cell level and across different genetic clones in human tumors, in situ, and at each stage of clinical progression, especially early in their development.

The present invention provides novel methods of identifying gene expression profiles representative of malignant, microenvironmental, or immunologic states of tumors and tissues, and of cells and cell types which they comprise. The invention further provides methods of diagnosing, prognosing and/or staging of tumors, tissues and cells. The invention also provides compositions and methods of modulating expression of genes and gene networks of tumors, tissues and cells, as well as methods of identifying, designing and selecting appropriate treatment regimens.

Citation or identification of any document in this application is not an admission that such document is available as prior art to the present invention.

### SUMMARY OF THE INVENTION

The invention relates to gene expression signatures and networks of tumors and tissues, as well as multicellular ecosystems of tumors and tissues and the cells and cell type which they comprise. Tumors are multicellular assemblies that encompass many distinct genotypic and phenotypic states. The invention provides methods of characterizing components, functions and interactions of tumors and tissues and the cells which they comprise. Single-cell RNA-seq was applied to thousands of malignant and non-malignant cells derived from melanomas, gliomas, head and neck cancer, brain metastases of breast cancer, and tumors in The Cancer Genome Atlas (TCGA) to examine tumor ecosystems.

The invention provides signature genes, gene products, and expression profiles of signature genes, gene networks, and gene products of tumors and component cells. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. In one embodiment, the patient is suffering from melanoma. The signature genes, gene products, and expression profiles are useful to identify components of tumors and tissues and states of such components, such as, without limitation, neoplastic cells, malignant cells, stem cells, immune cells, and malignant, microenvironmental, or immunologic states of such component cells.

Using single cell analysis in cancers including melanoma, glioma, brain metastases of breast cancer, and head and neck squamous cell carcinoma (HNSCC), as well as analyzing tumors in The Cancer Genome Atlas (TCGA), applicants have determined novel gene signature patterns and therapeutic targets.

Human tumor subclasses differ in genetic mutations, in non-genetic programs reflecting the cell-of-origin and associated pathways, and in the composition of the tumor microenvironment (TME). While cancer genomic studies such as those of The Cancer Genome Atlas (TCGA) identified genetic mutations that distinguish tumor subclasses, they provided only limited insight into developmental lineages and TME composition.

Using human oligodendrogliomas as a model, the inventors have profiled single cells from six patient tumors by RNA-seq and reconstructed their transcriptional architecture and related it to genetic mutations. It was surprisingly found that most cancer cells are differentiated along two specialized glial programs, while a rare subpopulation of cells is undifferentiated and associated with a neural stem cell/progenitor expression program. Surprisingly, cellular proliferation was highly enriched in this rare subpopulation, consistent with a model where a cancer stem cell/progenitor compartment is primarily responsible for fueling growth of oligodendrogliomas in humans. Analysis of sub-clonal genetic events shows that distinct clones within tumors span a similar cellular hierarchy, suggesting that the architecture of oligodendroglioma is primarily dictated by non-genetic developmental programs. These results provide unprecedented insight into the cellular composition of brain tumors at single-cell resolution and may help harmonize the cancer stem cell and the genetic models of cancer, with critical implications for disease management.

Moreover, Applicants also combined 9,879 single-cell RNA-seq profiles from ten IDH-mutant astrocytomas (IDH-A) with 4,347 single-cell profiles in the six IDH-mutant oligodendrogliomas (IDH-O) and 165 TCGA bulk RNA profiles to decouple genetic, epigenetic and TME effects of tumor composition and function across IDH-mutant gliomas. Differences in bulk profiles between IDH-A and IDH-O can be primarily explained by distinct TME composition and by signature genetic events, but not by distinct influences of glial lineages in the malignant cells of the two tumor types. Conversely, both tumor types share similar developmental hierarchies and lineages of glial differentiation, which differ from those of IDH-wildtype glioblastoma. Furthermore, as tumor grade increases, Applicants find both enhanced proliferation of malignant cells, a larger pool of undifferentiated gliomas cells and an increase in macrophage over microglia programs in the TME. These findings redefine the cellular composition of human IDH-mutant gliomas and outlines a general framework to dissect the differences between human tumor subclasses.

In one aspect, the invention relates to a method of treating glioma, comprising administering to a subject having glioma a therapeutically effective amount of an agent capable of reducing the expression or inhibiting the activity of one or more stem cell or progenitor cell signature genes or polypeptides; or capable of targeting or binding to one or more cell surface exposed stem cell or progenitor cell signature polypeptides. The agent may be capable of targeting or binding to one or more cell surface exposed stem cell or progenitor cell signature polypeptides and may be a CAR T cell capable of targeting or binding to one or more cell surface exposed stem cell or progenitor cell signature polypeptides.

In a further aspect, the invention relates to a method of treating glioma, comprising administering to a subject having glioma a therapeutically effective amount of an agent capable of inducing the expression or increasing the activity of one or more astrocyte and/or oligodendrocyte cell signature genes or polypeptides.

In an aspect, the invention relates to a method of treating glioma or enhancing treatment of glioma, which comprises administering an agent that increases or decreases expression of or the function of one or more signature genes or one or more products of one or more signature genes in one or more cell(s) of the glioma, wherein the one or more signature genes or one or more products of one or more signature genes comprises a signature gene as defined herein elsewhere. In certain embodiments astrocyte and/or oligodendrocyte signature gene expression or function/activity is increased. In certain embodiments, stem/progenitor cell signature gene expression or function/activity is decreased.

In certain embodiments, the level of expression, activity and/or function of one or more signature genes is determined by the level of expression of one or more products encoded by one or more signature genes in one or more cell(s) of the glioma. In certain embodiments, the level of expression of one or more products encoded by one or more signature genes is determined by a colorimetric assay or absorbance assay. In certain embodiments, the level of expression, activity and/or function of one or more signature genes or one or more products of one or more signature genes in one or more cell(s) of the glioma is determined by deconvolution of the bulk expression properties of a tumor.

As used herein, the term glioma has its ordinary meaning in the art. By means of further guidance, glioma refers to a tumor arising in the brain or spine, and is typically derived from or associated with glial cells. In certain embodiments, glioma as referred to herein includes without limitation oligodendrogliomas (derived from oligodendrocytes), ependymomas (derived from ependymal cells), astrocytomas (derived from astrocytes, and including glioblastoma (glioblastoma multiforme or grade IVV astrocytoma)), brainstem glioma (develops in the brain stem), optic nerve glioma (develops in or around the optic nerve), or mixed gliomas (such as oligoastrocytomas, containing cells from different types of glia). In a particular embodiment, glioma refers to oligoastrocytoma.

In certain embodiments, said glioma is low grade glioma. In certain embodiments, said glioma is high grade glioma. In certain embodiments, said glioma is grade I glioma. In certain embodiments, said glioma is grade II glioma. In certain embodiments, said glioma is grade III glioma. In certain embodiments, said glioma is grade IV glioma. In a preferred embodiment, said glioma is low grade glioma, or grade II glioma. Staging or grading or cancer in general and glioma in particular is well known in the art. By means of example, glioma may be graded according to the grading system of the World Health Organization (e.g. WHO grade II oligodendroglioma). In certain embodiments, glioma is primary glioma. In certain embodiments, glioma is metastatic (or secondary) glioma. In certain embodiments, glioma is recurrent glioma.

In certain embodiments, glioma as referred to herein is characterized by IDH1 and/or IDH2 (isocytrate dehydrogenase 1/2) mutations. In certain embodiments, the IDH1 mutation is R132H. In certain embodiments glioma as referred to herein is characterized by deletion of chromosome arms 1p and/or 19q. In certain embodiments, glioma as referred to herein is characterized by IDH1 and/or IDH2 mutations, such as IDH1 R132H mutation, and co-deletion of chromosome arms 1p and/or 19q. In certain embodiments, glioma is characterized by CIC (Protein capicua homolog) mutation. In certain embodiments, glioma as referred to herein is characterized by IDH1 and/or IDH2 mutations, such as IDH1 R132H mutation, and CIC mutation. In certain embodiments, glioma as referred to herein is characterized by deletion of chromosome arms 1p and/or 19q, and CIC mutation. In certain embodiments, glioma as referred to herein is characterized by IDH1 and/or IDH2 mutations, such as IDH1 R132H mutation, co-deletion of chromosome arms 1p and/or 19q, and CIC mutation. In certain embodiments, glioma as referred to herein is characterized by mutations in one or more genes selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36, one or more of which mutations may be present in the same cell or different cells of the tumor and may be present in the same cell or different cells of the tumor together with IDH1 and/or IDH2 mutations, such as IDH1 R132H mutation, co-deletion of chromosome arms 1p and/or 19q, and/or CIC mutation.

It will be understood that when referring to mutations in glioma, such mutations may be present in all or part of the tumor, such as for instance in all cells or in particular cell populations of the tumor. Hence a mutation is present or detected in at least part of the tumor or in at least part of the tumor cells. Mutation as referred to herein may refer to functional alteration of the affected gene, such as activation or inactivation of the gene or gene product, which may or may not be epigenetically.

In certain embodiments, the subject to be treated has not previously received chemotherapy and/or radiotherapy. In certain embodiments, the subject to be treated has previously received chemotherapy and/or radiotherapy.

In certain embodiments, treatment as referred to herein may comprise inducing differentiation of stem cells or progenitor cells comprised by or comprised in the glioma. In certain embodiments, said differentiation comprises induction of expression or activity of one or more astrocyte and/or oligodendrocyte signature genes or polypeptides in the stem cells or progenitor cells. In certain embodiments, treatment as referred to herein comprises reducing the viability of or rendering non-viable stem cells or progenitor cells comprised by or comprised in the glioma.

In an aspect, the invention relates to a method of diagnosing, prognosing, or stratifying or staging glioma, comprising determining expression or activity of one or more stem cell or progenitor cell signature genes or polypeptides in cells comprised by the glioma.

In an aspect, the invention relates to a method of diagnosing, prognosing, or stratifying or staging glioma, comprising determining expression or activity of one or more astrocyte signature genes or polypeptides in cells comprised by the glioma.

In an aspect, the invention relates to a method of diagnosing, prognosing, or stratifying or staging glioma, comprising determining expression or activity of one or more oligodendrocyte signature genes or polypeptides in cells comprised by the glioma.

In an aspect, the invention relates to a method of diagnosing, prognosing and/or staging a glioma, comprising detecting a first level of expression, activity and/or function of one or more signature genes or one or more products of one or more signature genes in one or more cell(s), population of cells or subpopulation of cells of the glioma and comparing the detected level to a control level of signature gene or gene product expression, activity and/or function, wherein a difference in the detected level and the control level indicates a malignant, microenvironmental, or immunologic state of the glioma.

In certain embodiments, such method comprises determining the relative expression level of one or more stem cell or progenitor cell signature genes or polypeptides compared to one or more astrocyte and/or oligodendrocyte signature genes or polypeptides in the cells comprised by or comprised in the glioma. In certain embodiments, such method comprises determining the fraction of the cells comprised by the glioma, which express one or more stem cell or progenitor cell signature genes or polypeptides. In certain embodiments, such method comprises determining the fraction of the cells comprised by the glioma, which express one or more astrocyte signature genes or polypeptides. In certain embodiments, such method comprises determining the fraction of the cells comprised by the glioma, which express one or more oligodendrocyte signature genes or polypeptides. In certain embodiments, such method comprises determining the fraction of the cells comprised by the glioma, which express one or more stem/progenitor cell, astrocyte, and oligodendrocyte signature genes or polypeptides. It will be understood that when referring to stem/progenitor cell, astrocyte, or oligodendrocyte signatures as referred to herein, such signatures may be specific for particular tumor cells or tumor cell (sub)populations having certain stem/progenitor, astrocyte, or oligodendrocyte characteristics, such as for instance as determined histologically or by means of identification of particular signatures characteristic of normal (i.e. non-cancerous) stem/progenitor, astrocyte, or oligodendrocyte cells. In certain embodiments, stem or progenitor cells as referred to herein refers to neural stem or progenitor cells.

In an aspect, the invention relates to a method of diagnosing, prognosing, stratifying or staging glioma, comprising identifying cells comprised by the glioma, which express one or more of CX3CR1, CD14, CD53, CD68, CD74, FCGR2A, HLA-DRA, or CSF1R, and/or one or more of MOBP, OPALIN, MBP, PLLP, CLDN11, MOG, or PLP1. In certain embodiments, these cells do not contain mutations, such as oncogenic mutations, in particular copy number variations (CNV). In certain embodiments, these cells do not contain IDH1 and/or IDH2 mutations, such as IDH1 R132H mutation, co-deletion of chromosome arms 1p and/or 19q, and CIC mutations. In certain embodiments, these cells do not contain mutations in FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36.

In an aspect, the invention relates to a method of identifying a therapeutic for glioma, comprising administering to a glioma cell, preferably in vitro, a candidate therapeutic and monitoring expression or activity of one or more stem cell or progenitor cell signature genes or polypeptides. In an aspect, the invention relates to a method of identifying a therapeutic for glioma, comprising administering to a glioma cell, preferably in vitro, a candidate therapeutic and monitoring expression or activity of one or more astrocyte cell signature genes or polypeptides. In an aspect, the invention relates to a method of identifying a therapeutic for glioma, comprising administering to a glioma cell, preferably in vitro, a candidate therapeutic and monitoring expression or activity of one or more oligodendrocyte signature genes or polypeptides. In an aspect, the invention relates to a method of identifying a therapeutic for glioma, comprising administering to a glioma cell, preferably in vitro, a candidate therapeutic and monitoring expression or activity of one or more stem cell or progenitor cell, astrocyte, and/or oligodendrocyte signature genes or polypeptides. As used herein, the term therapeutic refers to any agent suitable for therapy, as defined herein elsewhere.

In certain embodiments, reduction in expression or activity of said one or more stem cell or progenitor cell signature genes or polypeptides is indicative of a therapeutic effect. In certain embodiments, increase in expression or activity of said one or more astrocyte signature genes or polypeptides is indicative of a therapeutic effect. In certain embodiments, increase in expression or activity of said one or more oligodendrocyte signature genes or polypeptides is indicative of a therapeutic effect. In certain embodiments, reduction in expression or activity of said one or more stem cell or progenitor cell signature genes or polypeptides and concomitant increase in expression or activity of said one or more astrocyte and/or oligodendrocyte signature genes or polypeptides is indicative of a therapeutic effect.

In an aspect, the invention relates to a method of monitoring glioma treatment or evaluating glioma treatment efficacy, comprising determining expression or activity of one or more stem cell or progenitor cell signature genes or polypeptides in cells comprised by the glioma. In an aspect, the invention relates to a method of monitoring glioma treatment or evaluating glioma treatment efficacy, comprising determining expression or activity of one or more astrocyte signature genes or polypeptides in cells comprised by the glioma. In an aspect, the invention relates to a method of monitoring glioma treatment or evaluating glioma treatment efficacy, comprising determining expression or activity of one or more oligodendrocyte signature genes or polypeptides in cells comprised by the glioma. In an aspect, the invention relates to a method of monitoring glioma treatment or evaluating glioma treatment efficacy, comprising determining expression or activity of one or more stem cell or progenitor cell, astrocyte, and/or oligodendrocyte signature genes or polypeptides in cells comprised by the glioma.

In an aspect, the invention relates to a method for monitoring a subject undergoing a treatment or therapy for glioma comprising detecting a level of expression, activity and/or function of one or more signature genes or one or more products of one or more signature genes of the glioma (e.g. tumor stem/progenitor cell, astrocyte, and/or oligodendrocyte; as defined herein elsewhere) in the absence of the treatment or therapy and comparing the level of expression, activity and/or function of one or more signature genes or one or more products of one or more signature genes in the presence of the treatment or therapy, wherein a difference in the level of expression, activity and/or function of one or more signature genes or one or more products of one or more signature genes in the presence of the treatment or therapy indicates whether the patient is responsive to the treatment or therapy. In certain embodiments, the treatment or therapy modulates expression of one or more signature genes that indicates cell cycle state.

In certain embodiments, said monitoring methods comprises determining the relative expression level of one or more stem cell or progenitor cell signature genes or polypeptides compared to one or more astrocyte and/or oligodendrocyte signature genes or polypeptides in the cells comprised by the glioma. For instance, a decrease in expression of stem cell or progenitor cell signature genes or polypeptides and/or an increase of astrocyte and/or oligodendrocyte cell signature genes or polypeptides may be indicative of therapeutic effect.

In certain embodiments, said monitoring methods comprises determining the fraction of the cells comprised by the glioma, which express one or more stem cell or progenitor cell signature genes or polypeptides. In certain embodiments, said method comprises determining the fraction of the cells comprised by the glioma, which express one or more astrocyte cell signature genes or polypeptides. In certain embodiments, said method comprises determining the fraction of the cells comprised by the glioma, which express one or more oligodendrocyte cell signature genes or polypeptides. In certain embodiments, said method comprises determining the fraction of the cells comprised by the glioma, which express one or more stem cell or progenitor cell, astrocyte, and/or oligodendrocyte signature genes or polypeptides.

In certain embodiments of the invention, the stem cell or progenitor cell signature genes or polypeptides are not oligodendrocyte precursor cell signature genes or polypeptides.

In certain embodiments of the invention, the one or more stem cell or progenitor cell signature gene is selected from SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, COROIC, EIF4B, FBLIM1, SPDYE7P, TCF4, ORC6, SPDYEl, NCRUPAR, BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5, TNFAIP8L1, which are preferably expressed or upregulated.

In certain embodiments of the invention, the one or more stem cell or progenitor cell signature gene or polypeptide is selected from the group consisting of SOX4, SOX11, SOX2, NFIB, ASCL1, CDH7, CD24, BOC, and TCF4, which are preferably expressed or upregulated.

In certain embodiments of the invention, the one or more stem cell or progenitor cell signature gene or polypeptide is selected from the group consisting of SOX4, CCND2, SOX11, CDH7, CD24, NFIB, SOX2, TCF4, ASCL1, BOC, and EGFR, which are preferably expressed or upregulated.

In certain embodiments of the invention, the one or more stem cell or progenitor cell signature gene or polypeptide is selected from the group consisting of SOX11, SOX4, NFIB TCF4, SOX2, CDH7, BOC, and CCND2, which are preferably expressed or upregulated.

In certain embodiments of the invention, the one or more stem cell or progenitor cell signature gene or polypeptide is selected from the group consisting of SOX11, PTMA, NFIB, CCND2, SOX4, TCF4, CD24, CHD7, and SOX2, which are preferably expressed or upregulated.

In certain embodiments of the invention, the one or more stem cell or progenitor cell signature gene or polypeptide is selected from the group consisting of SOX2, SOX4, SOX11, MSI1, TERF2, CTNNB1, USP22, BRD3, CCND2, and PTEN, which are preferably expressed or upregulated.

In certain embodiments of the invention, the one or more stem cell or progenitor cell signature gene or polypeptide is selected from the SOX4, PTPRS, NFIB, CCND2, RBM6, SET, BAZ2B, TRA2A, which are preferably expressed or upregulated.

In certain embodiments of the invention, the stem cell or progenitor cell signature gene is selected from the group consisting of SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2, which are preferably expressed or upregulated.

In certain embodiments of the invention, the tumor stem cell or progenitor cell expresses or has an increased expression of one or more of NEDD4L, KCNQ1OT1, UGDH-AS1, ORC4, IGFBPL1, SHISA9, ASTN2, DCX, METTL21A, TMEM212, OPHN1, NRXN3, NREP, ARHGEF26-AS1, ODF2L, ABCC9, PEG10, SOX9, SOX4, TCF4, CHD7, UGT8, DLX5, XKR9, DLX6-AS1, SOX11, PDGFRA, DLX1, NPY, L2HGDH, PTPRS, GLIPR1L2, REXO1L1, CCL5, CTDSP2, SOX2, MAB21L3, TP53I1, GATS, ZFHX4, BAZ2B, DCLK2, GRIA2, LPAL2, CREBBP, MARCH6, PGM5P2, RERE, SPC25, GRIK3, CCDC88A, PVRIG, BRD3, GRIA3, MOXD1, SNTG1, TAGLN3, GSG1, DLX2, ATCAY, NUMA1, LMO1, POGZ, BPTF, CHRM3, RUFY3, SOX6, RPS11, TNFAIP8L1, FOXN3, DAPK1, DLL3, HERC2P4, TFDP2, GTF2IP1, DLX6, IGF1R, MLL3, NCAM1, CHL1, GNRHR2, CLIP3, FBLIM1, MATR3, CCNG2, NEK5, ETV1, KAT6B, SRRM2, FOXP1, DDX17, GOSR1, GATAD2B, MAP4K4, MIAT, CD24, ZNF638, HNRNPH1, BRD8, MLL, PCMTD1, AGPAT4, YPEL1, TNIK, PUM1, RFTN2, NNAT, MALAT1, GAD1, ZNF37BP, IRGQ, FXYD6, PRRC2B, FAM110B, YPEL3, ZMIZ1, CLASP1, SYNE2, BASP1, LYZ, ROCK1P1, DPY19L2P2, RSF1, HIP1, KANSL1, ELAVL4, TET3, ZEB2, ZBTB8A, MTSS1, TNRC6B, FOXO3, ANKRD12, MEIS3, JMJD1C, RICTOR, and MEST.

In certain embodiments of the invention, the tumor stem cell or progenitor cell expresses or has an increased expression of one or more of MAD2L1, ZWINT, MLF1IP, RRM2, CCNA2, TPX2, UBE2T, KIF11, MELK, NCAPG, MKI67, NUSAP1, CDK1, HMGB2, NCAPH, KIAA0101, FANCI, NUF2, TACC3, PRC1, CDCA5, FOXM1, CENPF, KIFC1, TOP2A, KIF2C, SMC2, AURKB, FAM64A, ASPM, DIAPH3, UBE2C, BUB1B, NDC80, ASF1B, KIF22, TK1, FANCD2, CASC5, GTSE1, RRM1, RACGAP1, TYMS, BIRC5, PBK, SPAG5, KIF23, TMPO, KIF15, DIFR, H2AFZ, ANLN, ORC6, ARHGAP11A, ESCO2, KIF4A, RNASEH2A, RAD51AP1, KIAA1524, SMC4, CENPN, KIF18B, VRK1, CCNB2, CKS1B, CKAP2L, SHCBP1, HISTIHIB, SGOL1, HIST1H3B, CENPM, CCNB1, BUB1, CENPK, HMGN2, ECT2, HMGB1, UHRF1, NCAPD2, HJURP, PKMYT1, MYBL2, CDC45, CDCA2, DLGAP5, TUBB, MCM10, ATAD2, MXD3, TUBAIB, SGOL2, DTYMK, CDC25C, TROAP, DTL, CDCA3, H2AFX, LIG1, TRIP13, HAUS8, KIF20B, NCAPG2, CDKN3, MIS18BP1, BRCA1, PLK4, CENPW, CDC20, SKA3, HIST1H4C, LMNB1, CDCA8, PLK1, RFC3, CENPO, DNMT1, EXO1, OIP5, CHAF1A, CENPE, POC1A, DEK, NUCKS1, MCM7, MIS18A, DEPDC1B, CHEK1, SPC24, GMNN, PTTG1, EZH2, MCM4, FEN1, GINS1, TTK, CDC6, RAD51, C19orf48, KIF20A, CKAP2, CDCA4, RFC5, SKA1, CENPQ, FANCA, PCNA, RFC4, PARP2, TMEM194A, FBXO5, TIMELESS, PSMC3IP, HIRIP3, POLA1, RANBP1, KIF18A, TCF19, USP1, LRR1, GGH, HMMR, CKS2, DNAJC9, SAE1, ITGB3BP, TMEM106C, FANCG, KPNA2, NCAPD3, HELLS, TMEM48, CBX5, SNRPB, KNTC1, NASP, MCM3, ZWILCH, RPA3, CHTF18, ANP32E, HIST1H3I, POLA2, MZT1, MCM2, DEPDC1, DUT, POLE, PHIP, PTMA, CSE1L, DSCC1, CDC7, HMGB3, TUBB4B, STMN1, RPA2, RCC1, CENPH, GINS2, EXOSC9, NCAPH2, NUDT15, SPC25, HNRNPA2B1, MND1, DSN1, MASTL, RAD21, PHGDH, ZNF331, RANGAP1, SAPCD2, PARPBP, ANP32B, SMC1A, NEK2, BARD1, NIF3L1, PRR11, HNRNPD, MCM5, SMC3, FAM111A, POLD1, CDK2, FUS, PHF19, ARHGAP33, NUP205, CDC25B, PA2G4, NUDT1, CHEK2, WDR34, H2AFY, HAUS1, BUB3, CHAF1B, PRIM2, CCDC34, POLE2, PRPS2, RFWD3, UBR7, CCNE2, RAN, DDX11, NUP50, CACYBP, HNRNPAB, DBF4, TMSB15A, AURKA, MAD2L2, GINS3, ASRGL1, PPIF, CKAP5, UBE2S, LMNB2, POLD3, TEX30, SUV39H1, CCP110, WHSC1, MCM6, ACYP1, GNG4, PRIM1, NSMCE4A, EXOSC8, COMMD4, SNRPD1, HAT1, H2AFV, CMC2, SSRP1, HIST1H1E, RBMX, LBR, RPL39L, EMP2, CENPL, CEP78, TRAIP, COPS3, LSM4, RBBP8, HIST1H1C, RPA1, RAD1, NUP210, HSPB11, RFC2, ACTL6A, SRRT, NUP107, GPN3, LSM3, SUV39H2, POLR2D, HAUS5, WDR76, LSM5, NXT1, TUBG1, C16orf59, REEP4, BTG3, RNASEH2B, TUBB6, PPIA, RBL1, ARL6IP6, COX17, SYNE2, GUSB, MSH5, CRNDE, DDX39A, SUPT16H, HNRNPUL1, POLE3, HAUS4, IDH2, H1FX, DCP2, NUP188, MPHOSPH9, PPIG, MAGOHB, RIF1, MLH1, MSH2, SNRNP40, HADH, GABPB1, NUDC, PHTF2, NUP85, NUP35, SKP2, THOC3, ANAPC11, TFAM, AKR1B1, ILF2, TMEM237, RAD54B, SMPD4, HMGN1, CBX3, TPRKB, GGCT, FBL, RFC1, CCT5, PRKDC, CDK5RAP2, SRSF2, CEP112, LDHA, SRSF3, HSP90AA1, SRSF7, HAUS6, CCHCR1, CEP57, HMGA1, UCHL5, C1orf174, CTPS1, ACOT7, SNHG1, PSMC3, ZNF93, PCM1, SFPQ, RMI1, NUP37, DCK, AHI1, SVIP, CHCHD2, ZNF714, XRCC5, NFATC2IP, SLC25A5, WRAP53, PSIP1, MRPS6, NT5DC2, and NOP58.

In certain embodiments, the one or more stem cell or progenitor cell signature gene is selected from the group consisting of SOX4, SOX11, HNRNPH1, PTMA, PTPRS, CHD7, CD24, SOX2, TFDP2, FBLIM1, TCF4, ORC6, BAZ2B, OPHN1, ZBTB8A, PGM5P2, MALAT1, CCL5, LYZ, NEK5, TNFAIP8L1, which are preferably expressed or upregulated.

In certain embodiments, the one or more stem cell or progenitor cell signature gene is selected from the group consisting of CCND2, RBM6, HNRNPL, TRA2A, SET, C6orf62, H3F3B, C14orf23, NFIB, SRGAP2C, STMN2, COROIC, EIF4B, SPDYE7P, SPDYEl, NCRUPAR, NELL2, SPHKAP, RAB42, LOH12CR2, ASCL1, BOC, ZNF793, TOX3, EGFR, EEF1A1, TATDN3, EVI2A, POU5F1, FBXO27, CAMK2N1, PABPC1, AFMID, QPCTL, MBOAT1, HAPLN1, LOC90834, LRTOMT, GATM-AS1, AZGP1, RAMP2-AS1, SPDYE5, which are preferably expressed or upregulated.

In certain embodiments, the stem cell or progenitor cell signature gene is selected from one or more of the group consisting of SOX4, SOX11, HNRNPH1, PTMA, PTPRS, CHD7, CD24, SOX2, TFDP2, FBLIM1, TCF4, ORC6, BAZ2B, OPHN1, ZBTB8A, PGM5P2, MALAT1, CCL5, LYZ, NEK5, TNFAIP8L1; and one or more of the group consisting of CCND2, RBM6, HNRNPL, TRA2A, SET, C6orf62, H3F3B, C14orf23, NFIB, SRGAP2C, STMN2, COROIC, EIF4B, SPDYE7P, SPDYEl, NCRUPAR, NELL2, SPHKAP, RAB42, LOH12CR2, ASCL1, BOC, ZNF793, TOX3, EGFR, EEF1A1, TATDN3, EVI2A, POU5F1, FBXO27, CAMK2N1, PABPC1, AFMID, QPCTL, MBOAT1, HAPLN1, LOC90834, LRTOMT, GATM-AS1, AZGP1, RAMP2-AS1, SPDYE5, which are preferably expressed or upregulated.

In certain embodiments of the invention, the tumor stem cell or progenitor cell further expresses or has an increased expression of one or more of G1/S signature genes or one or more G2/M signature genes. In certain embodiments of the invention, the tumor stem cell or progenitor cell further expresses or has an increased expression of one or more of MCM5, PCNA, TYMS, FEN1, MCM2, MCM4, RRM1, UNG, GINS2, MCM6, CDCA7, DTL, PRIM1, UHRF1, MLF1IP, HELLS, RFC2, RPA2, NASP, RAD51AP1, GMNN, WDR76, SLBP, CCNE2, UBR7, POLD3, MSH2, ATAD2, RAD51, RRM2, CDC45, CDC6, EXO1, TIPIN, DSCC1, BLM, CASP8AP2, USP1, CLSPN, POLA1, CHAF1B, BRIP1, E2F8, HMGB2, CDK1, NUSAP1, UBE2C, BIRC5, TPX2, TOP2A, NDC80, CKS2, NUF2, CKS1B, MKI67, TMPO, CENPF, TACC3, FAM64A, SMC4, CCNB2, CKAP2L, CKAP2, AURKB, BUB1, KIF11, ANP32E, TUBB4B, GTSE1, KIF20B, HJURP, HJURP, CDCA3, HN1, CDC20, TTK, CDC25C, KIF2C, RANGAP1, NCAPD2, DLGAP5, CDCA2, CDCA8, ECT2, KIF23, HMMR, AURKA, PSRC1, ANLN, LBR, CKAP5, CENPE, CTCF, NEK2, G2E3, GAS2L3, CBX5, CENPA.

In certain embodiments of the invention, the one or more astrocyte signature gene or polypeptide is selected from the group consisting of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1, and F3, which are preferably expressed or upregulated.

In certain embodiments of the invention, the one or more astrocyte signature gene or polypeptide is selected from the group consisting of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMIP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, and F3, which are preferably expressed or upregulated.

In certain embodiments of the invention, the one or more astrocyte signature gene or polypeptide is selected from the group consisting of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1, and DNAJB1, which are preferably expressed or upregulated.

In certain embodiments of the invention, the one or more oligodendrocyte signature gene or polypeptide is selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X, and PHACTR3, which are preferably expressed or upregulated.

In certain embodiments of the invention, the one or more oligodendrocyte signature gene or polypeptide is selected from the group consisting of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP, and CNP, which are preferably expressed or upregulated.

In certain embodiments of the invention, the one or more oligodendrocyte signature gene or polypeptide is selected from the group consisting of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL1, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X, and PHACTR3, which are preferably expressed or upregulated.

In certain embodiments of the invention, the tumor astrocyte does not express or has a reduced expression of one or more of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X, and PHACTR3.

In certain embodiments of the invention, the tumor astrocyte does not express or has a reduced expression of one or more of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP, and CNP.

In certain embodiments of the invention, the tumor astrocyte does not express or has a reduced expression of one or more of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL1, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X, and PHACTR3.

In certain embodiments of the invention, the tumor oligodendrocyte does not express or has a reduced expression of one or more of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1, and F3.

In certain embodiments of the invention, the tumor oligodendrocyte does not express or has a reduced expression (e.g. in CIC mutant cells compared to CIC wild type cells) of one or more of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, and F3.

In certain embodiments of the invention, the tumor oligodendrocyte does not express or has a reduced expression (e.g. in CIC mutant cells compared to CIC wild type cells) of one or more of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1, and DNAJB1.

In certain embodiments, the tumor stem/progenitor cell, astrocyte, and/or oligodendrocyte as referred to herein expresses or has an increased expression of one or more of ALG9, AP3S1, ARRDC3, BRAT1, CLN3, CNTNAP2, COL16A1, CTTN, DLD, DOCK10, DSEL, ECI2, EP300, ETV1, ETV5, FAR1, FOXRED1, FYTTD1, GATS, GFRA1, GLT25D2, GPR56, IGSF8, KANK1, KIAA1467, KIF22, LNX1, LPCAT1, ME3, MEGF11, MRPS16, NAV1, NFIA, NIN, NLGN3, NUP188, PCDH15, PCDHB9, PPP2R2B, PPWD1, PTN, RASD1, RNF214, SDC3, SEC24B, SLC38A10, STIM1, TMEM181, TTLL5, VARS, YJEFN3, ZNF451, and ZNF564.

In certain embodiments, the tumor stem/progenitor cell, astrocyte, and/or oligodendrocyte as referred to herein does not express or has an decreased expression of one or more of ANKMY2, ATF4, BRK1, BTF3L4, EIF3C, EVI2A, GFAP, MAD2L2, MPV17, MRPL46, NDUFV1, NFE2L2, RAB1A, RCOR3, RSL1D1, and TTC14.

In an aspect, the invention relates to an (isolated) cell characterized by comprising the expression of one or more a signature genes or polypeptide or combinations of signature genes/proteins as defined herein.

In a further aspect, the invention relates to a glioma gene expression signature characterized by one or more signature gene or polypeptide or combinations of signature genes/proteins as defined herein.

In certain embodiments, the gene signatures described herein encode surface exposed or transmembrane proteins, such that they can be targeted by CAR T cells, therapeutic antibodies or fragments thereof or antibody drug conjugates or fragments thereof.

In a further aspect, the invention relates to a method of monitoring an IDH-mutant glioma, comprising determining expression or activity of one or more macrophage and microglia signature genes or polypeptides in cells comprised by the IDH-mutant glioma, whereby an increase in macrophage over microglia programs in the tumor microenvironment indicates an increase in tumor grade and an increase in proliferation of malignant cells. The microglia signature genes may comprise CX3CR1, P2RY12, P2RY13 and SELPLG, and the macrophage signature genes may comprise CD163, CD74, TGFBI, IFITM2, IFITM3, F13A1, NPC2, TAGLN2 and FTH1.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention. Nothing herein is intended as a promise.

These and other embodiments are disclosed or are obvious from and encompassed by, the following

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings. Color versions of figures described herein are available in Tirosh et al., Single-cell RNA-seq supports a developmental hierarchy in human oligodendroglioma (Nature. (2016), vol. 539, pp. 309-313), herein incorporated by reference in its entirety.

FIG. 1A-IF. Expression differences between IDH-A and IDH-O are governed by the tumor microenvironment and genetics. (A) Single cell RNA-seq of 10 IDH-A tumors. Freshly-resected tumors were dissociated to single cell suspension, FACS-sorted and profiled by SmartSeq2 in 96-well plates. (B) Differential expression as defined by analysis of bulk TCGA tumors is only partially recapitulated in analysis of single cells. Shown are differentially expressed genes (rows), which are higher in either IDH-A (top) or IDH-A (bottom) bulk tumors. Relative expression is shown across all bulk tumors (left) or single malignant cells (middle). The corresponding differential expression between IDH-A and IDH-O after averaging all bulk tumors or all single cells is shown on the right. (C-E) TME contribution to differential expression. (C) Microglia/macrophage-specific genes (left column) and neuron-specific genes (right column) are enriched in genes with higher expression in IDH-A and IDH-O, respectively. (D) Distribution of expression differences between bulk IDH-A and IDH-O samples for microglia/macrophage-specific genes (black) and neuron-specific genes (grey). (E) Microglia/macrophage scores (X-axis) and neuron scores (Y-axis) (SOM), for bulk IDH-O (blue) and IDH-A (purple) tumors. (F) Most expression differences between IDH-A and IDH-O malignant cells are accounted for by genetic differences. Left: The assignment of differentially expressed genes (columns) (IDH-A vs. IDH-O) by both bulk and single cell analysis into four categories (top to bottom rows, SOM): (i) genes residing in chromosome arms 1p or 19q (which are co-deleted only in IDH-O), (ii) genes activated and (iii) genes repressed by CIC (for which one copy is lost in al IDH-O and a second copy is mutated in most IDH-O), and (iv) P53 target genes. Right: Observed and expected percentage of IDH-A specific genes and IDH-O specific genes which may be accounted by genetics; observed percentage was defined by inclusion in the first two and last two gene-sets for IDH-A and IDH-O specific genes, respectively. Expected percentage was defined by inclusion in the same gene-sets but across all genes rather than only the IDH-A and IDH-O specific genes.

FIG. 2A-2D. Differentiation programs are shared among IDH-A and IDH-O and account for most differences not explained by genetics. (A) Glia lineages do not distinguish IDH-A from IDH-O. Shown are the average expression levels of oligodendrocytic-specific (light blue) and astrocytic-specific (black) genes across all IDH-A (Y axis) and IDH-O (X axis) malignant cells. Line indicates equal expression in IDH-A and IDH-O. (B) Glia lineages distinguish cells within IDH-A. Shown are the correlations of oligodendrocytic-specific (light blue) and astrocyticspecific (black) genes with PC1 (X axis) and PC2 (Y axis) from a PCA of all IDH-A malignant cells. Line indicates equal expression in IDH-A and IDH-O. Selected genes are marked. (C) Classification of malignant cells (columns) from IDH-A (left panel) and IDH-O (right panel), based on the relative expression of the oligodendrocytic and astrocytic expression programs. Cells are ordered by the relative expression of the two programs. Top: significance of differential expression (Y axis, − log 10(P-value of a t-test)) between oligodendrocytic and astrocytic genes. Cells were sorted by significance from the most oligodendrocytic-like to the most astrocytic-like cells; dashed lines indicate a significance threshold of P<0.01. Bottom: Relative expression of 50 oligodendrocytic and 50 astrocytic genes for cells sorted by significance as in the top panel. (D) Lower differential expression between IDH-A and IDH-O among undifferentiated cells. For each malignant in IDH-A (purple) and IDH-O (blue), Applicants present its differentiation scores (Xaxis), defined as the maximum between the average expression of oligodendrocytic and astrocytic genes, vs. the average expression of IDH-A (left) or IDH-O specific (right) genes (Y axis), based on combined bulk and single cell analysis, but after excluding genes accounted by genetics. Lines indicate the corresponding local weighted smoothing regression (LOWESS), demonstrate the decrease in differences between IDH-A and IDH-O programs in cells with low glial differentiation scores.

FIG. 3A-3D. Undifferentiated cells in IDH-A and IDH-O are associated with cell cycle and a putative sternness program. (A) Inverse relation between cell cycle and cell differentiation. Shown is the percentage of cycling cells (Y axis) in any sliding window of 200 cells ranked by differentiation scores (X axis) for either IDH-A (purple) or IDH-O (blue) malignant cells. (B, C) Shared stemness program. (B) Shown are the Pearson correlation coefficients (color bar) between the expression profiles of genes (rows, columns) across undifferentiated IDH-A and IDH-O cells, for the ninety genes that were preferentially expressed in undifferentiated cells. Genes are ordered by their correlation with the highest-scoring cluster in each analysis (SOM). (C) Pearson correlations of the ninety genes in (B) with the highest-scoring clusters in (B) in IDH-A (X-axis) and IDH-O (Y-axis). The top consistent genes are marked. (D) In situ RNA hybridization (ISH) for the astrocytic markers APOE (apolipoprotein E, blue) and oligodendrocytic marker APOD (apolipoprotein D, red) shows expression of these lineage markers in distinct cells in IDH-A. APOE (blue) and the proliferation marker Ki67 (red, arrow) are mutually exclusive. The stem/progenitor markers SOX4 (SRY (sex determining region Y)— box4, blue) and Ki67 (red) are co-expressed in the same cells (arrow).

FIG. 4A-4H. Diversity and frequency of microglia and macrophages across IDH-mutant glioma. (A) Distinct microglia and macrophage programs. Shown are the expression levels of mouse orthologs of genes with high (red) and low (blue) scores in PC1 of a PCA across all microglia and macrophages in mice microglia (19) (Y-axis) and macrophages (X-axis; maximal expression across multiple macrophage types). (B,C) Relative expression of microglia- and macrophage-specific genes defines a spectrum of cellular states. (B) Top: distribution of scores defined by the average expression of microglia (PC1 high) vs. macrophage (PC1 low) genes (SOM). Bottom: differential expression of 10 representative genes (4 microglia- and 6 macrophage-specific) among all cells ranked by the scores at top. (C) For each tumor, shown is the fraction (color code) of cells in each bin of scores, as defined in (B, top). Macrophages from melanoma (5) are included for reference (top row). Tumor grades are shown on the right. (D, E) Endothelial expression program correlates with macrophage, but not with microglia, expression signatures across bulk TCGA samples. (D) Average endothelial scores (X-axis) vs. immune scores (Y-axis: macrophage, left; microglia, right) across IDH-A (purple filling) and IDH-O (blue filling) tumors partitioned by grade (gray, grade II; black, grade III; red, grade IV). Arrows indicate grade-specific changes that are associated with increased expression of the endothelial program. (E) Correlation between endothelial scores and immune scores (macrophage or microglia) across all IDH-A (purple) or IDH-O (blue) bulk TCGA tumors. (F,G) Factors associated with immune infiltration. (F) Top: correlation of the expression of each gene (column) with microglia or macrophage (row) scores across IDH-A (top two rows) and across IDH-O (bottom two rows) bulk tumors, for the 24 genes that are not expressed by microglia/macrophages but correlate significantly (P<0.05) with both microglia and macrophage scores. Bottom: differential expression of the same genes between IDH-A and IDH-O bulk tumors. Three genes from the complement system are marked. (G) Immune scores (X-axis: macrophage, left; microglia, right) correlate with the average expression of the 24 non-immune genes from (F) (Y-axis) across bulk IDH-A (purple) and IDH-O (blue) TCGA tumors. (H) In situ RNA hybridization (ISH) for microglia markers CX3CR1 (blue) and macrophage marker CD163 (red). Left panel shows MGH56, a tumor predominantly containing microglia-like signatures, as shown in (C). Central panels show MGH43, displaying both microglia-like cells, and macrophage-like cells circling blood vessels (arrow). Cells with intermediate status expressing both markers were also observed (arrow). The right panel shows MGH42, the tumor with the highest macrophage-like score in our cohort; accordingly, monocytic cells in that tumor are exclusively macrophage-like.

FIG. 5. Representative histology of our IDH-A cohort. Hematoxyline & Eosin stains of our tumors shows both intra- and inter-tumoral heterogeneity with various degrees of cellularity and cytonuclear pleomorphism. In MGH45, microvascular proliferations (arrow) and mitoses are observed, consistent with a grade IV tumor. Immunohistochemistry (IHC) for P53 in MGH42 shows strong nuclear staining consistent with the mutant protein. IHC for ATRX in MGH61 shows loss of expression in cancer cells (arrow) and retained nuclear expression in endothelial and immune cells (arrowheads).

FIG. 6A-6D. Cell classification by expression profiles and inferred CNVs. (A) Classification by expression profiles. Shown are Pearson correlation coefficients for the relative expression profiles of all analyzed genes, among all IDH-A single cells. Cells were ordered first based on assignment to three clusters as identified by hierarchical clustering; within each cluster cells were further ordered based on their tumor of origin, as indicated at the bottom panel, and within each sub-cluster that reflects a given tumor the cells were ordered based on hierarchical clustering. The three clusters were annotated as oligodendrocytes, microglia/macrophages, and malignant based on the top differentially expressed genes (Methods). (B-C) classification by CNVs. Applicants estimated CNVs based on the relative expression of genes in a sliding window of 100 genomically contiguous genes (Methods). (B) Shown are the estimated CNV values of all cells (rows) across all genomic positions (columns). Cells were sorted as in (A), demonstrating that the two clusters Applicants inferred as non-malignant have consistent CNV patterns despite harboring cells from different tumors, while the cells inferred as malignant have tumor-specific CNV patterns as expected for malignant cells. (C) Comparison of CNVs inferred from gene expression and averaged over cells from each tumor (RNA), to those defined from bulk whole-exome sequencing (WES) for three tumors. The consistency between CNV estimates was high in all three cases (Pearson R>0.6, P<10⁻¹⁶ in all cases); the remaining inconsistencies could reflect spatial differences between the tumor region used for single cell analysis and the one used for WES, as well as quantitative differences due to the limited sensitivities and caveats of both approaches. (D) Fluorescent in situ hybridization (FISH) analysis, demonstrating amplification of chromosome arm 7q and deletion of chromosome arm 10q by comparison of centromeric probes (CEP) to locus-specific probes (two probes for each chromosome arm).

FIG. 7A-7F. Consistency between expression and genetic analysis, and integrated cell classification. (A) Each cell was scored for the overall signal of CNVs (X-axis) and for the correlation between the CNV pattern of that cell and the average CNV pattern of all malignant cells from the same tumor (when malignant cells were determined based on gene expression clustering) (Y-axis). Cells are colored based on their assignment to the non-malignant expression clusters (blue and purple for microglia and oligodendrocytes, as well as 11 cells which Applicants identified as expressing a T-cell signature and are shown in green), while all other cells (malignant and unresolved cells) are colored in red. (B) Integrated expression and CNV classification. Shown are the expression and CNV scores for all cells which were retained for final analysis and classified as non-malignant (top) and malignant (bottom). Note that only three cells were excluded due to discordant classification by expression and CNV analysis, and 44 more cells were excluded due to unresolved assignment by both methods. (C-F) tSNE analysis of all cells profiled in this work, as well as fetal NPCs profiled recently (1), in which cells are grouped based on global similarity in gene expression (2). The same tSNE analysis is shown in each panel, but cells are colored by different criteria, demonstrating expression or genetic features. (C) Colors represent the tumor-of-origin of each cell, demonstrating that tumors primarily form distinct clusters, except for two main clusters that include cells from many tumors and correspond to immune cells (top left cluster) and oligodendrocytes (bottom left cluster) as demonstrated in (D). (D) Colors correspond to high expression levels (32-fold above average) of signatures specific to four cell types; the remaining cells that do not pass the threshold for any of the signatures are shown in red, and primarily correspond to cancer cells. (E) Colors represent cell classification as malignant based on CNV analysis: cells passing both thresholds in (A) are shown in red and all others are shown in black. (F) Cells in which an IDH1 mutation (p.R132C/H mutation) is identified in scRNA-seq reads are colored in red and all others are colored in black; right panel: Shown are the percentages of cells classified as malignant (left) and non-malignant (right) in which Applicants observed at least one read covering the site of the mutation and identified either a mutant allele (shown in red), a wild-type allele (blue), or both (purple). The ˜100-fold enrichment of mutant IDH reads in cells classified as malignant (P<10⁻¹⁶, hypergeometric test) further supports our classification, but these results also highlight the limited sensitivity of scRNA-seq mutation calling. This limitation is also observed for the wild-type allele, which is detected in a comparable fraction of cells as the mutant allele, indicating that scRNA-seq reads cover the exact site of the IDH1 mutation in <40% of the cells. Moreover, while the IDH mutation is heterozygote Applicants detect both the wild-type and the mutant alleles only in ˜5% of the malignant cells (and in none of the non-malignant cells), again reflecting the limited sensitivity and indicating that in most cells either none or one of the alleles is detected due to insufficient coverage of the mutation site (137).

FIG. 8A-8B. The tumor microenvironment contributes to bulk differences between IDH-A and IDH-O. (A) Analysis of the fraction of expression differences between IDH-A and IDH-O, identified in bulk analysis, which are recapitulated in single cell analysis, and those that may be accounted by the tumor-microenvironment (TME). The fraction of bulk differences recapitulated in single cell analysis depends on the threshold for defining expression differences in single cell analysis, while the fraction of remaining bulk differences that may be accounted by TME (i.e. preferential IDH-A expression of immune-specific genes and preferential IDH-O expression of neuron-specific genes) depends on the thresholds to assign genes as immune-specific and neuron-specific. Applicants thus examined these fractions with multiple fold-change thresholds for defining differential expression in single cell analysis, and for two stringencies of defining genes as cell type specific (strict and lenient, see Methods). For each fold-change threshold, the red line indicates the fraction of bulk differences recapitulated in single cell analysis above that threshold, while the dashed red line indicates the expected fraction based on the overall frequency of genes with IDH-A vs. IDH-O differences above that threshold in single cell analysis. The fraction of remaining differences which may be accounted by TME is shown for each fold-change threshold, based on the strict TME-specific genes definition (black line) and the lenient TME-specific gene definition (grey line); dashed black and grey lines indicate the expected fractions based on the overall number of immune-specific and neuron-specific genes. While the results are threshold dependent, in all cases a considerable fraction of bulk differences are not recapitulated by single cell analysis (38-64%) and, of those, most differences may be accounted by TME (53-75%). (B) Estimation of the relative abundance of microglia/macrophages (X-axis) and neurons (Y-axis) in bulk TCGA samples, based on the average expression of all genes specific to each cell type. Shown are IDH-A (purple) and IDH-O (blue) bulk samples separated by grade (grade II, top panel; grade III; bottom panel). The differences between IDH-A and IDH-O are significant within each of the grades, for both microglia/macrophages and neurons (P<0.01 in all comparisons, t-test).

FIG. 9A-9C. Glial differentiation programs are largely independent of technical (A) or batch (B) effects and are reproduced in an alternative single cell RNA-seq platform (C). (A) Technical complexity of RNA-seq libraries is reflected in the number of genes detected by at least one mapping read. The number of genes detected (Y-axis) is shown for all IDH-A (top) and IDH-O (bottom) malignant cells, which were ranked by glial differentiation scores, as shown in FIG. 2C; red lines indicate a moving average of 200 genes. (B) Each row corresponds to a distinct 96-well plate which corresponds to sorting and sequencing batches. Shown is the frequency of cells, log 2 (number of cells+1), in bins defined by glial differentiation scores. This analysis demonstrates that different batches for the same tumor are highly consistent, while some differences are observed between tumors which are shown as separate panels from top to bottom and tumor names are indicated at the right. (C) Glial differentiation programs are reproduced in single cell RNA-seq analysis using the 10× genomics platform (www.10×genomics.com/) that contains unique-molecular identifiers (UMIs). Applicants profiled two of the IDH-A tumors in our cohort (MGH103 and MGH107) with the 10× platform, processed the data with the 10× analysis pipeline (CellRanger), and identified malignant cells as described in FIG. 6. Shown is the expression of oligo-specific and astro-specific genes across all malignant cells, ranked by the relative expression of oligo-specific and astro-specific genes, as done in FIG. 2C for the main dataset with the Smart-Seq2 platform.

FIG. 10A-10E. Glial differentiation patterns. (A) Each tumor has a wide distribution of glial differentiation states, although many tumors are enriched with certain cellular states. IDH-A (top) and IDH-O (bottom) malignant cells were sorted from oligodendrocytic-like to astrocytic-like as shown in FIG. 2C. Shown is the frequency of cells in a sliding window of 250 cells that are derived from each tumor, where tumor names are indicated at the right. (B) The distribution of astrocytic scores (top) and oligodendrocytic scores (bottom) are shown for all malignant cells from IDH-O (blue), IDH-A (purple) and GBM (1) (black). (C) Average relative expression of IDH-A specific genes whose expression difference between IDH-A cells (purple) and IDH-O (blue) cells could be accounted by IDH-O specific genetic (i.e. co-deletion of chromosome arms 1p and 19q and CIC mutations); lines indicate a LOWESS regression with a window spanning 20% of the cells. (D-E) Astrocytic and oligodendrocytic scores are negatively correlated preferentially in IDH-O. (D) Density of cells in combinations of astrocytic scores (X-axis) and oligodendrocytic scores (Y-axis) are shown for all malignant cells from IDH-O (left panel) and IDH-A (right panel). The range of values for each score (oligo. and astro.) was divided to 70 equal bins, and the number of cells (N) in each combination of bins is color coded by log 2 (N+1). (E) Averaged astrocytic scores over cells primarily differentiated into the oligodendrocytic lineage in sliding windows of 200 IDH-A (purple) and IDH-O (blue) cells ranked by their oligodendrocytic scores. Undifferentiated cells (differentiation score below 0.3) and cells with higher astrocytic than oligodendrocytic scores were excluded from this analysis in order to focus on the oligodendrocytic compartment of the tumors. The Spearman correlations (R) between astrocytic and oligodendrocytic scores (before smoothing) and the P-values (P) from t-tests comparing astrocytic scores between third of the cells with the highest oligo scores and third of the cells with the lowest oligo scores, are also indicated at the left for both IDH-O (blue) and IDH-A (purple).

FIG. 11. Comparison of genome-wide DNA methylation profiles of IDH-O, IDH-A, IDH-wildtype glioma (112), IDH1/2-mutant AML (141), and IDH mutant chondrosarcoma (142). Heatmap representation of the 10,000 most variably methylated CpG probes (excluding probes mapping to chrX/Y) indicates high similarity of IDH-O and IDH-A relative to other tumor classes. CpG probes (rows) are ordered by hierarchical clustering. AML and Chondrosarcoma derive from distant developmental lineages, whereas differences to IDH-wildtype glioma (mostly gain of CpG methylation) could to a large degree be attributed to the G-CIMP phenotype.

FIG. 12. Cell cycle analysis. G1/S score (X-axis) and G2/M score (Y-axis) defined as the average relative expression of the corresponding gene-sets are shown for all IDH-A and IDH-O malignant cells. Cells defined as cycling were color coded in blue for IDH-O and purple for IDH-A cells.

FIG. 13A-13C. Combined TDH-A and IDH-O sternness program. (A-B) The putative stemness program, defined by enrichment and co-expression in undifferentiated IDH-A and IDH-O cells, is consistent with expression programs of neural progenitor cells (NPCs) and neural stem cells (NSCs). (A) Shown are the distribution of correlation values of genes (X axis) with the NPC (left panel) and NSC (right panel) expression programs, across all genes (gray), or across genes enriched but not co-expressed (black), or across genes enriched and co-expressed among the undifferentiated IDH-A and IDH-O cells. The NSC activation program was defined by single cell analysis of mice NSCs, as previously quantified by “pseudotime” (2). The NPC expression program was defined by the first principal component in analysis of single cell RNA-seq data of human fetal NPCs, as described previously (3). Correlations were defined across the respective single cell datasets (NPCs or NSCs). The genes enriched and co-expressed (red) have significantly higher correlations compared either to all other genes (grey) or to enriched but not co-expressed genes (black), with both the NPC and the NSC program (P<0.01 in all cases, t-test). (B) Correlations of genes enriched in undifferentiated IDH-A and IDH-O cells with the NSC (X-axis) and NPC (Y-axis) programs. Genes which are also co-expressed in IDH-A and IDH-O are marked in red and labeled. (C) Each panel shows the differentiation and stemness scores of malignant cells from a particular tumor. Tumor names are indicated at the right top corner with a one-letter code for tumor type (A and O for IDH-A and IDH-0, respectively).

FIG. 14A-14C. Apparent differences between IDH-O and IDH-A (in the frequency of cycling and undifferentiated cells and in the negative association between the two lineage scores) are correlated with grade in IDH-A, and may reflect the higher grades of IDH-A tumors in our cohort. (A-B) Shown are the percentages of undifferentiated cells (X-axis), cycling cells (A, Y-axis) or Astro-Oligo lineage correlation (B, Y-axis) for each of the IDH-O (circle) and IDH-A (square) tumors, which are also colored by grade (grey, grade II; black, grade III; red, grade IV). These analyses demonstrate that MGH107, a grade II IDH-A in our cohort, resembles IDH-O grade II tumors, and that the two IDH-A grade IV tumors are especially distinct from IDH-O grade II tumors, suggesting that the observed variability between IDH-A and IDH-O tumors may be derived from differences in grades. (C) Grade-related differences in cell cycle frequencies are recapitulated in analysis of bulk TCGA samples. Each bulk sample was scored for the expression of G1/S-specific (X-axis) and G2/M-specific (Y-axis) genes, and the average scores shown for sets of tumors with the same tumor type (IDH-A in squares and IDH-O in circles) and the same grade (grade II, III and IV, in grey, black and red, respectively). Cell cycle scores (for both G1/S and G2/M) were significantly different (P<0.05, t-test) in all comparisons between distinct grades for the same tumor types (as illustrated by dashed lines), and were not significant (P>0.05) in comparisons of the same grade across tumor types (for grade II and for grade III).

FIG. 15A-15C. Genetic intra-tumor heterogeneity identified by CNVs and associated differences in cell cycle and glial differentiation. CNV analysis (left panels) of three tumors—MGH44 (A), MGH103 (B), and MGH57 (C)—revealed large-scale CNVs which vary between cells of the same tumor. Applicants ranked cells (A,B) or clustered cells (C) based on their estimated copy numbers at these chromosomal regions and defined putative subclones, while excluding cells with intermediate values that cannot be assigned confidently (A). For each of the three tumors, Applicants then compared the two clones with respect to the distribution of glial differentiation scores (middle panels, showing astrocytic and oligodendrocytic scores), stemness vs. differentiation scores (top right panels, as defined in FIG. 13) and the fraction of cycling cells (right panels, showing the fraction in all cells, and in clone 1 and clone 2). In MGH57 Applicants focused on the two largest clones, since analysis of other clones was limited by cell numbers. In all three tumors Applicants found significant differences in differentiation patterns (Kolmogorov-Smirnov test; * and ** correspond to P<0.05 and P<0.001, respectively) and in one case (C) Applicants also found significant difference in fraction of cycling cells (hypergeometric test, P=0.004). In MGH44 (A) and MGH57 (C), there are small subsets of cells that may reflect stem cells, with low differentiation score and high stemness score (top left cells in the top right panel), while a similar subset is not found in MGH103 (B); In MGH57 this subset contains cells of both clones, and in MGH44 this subset may be biased to clone 1, although this cannot be determined confidently due to limited cell number and dependence on exact threshold for defining the subset.

FIG. 16A-16B. PCA of macrophage/microglia cells from three IDH-O (A,B) PCA was performed over all macrophage/microglia cells from the twelve tumors (shown in black) and each panel highlights the cells from one tumor (shown in red). PC2 reflects an inflammatory program, while PC1 reflects macrophage (PC1-low vs. microglia (PC1-high) expression programs. Four tumors that Applicants profiled (MGH60, MGH93, MGH97 and MGH103) are not included in this analysis since Applicants only sequenced CD45- plates after FACS sorting of those tumors and identified at most 2 macrophage/microglia cells per tumor.

FIG. 17A-17F Single-cell RNA-seq of cancer and non-cancer cells in six oligodendroglioma tumors. (A) Experimental workflow. (B,C) Copy-number variations (CNVs) inferred from single cell RNA-Seq. Rows: cells; columns: chromosomal locations (100 gene windows). Red: inferred amplification; blue: inferred deletion; white: normal karyotype. (b) CNV profiles inferred from single cell RNA-seq for each of six tumors (top panel) and measured by DNA whole-exome sequencing (WES) of five tumors (bottom panel). Top cluster (in top panel): non-tumoral cells that lack CNVs, 3 bottom clusters: remaining cells from each of the six tumors, with deletions of chromosomes 1p and 19q, as well as tumor-specific CNVs. MGH36 and MGH97 cells are ordered by their pattern of CNVs, indicating variability in the copy numbers of chromosomes 4, 11 and 12, with a zoomed in view on a fraction of cells in (C). (D) PCA of malignant cells. Shown are PC1 (X-axis) vs. PC2+PC3 (Y-axis) scores of cells from three tumors based on a single combined PCA. (E) AC-like and OC-like signatures. Relative expression of the genes most correlated positively (bottom) or negatively (top) with PC1, in cancer cells from each of the three tumors (marked as in (D)), ranked by PC1 scores. Selected AC and OC marker genes are highlighted. (F) Relative expression of the mice orthologs of genes most correlated positively (bottom) or negatively (top) with PC1 (as shown in (E)) in mice OCs and ACs (97) (log₂-ratio of the respective cell type compared to the average of four measured cell types: OC, AC, OPC and neurons). Abbreviations: AC: astrocyte; OC: oligodendrocyte.

FIG. 18A-18G Stemness expression program and a developmental hierarchy of oligodendroglioma cells. (A) Stemness program. Average relative expression of the genes most highly correlated with PC2+PC3 (top), as well as the selected AC and OC marker genes shown in FIG. 17e (bottom), in four subpopulations defined by PC scores: stem-like cells (high PC2+PC3, intermediate PC1); undifferentiated cells (undiff; low PC2+PC3, intermediate PC1); OC-like (high PC1); AC-like (low PC1). Genes were sorted by their relative expression in the stem-like cells. (B) Stemness program genes are also expressed in early human brain development. Relative expression of putative stemness genes correlated with PC2/3 (top) and OC/AC marker genes (bottom) across 524 human brain samples from the Human Developmental Transcriptome in the Allen Brain Atlas. Samples are ordered in columns by age, from early prenatal (left) to adults (right). (C) The stemness program is correlated to those of mouse activated NSC and human NPCs. Pearson correlation coefficients between the expression of PC2/3 genes (rows) and expression programs of mouse NSC (left) and human NPC (right) across single cells from the respective datasets; the NSC expression program reflects activation, and is quantified by “pseudotime” as defined previously (111); the NPC program reflects PC1 scores from a PCA analysis of 340 NPCs (FIG. 29). (D) Inferred developmental hierarchy in oligodendroglioma cells. Lineage scores (OC-like vs. AC-like expression program; X-axis, Methods) and stemness scores (stem-like vs. OC/AC-differentiation expression program; Y-axis, Methods) of malignant cells from the six tumors. Gray lines indicate the backbone (Methods) used to quantify density in FIG. 37B, 38A-B. (E) Density of cells (color bar) from each tumor across the backbone of the hierarchy in (A). For each position in the backbone, colors indicate the fraction of cells in each tumor that are within a Euclidean distance of 0.3. (F) Fraction of cancer cells in each of the compartment. Shown is the fraction of cells assigned to the different tumor compartments (Y axis, Methods) based on either single cell RNA-seq (blue) or RNA-ISH (orange), (example RNA-ISH shown in (G)). Circles: individual tumors; square and error bars: average and standard deviation across tumors, respectively, showing general agreement between scRNA-Seq and IHC estimates. (G) Tissue staining. Immunohistochemistry for Glial Fibrillary Acidic Protein (GFAP) and OLIG2 highlights astrocytic and oligodendroglial lineage differentiation, respectively, in subpopulations of cells in oligodendroglioma sample MGH54 (two top left panels). In situ RNA hybridization (ISH) for astrocytic markers APOE (apolipoprotein E, arrowhead) and oligodendrocytic marker OMG (oligodendrocyte myelin glycoprotein, arrow) confirms expression of these two lineage markers in distinct cells in oligodendroglioma. The stem/progenitor markers SOX4 (SRY (sex determining region Y)-box4) and CCND2 (cyclinD2), arrowheads, are co-expressed in the same cells and are mutually exclusive with the lineage marker ApoE (arrow).

FIG. 19A-19E. Cell cycle is enriched in the stem/progenitor cells in oligodendroglioma. (A) Cell cycle classification. Classification of cells to non-cycling (black) and three categories of cycling cells (color-coded by approximated phase as shown in inset) based on the relative expression of gene-sets associated with G1/S (X-axis) and G2/M (Y-axis) phases of the cell cycle. Thin light blue cells have intermediate scores and thus might reflect either early G1 phase, or possibly arrested or non-cycling cells. Blue, green and red cells have more significant expression of cell cycle genes and are thus more confidently defined as cycling cells. (B-D) Only stem/progenitor cells are cycling. (b) Hierarchy plot, as in FIG. 36d for MGH54 cells, with confidently-cycling cells color-coded as in (A). For Light blue (less confident) cells and the other tumors see FIG. 48. (C) Hierarchy plot for the six tumors, with each cell color-coded based on the fraction of neighboring cells, as defined with a Euclidean distance of 0.3, that are cycling (including light blue cells). (D) Left: ISH for Ki-67 (cell cycle marker) and SOX4 (stemness marker) showing co-expression in rare cells (arrows). A non-cycling Sox4+ cells is also highlighted (arrowhead). Right: Double immunohistochemistry for the differentiation marker GFAP (red) and the proliferation marker Ki-67 (brown), showing that proliferating cells (arrowheads) do not express differentiation markers (arrows). (E) Correlation between the average expression of cell cycle (Y-axis) and that of stemness genes (X-axis) across molecularly defined (IDH mutations, chromosome 1p and 19q co-deletion, and absence of P53 and ATRX mutations) oligodendrogliomas (circles) profiled by TCGA with bulk RNA-seq. Average expression was defined by centering the log 2-transformed RSEM gene quantifications. Also shown are the linear least-square regression and Pearson correlation coefficient.

FIG. 20A-20J. Intra-tumor genetic heterogeneity and association with expression states. Cells were classified to genetic subclones based on CNVs (A,B) or point-mutations (C-E), and examined for differences in gene expression states. (A,B) Both CNV clones in MGH36 and in MGH97 span all 3 tumor compartments. (A) Two clones (green and gray) in MGH36 and MGH97 based on CNV inference mapped to the cellular hierarchy defined by lineage (x-axis) and stemness (Y axis) scores. (B) Percentages of cycling cells (X axis) and of stem/progenitor cells (Y axis) in clone 1 (green) and clone 2 (gray) of MGH36 (square) and MGH97 (diamond). (C,D) Different clones defined by point mutations span all three tumor compartments. (C) Clones inferred by mutation analysis of single cell RNA-seq reads. Each panel shows lineage (X-axis) and stemness (Y-axis) scores for cells, colored by their mutation status (red: detected by single cell RNA-seq reads; black: not detected). Top left corner: mutation name, expected (E) fraction of mutant cells by ABSOLUTE (35), and fraction of single cells were the mutation was observed (O). (D) Clones determined by single cell mutation-specific qPCR. As in (C) but showing a wild-type CIC allele detected (green), a mutant CIC allele detected (orange) or neither one detected (black). (E) An expression signature for CIC-mutant cells. Shown is a heatmap of relative expression levels for CIC-dependent genes (rows) in CIC-mutant (right columns) and CIC-wild-type (left columns) cells. Key gene names are marked on left. Cells were classified to genetic subclones based on CNVs (F,G) or point-mutations (H-J), and examined for differences in gene expression states. (F,G) Both CNV clones in MGH36 span all 3 tumor compartments. (F) Two clones in MGH36 based on CNV inference mapped to the cellular hierarchy defined by lineage (x-axis) and stemness (Y axis) scores. (G) Density (color bar) of all cells (top) or only cycling cells (bottom) from the two clones of MGH36 across the backbone of the hierarchy as shown in FIG. 18D. Colors indicate the fraction of cells within a Euclidean distance of 0.3. (H,I) Different clones defined by point mutations span all 3 tumor compartments. (H) Clones inferred by mutation analysis of scRNA-Seq reads. Each panel shows lineage (X-axis) and stemness (Y-axis) scores for cells, colored by their mutation status based on scRNA-Seq reads (red: detected by scRNA-Seq; black: not detected). Top left corner: mutation name, expected (E) fraction of mutant cells by ABSOLUTE (35), and fraction of single cells were the mutation was observed (O). Top right corner: tumor ID. (I) Clones determined by single cell mutation-specific qPCR. As in (F) but showing a wild-type CIC allele detected (green), a mutant CIC allele detected (orange) or neither one detected (black). (J) An expression signature for CIC-mutant cells. Shown is a heatmap of relative expression levels for CIC-dependent genes (rows) in CIC-mutant (right columns) and CIC-wild-type (left columns) cells. Key gene names are marked on left.

FIG. 21. Molecular characterization of oligodendroglioma and validation of CNVs. Shown are IHC (top left) and FISH (all other panels) in a representative tumor (MGH36). All of the cases retain ATRX protein expression by immunohistochemistry (IHC) (top left) and show loss of chromosomes arms 1p (bottom left) and 19q (top right) by FISH. In addition, tumor specific CNVs identified by single-cell RNA-seq were confirmed by FISH (e.g., loss of chromosome 4 in MGH36, bottom right panel).

FIG. 22. Statistics of single cell RNA-seq experiments. Shown are the distributions of the total number of sequenced paired-end reads per cell (gray) and of paired-end reads that were mapped to the transcriptome and used to quantify gene expression (black).

FIG. 23A-23B. Two populations of non-cancer cells identified in oligodendroglioma. (A) Selected genes that are differentially expressed among the two populations of normal cells that lack CNVs (FIG. 17B, top), including markers of microglia (top) and oligodendrocytes (bottom). (B) Expression programs in microglia cells from the three tumors. The heatmap shows relative expression of genes (rows) across microglia cells (columns). Above the dashed line are microglia markers expressed in all microglia cells and below the line are the genes of a microglia activation program, which is variably expressed, and includes cytokines, chemokines, early response genes and other immune effectors. This latter gene set might reflect a microglia activation program that could either be a general microglia program or potentially specific to the context of oligodendroglioma. Microglia cells (columns) are rank ordered by their relative expression of the activation program. The tumor of origin of each cell is color-coded at the top panel.

FIG. 24A-24D. Principal component analysis. (A) PC2 and PC3 are associated with intermediate values of PC1. PC1 scores are shown along with PC2 (top) and PC3 (bottom) scores for cells in each of the three tumors profiled at high depth. Red line indicates local weighted regression (LOWESS) with a span of 5%, which demonstrates that PC2 and PC3 values tend to be highest in intermediate values of PC1 and to decrease in either high PC1 (i.e. OC-like cells) or low PC1 (i.e. AC-like cells). (B) Consistency of PCA across tumors. Shown are the Pearson correlations in gene loadings (over all analyzed genes) between the top three PCs in PCA of the three tumors profiled at high depth (y axis, as shown in FIG. 1) and the top four PCs in alternative PCA of either all six tumors (left), as well as of PCA of each individual tumor (right). PC1-3 are highly consistent between the three-tumor and six-tumor PCAs (R>0.9); PC1 is highly consistent (R>0.8) between the three-tumor analysis and all other analysis. (C) PC1 (x axis) and PC2+PC3 (y axis) scores of malignant cells from each of the three tumors profiled at intermediate depth, showing consistent patterns with those shown in FIG. 1d. (D) Distribution of differences in PC1 loadings between the original PCA and the shuffled PCA (see description in the Methods section, Principal component analysis) for all genes (black), OC-like genes (blue) and AC-like genes (green). This analysis demonstrates that OC-like and AC-like gene-sets are highly skewed in the original PCA and their loadings are not recapitulated by shuffled data reflecting the effect of complexity.

FIG. 25A-25C. OC-like, AC-like and stem-like cell clusters by hierarchical clustering. (A) Cell-cell correlation matrix based on all analyzed genes across all malignant cells in MGH54. Cells are ordered by average linkage hierarchical clustering, and colored boxes indicate distinct clusters. Clusters are marked based on the identity of differentially expressed genes as OC-like (blue), AC-like (yellow), cycling (pink) stem-like (purple) and intermediate cells that do not score highly for any of those expression programs (orange). (B) Top differently expressed genes. Shown is the average expression in each of the OC-like, AC-like, stem-like and intermediate cell clusters (columns) of differentially expressed genes (rows) defined by comparing cells from each of the OC-like, AC-like and stem-like clusters to cells from the remaining clusters with a two-sample t-test. Similar genes are highlighted as in PCA (FIG. 35): (OC-like: OMG, OLIG1/2, SOX8; AC-like: ALDOC, APOE, SOX9; Stem-like: SOX4/11, CCND2, SOX2). Stem-like genes also include CTNNB1, USP22, and MSI1. (C) Cell-cell correlation matrices, as in (A) for cells of MGH36 and MGH53. Boxes indicate OC-like and AC-like clusters.

FIG. 26A-26C. The stemness program in oligodendroglioma overlaps with expression programs of glioblastoma (GBM) cancer stem cells and normal neural stem/progenitor cells. (A) Overlap with human GBM stemness program. Applicants have previously (Patel et al. 2014) identified a GBM sternness program and determined the association of each gene with that program by the correlation between the expression of that gene and the average expression of the stemness program's genes across individual cells (“CSC gradient”) in each of five GBM tumors. Shown is the average correlation (X axis) of each analyzed gene (green dots) across the five cases and the p-values of those correlations as determined with a t-test (Y axis). Genes also identified in the oligodendroglioma stemness program (this work) are marked in black. Applicants considered genes with p<0.05 (marked by dashed line) and an average correlation above 0.1 as significant in the GBM analysis. Eight genes in the oligodendroglioma stemness program overlapped with the significant GBM genes, representing a significant enrichment (1.5*10-4, hypergeometric test). (B) Correlation with mouse activated NSC program. Shown is the distribution of correlation values (X axis) of either all genes (gray) or genes from the oligodendroglioma stemness program (black) with the expression program of mice NSC activation states, as previously quantified by “pseudotime”, across single mouse NSCs (Shin et al. 2015). The average correlation of the NSC activation program genes with oligodendroglioma stemness genes is significantly higher than with all other genes (P=3*10⁻⁶; t-test). (C) Correlation with human NPC program. Shown is the distribution of correlation values (X axis) of either all genes (gray) or genes from the oligodendroglioma stemness program (black) with an expression program of human NPCs identified by PCA (FIG. 25). Each gene's correlation to the average expression of the NPC program genes was calculated across single human NPCs. The average correlation with oligodendroglioma stemness genes is significantly higher than with all other genes (P=2*10⁻³, t-test).

FIG. 27. In vitro sphere forming assay in serum-free conditions. Spherogenic oligodendroglioma line BT54 (Kelly et al. 2010) with 1p/19q co-deletion and IDH1 mutation, was sorted for CD24 by flow cytometry and 20,000 cells were plated in serum-free medium supplemented with EGF and FGF, in duplicate (Methods). 14 days after sorting overall sphere formation was evaluated. Similar results were obtained in duplicate experiment. Representative example depicted.

FIG. 28. Preferential expression of the oligodendroglioma stemness program in neurons but not in OPCs. Genes expressed in the oligodendroglioma single cells were divided into six bins (bars) based on their relative expression (log₂-ratio) in stem-like cells with high PC2/3 and intermediate PC1 scores compared to all other cells. Bins were defined by expression intervals, (X-axis labels). Each panel shows for each bin the average relative expression in each of three normal brain cell types (Y axis) based on data from the Barres lab RNA-seq database (Zhang et al. 2014, Zhang et al. 2016): mice oligodendrocyte progenitor cells (mOPC, top), mouse neurons (mNeurons, middle), and human neurons (hNeurons, bottom). Relative expression of each gene in each CNS cell type was defined as the log 2-ratio between the respective cell type divided by the average over AC, OC and neurons. Error bars: standard error as defined by bootstrapping. Asterisks: bins with significantly different relative expression (in the respective normal cell type) compared to all genes expressed in oligodendroglioma, based on P<0.001 (by t-test) and average expression change of at least 30%.

FIG. 29A-29F. Analysis of human NPCs. (A-D) Differentiation potential of Human SVZ NPCs. Human SVZ NPCs isolated from 19 weeks old fetus form neurospheres in culture (A), and can be differentiated to neuronal (Neurofilament, B), oligodendrocytic (OLIG2, C), or astrocytic (GFAP, D) lineages in vitro. Scale bars: 25 um (A), 10 um (B-D). Applicants note that although OLIG2 can represent different cell types it is very lowly expressed in the fetal NPCs before differentiation (an average log 2(TPM+1) of 0.82, compared to a threshold of 4 that Applicants use to define expressed genes in our analysis, and zero cells with expression above this threshold). Thus, the undifferentiated NPCs do not express OLIG2 and Applicants interpret the expression of OLIG2 as a sign of oligodendroglial lineage differentiation. (E,F) Single cell RNA-Seq analysis of NPCs. (E) NPCs have an expression program similar to that of the oligodendroglioma stemness program; Heatmap shows the expression of genes (rows) most positively (top) or negatively (bottom) correlated with PC1 of a PCA of RNA-seq profiles for 431 single NPCs, across NPC cells (columns) rank ordered by their PC1 scores. Selected genes are indicated, and a full list of correlated genes for PC1 and PC2 is given in Table 3. (F) NPC cell scores for PC1 (Y-axis) and PC2 (X-axis). PC2 correlated genes (Table 3) are associated with the cell cycle. Cells with the highest PC1 scores tend to be non-cycling (low PC2 score), indicating that while the stemness program is coupled to the cell cycle in oligodendroglioma, it is decoupled from the cell cycle in NPCs.

FIG. 30A-30B. Stemness and lineage score for individual tumors. (A) Shown are plots as in FIG. 37b for each of the six tumors. Cycling cells are colored as in FIG. 37, with G1/S cells in blue, S/G2 cells in green, G2/M cells in red, and potential early G1 cells in light blue. (B) Lineage and stemness scores for the three tumors with high-depth profiling, colored based on sequencing batches, demonstrating the lack of considerable batch effects.

FIG. 31A-31G. Single cell RNA-seq of MGH60 reveals similar hierarchy to that of MGH36, 53 and 54. A fourth oligodendroglioma tumor (MGH60) was profiled by two protocols for single cell RNA-seq: the full-length SMART-Seq2 protocol (A,B) used to generate all single cell RNA-seq of MGH36, 53 and 54; and an alternative protocol (C,D) where only the 5′-ends of transcripts are analyzed while incorporating random molecular tags (RMTs, also known us unique molecular identifiers, or UMIs) that decrease the biases of PCR amplification. The same tumor was also analyzed by whole exome sequencing (E). (A,C) In data from both protocols, PC1 reflects an AC-like and OC-like distinction. Shown are heatmaps of the AC-like and OC-like specific genes (rows, as defined in Table 2 and restricted to genes with average expression log 2(TPM+1)>4 in each dataset) with cells ordered by their PC1 score. (B,D,E,F) In data from both protocols, Applicants observe a developmental hierarchy. Shown are the cells analyzed by each protocol by their lineage (X axis) and stemness (Y axis) scores (defined as in FIG. 36E). Cycling cells were found only in the cells analyzed by SMART-seq2, due to the limited number of sequenced cells with the 5′-end protocol, and are shown to be specific to stem/progenitor-like cells, as observed for the other three tumors (FIG. 37). (G) Copy number profiles of MGH60 cells as inferred from single cell RNA-seq (top panel), and as measured by WES (bottom panel), demonstrating the consistency between these approaches.

FIG. 32A-32B. Characterization of tumor subpopulations by histopathology and tissue staining. (A) Two predominant lineages of AC-like and OC-like cells. Shown is MGH53 with hematoxylin and Eosin (H&E, top left), immunohistochemistry for OLIG2 (oligodendrocytic lineage marker, top right) and GFAP (astrocytic marker, bottom left), as well as in situ RNA hybridization for astrocytic markers ApoE (apolipoprotein E, bottom right), with patterns similar to GFAP immunohistochemistry. (B) Cycling cells are enriched among stem-like cells. In situ RNA hybridization for the stem/progenitor markers SOX4 (left panel) and the proliferation marker Ki-67 (right panel) in MGH36 identifies cells positive for both markers (arrows). Immunohistochemistry for GFAP (arrowhead, right panel) and Ki-67 (arrow, right panel) in MGH36 shows mutually exclusive expression patterns.

FIG. 33A-33E. Cycling cancer cells identified by scoring G1/S and G2/M associated gene-sets. (A) A cell cycle trajectory. Shown are cells (dots) scored by the average levels of gene expression of genes-sets associated with G1/S (X axis) and G2/M (Y axis) (Methods). Cells were then rank ordered by identifying all putative cycling cells with at least a 2-fold upregulation and a t-test P-value <0.01 for either the G1/S or the G2/M gene-set, then manually partitioning those cells to distinct regions (color code), and finally estimating the direction of cell cycle progression in each region and ordering the cells in that region accordingly (edges; Methods). (B-E) High expression of G1/S and G2/M gene sets in distinct cycling cells. Shown is the average expression of G1/S (blue curve in B, D; top genes in C, E) and G2/M (green curve in B, D; bottom genes in C, E) genes in all cells (B,C) or only the putative cycling cells (D, E). Cells are rank ordered as in (A). Dashed lines in (D) separate the four subsets of cycling cells, corresponding to light blue, blue, green and red in (A).

FIG. 34A-34C. Agreement in proportion of cycling cells estimated from single-cell RNA-seq and Ki-67 staining. (A,B) Estimated proportion of cycling cells agrees between single cell RNA-Seq and Ki-76 immunohistochemistry. Shown are the estimates of proportion of cycling cells (Y axis) in each of 3 tumors (X axis) based on single cell RNA-Seq (A; different phases assessed by color code as in FIG. 33A) or Ki-67 immunohistochemistry (B). (C) Variation in cycling cells between regions of the same tumor. Shown is Ki-67 immunohistochemistry in two regions in MGH36. Such regional variability in proliferation complicates direct comparisons.

FIG. 35A-35C. Enrichment of cycling cells among stem-like and undifferentiated oligodendroglioma cells. (A,B) Cycling cells are enriched in stem-like and undifferentiated cells compared to differentiated cells. Shown is the percentage of cycling cells (Y axis) in oligodendroglioma cells divided into four bins based on stemness scores (A, Methods) or based on lineage scores (B, Methods). Black squares and error-bars correspond to the mean and standard deviation of the percentages in the three tumors profiled at high depth (MGH36, MGH53, MGH54), and red circles denote the percentages in individual tumors. The four bins in (A) correspond to stemness scores below −1.5 (n=711), between −1.5 and 0.5 (n=1,100), between −0.5 and 0.5 (n=939), and above 0.5 (n=274), respectively. The first two bins are significantly depleted with cycling cells, while the last two bins are significantly enriched (P<0.05, hypergeometric test). The five bins in (B) correspond to AC score above 1 (n=503), AC score between 0.5 and 1 (n=1013), AC and OC scores below 0.5 (n=1130), OC score between 0.5 and 1 (n=855), and OC score above 1 (n=597), respectively. The third bin is significantly enriched with cycling cells, while the four other bins are significantly depleted (P<0.05, hypergeometric test). (C) Specific enrichment of S/G2/M cells compared to G1 cells among stem-like or undifferentiated cells. Shown is the proportion (Y axis) of each marked category of cells among the stem-like or undifferentiated subpopulations. Significant enrichments are marked (P<0.01, hypergeometric test).

FIG. 36A-36D. CCND2 is associated with both cycling and non-cycling stem/progenitor cells. (A) CCND2, but not CCND1/3, is upregulated in non-cycling stem-like oligodendroglioma cells. Shown are the average expression levels (Y axis, log-scale) of three cyclin-D genes (X axis) in non-cycling cells classified as OC-like cells (light blue), undifferentiated cells (gray) and stem-like cells (purple). CCND2 is ˜4-fold higher in stem-like non-cycling cells than in OC-like and undifferentiated cells (P<0.001 by permutation test). Conversely, CCND1 and CCND3 are expressed at comparable levels in stem-like and OC-like cells. (B) Up-regulation of cyclin-D genes in cycling cells compared to non-cycling cells. As in (A) but for up regulation (log₂-ratio) in cycling cells vs. non-cycling cells. CCND2 levels further increase in cycling undifferentiated and stem-like cells but not in OC-like cells, while CCND1 and CCND3 levels increase in OC-like cycling cells more than in undifferentiated and stem-like cycling cells. (C) Distinct expression pattern of cyclin D genes in human brain development. Shown are the expression pattern of three cyclin-D genes (rows) in human brain samples at different points in pre- and post-natal development, sorted by age (columns; pre/post to left/right of dashed vertical line) from the Allen Brain Atlas (Miller et al.). CCND2 is associated with prenatal samples, whereas CCND1 and CCND3 are expressed mostly in childhood and adult samples. (D) CCND2 is upregulated in activated vs. quiescent NSCs (Shin et al. 2015) both among cycling and non-cycling cells. Activated NSCs were partitioned into non-cycling cells (black) and cycling cells in the G1/S (green) or G2/M (red) phases (Methods). Expression difference (Y axis) for each of three genes (X axis) was quantified for each of these subsets as the log 2-ratio of the average expression in the respective subset vs. the quiescent NSCs, and was significant for each of the three subsets (P<0.05 by permutation test). While CCND2 (left) is induced in both cycling and non-cycling activated NSCs, two canonical cell cycle genes (PCNA; middle, and AURKB, right) are not induced in non-cycling genes but were induced preferentially in G1/S and G2/M cells, respectively.

FIG. 37A-37B. Distribution of cellular states in distinct genetic clones of MGH36 and MGH97. (A) Shown are stemness (Y axis) and lineage (X axis) score plots for MGH36 (top) and MGH97 (bottom), each separated into clone 1 (left) and clone 2 (right) as determined by CNV analysis (FIG. 17B,C). Cycling cells are colored as in FIG. 19, with G1/S cells in blue, S/G2 cells in green, and G2/M cells in red. (B) Color-coded density of cells across the cellular hierarchy as shown in FIG. 18E, for the two clones (left: clone 1, right: clone 2) in each of the two tumors (top: MGH36, bottom: MGH97).

FIG. 38. Multiple subclonal mutations each span the cellular hierarchy. Each panel shows lineage (X axis) and sternness (Y axis) scores of cells in which Applicants ascertained by single cell RNA-seq a mutant (red), a wild-type (blue) or none (black) of the alleles. Included are mutations for which at least three cells were identified as mutants and that were identified by WES as subclonal (fraction<60%). The gene names, tumor name, ABSOLUTE-derived fraction of mutant cells (E, for Expected fraction) and the fraction of cells detected as mutant by RNA-seq (0, for Observed) are also indicated within each panel. Applicants note that identification of a wild-type allele (blue) does not imply a wild-type cell because mutations may be heterozygous and thus cells could contain both alleles while only one may be detected by single cell RNA-seq. The observed fraction of mutations (O) is much lower than expected (E) due to limited coverage of the single cell RNA-seq data as well as due to heterozygosity. The vast majority of mutations (20 of 22) are distributed across the hierarchy and span multiple compartments. Two remaining mutations (H2AFV and EIF2AK2) appear more restricted to the “undifferentiated” region (intermediate lineage and stemness scores), which could reflect our limited detection rate of mutant cells and/or a bias of the mutation to a particular region. To test the significance of potential biases in the distribution of mutations Applicants calculated, for each mutation, a Euclidean distance among all pairs of mutant cells (based on their lineage and stemness scores), and compared the average pairwise distances among mutant cells to that among randomly selected subsets of the same number of cells. None of the mutations were significant with a false discovery rate (FDR) of 0.1, although this could reflect our limited statistical power and Applicants cannot exclude a potential bias. Applicants note, however, that even if a subset of mutations are biased in their distribution (as Applicants show for clone 1 in MGH36, FIG. 20A,B), the wide distribution of expression states for most mutations, as well as for the CNV clones (FIG. 20A,B) and for the LOH-clones (FIG. 39), is highly inconsistent with a model in which the hierarchy is driven by genetics, which would predict that all low-frequency subclones would be restricted to regions of the hierarchy, as Applicants discuss in FIG. 40. The apparent bias of mutant cells to the OC lineage over the AC lineage (i.e. positive vs. negative lineage scores) reflects the lower frequencies of AC-like cells compared to OC-like cells in MGH53 and MGH54 (MGH53: 17% AC vs. 39% OC; MGH54: 23% AC vs. 45% OC); this bias is also observed for the detection of wild-type alleles (blue) further demonstrating that there is no bias against mutation detection in the AC lineage.

FIG. 39A-39B. Loss-of-heterozygosity (LOH) event in MGH54 reveals two clones that span the cellular hierarchy. (A) Chromosome 18 LOH in MGH54. Allelic fraction analysis of MGH54 SNPs from WES shows an imbalance (red and blue dots) in the frequency of alternative alleles in chromosome 1p, 19q, as well as chromosome 18, despite the normal copy number at this chromosome (FIG. 17B). This is consistent with an LOH event in which presumably one copy of chromosome 18 was deleted, and the other copy amplified. The weaker imbalance compared to chromosomes 1p and 19q further indicates that this is a subclonal event. (B) Each of two clones defined by Chr. 18 LOH status spans the full hierarchy. Shown are the lineage (X axis) and stemness (Y axis) scores for each cell from MGH54 classified as pre-LOH (red), post-LOH (blue) and unresolved (black) based on RNA-seq reads that map to SNPs in the minor (i.e. deleted) chromosome. Both the pre- and post-LOH clones span the different tumor subpopulations. Pre-LOH cells were defined as all cells with reads that map to minor alleles in chromosome 18; post-LOH cells were defined as all cells with reads that map to at least five different major alleles, but no reads that map to minor alleles in chromosome 18; all other cells were defined as unresolved.

FIG. 40A-40E. The observed distribution of mutations is highly inconsistent with a model of genetically-driven hierarchy. (A) Phylogenetic tree for a hypothetical tumor, where each circle corresponds to a cell. Six subclonal mutations are shown (black arrows), each defining a genetic subclone. (B) Under a genetically-driven hierarchy, specific subclones would correspond to subpopulations with distinct expression states, such that all cells in those subclones map into a specific expression state. Shown are schemes of the cellular hierarchy in oligondroglioma (i.e. the two lower branches reflect the AC-like and OC-like lineages and the top part reflect stem-like cells), with cells from a given subclone marked in red and confined to specific transcriptional states. Importantly, the restriction of a subclone to a specific expression state holds true not only for the subclones which are defined by the mutation that is causal for an expression state but also for any other subclone that is contained within it. For example, assuming that subclones 1 and 4 reflect the mutations that are causal for the OC-like and AC-like expression states, subclones 2 and 5 would also be confined to either the OC-like or the AC-like states. This is especially true for small subclones (i.e., mutations with a low clonal fraction), as these should be confined to a small branch in the phylogenetic tree that is unlikely to cover multiple subpopulations. Small subclones that nevertheless cover all three subpopulations are especially unlikely by this model, although these are observed in the data (e.g. ZEB2, FRG1, FTH1 and EEF1B2 in FIG. 20C all have a clonal fraction of 11% or less but span the three compartments of the hierarchy). Such cases could theoretically be explained by an identical mutation that occurs independently in multiple branches and thereby covers small subsets of cells from multiple braches. However, this is highly unlikely to account for the mutations that Applicants observe, as none of these mutations with the potential exception of the CIC mutation is a known “hot-spot” mutation that is expected to recur (and even the specific CIC mutation Applicants find is one of many mutations for this gene, and reported for 4 of 66 CIC-mutated TCGA patient samples). Thus, even convergent evolution is unlikely to result in these mutations occurring independently in different branches of the phylogenetic tree. Furthermore, Applicants identified three cases of compound chromosomal aberrations (two concurrent chromosomal deletions in MGH36, a chromosomal deletion and gain in MGH97, and a chromosome-wide LOH in MGH54 that requires two distinct genetic events) that in each case define two distinct clones, each of which spanning the different expression-based subpopulations; these events are highly unlikely to occur independently in different branches. (C) Under a non-genetic driven hierarchy, individual subclones tend to span the different expression states represented by the cellular hierarchy, consistent with the data herein. Applicants note that this model does not exclude the possibility that subclones would be biased towards (or against) a certain cellular state, as genetic evolution could interact with non-genetic states and influence their prevalence. (D) Phylogenetic tree for a hypothetical tumor, where each circle corresponds to a cell. According to the model of genetically-driven hierarchy, specific regions in the tree would correspond to subpopulations with distinct expression states. Shown are examples of three such potential subpopulations. (E) Mutations acquired during tumor evolution (numbered arrows) generate tumor subclones that harbor these mutations (indicated as numbered circles) and are confined to specific branches of the tree. Therefore, according to the model of genetically-driven hierarchy, subclonal mutations are expected to be present only in cells from a specific subpopulation, as defined by expression states. This is especially true for small subclones (i.e. mutations with a low clonal fraction), as these should be confined to a small branch that is unlikely to cover multiple subpopulations. Small subclones that nevertheless cover all three subpopulations are especially unlikely by this model (such as ZEB2, FRG1 and EEF1B2 shown in FIG. 20; all with clonal fraction of 11% or less but span the three compartments of the hierarchy). Such cases could theoretically be explained by an identical mutation that occurs independently in multiple branches and thereby covers small subsets of cells from multiple braches. However, this is highly unlikely to account for the mutations that Applicants observe, as none of these mutations, except for CIC, is a known “hot-spot” mutation that is expected to recur. Thus, even convergent evolution is unlikely to result in these mutations occurring independently in different branches of the phylogenetic tree. Furthermore, Applicants identified two cases of large chromosomal aberrations (two concurrent chromosomal deletions in MGH36, and a chromosome-wide LOH in MGH54) that in each case define two distinct clones, and each of which spans the different expression-based subpopulations; these events are highly unlikely to occur independently in different branches.

FIG. 41. Model for oligodendroglioma architecture and clonal evolution. Early in their pathogenesis (left), tumors are composed of a single genetic clone and hierarchically organized, such that a subpopulation of cycling stem/progenitor cells gives rise to differentiated progeny in two glial lineages. As the tumor evolves (right), multiple genetic clones are generated and co-exist, with each genetic clone maintaining a hierarchical organization where the relative distribution of the different compartment may vary due to genetic effects but is overall similar.

FIG. 42. For each of the three tumors profiled at high depth (horizontal panels) and for the two lineages (vertical panels) Applicants calculated the significance of co-expression among sets of AC-related and OC-related genes within limited ranges of lineage scores (between the value of the X axis and that of the Y axis). Significance was calculated by comparison to 100,000 control gene-sets with similar number of genes and distribution of average expression levels, and is indicated by color. The significant co-expression patterns within limited ranges of lineage scores suggest that variability of lineage scores in these ranges cannot be driven by noise alone, and implies the existence of multiple states within each lineage, presumably reflecting intermediate differentiation states (see Note 2).

### DETAILED DESCRIPTION

The invention relates to gene expression signatures and networks of tumors and tissues, as well as multicellular ecosystems of tumors and tissues and the cells and cell type which they comprise. The invention provides methods of characterizing components, functions and interactions of tumors and tissues and the cells which they comprise.

The invention further relates to controlling an immune response by modulating the activity of a component of the complement system. Cancer is but a single exemplary condition that can be controlled by an immune reaction. The present invention describes for how complement expression in the microenvironment can control the abundance of immune cells at a site of disease or condition requiring a shift in balance of an immune response.

The invention provides signature genes, gene products, and expression profiles of signature genes, gene networks, and gene products of tumors and component cells, and including especially melanoma tumors, gliomas, head and neck cancer, brain metastases of breast cancer, and tumors in The Cancer Genome Atlas (TCGA) and tissues. This invention further relates generally to compositions and methods for identifying genes and gene networks that respond to, modulate, control or otherwise influence tumors and tissues, including cells and cell types of the tumors and tissues, and malignant, microenvironmental, or immunologic states of the tumor cells and tissues. The invention also relates to methods of diagnosing, prognosing and/or staging of tumors, tissues and cells, and provides compositions and methods of modulating expression of genes and gene networks of tumors, tissues and cells, as well as methods of identifying, designing and selecting appropriate treatment regimens.

### Use of Signature Genes

As used herein a signature may encompass any gene or genes, protein or proteins, or epigenetic element(s) whose expression profile or whose occurrence is associated with a specific cell type, subtype, or cell state of a specific cell type or subtype within a population of cells. Increased or decreased expression or activity or prevalence may be compared between different cells in order to characterize or identify for instance specific cell (sub)populations. A gene signature as used herein, may thus refer to any set of up- and down-regulated genes between different cells or cell (sub)populations derived from a gene-expression profile. For example, a gene signature may comprise a list of genes differentially expressed in a distinction of interest. It is to be understood that also when referring to proteins (e.g. differentially expressed proteins), such may fall within the definition of “gene” signature.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease, or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall tumor composition, such as tumor cell composition, such as tumor cell subpopulation composition or distribution, or functionality.

As used herein the term “signature gene” means any gene or genes whose expression profile is associated with a specific cell type, subtype, or cell state of a specific cell type or subtype within a population of cells. The signature gene can be used to indicate the presence of a cell type, a subtype of the cell type, the state of the microenvironment of a population of cells, and/or the overall status of the entire cell population. Furthermore, the signature genes may be indicative of cells within a population of cells in vivo. The signature genes of the present invention were discovered by analysis of expression profiles of single-cells within a population of cells from freshly isolated tumors, thus allowing the discovery of novel cell subtypes that were previously invisible in a population of cells within a tumor. The presence of subtypes may be determined by subtype specific signature genes. The presence of these specific cell types may be determined by applying the signature genes to bulk sequencing data in a patient tumor. Not being bound by a theory, a tumor is a conglomeration of many cells that make up a tumor microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tumor. Not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory the presence of specific cells and cell subtypes are indicative of tumor growth and resistance to treatment. The signature gene may indicate the presence of one particular cell type. In one embodiment, the signature genes may indicate that tumor infiltrating T-cells are present. The presence of cell types within a tumor may indicate that the tumor will be resistant to a treatment. In one embodiment, the signature genes of the present invention are applied to bulk sequencing data from a tumor sample to transform the data into information relating to disease outcome and personalized treatments. In one embodiment, the novel signature genes are used to detect multiple cell states that occur in a subpopulation of tumor cells that are linked to resistance to targeted therapies and progressive tumor growth.

In one embodiment, the signature genes are detected by immunofluorescence, by mass cytometry (CyTOF), drop-seq, single cell qPCR, MERFISH (multiplex (in situ) RNA FISH) and/or by in situ hybridization. Other methods including absorbance assays and colorimetric assays are known in the art and may be used herein.

In one embodiment, tumor cells are stained for cell subtype specific signature genes. In one embodiment, the cells are fixed. In another embodiment, the cells are formalin fixed and paraffin embedded. Not being bound by a theory, the presence of the cell subtypes in a tumor indicate outcome and personalized treatments. Not being bound by a theory, the cell subtypes may be quantitated in a section of a tumor and the number of cells indicates an outcome and personalized treatment. In preferred embodiments, cancer stem cells according to the present invention are detected.

The gene signatures described herein are useful in methods of monitoring a cancer in a subject by detecting a level of expression, activity and/or function of one or more signature genes or one or more products of one or more signature genes at a first time point, detecting a level of expression, activity and/or function of one or more signature genes or one or more products of one or more signature genes at a second time point, and comparing the first detected level of expression, activity and/or function with the second detected level of expression, activity and/or function, wherein a change in the first and second detected levels indicates a change in the cancer in the subject.

One unique aspect of the invention is the ability to relate expression of one gene or a gene signature in one cell type to that of another gene or signature in another cell type in the same tumor. In one embodiment, the methods and signatures of the invention are useful in patients with complex cancers, heterogeneous cancers or more than one cancer.

In an embodiment of the invention, these signatures are useful in monitoring subjects undergoing treatments and therapies for cancer to determine efficaciousness of the treatment or therapy. In an embodiment of the invention, these signatures are useful in monitoring subjects undergoing treatments and therapies for cancer to determine whether the patient is responsive to the treatment or therapy. In an embodiment of the invention, these signatures are also useful for selecting or modifying therapies and treatments that would be efficacious in treating, delaying the progression of or otherwise ameliorating a symptom of cancer. In an embodiment of the invention, the signatures provided herein are used for selecting a group of patients at a specific state of a disease with accuracy that facilitates selection of treatments.

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; and Habib et al., 2016, “Div-Seq: Single-nucleus RNA-Seq reveals dynamics of rare adult newborn neurons” Science, Vol. 353, Issue 6302, pp. 925-928, both of which are herein incorporated by reference in their entirety.

In certain embodiments, single cells of a subject are sequenced to determine cell types and gene signatures present in the subject. In one embodiment, sequencing is targeted for gene signatures of a specific cell type. Cells may be quantitated based on the sequencing of a cell specific gene signature. In certain embodiments, the depth of sequencing may be adjusted, such that cells having a particular gene signature can be detected. The term “depth (coverage)” as used herein refers to the number of times a nucleotide is read during the sequencing process. Depth can be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy. This parameter also enables one to estimate other quantities, such as the percentage of the genome covered by reads (sometimes also called coverage). A high coverage in shotgun sequencing is desired because it can overcome errors in base calling and assembly. The subject of DNA sequencing theory addresses the relationships of such quantities. Even though the sequencing accuracy for each individual nucleotide is very high, the very large number of nucleotides in the genome means that if an individual genome is only sequenced once, there will be a significant number of sequencing errors. Furthermore rare single-nucleotide polymorphisms (SNPs) are common. Hence to distinguish between sequencing errors and true SNPs, it is necessary to increase the sequencing accuracy even further by sequencing individual genomes a large number of times.

The term “deep sequencing” as used herein indicates that the total number of reads is many times larger than the length of the sequence under study. The term “deep” as used herein refers to a wide range of depths greater than or equal to 1× up to 100×.

It will be understood by the skilled person that treating as referred to herein encompasses enhancing treatment, or improving treatment efficacy. Treatment may include tumor regression as well as inhibition of tumor growth or tumor cell proliferation, or inhibition or reduction of otherwise deleterious effects associated with the tumor.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include a T cell modulating agent, targeted therapies and checkpoint inhibitors, are used to treat or alleviate a symptom associated with a cancer. The present invention also provides methods of treating or alleviating a symptom associated with cancer. A therapeutic regimen is carried out by identifying a subject, e.g., a human patient suffering from cancer, using standard methods.

Efficaciousness of treatment is determined in association with any known method for diagnosing or treating the particular cancer. The invention comprehends a treatment method or Drug Discovery method or method of formulating or preparing a treatment comprising any one of the methods or uses herein discussed.

The phrase “therapeutically effective amount” as used herein refers to a nontoxic but sufficient amount of a drug, agent, or compound to provide a desired therapeutic effect.

As used herein “patient” refers to any human being receiving or who may receive medical treatment.

A “polymorphic site” refers to a polynucleotide that differs from another polynucleotide by one or more single nucleotide changes.

A “somatic mutation” refers to a change in the genetic structure that is not inherited from a parent, and also not passed to offspring.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing a cancer (e.g., a person who is genetically predisposed) may receive prophylactic treatment to inhibit or delay symptoms of the disease.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for oral, rectal, intravenous, intramuscular, subcutaneous, inhalation, nasal, topical or transdermal, vaginal, or ophthalmic administration. Thus, the medicament may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols.

Aspects of the invention involve targeting proliferating glioma cell types. In certain embodiments, targeting reduces the viability of or renders non-viable stem cells or progenitor cells comprised by the glioma. Targeting may be by use of antibodies, antibody fragments and antibody conjugates and single-chain immunotoxins reactive with human glioma cells. Antibody drug conjugates are well known in the art.

Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

Aspects of the invention involve the adoptive transfer of immune system cells, such as T cells. In certain embodiments, immune cells are specific for cell surface markers present on cells having a stem cell signature as described herein. The immune cells may be modified to express a chimeric antigen receptor specific for a marker. In other embodiments, cells specific for cells having a stem cell signature as described herein are activated and transferred to the patient. Immune cells may also be specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs may be characterized as belonging to successive generations. First-generation CARs typically consist of a single-chain variable fragment of an antibody specific for an antigen, for example comprising a V_{L} linked to a V_{H} of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CAR T cells of this kind may for example be used in animal models, for example to threat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction).

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 104-10 cells per kg body weight, preferably 10 to 10⁶ cells/kg body weight including all integer values of cell numbers within those ranges. Dosing in CAR T cell therapies may for example involve administration of from 10⁶ to 10 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). Cells may be edited using any CRISPR system, TALE, TALEN, or Zinc finger protein and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and ρ chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SIP-1 or TIM-3. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Cell therapy methods often involve the ex-vivo activation and expansion of T-cells. In one embodiment T cells are activated before administering them to a subject in need thereof. Activation or stimulation methods have been described herein and is preferably required before T cells are administered to a subject in need thereof. Examples of these type of treatments include the use tumor infiltrating lymphocyte (TIL) cells (see U.S. Pat. No. 5,126,132), cytotoxic T-cells (see U.S. Pat. Nos. 6,255,073; and 5,846,827), expanded tumor draining lymph node cells (see U.S. Pat. No. 6,251,385), and various other lymphocyte preparations (see U.S. Pat. Nos. 6,194,207; 5,443,983; 6,040,177; and 5,766,920). These patents are herein incorporated by reference in their entirety.

For maximum effectiveness of T-cells in cell therapy protocols, the ex vivo activated T-cell population should be in a state that can maximally orchestrate an immune response to cancer, infectious diseases, or other disease states. For an effective T-cell response, the T-cells first must be activated. For activation, at least two signals are required to be delivered to the T-cells. The first signal is normally delivered through the T-cell receptor (TCR) on the T-cell surface. The TCR first signal is normally triggered upon interaction of the TCR with peptide antigens expressed in conjunction with an MHC complex on the surface of an antigen-presenting cell (APC). The second signal is normally delivered through co-stimulatory receptors on the surface of T-cells. Co-stimulatory receptors are generally triggered by corresponding ligands or cytokines expressed on the surface of APCs.

Due to the difficulty in maintaining large numbers of natural APC in cultures of T-cells being prepared for use in cell therapy protocols, alternative methods have been sought for ex-vivo activation of T-cells. One method is to by-pass the need for the peptide-MHC complex on natural APCs by instead stimulating the TCR (first signal) with polyclonal activators, such as immobilized or cross-linked anti-CD3 or anti-CD2 monoclonal antibodies (mAbs) or superantigens. The most investigated co-stimulatory agent (second signal) used in conjunction with anti-CD3 or anti-CD2 mAbs has been the use of immobilized or soluble anti-CD28 mAbs. The combination of anti-CD3 mAb (first signal) and anti-CD28 mAb (second signal) immobilized on a solid support such as paramagnetic beads (see U.S. Pat. No. 6,352,694, herein incorporated by reference in its entirety) has been used to substitute for natural APCs in inducing ex-vivo T-cell activation in cell therapy protocols (Levine, Bernstein et al., 1997 Journal of Immunology:159:5921-5930; Garlie, LeFever et al., 1999 J Immunother. July; 22(4):336-45; Shibuya, Wei et al., 2000 Arch Otolaryngol Head Neck Surg. 126(4):473-9).

In one embodiment T cells that have infiltrated a tumor are isolated. T cells may be removed during surgery. T cells may be isolated after removal of tumor tissue by biopsy. T cells may be isolated by any means known in the art. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

In one embodiment of the present invention, any combination of therapeutic, not limited to a small molecule, compound, mixture, nucleic acid, vector, or protein, is administered to a subject in order to increase or decrease the activity of the complement system. Exemplary embodiments for activation of complement are natural products such as snake venom and caterpillar bristles (PLoS Negl Trop Dis. 2013 Oct. 31; 7(10):e2519; and PLoS One. 2015 Mar. 11; 10(3):e0118615). Other molecules capable of activating complement have been described, such as C-reactive protein (CRP). Pharmaceutical grade CRP has been described previously (Circulation Research. 2014; 114: 672-676). Additionally, therapeutic antibodies may be used to activate or inhibit complement. In one embodiment, antibody drug conjugates may be used. In other embodiments, dual targeting compounds and/or antibodies may be used. Not being bound by a theory, a dual antibody may bind complement in one aspect and, for example, a tumor in another aspect, so as to localize the complement to a tumor. An antibody of the present invention may be an antibody fragment. The antibody fragment may be a nanobody, Fab, Fab′, (Fab′)2, Fv, ScFv, diabody, triabody, tetrabody, Bis-scFv, minibody, Fab2, or Fab3 fragment.

Inhibitors of the complement system are well known in the art and are useful for the practice of the present invention (see, e.g., Ricklin et al., Progress and trends in complement therapeutics. Adv Exp Med Biol. 2013; 735:1-22.; Ricklin et al., Complement-targeted therapeutics. Nat Biotechnol. 2007 Nov.; 25(11): 1265-1275; and Reis et al., Applying complement therapeutics to rare diseases. Clin Immunol. 2015 December; 161(2):225-40, herein incorporated by reference in their entirety).

A “complement inhibitor” is a molecule that prevents or reduces activation and/or propagation of the complement cascade that results in the formation of C3a or signaling through the C3a receptor, or C5a or signaling through the C5a receptor. A complement inhibitor can operate on one or more of the complement pathways, i.e., classical, alternative or lectin pathway. A “C3 inhibitor” is a molecule or substance that prevents or reduces the cleavage of C3 into C3a and C3b. A “C5a inhibitor” is a molecule or substance that prevents or reduces the activity of C5a. A “C5aR inhibitor” is a molecule or substance that prevents or reduces the binding of C5a to the C5a receptor. A “C3aR inhibitor” is a molecule or substance that prevents or reduces binding of C3a to the C3a receptor. A “factor D inhibitor” is a molecule or substance that prevents or reduces the activity of Factor D. A “factor B inhibitor” is a molecule or substance that prevents or reduces the activity of factor B. A “C4 inhibitor” is a molecule or substance that prevents or reduces the cleavage of C4 into C4b and C4a. A “C1q inhibitor” is a molecule or substance that prevents or reduces C1q binding to antibody-antigen complexes, virions, infected cells, or other molecules to which C1q binds to initiate complement activation. Any of the complement inhibitors described herein may comprise antibodies or antibody fragments, as would be understood by the person of skill in the art.

Antibodies useful in the present invention, such as antibodies that specifically bind to either C4, C3 or C5 and prevent cleavage, or antibodies that specifically bind to factor D, factor B, C1q, or the C3a or C5a receptor, can be made by the skilled artisan using methods known in the art. Anti-C3 and anti-C5 antibodies are also commercially available.

A “complement activator” is a molecule that activates or increases activation and/or propagation of the complement cascade that results in the formation of C3a or signaling through the C3a receptor, or C5a or signaling through the C5a receptor. A complement activator can operate on one or more of the complement pathways, i.e., classical, alternative or lectin pathway.

Inhibitors or activators of the complement system may be administered by any known means in the art and by any means described herein. The inhibitors or activators may be targeted to a specific site of disease, such as, but not limited to a tumor. Monitoring by any means described herein may be used to determine if the therapy is effective. Such combination of a therapeutic targeting complement and monitoring provides advantages over any methods known in the art. Not being bound by a theory, the infiltration of cell populations, such as CAFs, T cells, macrophages, B cells may be monitored during treatment with an agent that activates or inhibits a component of the complement system. Not being bound by a theory a gene signature within a specific cell population as described herein may be monitored during treatment with an agent that activates or inhibits a component of the complement system. Not being bound by a theory, the present invention is provided by the Applicants discovery of cell specific gene expression signatures of cells within different cancers correlating to immune status, tumor status, and immune cell abundance. Moreover, applicants discovery of the correlation of complement gene expression in specific cell types to immune cell abundance allows for activating or inhibiting complement in order to modulate the microenvironment, including an immune response, for treatment of a disease. As illustrated by the examples, Applicants show that the expression of complement in relation to an immune response, and specifically, immune cell abundance is not limited to a specific cancer. Applicants provide data showing consistent gene expression patterns of complement components in single cells for melanoma, head and neck cancer, glioma, metastases to the brain, and across the TCGA tumors (see Examples). Not being bound by a theory, immune cell abundance is and gene expression signatures in single cells part of the microenvironment is a general phenomena that provides for activating and inhibiting complement in relation to many diseases and conditions, preferably cancer.

The terms “complement,” “complement system” and “complement components” as used herein refer to proteins and protein fragments, including serum proteins, serosal proteins, and cell membrane receptors that are part of any of the classical complement pathway, the alternative complement pathway, and the lectin pathway. The terms “complement,” “complement system” and “complement components” also includes the defense molecules (protection molecules) CD46, CD55 and CD59.

The classical pathway is triggered by activation of the C1-complex. The C1-complex is composed of 1 molecule of C1q, 2 molecules of C1r and 2 molecules of C1s, or C1qr2s2. This occurs when C1q binds to IgM or IgG complexed with antigens. A single pentameric IgM can initiate the pathway, while several, ideally six, IgGs are needed. This also occurs when C1q binds directly to the surface of the pathogen. Such binding leads to conformational changes in the C1q molecule, which leads to the activation of two C1r molecules. C1r is a serine protease. They then cleave C1s (another serine protease). The C1r2s2 component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex). The inhibition of C1r and C1s is controlled by C1-inhibitor (SERPING1).

The alternative pathway is continuously activated at a low level as a result of spontaneous C3 hydrolysis due to the breakdown of the internal thioester bond. The alternative pathway does not rely on pathogen-binding antibodies like the other pathways. C3b that is generated from C3 by a C3 convertase enzyme complex in the fluid phase is rapidly inactivated by factor H and factor I, as is the C3b-like C3 that is the product of spontaneous cleavage of the internal thioester. In contrast, when the internal thioester of C3 reacts with a hydroxyl or amino group of a molecule on the surface of a cell or pathogen, the C3b that is now covalently bound to the surface is protected from factor H-mediated inactivation. The surface-bound C3b may now bind factor B to form C3bB. This complex in the presence of factor D will be cleaved into Ba and Bb. Bb will remain associated with C3b to form C3bBb, which is the alternative pathway C3 convertase.

The C3bBb complex is stabilized by binding oligomers of factor P (Properdin). The stabilized C3 convertase, C3bBbP, then acts enzymatically to cleave much more C3, some of which becomes covalently attached to the same surface as C3b. This newly bound C3b recruits more B, D and P activity and greatly amplifies the complement activation. When complement is activated on a cell surface, the activation is limited by endogenous complement regulatory proteins, which include CD35, CD46, CD55 and CD59, depending on the cell. Pathogens, in general, don't have complement regulatory proteins Thus, the alternative complement pathway is able to distinguish self from non-self on the basis of the surface expression of complement regulatory proteins. Host cells don't accumulate cell surface C3b (and the proteolytic fragment of C3b called iC3b) because this is prevented by the complement regulatory proteins, while foreign cells, pathogens and abnormal surfaces may be heavily decorated with C3b and iC3b. Accordingly, the alternative complement pathway is one element of innate immunity.

Once the alternative C3 convertase enzyme is formed on a pathogen or cell surface, it may bind covalently another C3b, to form C3bBbC3bP, the C5 convertase. This enzyme then cleaves C5 to C5a, a potent anaphylatoxin, and C5b. The C5b then recruits and assembles C6, C7, C8 and multiple C9 molecules to assemble the membrane attack complex. This creates a hole or pore in the membrane that can kill or damage the pathogen or cell.

The lectin pathway is homologous to the classical pathway, but with the opsonin, mannose-binding lectin (MBL), and ficolins, instead of C1q. This pathway is activated by binding of MBL to mannose residues on the pathogen surface, which activates the MBL-associated serine proteases, MASP-1, and MASP-2 (very similar to C1r and Cis, respectively), which can then split C4 into C4a and C4b and C2 into C2a and C2b. C4b and C2a then bind together to form the classical C3-convertase, as in the classical pathway. Ficolins are homologous to MBL and function via MASP in a similar way. Several single-nucleotide polymorphisms have been described in M-ficolin in humans, with effect on ligand-binding ability and serum levels. Historically, the larger fragment of C2 was named C2a, but it is now referred as C2b. In invertebrates without an adaptive immune system, ficolins are expanded and their binding specificities diversified to compensate for the lack of pathogen-specific recognition molecules.

In certain embodiments, combination therapies are administered to a patient in need thereof. In one preferred embodiment, the administration of an immunotherapy, such as adoptive cell transfer, may be enhanced by the addition of a checkpoint inhibitor. Not being bound by a theory, the addition of a checkpoint inhibitor may enhance an immune response against a targeted cell type.

The term “MDSC” (myeloid-derived suppressor cells) refers to a heterogenous group of immune cells from the myeloid lineage (a family of cells that originate from bone marrow stem cells), to which dendritic cells, macrophages and neutrophils also belong. MDSCs strongly expand in pathological situations such as chronic infections and cancer, as a result of an altered hematopoiesis. Thus, it is yet unclear whether MDSCs represent a group of immature myeloid cell types that have stopped their differentiation towards DCs, macrophages or granulocytes, or if they represent a myeloid lineage apart. MDSCs are however discriminated from other myeloid cell types in which they possess strong immunosuppressive activities rather than immunostimulatory properties. Similarly to other myeloid cells, MDSCs interact with other immune cell types including T cells (the effector immune cells that kill pathogens, infected and cancer cells), dendritic cells, macrophages and NK cells to regulate their functions. Their mechanisms of action are beginning to be understood although they are still under heated debate and close examination by the scientific community. Nevertheless, clinical and experimental evidence has shown that cancer tissues with high infiltration of MDSC are associated with poor patient prognosis and resistance to therapies.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 6/10/14; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure™, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014 (2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh 00, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al., “Cpf1 Is a Single RNA-Guided Endonuclease of a Class 2 CRISPR-Cas System,” Cell 163, 1-13 (Oct. 22, 2015)
Shmakov et al., “Discovery and Functional Characterization of Diverse Class 2 CRISPR-Cas Systems,” Molecular Cell 60, 1-13 (Available online Oct. 22, 2015).
   each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Zetsche et al. (2015) reported the characterization of Cpf1, a putative class 2 CRISPR effector. It was demonstrated that Cpf1 mediates robust DNA interference with features distinct from Cas9. Identifying this mechanism of interference broadens our understanding of CRISPR-Cas systems and advances their genome editing applications.
Shmakov et al. (2015) reported the characterization of three distinct Class 2 CRISPR-Cas systems. The effectors of two of the identified systems, C2c1 and C2c3, contain RuvC like endonuclease domains distantly related to Cpf1. The third system, C2c2, contains an effector with two predicted HEPN RNase domains.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

In addition, mention is made of PCT application PCT/US14/70057, Attorney Reference 47627.99.2060 and BI-2013/107 entitiled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas9 protein (and optionally HDR template) with a mixture comprising or consisting essentially of or consisting of surfactant, phospholipid, biodegradable polymer, lipoprotein and alcohol; and particles from such a process. For example, wherein Cas9 protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C1-6 alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas9 protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA, Cas9 protein and components that form a particle; as well as particles from such admixing. Aspects of the instant invention can involve particles; for example, particles using a process analogous to that of the Particle Delivery PCT, e.g., by admixing a mixture comprising sgRNA and/or Cas9 as in the instant invention and components that form a particle, e.g., as in the Particle Delivery PCT, to form a particle and particles from such admixing (or, of course, other particles involving sgRNA and/or Cas9 as in the instant invention).

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, a tracr (transactivating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In a classic CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it will be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

The nucleic acid molecule encoding a Cas is advantageously codon optimized Cas. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P A), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also, the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al.. (2009).

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK) (SEQ ID NO: 2); the c-myc NLS having the amino acid sequence PAAKRVKLD (SEQ ID NO: 3) or RQRRNELKRSP (SEQ ID NO: 4); the hRNPA1 M9 NLS having the sequence NQSSNFGPMKGGNFGGRSSGPYGGGGQYFAKPRNQGGY(SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence PQPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 12) of the influenza virus NS1; the sequence RKLKKKIKKL (SEQ ID NO: 13) of the Hepatitis virus delta antigen; the sequence REKKKFLKRR (SEQ ID NO: 14) of the mouse Mx1 protein; the sequence KRKGDEVDGVDEVAKKKSKK (SEQ ID NO: 15) of the human poly(ADP-ribose) polymerase; and the sequence RKCLQAGMNLEARKTKK (SEQ ID NO: 16) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs.

In certain aspects, the invention involves vectors, e.g. for delivering or introducing in a cell the DNA targeting agent according to the invention as described herein, such as by means of example Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s) (e.g., sgRNAs); and, when a single vector provides for more than 16 RNA(s) (e.g., sgRNAs), one or more promoter(s) can drive expression of more than one of the RNA(s) (e.g., sgRNAs), e.g., when there are 32 RNA(s) (e.g., sgRNAs), each promoter can drive expression of two RNA(s) (e.g., sgRNAs), and when there are 48 RNA(s) (e.g., sgRNAs), each promoter can drive expression of three RNA(s) (e.g., sgRNAs). By simple arithmetic and well established cloning protocols and the teachings in this disclosure one skilled in the art can readily practice the invention as to the RNA(s) (e.g., sgRNA(s) for a suitable exemplary vector such as AAV, and a suitable promoter such as the U6 promoter, e.g., U6-sgRNAs. For example, the packaging limit of AAV is ˜4.7 kb. The length of a single U6-sgRNA (plus restriction sites for cloning) is 361 bp. Therefore, the skilled person can readily fit about 12-16, e.g., 13 U6-sgRNA cassettes in a single vector. This can be assembled by any suitable means, such as a golden gate strategy used for TALE assembly (www.genome-engineering.org/taleffectors/). The skilled person can also use a tandem guide strategy to increase the number of U6-sgRNAs by approximately 1.5 times, e.g., to increase from 12-16, e.g., 13 to approximately 18-24, e.g., about 19 U6-sgRNAs. Therefore, one skilled in the art can readily reach approximately 18-24, e.g., about 19 promoter-RNAs, e.g., U6-sgRNAs in a single vector, e.g., an AAV vector. A further means for increasing the number of promoters and RNAs, e.g., sgRNA(s) in a vector is to use a single promoter (e.g., U6) to express an array of RNAs, e.g., sgRNAs separated by cleavable sequences. And an even further means for increasing the number of promoter-RNAs, e.g., sgRNAs in a vector, is to express an array of promoter-RNAs, e.g., sgRNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjoumals.org/content/34/7/e53.short, www.nature.com/mt/journal/v16/n9/abs/mt2008144a.html). In an advantageous embodiment, AAV may package U6 tandem sgRNA targeting up to about 50 genes. Accordingly, from the knowledge in the art and the teachings in this disclosure the skilled person can readily make and use vector(s), e.g., a single vector, expressing multiple RNAs or guides or sgRNAs under the control or operatively or functionally linked to one or more promoters-especially as to the numbers of RNAs or guides or sgRNAs discussed herein, without any undue experimentation.

A poly nucleic acid sequence encoding the DNA targeting agent according to the invention as described herein, such as by means of example guide RNA(s), e.g., sgRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Through this disclosure and the knowledge in the art, the DNA targeting agent as described herein, such as, TALEs, CRISPR-Cas systems, etc., or components thereof or nucleic acid molecules thereof (including, for instance HDR template) or nucleic acid molecules encoding or providing components thereof may be delivered by a delivery system herein described both generally and in detail.

Vector delivery, e.g., plasmid, viral delivery: By means of example, the CRISPR enzyme, for instance a Cas9, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. The DNA targeting agent as described herein, such as Cas9 and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10¹-1×10¹² particles), and most preferably at least about 1×10⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10¹¹ particles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10¹¹ pu, about 4×10¹¹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10¹¹ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a DNA targeting agent as described herein, such as a comprising a CRISPR enzyme, operably linked to said promoter; (iii) a selectable marker; (iv) an origin of replication; and (v) a transcription terminator downstream of and operably linked to (ii). The plasmid can also encode the RNA components of a CRISPR complex, but one or more of these may instead be encoded on a different vector.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

In some embodiments the RNA molecules of the invention are delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference. Delivery systems aimed specifically at the enhanced and improved delivery of siRNA into mammalian cells have been developed, (see, for example, Shen et al FEBS Let. 2003, 539:111-114; Xia et al., Nat. Biotech. 2002, 20:1006-1010; Reich et al., Mol. Vision. 2003, 9: 210-216; Sorensen et al., J. Mol. Biol. 2003, 327: 761-766; Lewis et al., Nat. Gen. 2002, 32: 107-108 and Simeoni et al., NAR 2003, 31, 11: 2717-2724) and may be applied to the present invention. siRNA has recently been successfully used for inhibition of gene expression in primates (see for example. Tolentino et al., Retina 24(4):660 which may also be applied to the present invention.

Indeed, RNA delivery is a useful method of in vivo delivery. It is possible to deliver the DNA targeting agent as described herein, such as Cas9 and gRNA (and, for instance, HR repair template) into cells using liposomes or particles. Thus delivery of the CRISPR enzyme, such as a Cas9 and/or delivery of the RNAs of the invention may be in RNA form and via microvesicles, liposomes or particles. For example, Cas9 mRNA and gRNA can be packaged into liposomal particles for delivery in vivo. Liposomal transfection reagents such as lipofectamine from Life Technologies and other reagents on the market can effectively deliver RNA molecules into the liver.

Means of delivery of RNA also preferred include delivery of RNA via nanoparticles (Cho, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder, A., Levins, C., Cortez, C., Langer, R., and Anderson, D., Lipid-based nanotherapeutics for siRNA delivery, Journal of Internal Medicine, 267: 9-21, 2010, PMID: 20059641). Indeed, exosomes have been shown to be particularly useful in delivery siRNA, a system with some parallels to the CRISPR system. For instance, El-Andaloussi S, et al. (“Exosome-mediated delivery of siRNA in vitro and in vivo.” Nat Protoc. 2012 Dec.; 7(12):2112-26. doi: 10.1038/nprot.2012.131. Epub 2012 Nov. 15.) describe how exosomes are promising tools for drug delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purify and characterized from transfected cell supernatant, then RNA is loaded into the exosomes. Delivery or administration according to the invention can be performed with exosomes, in particular but not limited to the brain. Vitamin E (α-tocopherol) may be conjugated with CRISPR Cas and delivered to the brain along with high density lipoprotein (HDL), for example in a similar manner as was done by Uno et al. (HUMAN GENE THERAPY 22:711-719 (June 2011)) for delivering short-interfering RNA (siRNA) to the brain. Mice were infused via Osmotic minipumps (model 1007D; Alzet, Cupertino, Calif.) filled with phosphate-buffered saline (PBS) or free TocsiBACE or Toc-siBACE/HDL and connected with Brain Infusion Kit 3 (Alzet). A brain-infusion cannula was placed about 0.5 mm posterior to the bregma at midline for infusion into the dorsal third ventricle. Uno et al. found that as little as 3 nmol of Toc-siRNA with HDL could induce a target reduction in comparable degree by the same ICV infusion method. A similar dosage of CRISPR Cas conjugated to α-tocopherol and co-administered with HDL targeted to the brain may be contemplated for humans in the present invention, for example, about 3 nmol to about 3 μmol of CRISPR Cas targeted to the brain may be contemplated. Zou et al. ((HUMAN GENE THERAPY 22:465-475 (April 2011)) describes a method of lentiviral-mediated delivery of short-hairpin RNAs targeting PKCγ for in vivo gene silencing in the spinal cord of rats. Zou et al. administered about 10 μl of a recombinant lentivirus having a titer of 1×10⁹ transducing units (TU)/ml by an intrathecal catheter. A similar dosage of CRISPR Cas expressed in a lentiviral vector targeted to the brain may be contemplated for humans in the present invention, for example, about 10-50 ml of CRISPR Cas targeted to the brain in a lentivirus having a titer of 1×10⁹ transducing units (TU)/ml may be contemplated.

In terms of local delivery to the brain, this can be achieved in various ways. For instance, material can be delivered intrastriatally e.g. by injection. Injection can be performed stereotactically via a craniotomy.

Enhancing NHEJ or HR efficiency is also helpful for delivery. It is preferred that NHEJ efficiency is enhanced by co-expressing end-processing enzymes such as Trex2 (Dumitrache et al. Genetics. 2011 August; 188(4): 787-797). It is preferred that HR efficiency is increased by transiently inhibiting NHEJ machineries such as Ku70 and Ku86. HR efficiency can also be increased by co-expressing prokaryotic or eukaryotic homologous recombination enzymes such as RecBCD, RecA.

Packaging and Promoters Generally

Ways to package nucleic acid molecules, in particular the DNA targeting agent according to the invention as described herein, such as Cas9 coding nucleic acid molecules, e.g., DNA, into vectors, e.g., viral vectors, to mediate genome modification in vivo include:
To achieve NHEJ-mediated gene knockout:
   Single virus vector:
      Vector containing two or more expression cassettes:
      Promoter-Cas9 coding nucleic acid molecule-terminator
      Promoter-gRNA1-terminator
      Promoter-gRNA2-terminator
      Promoter-gRNA(N)-terminator (up to size limit of vector)
   Double virus vector:
      Vector 1 containing one expression cassette for driving the expression of Cas9
      Promoter-Cas9 coding nucleic acid molecule-terminator
      Vector 2 containing one more expression cassettes for driving the expression of one or more guideRNAs
      Promoter-gRNA1-terminator
      Promoter-gRNA(N)-terminator (up to size limit of vector)
To mediate homology-directed repair.
   In addition to the single and double virus vector approaches described above, an additional vector is used to deliver a homology-direct repair template.

The promoter used to drive Cas9 coding nucleic acid molecule expression can include:
AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of Cas9.
For ubiquitous expression, can use promoters: CMV, CAG, CBh, PGK, SV40, Ferritin heavy or light chains, etc.
For brain or other CNS expression, can use promoters: SynapsinI for all neurons, CaMKIIalpha for excitatory neurons, GAD67 or GAD65 or VGAT for GABAergic neurons, etc.
For liver expression, can use Albumin promoter.
For lung expression, can use SP-B.
For endothelial cells, can use ICAM.
For hematopoietic cells can use IFNbeta or CD45.
For Osteoblasts can use OG-2.

The promoter used to drive guide RNA can include:
Pol III promoters such as U6 or H1
Use of Pol II promoter and intronic cassettes to express gRNA

### Adeno Associated Virus (AAV)

The DNA targeting agent according to the invention as described herein, such as by means of example Cas9 and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of the DNA targeting agent according to the invention as described herein, such as by means of example Cas9 can be driven by a cell-type specific promoter. For example, liver-specific expression might use the Albumin promoter and neuron-specific expression (e.g. for targeting CNS disorders) might use the Synapsin I promoter.

In terms of in vivo delivery, AAV is advantageous over other viral vectors for a couple of reasons:
Low toxicity (this may be due to the purification method not requiring ultra centrifugation of cell particles that can activate the immune response)
Low probability of causing insertional mutagenesis because it doesn't integrate into the host genome.

AAV has a packaging limit of 4.5 or 4.75 Kb. This means that for instance Cas9 as well as a promoter and transcription terminator have to be all fit into the same viral vector. Constructs larger than 4.5 or 4.75 Kb will lead to significantly reduced virus production. SpCas9 is quite large, the gene itself is over 4.1 Kb, which makes it difficult for packing into AAV. Therefore embodiments of the invention include utilizing homologs of Cas9 that are shorter. For example:
Species Cas9 Size
Corynebacter diphtheriae 3252
Eubacterium ventriosum 3321
Streptococcus pasteurianus 3390
Lactobacillus farciminis 3378
Sphaerochaeta globus 3537
Azospirillum B510 3504
Gluconacetobacter diazotrophicus 3150
Neisseria cinerea 3246
Roseburia intestinalis 3420
Parvibaculum lavamentivorans 3111
Staphylococcus aureus 3159
Nitratifractor salsuginis DSM 16511 3396
Campylobacter lari CF89-12 3009
Streptococcus thermophilus LMD-9 3396

These species are therefore, in general, preferred Cas9 species.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually. A tabulation of certain AAV serotypes as to these cells (see Grimm, D. et al, J. Virol. 82: 5887-5911 (2008)) is as follows:

| | AAV- | AAV- | AAV- | AAV- | AAV- | AAV- | AAV- | AAV- |
|---|---|---|---|---|---|---|---|---|
| Cell Line | 1 | 2 | 3 | 4 | 5 | 6 | 8 | 9 |
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

Lentivirus

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared as follows, by means of example for Cas delivery. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMEM (serum-free) media and transfection was done 4 hours later. Cells were transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection was done in 4 mL OptiMEM with a cationic lipid delivery agent (50 uL Lipofectamine 2000 and 100 ul Plus reagent). After 6 hours, the media was changed to antibiotic-free DMEM with 10% fetal bovine serum. These methods use serum during cell culture, but serum-free methods are preferred.

Lentivirus may be purified as follows. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 ul of DMEM overnight at 4C. They were then aliquotted and immediately frozen at −80° C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285). In another embodiment, RetinoStat®, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et al., HUMAN GENE THERAPY 23:980-991 (September 2012)) and this vector may be modified for the CRISPR-Cas system of the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the CRISPR-Cas system of the present invention. A minimum of 2.5×10⁶ CD34+ cells per kilogram patient weight may be collected and prestimulated for 16 to 20 hours in X-VIVO 15 medium (Lonza) containing 2 μmol/L-glutamine, stem cell factor (100 ng/ml), Flt-3 ligand (Flt-3L) (100 ng/ml), and thrombopoietin (10 ng/ml) (CellGenix) at a density of 2×10⁶ cells/ml. Prestimulated cells may be transduced with lentiviral at a multiplicity of infection of 5 for 16 to 24 hours in 75-cm² tissue culture flasks coated with fibronectin (25 mg/cm²) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015.

### RNA Delivery

RNA delivery: The DNA targeting agent according to the invention as described herein, such as the CRISPR enzyme, for instance a Cas9, and/or any of the present RNAs, for instance a guide RNA, can also be delivered in the form of RNA. Cas9 mRNA can be generated using in vitro transcription. For example, Cas9 mRNA can be synthesized using a PCR cassette containing the following elements: T7_promoter-kozak sequence (GCCACC)-Cas9-3′ UTR from beta globin-polyA tail (a string of 120 or more adenines). The cassette can be used for transcription by T7 polymerase. Guide RNAs can also be transcribed using in vitro transcription from a cassette containing T7_promoter-GG-guide RNA sequence.

To enhance expression and reduce possible toxicity, the CRISPR enzyme-coding sequence and/or the guide RNA can be modified to include one or more modified nucleoside e.g. using pseudo-U or 5-Methyl-C.

mRNA delivery methods are especially promising for liver delivery currently.

Much clinical work on RNA delivery has focused on RNAi or antisense, but these systems can be adapted for delivery of RNA for implementing the present invention. References below to RNAi etc. should be read accordingly.

Particle Delivery Systems and/or Formulations:

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g. diameter) of less than 100 microns (m). In some embodiments, inventive particles have a greatest dimension of less than 10 m. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo (herein cargo refers to e.g., one or more components of for instance CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, concerning particles, methods of making and using them and measurements thereof.

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

### Particles

The DNA targeting agent according to the invention as described herein, such as by means of example CRISPR enzyme mRNA and guide RNA may be delivered simultaneously using particles or lipid envelopes; for instance, CRISPR enzyme and RNA of the invention, e.g., as a complex, can be delivered via a particle as in Dahlman et al., WO2015089419 A2 and documents cited therein, such as 7C1 (see, e.g., James E. Dahlman and Carmen Barnes et al. Nature Nanotechnology (2014) published online 11 May 2014, doi:10.1038/nnano.2014.84), e.g., delivery particle comprising lipid or lipidoid and hydrophilic polymer, e.g., cationic lipid and hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5), wherein particles are formed using an efficient, multistep process wherein first, effector protein and RNA are mixed together, e.g., at a 1:1 molar ratio, e.g., at room temperature, e.g., for 30 minutes, e.g., in sterile, nuclease free 1×PBS; and separately, DOTAP, DMPC, PEG, and cholesterol as applicable for the formulation are dissolved in alcohol, e.g., 100% ethanol; and, the two solutions are mixed together to form particles containing the complexes).

For example, Su X, Fricke J, Kavanagh D G, Irvine D J (“In vitro and in vivo mRNA delivery using lipid-enveloped pH-responsive polymer nanoparticles” Mol Pharm. 2011 Jun. 6; 8(3):774-87. doi: 10.1021/mp100390w. Epub 2011 Apr. 1) describes biodegradable core-shell structured particles with a poly(O-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

In one embodiment, particles based on self assembling bioadhesive polymers are contemplated, which may be applied to oral delivery of peptides, intravenous delivery of peptides and nasal delivery of peptides, all to the brain. Other embodiments, such as oral absorption and ocular delivery of hydrophobic drugs are also contemplated. The molecular envelope technology involves an engineered polymer envelope which is protected and delivered to the site of the disease (see, e.g., Mazza, M. et al. ACSNano, 2013. 7(2): 1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Pharm, 2012. 9(6):1764-74; Garrett, N. L., et al. J Biophotonics, 2012. 5(5-6):458-68; Garrett, N. L., et al. J Raman Spect, 2012. 43(5):681-688; Ahmad, S., et al. J Royal Soc Interface 2010. 7:S423-33; Uchegbu, I.F. Expert Opin Drug Deliv, 2006. 3(5):629-40; Qu, X., et al. Biomacromolecules, 2006. 7(12):3452-9 and Uchegbu, I. F., et al. Int J Pharm, 2001. 224:185-199). Doses of about 5 mg/kg are contemplated, with single or multiple doses, depending on the target tissue.

In one embodiment, particles that can deliver DNA targeting agents according to the invention as described herein, such as RNA to a cancer cell to stop tumor growth developed by Dan Anderson's lab at MIT may be used/and or adapted to the CRISPR Cas system according to certain embodiments of the present invention. In particular, the Anderson lab developed fully automated, combinatorial systems for the synthesis, purification, characterization, and formulation of new biomaterials and nanoformulations. See, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93.

US patent application 20110293703 relates to lipidoid compounds are also particularly useful in the administration of polynucleotides, which may be applied to deliver the DNA targeting agent according to the invention, such as for instance the CRISPR Cas system according to certain embodiments of the present invention. In one aspect, the aminoalcohol lipidoid compounds are combined with an agent to be delivered to a cell or a subject to form microparticles, particles, liposomes, or micelles. The agent to be delivered by the particles, liposomes, or micelles may be in the form of a gas, liquid, or solid, and the agent may be a polynucleotide, protein, peptide, or small molecule. The minoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Publication No. 20110293703 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30-100° C., preferably at approximately 50-90° C. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or other alkylating agent, and/or they may be acylated.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to the DNA targeting agent according to the invention, such as for instance the CRISPR Cas system according to certain embodiments of the present invention.

In another embodiment, lipid particles (LNPs) are contemplated. An antitransthyretin small interfering RNA has been encapsulated in lipid particles and delivered to humans (see, e.g., Coelho et al., N Engl J Med 2013; 369:819-29), and such a system may be adapted and applied to the CRISPR Cas system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP or by means of example CRISPR-Cas RNA in or associated with the LNP may be contemplated, especially for a formulation containing DLinKC2-DMA.

Preparation of LNPs and the DNA targeting agent according to the invention as described herein, such as by means of example CRISPR Cas encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(o-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprised of cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Particle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be ˜70 nm in diameter. RNA encapsulation efficiency may be determined by removal of free RNA using VivaPureD MiniH columns (Sartorius Stedim Biotech) from samples collected before and after dialysis. The encapsulated RNA may be extracted from the eluted particles and quantified at 260 nm. RNA to lipid ratio was determined by measurement of cholesterol content in vesicles using the Cholesterol E enzymatic assay from Wako Chemicals USA (Richmond, Va.). In conjunction with the herein discussion of LNPs and PEG lipids, PEGylated liposomes or LNPs are likewise suitable for delivery of a CRISPR-Cas system or components thereof.

Preparation of large LNPs may be used/and or adapted from Rosin et al, Molecular Therapy, vol. 19, no. 12, pages 1286-2200, December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/l, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano ZS, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an RNA to total lipid ratio of approximately 1:10 (wt:wt), followed by incubation for 30 minutes at 37° C. to form loaded LNPs. The mixture may be subsequently dialyzed overnight in PBS and filtered with a 0.45-μm syringe filter.

Spherical Nucleic Acid (SNA™) constructs and other particles (particularly gold particles) are also contemplated as a means to deliver the DNA targeting agent according to the invention as described herein, such as by means of example CRISPR-Cas system to intended targets. Significant data show that AuraSense Therapeutics' Spherical Nucleic Acid (SNA™) constructs, based upon nucleic acid-functionalized gold particles, are useful.

Literature that may be employed in conjunction with herein teachings include: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, 10:186-192.

Self-assembling particles with RNA may be constructed with polyethyleneimine (PEI) that is PEGylated with an Arg-Gly-Asp (RGD) peptide ligand attached at the distal end of the polyethylene glycol (PEG). This system has been used, for example, as a means to target tumor neovasculature expressing integrins and deliver siRNA inhibiting vascular endothelial growth factor receptor-2 (VEGF R2) expression and thereby achieve tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling particles of Schiffelers et al.

The nanoplexes of Bartlett et al. (PNAS, Sep. 25, 2007,vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA particles may be formed by using cyclodextrin-containing polycations. Typically, particles were formed in water at a charge ratio of 3 (+/−) and an siRNA concentration of 0.5 g/liter. One percent of the adamantane-PEG molecules on the surface of the targeted particles were modified with Tf (adamantane-PEG-Tf). The particles were suspended in a 5% (wt/vol) glucose carrier solution for injection.

Davis et al. (Nature, Vol 464, 15 Apr. 2010) conducts a RNA clinical trial that uses a targeted particle-delivery system (clinical trial registration number NCT00689065). Patients with solid cancers refractory to standard-of-care therapies are administered doses of targeted particles on days 1, 3, 8 and 10 of a 21-day cycle by a 30-min intravenous infusion. The particles consist of a synthetic delivery system containing: (1) a linear, cyclodextrin-based polymer (CDP), (2) a human transferrin protein (TF) targeting ligand displayed on the exterior of the particle to engage TF receptors (TFR) on the surface of the cancer cells, (3) a hydrophilic polymer (polyethylene glycol (PEG) used to promote particle stability in biological fluids), and (4) siRNA designed to reduce the expression of the RRM2 (sequence used in the clinic was previously denoted siR2B+5). The TFR has long been known to be upregulated in malignant cells, and RRM2 is an established anti-cancer target. These particles (clinical version denoted as CALAA-01) have been shown to be well tolerated in multi-dosing studies in non-human primates. Although a single patient with chronic myeloid leukaemia has been administered siRNAby liposomal delivery, Davis et al.'s clinical trial is the initial human trial to systemically deliver siRNA with a targeted delivery system and to treat patients with solid cancer. To ascertain whether the targeted delivery system can provide effective delivery of functional siRNA to human tumours, Davis et al. investigated biopsies from three patients from three different dosing cohorts; patients A, B and C, all of whom had metastatic melanoma and received CALAA-01 doses of 18, 24 and 30 mg m² siRNA, respectively. Similar doses may also be contemplated for the CRISPR Cas system of the present invention. The delivery of the invention may be achieved with particles containing a linear, cyclodextrin-based polymer (CDP), a human transferrin protein (TF) targeting ligand displayed on the exterior of the particle to engage TF receptors (TFR) on the surface of the cancer cells and/or a hydrophilic polymer (for example, polyethylene glycol (PEG) used to promote particle stability in biological fluids).

In terms of this invention, it is preferred to have one or more components of the DNA targeting agent according to the invention as described herein, such as by means of example the CRISPR complex, e.g., CRISPR enzyme or mRNA or guide RNA delivered using particles or lipid envelopes. Other delivery systems or vectors are may be used in conjunction with the particle aspects of the invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, particles of the invention have a greatest dimension ranging between 35 nm and 60 nm. In other preferred embodiments, the particles of the invention are not nanoparticles.

Particles encompassed in the present invention may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Particles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft particles have been manufactured, and are within the scope of the present invention. A prototype particle of semi-solid nature is the liposome. Various types of liposome particles are currently used clinically as delivery systems for anticancer drugs and vaccines. Particles with one half hydrophilic and the other half hydrophobic are termed Janus particles and are particularly effective for stabilizing emulsions. They can self-assemble at water/oil interfaces and act as solid surfactants.

U.S. Pat. No. 8,709,843, incorporated herein by reference, provides a drug delivery system for targeted delivery of therapeutic agent-containing particles to tissues, cells, and intracellular compartments. The invention provides targeted particles comprising polymer conjugated to a surfactant, hydrophilic polymer or lipid. U.S. Pat. No. 6,007,845, incorporated herein by reference, provides particles which have a core of a multiblock copolymer formed by covalently linking a multifunctional compound with one or more hydrophobic polymers and one or more hydrophilic polymers, and contain a biologically active material. U.S. Pat. No. 5,855,913, incorporated herein by reference, provides a particulate composition having aerodynamically light particles having a tap density of less than 0.4 g/cm3 with a mean diameter of between 5 μm and 30 μm, incorporating a surfactant on the surface thereof for drug delivery to the pulmonary system. U.S. Pat. No. 5,985,309, incorporated herein by reference, provides particles incorporating a surfactant and/or a hydrophilic or hydrophobic complex of a positively or negatively charged therapeutic or diagnostic agent and a charged molecule of opposite charge for delivery to the pulmonary system. U.S. Pat. No. 5,543,158, incorporated herein by reference, provides biodegradable injectable particles having a biodegradable solid core containing a biologically active material and poly(alkylene glycol) moieties on the surface. WO2012135025 (also published as US20120251560), incorporated herein by reference, describes conjugated polyethyleneimine (PEI) polymers and conjugated aza-macrocycles (collectively referred to as “conjugated lipomer” or “lipomers”). In certain embodiments, it can envisioned that such conjugated lipomers can be used in the context of the CRISPR-Cas system to achieve in vitro, ex vivo and in vivo genomic perturbations to modify gene expression, including modulation of protein expression.

In one embodiment, the particle may be epoxide-modified lipid-polymer, advantageously 7C1 (see, e.g., James E. Dahlman and Carmen Barnes et al. Nature Nanotechnology (2014) published online 11 May 2014, doi:10.1038/nnano.2014.84). C71 was synthesized by reacting C15 epoxide-terminated lipids with PEI600 at a 14:1 molar ratio, and was formulated with C14PEG2000 to produce particles (diameter between 35 and 60 nm) that were stable in PBS solution for at least 40 days.

An epoxide-modified lipid-polymer may be utilized to deliver the CRISPR-Cas system of the present invention to pulmonary, cardiovascular or renal cells, however, one of skill in the art may adapt the system to deliver to other target organs. Dosage ranging from about 0.05 to about 0.6 mg/kg are envisioned. Dosages over several days or weeks are also envisioned, with a total dosage of about 2 mg/kg.

### Exosomes

Exosomes are endogenous nano-vesicles that transport RNAs and proteins, and which can deliver RNA to the brain and other target organs. To reduce immunogenicity, Alvarez-Erviti et al. (2011, Nat Biotechnol 29: 341) used self-derived dendritic cells for exosome production. Targeting to the brain was achieved by engineering the dendritic cells to express Lamp2b, an exosomal membrane protein, fused to the neuron-specific RVG peptide. Purified exosomes were loaded with exogenous RNA by electroporation. Intravenously injected RVG-targeted exosomes delivered GAPDH siRNA specifically to neurons, microglia, oligodendrocytes in the brain, resulting in a specific gene knockdown. Pre-exposure to RVG exosomes did not attenuate knockdown, and non-specific uptake in other tissues was not observed. The therapeutic potential of exosome-mediated siRNA delivery was demonstrated by the strong mRNA (60%) and protein (62%) knockdown of BACE1, a therapeutic target in Alzheimer's disease.

To obtain a pool of immunologically inert exosomes, Alvarez-Erviti et al. harvested bone marrow from inbred C57BL/6 mice with a homogenous major histocompatibility complex (MHC) haplotype. As immature dendritic cells produce large quantities of exosomes devoid of T-cell activators such as MHC-II and CD86, Alvarez-Erviti et al. selected for dendritic cells with granulocyte/macrophage-colony stimulating factor (GM-CSF) for 7 d. Exosomes were purified from the culture supernatant the following day using well-established ultracentrifugation protocols. The exosomes produced were physically homogenous, with a size distribution peaking at 80 nm in diameter as determined by particle tracking analysis (NTA) and electron microscopy. Alvarez-Erviti et al. obtained 6-12 μg of exosomes (measured based on protein concentration) per 10⁶ cells.

Next, Alvarez-Erviti et al. investigated the possibility of loading modified exosomes with exogenous cargoes using electroporation protocols adapted for nanoscale applications. As electroporation for membrane particles at the nanometer scale is not well-characterized, nonspecific Cy5-labeled RNA was used for the empirical optimization of the electroporation protocol. The amount of encapsulated RNA was assayed after ultracentrifugation and lysis of exosomes. Electroporation at 400 V and 125 μF resulted in the greatest retention of RNA and was used for all subsequent experiments.

Alvarez-Erviti et al. administered 150 μg of each BACE1 siRNA encapsulated in 150 μg of RVG exosomes to normal C57BL/6 mice and compared the knockdown efficiency to four controls: untreated mice, mice injected with RVG exosomes only, mice injected with BACE1 siRNA complexed to an in vivo cationic liposome reagent and mice injected with BACE1 siRNA complexed to RVG-9R, the RVG peptide conjugated to 9 D-arginines that electrostatically binds to the siRNA. Cortical tissue samples were analyzed 3 d after administration and a significant protein knockdown (45%, P<0.05, versus 62%, P<0.01) in both siRNA-RVG-9R-treated and siRNARVG exosome-treated mice was observed, resulting from a significant decrease in BACE1 mRNA levels (66% [+ or −] 15%, P<0.001 and 61% [+ or −] 13% respectively, P<0.01). Moreover, Applicants demonstrated a significant decrease (55%, P<0.05) in the total [beta]-amyloid 1-42 levels, a main component of the amyloid plaques in Alzheimer's pathology, in the RVG-exosome-treated animals. The decrease observed was greater than the β-amyloid 1-40 decrease demonstrated in normal mice after intraventricular injection of BACE1 inhibitors. Alvarez-Erviti et al. carried out 5′-rapid amplification of cDNA ends (RACE) on BACE1 cleavage product, which provided evidence of RNAi-mediated knockdown by the siRNA.

Finally, Alvarez-Erviti et al. investigated whether RNA-RVG exosomes induced immune responses in vivo by assessing IL-6, IP-10, TNFα and IFN-α serum concentrations. Following exosome treatment, nonsignificant changes in all cytokines were registered similar to siRNA-transfection reagent treatment in contrast to siRNA-RVG-9R, which potently stimulated IL-6 secretion, confirming the immunologically inert profile of the exosome treatment. Given that exosomes encapsulate only 20% of siRNA, delivery with RVG-exosome appears to be more efficient than RVG-9R delivery as comparable mRNA knockdown and greater protein knockdown was achieved with fivefold less siRNA without the corresponding level of immune stimulation. This experiment demonstrated the therapeutic potential of RVG-exosome technology, which is potentially suited for long-term silencing of genes related to neurodegenerative diseases. The exosome delivery system of Alvarez-Erviti et al. may be applied to deliver the DNA targeting agent according to the invention as described herein, such as by means of example the CRISPR-Cas system of the present invention to therapeutic targets, especially neurodegenerative diseases. A dosage of about 100 to 1000 mg of CRISPR Cas encapsulated in about 100 to 1000 mg of RVG exosomes may be contemplated for the present invention.

El-Andaloussi et al. (Nature Protocols 7, 2112-2126(2012)) discloses how exosomes derived from cultured cells can be harnessed for delivery of RNA in vitro and in vivo. This protocol first describes the generation of targeted exosomes through transfection of an expression vector, comprising an exosomal protein fused with a peptide ligand. Next, El-Andaloussi et al. explain how to purify and characterize exosomes from transfected cell supernatant. Next, El-Andaloussi et al. detail crucial steps for loading RNA into exosomes. Finally, El-Andaloussi et al. outline how to use exosomes to efficiently deliver RNA in vitro and in vivo in mouse brain. Examples of anticipated results in which exosome-mediated RNA delivery is evaluated by functional assays and imaging are also provided. The entire protocol takes ˜3 weeks. Delivery or administration according to the invention may be performed using exosomes produced from self-derived dendritic cells. From the herein teachings, this can be employed in the practice of the invention.

In another embodiment, the plasma exosomes of Wahlgren et al. (Nucleic Acids Research, 2012, Vol. 40, No. 17 e130) are contemplated. Exosomes are nano-sized vesicles (30-90 nm in size) produced by many cell types, including dendritic cells (DC), B cells, T cells, mast cells, epithelial cells and tumor cells. These vesicles are formed by inward budding of late endosomes and are then released to the extracellular environment upon fusion with the plasma membrane. Because exosomes naturally carry RNA between cells, this property may be useful in gene therapy, and from this disclosure can be employed in the practice of the instant invention.

Exosomes from plasma can be prepared by centrifugation of buffy coat at 900 g for 20 min to isolate the plasma followed by harvesting cell supernatants, centrifuging at 300 g for 10 min to eliminate cells and at 16 500 g for 30 min followed by filtration through a 0.22 mm filter. Exosomes are pelleted by ultracentrifugation at 120 000 g for 70 min. Chemical transfection of siRNA into exosomes is carried out according to the manufacturer's instructions in RNAi Human/Mouse Starter Kit (Quiagen, Hilden, Germany). siRNA is added to 100 ml PBS at a final concentration of 2 mmol/ml. After adding HiPerFect transfection reagent, the mixture is incubated for 10 min at RT. In order to remove the excess of micelles, the exosomes are re-isolated using aldehyde/sulfate latex beads. The chemical transfection of CRISPR Cas into exosomes may be conducted similarly to siRNA. The exosomes may be co-cultured with monocytes and lymphocytes isolated from the peripheral blood of healthy donors. Therefore, it may be contemplated that exosomes containing the DNA targeting agent according to the invention as described herein, such as by means of example CRISPR Cas may be introduced to monocytes and lymphocytes of and autologously reintroduced into a human. Accordingly, delivery or administration according to the invention may be performed using plasma exosomes.

### Liposomes

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-dioleoyl-sn-glycero-3-phosphoethanolamine (DOPE) increases the stability (see, e.g., Spuch and Navarro, Journal of Drug Delivery, vol. 2011, Article ID 469679, 12 pages, 2011. doi:10.1155/2011/469679 for review).

In a particularly advantageous embodiment, Trojan Horse liposomes (also known as Molecular Trojan Horses) are desirable and protocols may be found at cshprotocols.cshlp.org/content/2010/4/pdb.prot5407.long. These particles allow delivery of a transgene to the entire brain after an intravascular injection. Without being bound by limitation, it is believed that neutral lipid particles with specific antibodies conjugated to surface allow crossing of the blood brain barrier via endocytosis. Applicant postulates utilizing Trojan Horse Liposomes to deliver the DNA targeting agent according to the invention as described herein, such as by means of example the CRISPR family of nucleases to the brain via an intravascular injection, which would allow whole brain transgenic animals without the need for embryonic manipulation. About 1-5 g of DNA or RNA may be contemplated for in vivo administration in liposomes.

In another embodiment, the DNA targeting agent according to the invention as described herein, such as by means of example the CRISPR Cas system may be administered in liposomes, such as a stable nucleic-acid-lipid particle (SNALP) (see, e.g., Morrissey et al., Nature Biotechnology, Vol. 23, No. 8, August 2005). Daily intravenous injections of about 1, 3 or 5 mg/kg/day of a specific CRISPR Cas targeted in a SNALP are contemplated. The daily treatment may be over about three days and then weekly for about five weeks. In another embodiment, a specific CRISPR Cas encapsulated SNALP) administered by intravenous injection to at doses of about 1 or 2.5 mg/kg are also contemplated (see, e.g., Zimmerman et al., Nature Letters, Vol. 441, 4 May 2006). The SNALP formulation may contain the lipids 3-N-[(wmethoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio (see, e.g., Zimmerman et al., Nature Letters, Vol. 441, 4 May 2006).

In another embodiment, stable nucleic-acid-lipid particles (SNALPs) have proven to be effective delivery molecules to highly vascularized HepG2-derived liver tumors but not in poorly vascularized HCT-116 derived liver tumors (see, e.g., Li, Gene Therapy (2012) 19, 775-780). The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulted SNALP liposomes are about 80-100 nm in size.

In yet another embodiment, a SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane (see, e.g., Geisbert et al., Lancet 2010; 375: 1896-905). A dosage of about 2 mg/kg total CRISPR Cas per dose administered as, for example, a bolus intravenous infusion may be contemplated.

In yet another embodiment, a SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA) (see, e.g., Judge, J. Clin. Invest. 119:661-673 (2009)). Formulations used for in vivo studies may comprise a final lipid/RNA mass ratio of about 9:1.

The safety profile of RNAi nanomedicines has been reviewed by Barros and Gollob of Alnylam Pharmaceuticals (see, e.g., Advanced Drug Delivery Reviews 64 (2012) 1730-1737). The stable nucleic acid lipid particle (SNALP) is comprised of four different lipids an ionizable lipid (DLinDMA) that is cationic at low pH, a neutral helper lipid, cholesterol, and a diffusible polyethylene glycol (PEG)-lipid. The particle is approximately 80 nm in diameter and is charge-neutral at physiologic pH. During formulation, the ionizable lipid serves to condense lipid with the anionic RNA during particle formation. When positively charged under increasingly acidic endosomal conditions, the ionizable lipid also mediates the fusion of SNALP with the endosomal membrane enabling release of RNA into the cytoplasm. The PEG-lipid stabilizes the particle and reduces aggregation during formulation, and subsequently provides a neutral hydrophilic exterior that improves pharmacokinetic properties.

To date, two clinical programs have been initiated using SNALP formulations with RNA. Tekmira Pharmaceuticals recently completed a phase I single-dose study of SNALP-ApoB in adult volunteers with elevated LDL cholesterol. ApoB is predominantly expressed in the liver and jejunum and is essential for the assembly and secretion of VLDL and LDL. Seventeen subjects received a single dose of SNALP-ApoB (dose escalation across 7 dose levels). There was no evidence of liver toxicity (anticipated as the potential dose-limiting toxicity based on preclinical studies). One (of two) subjects at the highest dose experienced flu-like symptoms consistent with immune system stimulation, and the decision was made to conclude the trial.

Alnylam Pharmaceuticals has similarly advanced ALN-TTR01, which employs the SNALP technology described above and targets hepatocyte production of both mutant and wild-type TTR to treat TTR amyloidosis (ATTR). Three ATTR syndromes have been described: familial amyloidotic polyneuropathy (FAP) and familial amyloidotic cardiomyopathy (FAC) both caused by autosomal dominant mutations in TTR; and senile systemic amyloidosis (SSA) cause by wildtype TTR. A placebo-controlled, single dose-escalation phase I trial of ALN-TTR01 was recently completed in patients with ATTR. ALN-TTR01 was administered as a 15-minute IV infusion to 31 patients (23 with study drug and 8 with placebo) within a dose range of 0.01 to 1.0 mg/kg (based on siRNA). Treatment was well tolerated with no significant increases in liver function tests. Infusion-related reactions were noted in 3 of 23 patients at >0.4 mg/kg; all responded to slowing of the infusion rate and all continued on study. Minimal and transient elevations of serum cytokines IL-6, IP-10 and IL-Ira were noted in two patients at the highest dose of 1 mg/kg (as anticipated from preclinical and NHP studies). Lowering of serum TTR, the expected pharmacodynamics effect of ALN-TTR01, was observed at 1 mg/kg.

In yet another embodiment, a SNALP may be made by solubilizing a cationic lipid, DSPC, cholesterol and PEG-lipid e.g., in ethanol, e.g., at a molar ratio of 40:10:40:10, respectively (see, Semple et al., Nature Niotechnology, Volume 28 Number 2 Feb. 2010, pp. 172-177). The lipid mixture was added to an aqueous buffer (50 mM citrate, pH 4) with mixing to a final ethanol and lipid concentration of 30% (vol/vol) and 6.1 mg/ml, respectively, and allowed to equilibrate at 22° C. for 2 min before extrusion. The hydrated lipids were extruded through two stacked 80 nm pore-sized filters (Nuclepore) at 22° C. using a Lipex Extruder (Northern Lipids) until a vesicle diameter of 70-90 nm, as determined by dynamic light scattering analysis, was obtained. This generally required 1-3 passes. The siRNA (solubilized in a 50 mM citrate, pH 4 aqueous solution containing 30% ethanol) was added to the pre-equilibrated (35° C.) vesicles at a rate of ˜5 ml/min with mixing. After a final target siRNA/lipid ratio of 0.06 (wt/wt) was reached, the mixture was incubated for a further 30 min at 35° C. to allow vesicle reorganization and encapsulation of the siRNA. The ethanol was then removed and the external buffer replaced with PBS (155 mM NaCl, 3 mM Na₂HPO₄, 1 mM KH₂PO₄, pH 7.5) by either dialysis or tangential flow diafiltration. siRNA were encapsulated in SNALP using a controlled step-wise dilution method process. The lipid constituents of KC2-SNALP were DLin-KC2-DMA (cationic lipid), dipalmitoylphosphatidylcholine (DPPC; Avanti Polar Lipids), synthetic cholesterol (Sigma) and PEG-C-DMA used at a molar ratio of 57.1:7.1:34.3:1.4. Upon formation of the loaded particles, SNALP were dialyzed against PBS and filter sterilized through a 0.2 m filter before use. Mean particle sizes were 75-85 nm and 90-95% of the siRNA was encapsulated within the lipid particles. The final siRNA/lipid ratio in formulations used for in vivo testing was ˜0.15 (wt/wt). LNP-siRNA systems containing Factor VII siRNA were diluted to the appropriate concentrations in sterile PBS immediately before use and the formulations were administered intravenously through the lateral tail vein in a total volume of 10 ml/kg. This method and these delivery systems may be extrapolated to the CRISPR Cas system of the present invention.

### Other Lipids

Other cationic lipids, such as amino lipid 2,2-dilinoleyl-4-dimethylaminoethyl-[1,3]-dioxolane (DLin-KC2-DMA) may be utilized to encapsulate the DNA targeting agent according to the invention as described herein, such as by means of example CRISPR Cas or components thereof or nucleic acid molecule(s) coding therefor e.g., similar to SiRNA (see, e.g., Jayaraman, Angew. Chem. Int. Ed. 2012, 51, 8529-8533), and hence may be employed in the practice of the invention. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11+0.04 (n=56), the particles may be extruded up to three times through 80 nm membranes prior to adding the CRISPR Cas RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Michael S D Kormann et al. (“Expression of therapeutic proteins after delivery of chemically modified mRNA in mice: Nature Biotechnology, Volume:29, Pages: 154-157 (2011)) describes the use of lipid envelopes to deliver RNA. Use of lipid envelopes is also preferred in the present invention.

In another embodiment, lipids may be formulated with the CRISPR Cas system of the present invention to form lipid particles (LNPs). Lipids include, but are not limited to, DLin-KC2-DMA4, C12-200 and colipids disteroylphosphatidyl choline, cholesterol, and PEG-DMG may be formulated with CRISPR Cas instead of siRNA (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). The final lipid:siRNA weight ratio may be ˜12:1 and 9:1 in the case of DLin-KC2-DMA and C12-200 lipid particles (LNPs), respectively. The formulations may have mean particle diameters of −80 nm with >90% entrapment efficiency. A 3 mg/kg dose may be contemplated.

Tekmira has a portfolio of approximately 95 patent families, in the U.S. and abroad, that are directed to various aspects of LNPs and LNP formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316), all of which may be used and/or adapted to the present invention.

The DNA targeting agent according to the invention as described herein, such as by means of example CRISPR Cas system or components thereof or nucleic acid molecule(s) coding therefor may be delivered encapsulated in PLGA Microspheres such as that further described in US published applications 20130252281 and 20130245107 and 20130244279 (assigned to Moderna Therapeutics) which relate to aspects of formulation of compositions comprising modified nucleic acid molecules which may encode a protein, a protein precursor, or a partially or fully processed form of the protein or a protein precursor. The formulation may have a molar ratio 50:10:38.5:1.5-3.0 (cationic lipid:fusogenic lipid:cholesterol:PEG lipid). The PEG lipid may be selected from, but is not limited to PEG-c-DOMG, PEG-DMG. The fusogenic lipid may be DSPC. See also, Schrum et al., Delivery and Formulation of Engineered Nucleic Acids, US published application 20120251618.

Nanomerics' technology addresses bioavailability challenges for a broad range of therapeutics, including low molecular weight hydrophobic drugs, peptides, and nucleic acid based therapeutics (plasmid, siRNA, miRNA). Specific administration routes for which the technology has demonstrated clear advantages include the oral route, transport across the blood-brain-barrier, delivery to solid tumours, as well as to the eye. See, e.g., Mazza et al., 2013, ACS Nano. 2013 Feb. 26; 7(2):1016-26; Uchegbu and Siew, 2013, J Pharm Sci. 102(2):305-10 and Lalatsa et al., 2012, J Control Release. 2012 Jul. 20; 161(2):523-36.

US Patent Publication No. 20050019923 describes cationic dendrimers for delivering bioactive molecules, such as polynucleotide molecules, peptides and polypeptides and/or pharmaceutical agents, to a mammalian body. The dendrimers are suitable for targeting the delivery of the bioactive molecules to, for example, the liver, spleen, lung, kidney or heart (or even the brain). Dendrimers are synthetic 3-dimensional macromolecules that are prepared in a step-wise fashion from simple branched monomer units, the nature and functionality of which can be easily controlled and varied. Dendrimers are synthesised from the repeated addition of building blocks to a multifunctional core (divergent approach to synthesis), or towards a multifunctional core (convergent approach to synthesis) and each addition of a 3-dimensional shell of building blocks leads to the formation of a higher generation of the dendrimers. Polypropylenimine dendrimers start from a diaminobutane core to which is added twice the number of amino groups by a double Michael addition of acrylonitrile to the primary amines followed by the hydrogenation of the nitriles. This results in a doubling of the amino groups. Polypropylenimine dendrimers contain 100% protonable nitrogens and up to 64 terminal amino groups (generation 5, DAB 64). Protonable groups are usually amine groups which are able to accept protons at neutral pH. The use of dendrimers as gene delivery agents has largely focused on the use of the polyamidoamine and phosphorous containing compounds with a mixture of amine/amide or N—P(O₂)S as the conjugating units respectively with no work being reported on the use of the lower generation polypropylenimine dendrimers for gene delivery. Polypropylenimine dendrimers have also been studied as pH sensitive controlled release systems for drug delivery and for their encapsulation of guest molecules when chemically modified by peripheral amino acid groups. The cytotoxicity and interaction of polypropylenimine dendrimers with DNA as well as the transfection efficacy of DAB 64 has also been studied.

US Patent Publication No. 20050019923 is based upon the observation that, contrary to earlier reports, cationic dendrimers, such as polypropylenimine dendrimers, display suitable properties, such as specific targeting and low toxicity, for use in the targeted delivery of bioactive molecules, such as genetic material. In addition, derivatives of the cationic dendrimer also display suitable properties for the targeted delivery of bioactive molecules. See also, Bioactive Polymers, US published application 20080267903, which discloses “Various polymers, including cationic polyamine polymers and dendrimeric polymers, are shown to possess anti-proliferative activity, and may therefore be useful for treatment of disorders characterised by undesirable cellular proliferation such as neoplasms and tumours, inflammatory disorders (including autoimmune disorders), psoriasis and atherosclerosis. The polymers may be used alone as active agents, or as delivery vehicles for other therapeutic agents, such as drug molecules or nucleic acids for gene therapy. In such cases, the polymers' own intrinsic anti-tumour activity may complement the activity of the agent to be delivered.” The disclosures of these patent publications may be employed in conjunction with herein teachings for delivery of CRISPR Cas system(s) or component(s) thereof or nucleic acid molecule(s) coding therefor.

### Supercharged Proteins

Supercharged proteins are a class of engineered or naturally occurring proteins with unusually high positive or negative net theoretical charge and may be employed in delivery of the DNA targeting agent according to the invention as described herein, such as by means of example CRISPR Cas system(s) or component(s) thereof or nucleic acid molecule(s) coding therefor. Both supernegatively and superpositively charged proteins exhibit a remarkable ability to withstand thermally or chemically induced aggregation. Superpositively charged proteins are also able to penetrate mammalian cells. Associating cargo with these proteins, such as plasmid DNA, RNA, or other proteins, can enable the functional delivery of these macromolecules into mammalian cells both in vitro and in vivo. David Liu's lab reported the creation and characterization of supercharged proteins in 2007 (Lawrence et al., 2007, Journal of the American Chemical Society 129, 10110-10112).

The nonviral delivery of RNA and plasmid DNA into mammalian cells are valuable both for research and therapeutic applications (Akinc et al., 2010, Nat. Biotech. 26, 561-569). Purified +36 GFP protein (or other superpositively charged protein) is mixed with RNAs in the appropriate serum-free media and allowed to complex prior addition to cells. Inclusion of serum at this stage inhibits formation of the supercharged protein-RNA complexes and reduces the effectiveness of the treatment. The following protocol has been found to be effective for a variety of cell lines (McNaughton et al., 2009, Proc. Natl. Acad. Sci. USA 106, 6111-6116) (However, pilot experiments varying the dose of protein and RNA should be performed to optimize the procedure for specific cell lines): (1) One day before treatment, plate 1×10⁵ cells per well in a 48-well plate. (2) On the day of treatment, dilute purified +36 GFP protein in serumfree media to a final concentration 200 nM. Add RNA to a final concentration of 50 nM. Vortex to mix and incubate at room temperature for 10 min. (3) During incubation, aspirate media from cells and wash once with PBS. (4) Following incubation of +36 GFP and RNA, add the protein-RNA complexes to cells. (5) Incubate cells with complexes at 37° C. for 4h. (6) Following incubation, aspirate the media and wash three times with 20 U/mL heparin PBS. Incubate cells with serum-containing media for a further 48h or longer depending upon the assay for activity. (7) Analyze cells by immunoblot, qPCR, phenotypic assay, or other appropriate method.

David Liu's lab has further found +36 GFP to be an effective plasmid delivery reagent in a range of cells. As plasmid DNA is a larger cargo than siRNA, proportionately more +36 GFP protein is required to effectively complex plasmids. For effective plasmid delivery Applicants have developed a variant of +36 GFP bearing a C-terminal HA2 peptide tag, a known endosome-disrupting peptide derived from the influenza virus hemagglutinin protein. The following protocol has been effective in a variety of cells, but as above it is advised that plasmid DNA and supercharged protein doses be optimized for specific cell lines and delivery applications: (1) One day before treatment, plate 1×10⁵ per well in a 48-well plate. (2) On the day of treatment, dilute purified p36 GFP protein in serumfree media to a final concentration 2 mM. Add 1 mg of plasmid DNA. Vortex to mix and incubate at room temperature for 10 min. (3) During incubation, aspirate media from cells and wash once with PBS. (4) Following incubation of p36 GFP and plasmid DNA, gently add the protein-DNA complexes to cells. (5) Incubate cells with complexes at 37 C for 4h. (6) Following incubation, aspirate the media and wash with PBS. Incubate cells in serum-containing media and incubate for a further 24-48h. (7) Analyze plasmid delivery (e.g., by plasmid-driven gene expression) as appropriate. See also, e.g., McNaughton et al., Proc. Natl. Acad. Sci. USA 106, 6111-6116 (2009); Cronican et al., ACS Chemical Biology 5, 747-752 (2010); Cronican et al., Chemistry & Biology 18, 833-838 (2011); Thompson et al., Methods in Enzymology 503, 293-319 (2012); Thompson, D. B., et al., Chemistry & Biology 19 (7), 831-843 (2012). The methods of the super charged proteins may be used and/or adapted for delivery of the CRISPR Cas system of the present invention. These systems of Dr. Lui and documents herein in inconjunction with herein teachints can be employed in the delivery of the DNA targeting agent according to the invention as described herein, such as by means of example CRISPR Cas system(s) or component(s) thereof or nucleic acid molecule(s) coding therefor.

### Cell Penetrating Peptides (CPPs)

In yet another embodiment, cell penetrating peptides (CPPs) are contemplated for the delivery of the DNA targeting agent according to the invention as described herein, such as by means of example CRISPR Cas system. CPPs are short peptides that facilitate cellular uptake of various molecular cargo (from nanosize particles to small chemical molecules and large fragments of DNA). The term “cargo” as used herein includes but is not limited to the group consisting of therapeutic agents, diagnostic probes, peptides, nucleic acids, antisense oligonucleotides, plasmids, proteins, particles, liposomes, chromophores, small molecules and radioactive materials. In aspects of the invention, the cargo may also comprise any component of the DNA targeting agent according to the invention as described herein, such as by means of example CRISPR Cas system or the entire functional CRISPR Cas system. Aspects of the present invention further provide methods for delivering a desired cargo into a subject comprising: (a) preparing a complex comprising the cell penetrating peptide of the present invention and a desired cargo, and (b) orally, intraarticularly, intraperitoneally, intrathecally, intrarterially, intranasally, intraparenchymally, subcutaneously, intramuscularly, intravenously, dermally, intrarectally, or topically administering the complex to a subject. The cargo is associated with the peptides either through chemical linkage via covalent bonds or through non-covalent interactions.

The function of the CPPs are to deliver the cargo into cells, a process that commonly occurs through endocytosis with the cargo delivered to the endosomes of living mammalian cells. Cell-penetrating peptides are of different sizes, amino acid sequences, and charges but all CPPs have one distinct characteristic, which is the ability to translocate the plasma membrane and facilitate the delivery of various molecular cargoes to the cytoplasm or an organelle. CPP translocation may be classified into three main entry mechanisms: direct penetration in the membrane, endocytosis-mediated entry, and translocation through the formation of a transitory structure. CPPs have found numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots. CPPs hold great potential as in vitro and in vivo delivery vectors for use in research and medicine. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. One of the initial CPPs discovered was the transactivating transcriptional activator (Tat) from Human Immunodeficiency Virus 1 (HIV-1) which was found to be efficiently taken up from the surrounding media by numerous cell types in culture. Since then, the number of known CPPs has expanded considerably and small molecule synthetic analogues with more effective protein transduction properties have been generated. CPPs include but are not limited to Penetratin, Tat (48-60), Transportan, and (R-AhX-R4) (Ahx=aminohexanoyl).

U.S. Pat. No. 8,372,951, provides a CPP derived from eosinophil cationic protein (ECP) which exhibits highly cell-penetrating efficiency and low toxicity. Aspects of delivering the CPP with its cargo into a vertebrate subject are also provided. Further aspects of CPPs and their delivery are described in U.S. Pat. Nos. 8,575,305; 8,614,194 and 8,044,019. CPPs can be used to deliver the CRISPR-Cas system or components thereof. That CPPs can be employed to deliver the CRISPR-Cas system or components thereof is also provided in the manuscript “Gene disruption by cell-penetrating peptide-mediated delivery of Cas9 protein and guide RNA”, by Suresh Ramakrishna, Abu-Bonsrah Kwaku Dad, Jagadish Beloor, et al. Genome Res. 2014 Apr. 2. [Epub ahead of print], incorporated by reference in its entirety, wherein it is demonstrated that treatment with CPP-conjugated recombinant Cas9 protein and CPP-complexed guide RNAs lead to endogenous gene disruptions in human cell lines. In the paper the Cas9 protein was conjugated to CPP via a thioether bond, whereas the guide RNA was complexed with CPP, forming condensed, positively charged particles. It was shown that simultaneous and sequential treatment of human cells, including embryonic stem cells, dermal fibroblasts, HEK293T cells, HeLa cells, and embryonic carcinoma cells, with the modified Cas9 and guide RNA led to efficient gene disruptions with reduced off-target mutations relative to plasmid transfections.

### Implantable Devices

In another embodiment, implantable devices are also contemplated for delivery of the DNA targeting agent according to the invention as described herein, such as by means of example the CRISPR Cas system or component(s) thereof or nucleic acid molecule(s) coding therefor. For example, US Patent Publication 20110195123 discloses an implantable medical device which elutes a drug locally and in prolonged period is provided, including several types of such a device, the treatment modes of implementation and methods of implantation. The device comprising of polymeric substrate, such as a matrix for example, that is used as the device body, and drugs, and in some cases additional scaffolding materials, such as metals or additional polymers, and materials to enhance visibility and imaging. An implantable delivery device can be advantageous in providing release locally and over a prolonged period, where drug is released directly to the extracellular matrix (ECM) of the diseased area such as tumor, inflammation, degeneration or for symptomatic objectives, or to injured smooth muscle cells, or for prevention. One kind of drug is RNA, as disclosed above, and this system may be used/and or adapted to the DNA targeting agent according to the invention as described herein, such as by means of example CRISPR Cas system of the present invention. The modes of implantation in some embodiments are existing implantation procedures that are developed and used today for other treatments, including brachytherapy and needle biopsy. In such cases the dimensions of the new implant described in this invention are similar to the original implant. Typically a few devices are implanted during the same treatment procedure.

As described in US Patent Publication 20110195123, there is provided a drug delivery implantable or insertable system, including systems applicable to a cavity such as the abdominal cavity and/or any other type of administration in which the drug delivery system is not anchored or attached, comprising a biostable and/or degradable and/or bioabsorbable polymeric substrate, which may for example optionally be a matrix. It should be noted that the term “insertion” also includes implantation. The drug delivery system is preferably implemented as a “Loder” as described in US Patent Publication 20110195123.

The polymer or plurality of polymers are biocompatible, incorporating an agent and/or plurality of agents, enabling the release of agent at a controlled rate, wherein the total volume of the polymeric substrate, such as a matrix for example, in some embodiments is optionally and preferably no greater than a maximum volume that permits a therapeutic level of the agent to be reached. As a non-limiting example, such a volume is preferably within the range of 0.1 m³ to 1000 mm³, as required by the volume for the agent load. The Loder may optionally be larger, for example when incorporated with a device whose size is determined by functionality, for example and without limitation, a knee joint, an intra-uterine or cervical ring and the like.

The drug delivery system (for delivering the composition) is designed in some embodiments to preferably employ degradable polymers, wherein the main release mechanism is bulk erosion; or in some embodiments, non degradable, or slowly degraded polymers are used, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the surface is preferably maintained effectively constant during a significant period of the total drug releasing period, and therefore the diffusion rate is effectively constant (termed “zero mode” diffusion). By the term “constant” it is meant a diffusion rate that is preferably maintained above the lower threshold of therapeutic effectiveness, but which may still optionally feature an initial burst and/or may fluctuate, for example increasing and decreasing to a certain degree. The diffusion rate is preferably so maintained for a prolonged period, and it can be considered constant to a certain level to optimize the therapeutically effective period, for example the effective silencing period.

The drug delivery system optionally and preferably is designed to shield the nucleotide based therapeutic agent from degradation, whether chemical in nature or due to attack from enzymes and other factors in the body of the subject.

The drug delivery system as described in US Patent Publication 20110195123 is optionally associated with sensing and/or activation appliances that are operated at and/or after implantation of the device, by non and/or minimally invasive methods of activation and/or acceleration/deceleration, for example optionally including but not limited to thermal heating and cooling, laser beams, and ultrasonic, including focused ultrasound and/or RF (radiofrequency) methods or devices.

According to some embodiments of US Patent Publication 20110195123, the site for local delivery may optionally include target sites characterized by high abnormal proliferation of cells, and suppressed apoptosis, including tumors, active and or chronic inflammation and infection including autoimmune diseases states, degenerating tissue including muscle and nervous tissue, chronic pain, degenerative sites, and location of bone fractures and other wound locations for enhancement of regeneration of tissue, and injured cardiac, smooth and striated muscle.

The site for implantation of the composition, or target site, preferably features a radius, area and/or volume that is sufficiently small for targeted local delivery. For example, the target site optionally has a diameter in a range of from about 0.1 mm to about 5 cm.

The location of the target site is preferably selected for maximum therapeutic efficacy. For example, the composition of the drug delivery system (optionally with a device for implantation as described above) is optionally and preferably implanted within or in the proximity of a tumor environment, or the blood supply associated thereof.

For example the composition (optionally with the device) is optionally implanted within or in the proximity to pancreas, prostate, breast, liver, via the nipple, within the vascular system and so forth.

The target location is optionally selected from the group consisting of (as non-limiting examples only, as optionally any site within the body may be suitable for implanting a Loder): 1. brain at degenerative sites like in Parkinson or Alzheimer disease at the basal ganglia, white and gray matter; 2. spine as in the case of amyotrophic lateral sclerosis (ALS); 3. uterine cervix to prevent HPV infection; 4. active and chronic inflammatory joints; 5. dermis as in the case of psoriasis; 6. sympathetic and sensoric nervous sites for analgesic effect; 7. Intra osseous implantation; 8. acute and chronic infection sites; 9. Intra vaginal; 10. Inner ear--auditory system, labyrinth of the inner ear, vestibular system; 11. Intra tracheal; 12. Intra-cardiac; coronary, epicardiac; 13. urinary bladder; 14. biliary system; 15. parenchymal tissue including and not limited to the kidney, liver, spleen; 16. lymph nodes; 17. salivary glands; 18. dental gums; 19. Intra-articular (into joints); 20. Intra-ocular; 21. Brain tissue; 22. Brain ventricles; 23. Cavities, including abdominal cavity (for example but without limitation, for ovary cancer); 24. Intra esophageal and 25. Intra rectal.

Optionally insertion of the system (for example a device containing the composition) is associated with injection of material to the ECM at the target site and the vicinity of that site to affect local pH and/or temperature and/or other biological factors affecting the diffusion of the drug and/or drug kinetics in the ECM, of the target site and the vicinity of such a site.

Optionally, according to some embodiments, the release of said agent could be associated with sensing and/or activation appliances that are operated prior and/or at and/or after insertion, by non and/or minimally invasive and/or else methods of activation and/or acceleration/deceleration, including laser beam, radiation, thermal heating and cooling, and ultrasonic, including focused ultrasound and/or RF (radiofrequency) methods or devices, and chemical activators.

According to other embodiments of US Patent Publication 20110195123, the drug preferably comprises a RNA, for example for localized cancer cases in breast, pancreas, brain, kidney, bladder, lung, and prostate as described below. Although exemplified with RNAi, many drugs are applicable to be encapsulated in Loder, and can be used in association with this invention, as long as such drugs can be encapsulated with the Loder substrate, such as a matrix for example, and this system may be used and/or adapted to deliver the CRISPR Cas system of the present invention.

As another example of a specific application, neuro and muscular degenerative diseases develop due to abnormal gene expression. Local delivery of RNAs may have therapeutic properties for interfering with such abnormal gene expression. Local delivery of anti apoptotic, anti inflammatory and anti degenerative drugs including small drugs and macromolecules may also optionally be therapeutic. In such cases the Loder is applied for prolonged release at constant rate and/or through a dedicated device that is implanted separately. All of this may be used and/or adapted to the DNA targeting agent according to the invention as described herein, such as by means of example CRISPR Cas system of the present invention.

As yet another example of a specific application, psychiatric and cognitive disorders are treated with gene modifiers. Gene knockdown is a treatment option. Loders locally delivering agents to central nervous system sites are therapeutic options for psychiatric and cognitive disorders including but not limited to psychosis, bi-polar diseases, neurotic disorders and behavioral maladies. The Loders could also deliver locally drugs including small drugs and macromolecules upon implantation at specific brain sites. All of this may be used and/or adapted to the CRISPR Cas system of the present invention.

As another example of a specific application, silencing of innate and/or adaptive immune mediators at local sites enables the prevention of organ transplant rejection. Local delivery of RNAs and immunomodulating reagents with the Loder implanted into the transplanted organ and/or the implanted site renders local immune suppression by repelling immune cells such as CD8 activated against the transplanted organ. All of this may be used/and or adapted to the DNA targeting agent according to the invention as described herein, such as by means of example CRISPR Cas system of the present invention.

As another example of a specific application, vascular growth factors including VEGFs and angiogenin and others are essential for neovascularization. Local delivery of the factors, peptides, peptidomimetics, or suppressing their repressors is an important therapeutic modality; silencing the repressors and local delivery of the factors, peptides, macromolecules and small drugs stimulating angiogenesis with the Loder is therapeutic for peripheral, systemic and cardiac vascular disease.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as ERCP, stereotactic methods into the brain tissue, Laparoscopy, including implantation with a laparoscope into joints, abdominal organs, the bladder wall and body cavities.

Implantable device technology herein discussed can be employed with herein teachings and hence by this disclosure and the knowledge in the art, the DNA targeting agent according to the invention as described herein, such as by means of example CRISPR-Cas system or components thereof or nucleic acid molecules thereof or encoding or providing components may be delivered via an implantable device.

The present application also contemplates an inducible CRISPR Cas system. Reference is made to international patent application Serial No. PCT/US13/51418 filed Jul. 21, 2013, which published as WO2014/018423 on Jan. 30, 2014.

In one aspect the invention provides a DNA targeting agent according to the invention as described herein, such as by means of example a non-naturally occurring or engineered CRISPR Cas system which may comprise at least one switch wherein the activity of said CRISPR Cas system is controlled by contact with at least one inducer energy source as to the switch. In an embodiment of the invention the control as to the at least one switch or the activity of said CRISPR Cas system may be activated, enhanced, terminated or repressed. The contact with the at least one inducer energy source may result in a first effect and a second effect.

The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said the DNA targeting agent according to the invention as described herein, such as by means of example CRISPR Cas system. In one embodiment the first effect and the second effect may occur in a cascade.

The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (40HT), estrogen or ecdysone.

The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 40HT/estrogen inducible systems, ecdysone-based inducible systems and FKBP12/FRAP (FKBP12-rapamycin complex) inducible systems.

In one aspect of the invention the inducer energy source is electromagnetic energy.

The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm2, or more preferably at least 4 mW/cm2. In another embodiment, the component of visible light may have a wavelength in the range of 620-700 nm and is red light.

In a further aspect, the invention provides a method of controlling the DNA targeting agent according to the invention as described herein, such as by means of example a non-naturally occurring or engineered CRISPR Cas system, comprising providing said CRISPR Cas system comprising at least one switch wherein the activity of said CRISPR Cas system is controlled by contact with at least one inducer energy source as to the switch.

In an embodiment of the invention, the invention provides methods wherein the control as to the at least one switch or the activity of said the DNA targeting agent according to the invention as described herein, such as by means of example CRISPR Cas system may be activated, enhanced, terminated or repressed. The contact with the at least one inducer energy source may result in a first effect and a second effect. The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said CRISPR Cas system. In one embodiment the first effect and the second effect may occur in a cascade.

The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (40HT), estrogen or ecdysone. The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 40HT/estrogen inducible systems, ecdysone-based inducible systems and FKBP12/FRAP (FKBP12-rapamycin complex) inducible systems.

In one aspect of the methods of the invention the inducer energy source is electromagnetic energy. The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm2, or more preferably at least 4 mW/cm2. In another embodiment, the component of visible light may have a wavelength in the range of 620-700 nm and is red light.

In another preferred embodiment of the invention, the inducible effector may be a Light Inducible Transcriptional Effector (LITE). The modularity of the LITE system allows for any number of effector domains to be employed for transcriptional modulation. In yet another preferred embodiment of the invention, the inducible effector may be a chemical. The invention also contemplates an inducible multiplex genome engineering using CRISPR (clustered regularly interspaced short palindromic repeats)/Cas systems.

### Self-Inactivating Systems

Once all copies of a gene in the genome of a cell have been edited, continued CRISRP/Cas9 expression in that cell is no longer necessary. Indeed, sustained expression would be undesirable in case of off-target effects at unintended genomic sites, etc. Thus time-limited expression would be useful. Inducible expression offers one approach, but in addition Applicants have engineered a Self-Inactivating CRISPR-Cas9 system that relies on the use of a non-coding guide target sequence within the CRISPR vector itself. Thus, after expression begins, the CRISPR system will lead to its own destruction, but before destruction is complete it will have time to edit the genomic copies of the target gene (which, with a normal point mutation in a diploid cell, requires at most two edits). Simply, the self inactivating CRISPR-Cas system includes additional RNA (i.e., guide RNA) that targets the coding sequence for the CRISPR enzyme itself or that targets one or more non-coding guide target sequences complementary to unique sequences present in one or more of the following:

(a) within the promoter driving expression of the non-coding RNA elements,
(b) within the promoter driving expression of the Cas9 gene,
(c) within 100 bp of the ATG translational start codon in the Cas9 coding sequence,
(d) within the inverted terminal repeat (iTR) of a viral delivery vector, e.g., in the AAV genome.

Furthermore, that RNA can be delivered via a vector, e.g., a separate vector or the same vector that is encoding the CRISPR complex. When provided by a separate vector, the CRISPR RNA that targets Cas expression can be administered sequentially or simultaneously. When administered sequentially, the CRISPR RNA that targets Cas expression is to be delivered after the CRISPR RNA that is intended for e.g. gene editing or gene engineering. This period may be a period of minutes (e.g. 5 minutes, 10 minutes, 20 minutes, 30 minutes, 45 minutes, 60 minutes). This period may be a period of hours (e.g. 2 hours, 4 hours, 6 hours, 8 hours, 12 hours, 24 hours). This period may be a period of days (e.g. 2 days, 3 days, 4 days, 7 days). This period may be a period of weeks (e.g. 2 weeks, 3 weeks, 4 weeks). This period may be a period of months (e.g. 2 months, 4 months, 8 months, 12 months). This period may be a period of years (2 years, 3 years, 4 years). In this fashion, the Cas enzyme associates with a first gRNA/chiRNA capable of hybridizing to a first target, such as a genomic locus or loci of interest and undertakes the function(s) desired of the CRISPR-Cas system (e.g., gene engineering); and subsequently the Cas enzyme may then associate with the second gRNA/chiRNA capable of hybridizing to the sequence comprising at least part of the Cas or CRISPR cassette. Where the gRNA/chiRNA targets the sequences encoding expression of the Cas protein, the enzyme becomes impeded and the system becomes self inactivating. In the same manner, CRISPR RNA that targets Cas expression applied via, for example liposome, lipofection, nanoparticles, microvesicles as explained herein, may be administered sequentially or simultaneously. Similarly, self-inactivation may be used for inactivation of one or more guide RNA used to target one or more targets.

In some aspects, a single gRNA is provided that is capable of hybridization to a sequence downstream of a CRISPR enzyme start codon, whereby after a period of time there is a loss of the CRISPR enzyme expression. In some aspects, one or more gRNA(s) are provided that are capable of hybridization to one or more coding or non-coding regions of the polynucleotide encoding the CRISPR-Cas system, whereby after a period of time there is a inactivation of one or more, or in some cases all, of the CRISPR-Cas system. In some aspects of the system, and not to be limited by theory, the cell may comprise a plurality of CRISPR-Cas complexes, wherein a first subset of CRISPR complexes comprise a first chiRNA capable of targeting a genomic locus or loci to be edited, and a second subset of CRISPR complexes comprise at least one second chiRNA capable of targeting the polynucleotide encoding the CRISPR-Cas system, wherein the first subset of CRISPR-Cas complexes mediate editing of the targeted genomic locus or loci and the second subset of CRISPR complexes eventually inactivate the CRISPR-Cas system, thereby inactivating further CRISPR-Cas expression in the cell.

Thus the invention provides a CRISPR-Cas system comprising one or more vectors for delivery to a eukaryotic cell, wherein the vector(s) encode(s): (i) a CRISPR enzyme; (ii) a first guide RNA capable of hybridizing to a target sequence in the cell; (iii) a second guide RNA capable of hybridizing to one or more target sequence(s) in the vector which encodes the CRISPR enzyme; (iv) at least one tracr mate sequence; and (v) at least one tracr sequence, The first and second complexes can use the same tracr and tracr mate, thus differing only by the guide sequence, wherein, when expressed within the cell: the first guide RNA directs sequence-specific binding of a first CRISPR complex to the target sequence in the cell; the second guide RNA directs sequence-specific binding of a second CRISPR complex to the target sequence in the vector which encodes the CRISPR enzyme; the CRISPR complexes comprise (a) a tracr mate sequence hybridised to a tracr sequence and (b) a CRISPR enzyme bound to a guide RNA, such that a guide RNA can hybridize to its target sequence; and the second CRISPR complex inactivates the CRISPR-Cas system to prevent continued expression of the CRISPR enzyme by the cell.

Further characteristics of the vector(s), the encoded enzyme, the guide sequences, etc. are disclosed elsewhere herein. For instance, one or both of the guide sequence(s) can be part of a chiRNA sequence which provides the guide, tracr mate and tracr sequences within a single RNA, such that the system can encode (i) a CRISPR enzyme; (ii) a first chiRNA comprising a sequence capable of hybridizing to a first target sequence in the cell, a first tracr mate sequence, and a first tracr sequence; (iii) a second guide RNA capable of hybridizing to the vector which encodes the CRISPR enzyme, a second tracr mate sequence, and a second tracr sequence. Similarly, the enzyme can include one or more NLS, etc.

The various coding sequences (CRISPR enzyme, guide RNAs, tracr and tracr mate) can be included on a single vector or on multiple vectors. For instance, it is possible to encode the enzyme on one vector and the various RNA sequences on another vector, or to encode the enzyme and one chiRNA on one vector, and the remaining chiRNA on another vector, or any other permutation. In general, a system using a total of one or two different vectors is preferred.

Where multiple vectors are used, it is possible to deliver them in unequal numbers, and ideally with an excess of a vector which encodes the first guide RNA relative to the second guide RNA, thereby assisting in delaying final inactivation of the CRISPR system until genome editing has had a chance to occur.

The first guide RNA can target any target sequence of interest within a genome, as described elsewhere herein. The second guide RNA targets a sequence within the vector which encodes the CRISPR Cas9 enzyme, and thereby inactivates the enzyme's expression from that vector. Thus the target sequence in the vector must be capable of inactivating expression. Suitable target sequences can be, for instance, near to or within the translational start codon for the Cas9 coding sequence, in a non-coding sequence in the promoter driving expression of the non-coding RNA elements, within the promoter driving expression of the Cas9 gene, within 100 bp of the ATG translational start codon in the Cas9 coding sequence, and/or within the inverted terminal repeat (iTR) of a viral delivery vector, e.g., in the AAV genome. A double stranded break near this region can induce a frame shift in the Cas9 coding sequence, causing a loss of protein expression. An alternative target sequence for the “self-inactivating” guide RNA would aim to edit/inactivate regulatory regions/sequences needed for the expression of the CRISPR-Cas9 system or for the stability of the vector. For instance, if the promoter for the Cas9 coding sequence is disrupted then transcription can be inhibited or prevented. Similarly, if a vector includes sequences for replication, maintenance or stability then it is possible to target these. For instance, in a AAV vector a useful target sequence is within the iTR. Other useful sequences to target can be promoter sequences, polyadenlyation sites, etc.

Furthermore, if the guide RNAs are expressed in array format, the “self-inactivating” guide RNAs that target both promoters simultaneously will result in the excision of the intervening nucleotides from within the CRISPR-Cas expression construct, effectively leading to its complete inactivation. Similarly, excision of the intervening nucleotides will result where the guide RNAs target both ITRs, or targets two or more other CRISPR-Cas components simultaneously. Self-inactivation as explained herein is applicable, in general, with CRISPR-Cas9 systems in order to provide regulation of the CRISPR-Cas9. For example, self-inactivation as explained herein may be applied to the CRISPR repair of mutations, for example expansion disorders, as explained herein. As a result of this self-inactivation, CRISPR repair is only transiently active.

Addition of non-targeting nucleotides to the 5′ end (e.g. 1-10 nucleotides, preferably 1-5 nucleotides) of the “self-inactivating” guide RNA can be used to delay its processing and/or modify its efficiency as a means of ensuring editing at the targeted genomic locus prior to CRISPR-Cas9 shutdown.

In one aspect of the self-inactivating AAV-CRISPR-Cas9 system, plasmids that co-express one or more sgRNA targeting genomic sequences of interest (e.g. 1-2, 1-5, 1-10, 1-15, 1-20, 1-30) may be established with “self-inactivating” sgRNAs that target an SpCas9 sequence at or near the engineered ATG start site (e.g. within 5 nucleotides, within 15 nucleotides, within 30 nucleotides, within 50 nucleotides, within 100 nucleotides). A regulatory sequence in the U6 promoter region can also be targeted with an sgRNA. The U6-driven sgRNAs may be designed in an array format such that multiple sgRNA sequences can be simultaneously released. When first delivered into target tissue/cells (left cell) sgRNAs begin to accumulate while Cas9 levels rise in the nucleus. Cas9 complexes with all of the sgRNAs to mediate genome editing and self-inactivation of the CRISPR-Cas9 plasmids.

One aspect of a self-inactivating CRISPR-Cas9 system is expression of singly or in tandam array format from 1 up to 4 or more different guide sequences; e.g. up to about 20 or about 30 guides sequences. Each individual self inactivating guide sequence may target a different target. Such may be processed from, e.g. one chimeric pol3 transcript. Pol3 promoters such as U6 or H1 promoters may be used. Pol2 promoters such as those mentioned throughout herein. Inverted terminal repeat (iTR) sequences may flank the Pol3 promoter-sgRNA(s)-Pol2 promoter-Cas9.

One aspect of a chimeric, tandem array transcript is that one or more guide(s) edit the one or more target(s) while one or more self inactivating guides inactivate the CRISPR/Cas9 system. Thus, for example, the described CRISPR-Cas9 system for repairing expansion disorders may be directly combined with the self-inactivating CRISPR-Cas9 system described herein. Such a system may, for example, have two guides directed to the target region for repair as well as at least a third guide directed to self-inactivation of the CRISPR-Cas9. Reference is made to Application Ser. No. PCT/US2014/069897, entitled “Compositions And Methods Of Use Of Crispr-Cas Systems In Nucleotide Repeat Disorders,” published Dec. 12, 2014 as WO/2015/089351.

One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In advantageous embodiments of the invention, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), monomers with an RVD of NG preferentially bind to thymine (T), monomers with an RVD of HD preferentially bind to cytosine (C) and monomers with an RVD of NN preferentially bind to both adenine (A) and guanine (G). In yet another embodiment of the invention, monomers with an RVD of IG preferentially bind to T. Thus, the number and order of the polypeptide monomer repeats in the nucleic acid binding domain of a TALE determines its nucleic acid target specificity. In still further embodiments of the invention, monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, monomers having RVDs of H*, HA, KA, N*, NA, NC, NS, RA, and S* bind to adenine, guanine, cytosine and thymine with comparable affinity.

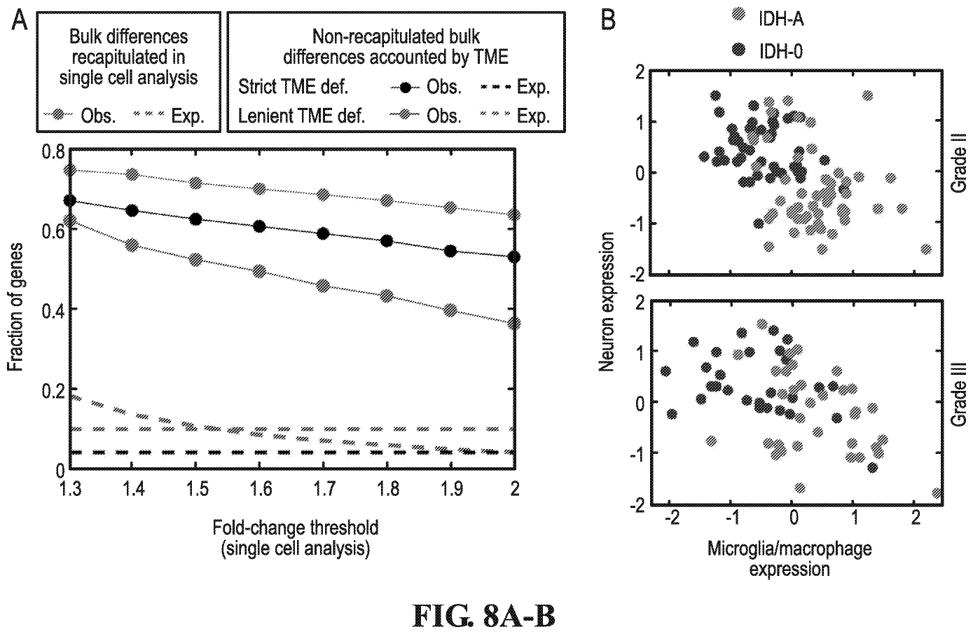

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8). Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

(SEQ ID NO: 17)
M D P I R S R T P S P A R E L L S G P Q P D G V Q P T A D R G V S P
P A G G P L D G L P A R R T M S R T R L P S P P A P S P A F S A D S
F S D L L R Q F D P S L F N T S L F D S L P P F G A H H T E A A T G
E W D E V Q S G L R A A D A P P P T M R V A V T A A R P P R A K P
A
P R R R A A Q P S D A S P A A Q V D L R T L G Y S Q Q Q Q E K I K
P
K V R S T V A Q H H E A L V G H G F T H A H I V A L S Q H P A A L
G
T V A V K Y Q D M I A A L P E A T H E A I V G V G K Q W S G A R
A L
E A L L T V A G E L R G P P L Q L D T G Q L L K I A K R G G V T A
V
E A V H A W R N A L T G A P L N

An exemplary amino acid sequence of a N-terminal capping region is:

(SEQ ID NO: 18)
R P A L E S I V A Q L S R P D P A L A A L T N D H L V A L A C L G
G R P A L D A V K K G L P H A P A L I K R T N R R I P E R T S H R
V A D H A Q V V R V L G F F Q C H S H P A Q A F D D A M T Q F G
M
S R H G L L Q L F R R V G V T E L E A R S G T L P P A S Q R W D R
I L Q A S G M K R A K P S P T S T Q T P D Q A S L H A F A D S L E
R D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

Applicants have previously developed methods and tools for genome-scale screening of perturbations in single cells using CRISPR-Cas9, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; and Adamson et al., “A Multiplexed Single-Cell CRISPR Screening Platform Enables Systematic Dissection of the Unfolded Protein Response” 2016, Cell 167, 1867-1882). The present invention is compatible with perturb-seq, such that signature genes may be perturbed and the perturbation may be identified and assigned to the proteomic and gene expression readouts of single cells.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by Drop-seq, as described herein. In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence and reduces the chance of false guide RNA assignment. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein for Drop-seq methods. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed in is derived from more than one protein binding event or transcript. In preferred embodiments, Perturb-seq is performed using a guide RNA barcode expressed as a polyadenylated transcript, a cell barcode, and a UMI.

Perturb-seq combines emerging technologies in the field of genome engineering, and single-cell analysis, in particular the CRISPR-Cas9 system and droplet single-cell sequencing analysis. In certain embodiments, a CRISPR system is used to create an INDEL at a target gene. In other embodiments, epigenetic screening is performed by applying CRISPRa/i technology. Numerous genetic variants associated with disease phenotypes are found to be in non-coding region of the genome, and frequently coincide with transcription factor (TF) binding sites and non-coding RNA genes. Not being bound by a theory, CRISPRa/i approaches may be used to achieve a more thorough and precise understanding of the implication of epigenetic regulation.

In certain embodiments, other CRISPR-based perturbations are readily compatible with Perturb-seq, including alternative editors such as CRISPR/Cpf1. In certain embodiments, Perturb-seq uses Cpf1 as the CRISPR enzyme for introducing perturbations. Not being bound by a theory, Cpf1 does not require Tracr RNA and is a smaller enzyme, thus allowing higher combinatorial perturbations to be tested.

The cell(s) may comprise a cell in a model non-human organism, a model non-human mammal that expresses a Cas protein, a mouse that expresses a Cas protein, a mouse that expresses Cpf1, a cell in vivo or a cell ex vivo or a cell in vitro. The cell(s) may also comprise a human cell.

In one embodiment, CRISPR/Cas9 may be used to perturb protein-coding genes or non-protein-coding DNA. CRISPR/Cas9 may be used to knockout protein-coding genes by frameshifts, point mutations, inserts, or deletions. An extensive toolbox may be used for efficient and specific CRISPR/Cas9 mediated knockout as described herein, including a double-nicking CRISPR to efficiently modify both alleles of a target gene or multiple target loci and a smaller Cas9 protein for delivery on smaller vectors (Ran, F. A., et al., In vivo genome editing using Staphylococcus aureus Cas9. Nature. 520, 186-191 (2015)). A genome-wide sgRNA mouse library (10 sgRNAs/gene) may also be used in a mouse that expresses a Cas9 protein.

In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In one embodiment, perturbation is by deletion of regulatory elements. Non-coding elements may be targeted by using pairs of guide RNAs to delete regions of a defined size, and by tiling deletions covering sets of regions in pools.

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus.

Applicants have developed and optimized methods and conditions for delivery of a CRISPR system to primary mouse T-cells. Applicants have achieved over 80% transduction efficiency with Lenti-CRISPR constructs in CD4 and CD8 T-cells. Despite success with lentiviral delivery, recent work by Hendel et al, (Nature Biotechnology 33, 985-989 (2015) doi:10.1038/nbt.3290) showed the efficiency of editing human T-cells with chemically modified RNA, and direct RNA delivery to T-cells via electroporation. In certain embodiments, perturbation in mouse primary T-cells may use these methods.

In certain embodiments, whole genome screens can be used for understanding the phenotypic readout of perturbing potential target genes. In preferred embodiments, perturbations target expressed genes as defined by RNA-seq using a focused sgRNA library. Libraries may be focused on expressed genes in specific networks or pathways. In other preferred embodiments, regulatory drivers are perturbed. Applicants can use gene expression profiling data to define the target of interest and perform follow-up single-cell and population RNA-seq analysis. Not being bound by a theory, this approach will accelerate the development of therapeutics for human disorders, in particular gliomas.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R.I. Freshney, ed. (1987)).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

The present invention also comprises a kit with a detection reagent that binds to one or more signature genes. In one embodiment, nucleic acids are detected. Nucleic acids may be detected by RNA FISH. In preferred embodiments, proteins are detected. Most preferably cell surface markers are detected. Thus, the present invention provides for detection reagents to be used in the detection of proteins, such as, but not limited to antibodies specific for signature genes. Not being bound by a theory, antibodies may be used to detect cells by FACS or immunohistochemistry. In certain embodiments, the invention provides for an array of detection reagents, e.g., oligonucleotides that can bind to one or more signature nucleic acids, or antibodies specific to one or more proteins. Suitable detection reagents include antibodies or fragments thereof, aptamers, or oligonucleotides packaged together in the form of a kit. The oligonucleotides can be fragments of the signature genes. For example the oligonucleotides can be 200, 150, 100, 50, 25, 10 or fewer nucleotides in length. The kit may contain in separate container or packaged separately with reagents for binding any of the detection reagents to a matrix. The kit may contain control formulations (positive and/or negative), and/or a detectable label such as fluorescein, green fluorescent protein, rhodamine, cyanine dyes, Alexa dyes, luciferase, radiolabels, among others. Instructions (e.g., written, online, etc.) for carrying out the assay may be included in the kit. The assay may for example be in the form of a FISH assay, FACS assay, CyTOF assay, ELISA assay, or any other method as known in the art. Alternatively, the kit contains a nucleic acid substrate array comprising one or more nucleic acid sequences.

These and other technologies may be employed in or as to the practice of the instant invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

### Example 1: Methods

### Tumor Dissociation

Patients at the Massachusetts General Hospital were consented preoperatively in all cases according to the Institutional Review Board Protocol 1999P008145. Fresh tumors were collected at time of resection and presence of malignant cells was confirmed by frozen section. Fresh tumor tissue was mechanically and enzymatically dissociated using a papain-based brain tumor dissociation kit (Miltenyi Biotec). Large pieces of debris were removed with a 100 micron strainer, and dissociated cells were layered onto a 5 mL density gradient (Lympholyte-H, Cedar Lane labs), which was centrifuged at 2,000 rpm for 10 min at room temperature to pellet dead cells and red blood cells. The interface containing live cells was saved and used for staining and flow cytometry. Viability was measured using trypan blue exclusion, which confirmed >90% cell viability.

### Fluorescence-Activated Cell Sorting (FACS)

Primary tumor sorting: Tumor cells were blocked in 1% bovine serum albumin in Hanks buffered saline solution (BSA/HBSS), and then stained first with CD45-Vioblue direct antibody conjugate (Miltenyi Biotec) for 30 min at 4° C. Cells were washed with cold PBS, and then resuspended in 1 mL of BSA/HBSS containing 1 uM calcein AM (Life Technologies) and 0.33 uM TO-PRO-3 iodide (Life Technologies) to co-stain for 30 min before sorting. FACS was performed on FACSAria Fusion Special Order System (Becton Dickinson) using 488 nm (calcein AM, 530/30 filter), 640 nm (TO-PRO-3, 670/14 filter), and 405 nm (Vioblue, 450/50 filter) lasers. Fluorescence-minus-one controls were included with all tumors, as well as heat killed controls in early pilot experiments, which were crucial to ensure proper identification of the TO-PRO-3 positive compartment and ensure sorting of the live cell population. Standard, strict forward scatter height versus area criteria were used to discriminate doublets and gate only singleton cells. Viable cells were identified by staining positive with calcein AM but negative for TOPRO-3. Single cells were sorted into 96-well plates containing cold TCL buffer (Qiagen) containing 1% beta-mercaptoethanol, snap frozen on dry ice, and then stored at −80° C. prior to whole transcriptome amplification, library preparation and sequencing. Sorting of cell cultures: The BT54 oligodendroglioma cell line (107) was grown in serum-free conditions [Neurobasal media containing 3 mM glutaMAX, B27 supplement, N2 supplement and penicillin-streptomycin (Life Technologies); 100 ng/mL EGF and 40 ng/mL FGF (R&D Systems). Cells dissociated in TrypLE (ThermoFisher Scientific) were blocked in PBS containing 1% BSA (BSA/PBS), stained for 20 min with CD24-PE direct antibody conjugate (Miltenyi), washed, and resuspended in BSA/PBS containing calcein and TO-PRO-3 to identify live cells as above. Cells in the top and bottom ˜15% of CD24 staining were sorted and cultured in CSC media at a concentration of 20,000 cells per mL in duplicate to monitor spherogenic growth.

### Whole Transcriptome Amplification, Library Construction, Sequencing, and Processing

Libraries from isolated single cells were generated based on the Smart-seq2 protocol (93) with the following modifications. RNA from single cells was first purified with Agencourt RNAClean XP beads (Beckman Coulter) prior to oligo-dT primed reverse transcription with Maxima reverse transcriptase and locked TSO oligonucleotide, which was followed by 20 cycle PCR amplification using KAPA HiFi HotStart ReadyMix (KAPA Biosystems) with subsequent Agencourt AMPure XP bead purification as described. Libraries were tagmented using the Nextera XT Library Prep kit (Illumina) with custom barcode adapters (sequences available upon request). Libraries from 384 cells with unique barcodes were combined and sequenced using a NextSeq 500 sequencer (Illumina).

Applicants also analyzed 96 cells from MGH60 with an alternative protocol that incorporates random molecular tags (RMTs, also known us unique molecular identifiers, or UMIs) in order to control for PCR amplification bias, as described previously (119) and obtained similar results.

Paired-end, 38-base reads were mapped to the UCSC hg19 human transcriptome using Bowtie (59) with parameters “-q --phred33-quals -n 1 -e 99999999-1 25 -I 1 -X 2000 -a -m 15 -S -p 6”, which allows alignment of sequences with single base changes. Expression values were calculated from SAM files using RSEM v1.2.3 (60) in paired-end mode using parameters “-- estimate-rspd --paired end -sam -p 6”, from which TPM values for each gene were extracted.

### Immunohistochemistry

Hematoxylin and eosin and single antibody staining (GFAP, Ki67) was done by the clinical pathology laboratory at the Massachusetts General Hospital per routine protocol. For double GFAP/Ki67 double immunohistochemistry, paraffin-embedded sections were mounted on glass slides, deparaffinized in xylene, treated with 0.5% peroxide in methanol, and rehydrated. Antigen retrieval was done using sodium citrate-based, heat-induced antigen retrieval at pH 6.0. The Dako EnVision G/2 double stain system was used for blocking, staining, and development using rabbit anti-Ki67 antibody (Abcam ab15580 at 1:300) and mouse anti-GFAP antibody (Dako M0761 at 1:100).

### Analysis of Bulk DNA Methylation Profiles

Raw Illumina Human Methylation 450 array data from the TCGA LGG and AML projects were downloaded from the Genomic Data Commons Legacy Archive (gdc-portal.nci.nih.gov/legacy-archive). Annotation for IDH mutational status and 1p/19q co-deletion were obtained from published TCGA studies (112, 141). Methylation data and IDH mutational status from Guilhamon et al., 2013 were downloaded from the Gene Expression Omnibus (www.ncbi.nlm.nih.gov/geo), accession number GSE40853 (142). TCGA data was processed from idat files in R using the minfi Bioconductor package with default parameters (143), and beta-values were used for subsequent analysis. Of the 482,421 CpG probes present on the array, the following were removed: probes targeting the X and Y chromosomes (n=11,551), probes containing a single-nucleotide polymorphism (dbSNP132 Common) within five base pairs of and including the targeted CpG-site (n=7,998), and probes not mapping uniquely to the human reference genome (hg19) allowing for one mismatch (n=3,965). In total, 459,226 probes were kept for analysis. For heatmap representation, data from the TCGA LGG project was downsampled to 25 samples per group, and the 10,000 most variable CpGs (by standard deviation) across groups were selected.

### RNA In Situ Hybridization

Paraffin-embedded tissue sections from human tumors from Massachusetts General Hospital were obtained according to an Institutional Review Board-approved protocol (1999P008145 and 2011P002334) mounted on glass slides and stored at −80° C. Slides were stained using the RNAscope 2.5 HD Duplex Detection Kit (Advanced Cell Technologies, Cat. No. 322430). Slides were baked for 1 hour at 60° C., deparaffinized and dehydrated with xylene and ethanol. The tissue was pretreated with RNAscope Hydrogen Peroxide (Cat. No. 322335) for 10 minutes at room temperature and RNAscope Target Retrieval Reagent (Cat. No. 322000) for 15 minutes at 98° C. RNAscope Protease Plus (Cat. No. 322331) was then applied to the tissue for 30 minutes at 40° C. Hybridization probes were prepared by diluting the C2 probe (red) 1:50 into the C1 probe (green). Advanced Cell Technologies RNAscope Target Probes used included SOX4 (C1, Cat. No. 469911), MKI67 (C2, Cat. No. 591771-C2), CX3CR1 (C1, Cat. No. 411251), and CD163 (C2, Cat, No. 417061-C2). Probes were added to the tissue and hybridized for 2 hours at 40° C. A series of 10 amplification steps were performed using instructions and reagents provided in the RNAscope 2.5 HD Duplex Detection Kit. Tissue was counterstained with Gill's hematoxylin for 25 seconds at room temperature followed by mounting with VectaMount mounting media (Vector Laboratories).

For a subset of slides, Applicants used the ViewRNA technology (Affymetrix) for manual format RNA in situ hybridization. Briefly, slides were baked at 60° C. for 1 hour, then denatured at 80° C. for 3 min, deparaffinized with Histoclear and ethanol dehydration. RNA targets in dewaxed sections were unmasked by treating with pretreatment buffer at 95C for 10 min and digested with 1:100 dilution protease at 40° C. for 10 min, followed by fixation with 10% formalin for 5 min at room temperature. Probe concentration was 1:40 for both type 1 (red) and type 6 (blue) probe sets. Probes were incubated on sections for 2 hr at 40° C. and then washed serially. Affymetrix Panomics probes included ApoE (type 6, catalogue number VA6-16904 and type 1, catalogue number VA1-18265) and ApoD (type 1, VX6-99999-01). Signal was amplified using PreAmplifier mix QT for 25 min at 40° C. followed by Amplifier mix QT for 15 min at 40° C., and then signal was hybridized with labeled probe at 1:1000 dilution for 15 min at 40° C. Color was developed using Fast Blue substrate for Type 6 probes and Fast Red substrate for Type 1 probes for 30 min at 40° C. Tissue was counterstained with Gill's hematoxylin for 25 sec at room temperature followed by mounting with ADVANTAGE mounting media (Innovex). For quantification of compartments by ISH, at least 1,000 cells were counted in representative areas of the tumors.

In alternative methods, tissue sections mounted on glass slides were stored at −80C until ready for hybridization. Slides were baked at 60C for 1 hour, then denatured at 80C for 3 min, deparaffinized with Histoclear and ethanol dehydration. RNA targets in dewaxed sections were unmasked by treating with pretreatment buffer at 95C for 10 min and digested with 1:100 dilution protease at 40C for 10 min, followed by fixation with 10% formalin for 5 min at room temperature. Probe concentrations were 1:40 for both type 1 (red) and type 6 (blue) probe sets, except that the ApoE probe was used at 1:80 dilution. Probe was incubated on sections for 2 hr at 40C and then washed serially. Affymetrix Panomics probes included ApoE (type 6, catalogue number VA6-16904 and type 1, catalogue number VA1-18265), OMG (type 1, catalogue number VA1-18161), Sox4 (type 6, catalogue number VA6-18162), CCND2 (type 6, catalogue number VA6-18266), Ki67 (type 1, catalogue number VA1-11033). Signal was amplified using PreAmplifier mix QT for 25 min at 40C followed by Amplifier mix QT for 15 min at 40C, and then signal was hybridized with labeled probe at 1:1000 dilution for 15 min at 40C. Color was developed using Fast Blue substrate for Type 6 probes and Fast Red substrate for Type 1 probes for 30 min at 40C. Tissue was counterstained with Gill's hematoxylin for 25 sec at room temperature followed by mounting with ADVANTAGE mounting media (Innovex). For quantification of compartments by ISH, at least 1,000 cells were counted in representative areas of the tumors.

### DNA Fluorescent In Situ Hybridization

The probes used in this study consisted of centromeric (CEP) and locus-specific identifiers (LSI) probes. Control probes included: centromere (CEP) 1 (10p11.1-q11.1, spectrum orange), CEP4 (4p11-q11, spectrum aqua), CEP7 (7p11.1-q11.1, spectrum aqua), CEP10 (10p11.1-q11.1, spectrum aqua) and chromosome 19 control enumeration probe (19p13, Green 5-Fluorescein) except for chr19 enumeration probe that was purchased from Empire Genomic (Buffalo, N.Y.), all others were obtained from Abbott Molecular, Inc. (Des Plaines, Ill.). CEP probes included: CEP2 (2p11.1-q11.1, spectrum orange), CEP4 (4p11-q11, spectrum aqua), CEP9 (9p1-q11, spectrum aqua), CEP12 (12p1.1-q11, spectrum green), CEP17 (17p11.1-q11.1, spectrum aqua) and Y (Yp11.1-q11.1, spectrum green) all obtained from Abbott Molecular, Inc. (Des Plaines, Ill.). LSI probes were 1p36/1q25 and 19q13/19p13 dual-color probe set (Abbott), bacterial artificial chromosomes RP11-626F2 (19q13.2), RP11-112J7 (4q32.1), RP11-1065D4 (7q34), RP11-165M8 (10q23.31) labeled spectrum orange, RP11-54A4 (1q21.2-1q21.3), RP11-1061117 (1q44), RP11-11406 (7q31.2), RP11-1053E10 (10q25.1) labeled spectrum green all obtained from Children's Hospital Oakland Research Institute (CHORI, Oakland, Calif.). LSI probes were also bacterial artificial chromosome RP11-351D16 (10q11.21, spectrum red or green; CHORI, Oakland, Calif.).

FISH was performed as described previously (120). Briefly, 5- m sections of formalin-fixed, paraffin-embedded tumor material were deparaffinized, hydrated, and pretreated with 0.1% pepsin for 1 hour. Slides were then washed in 2× saline-sodium citrate buffer (SSC), dehydrated, air dried, and co-denatured at 80° C. for 5 minutes with a two or three-color probe panel and hybridized at 40° C. overnight using the Hybrite Hybridization System (Abbott). Two 2-3 min post-hybridization washes were performed in 2×SSC/0.3% NP40 at 72° C. followed by one 1 min wash in 2×SSC at room temperature. Slides were mounted with Vectashield containing 4′,6-diamidino-2-phenylindole (Vector, Burlingame, Calif., USA). Entire sections were observed with an Olympus BX61 fluorescent microscope equipped with a charge-coupled device camera and analysed with Cytovision software (Leica Biosystems, Buffalo Grove, Ill.). The LSI and control (CEP) signals were quantified in 50 randomly selected, non-overlapping nuclei and mean numbers of LSI copies and control (CEP) per nucleus were calculated. Scores were calculated and amplification was considered when LSI/control CEP ratio ≥2.0 and deletion was considered for ratio ≤0.75.

### Human NPC Culturing

Human NPCs were dissociated from the subventricular zone of 19 week fetal tissue and resulting neurospheres were expanded as previously described in a 50/50 mixture of DMEM/F12 and Neurobasal A (Invitrogen), supplemented with B27 lacking vitamin A, EGF, FGF, and heparin. Single live NPCs were isolated by FACS from a passage 8 culture and sorted into 96 well plates containing Buffer TCL (Qiagen)+1% beta-mercaptoethanol. For differentiation assays, NPCs were plated in chamber slides coated with poly-d-lysine and laminin, and proliferation media was exchanged over a period of 3 days with base media supplemented with either 1% FBS, 1% FBS+60 ng/mL T3, or FBS+100 nM trans-retinoic acid and 10 ng/mL NT3. Multipotency was confirmed by indirect immunofluorescence after 7 days of differentiation with GFAP (Abcam ab53554), Olig2 (Millipore AB9610), and Neurofilament (Aves).

### Single Cell RNA-Seq Data Processing

Expression levels were quantified as E_{i,j}=log₂(TPM_{i,j}/10+1), where TPM_{i,j} refers to transcript-per-million for gene i in sample j, as calculated by RSEM (60). TPM values are divided by 10 since Applicants estimate the complexity of single cell libraries in the order of 100,000 transcripts and would like to avoid counting each transcript ˜10 times, as would be the case with TPM, which may inflate the difference between the expression level of a gene in cells in which the gene is detected and those in which it is not detected.

For each cell, Applicants quantified two quality measures: the number of genes for which at least one read was mapped, and the average expression level of a curated list of housekeeping genes. Applicants then conservatively excluded all cells with either fewer than 3,000 detected genes or an average housekeeping expression level (E, as defined above) below 2.5. For the remaining cells, Applicants calculated the aggregate expression of each gene as Eₐ(i)log 2(average(TPM_{i,1 . . . n})+1), and excluded genes with Eₐ<4. For the remaining cells and genes, Applicants defined relative expression by centering the expression levels, Er_{i,j}=E_{i,j}−average[E_{i,1 . . . n}]. Centering was performed within each tumor separately in order to decrease the impact of inter-tumoral variability on the combined analysis of the tumors.

Analysis of Bulk RNA-Seq Profiles from Glioma Tumors from TCGA.

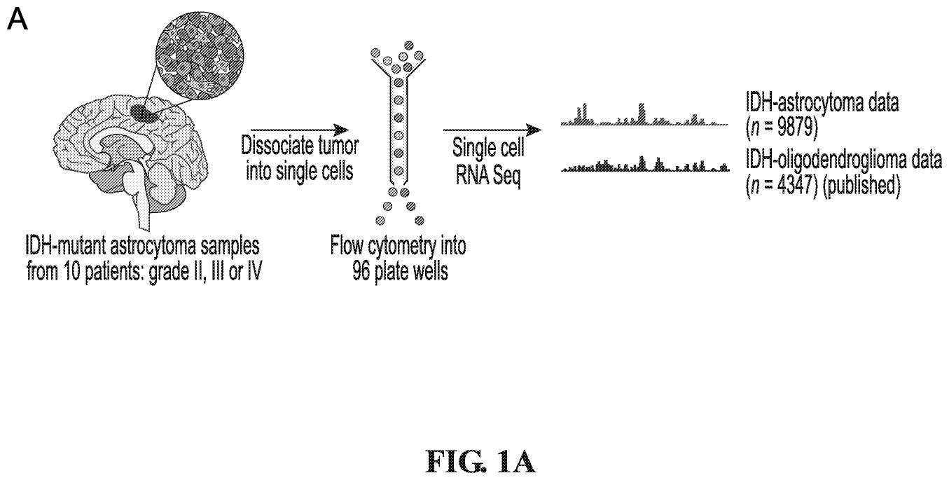

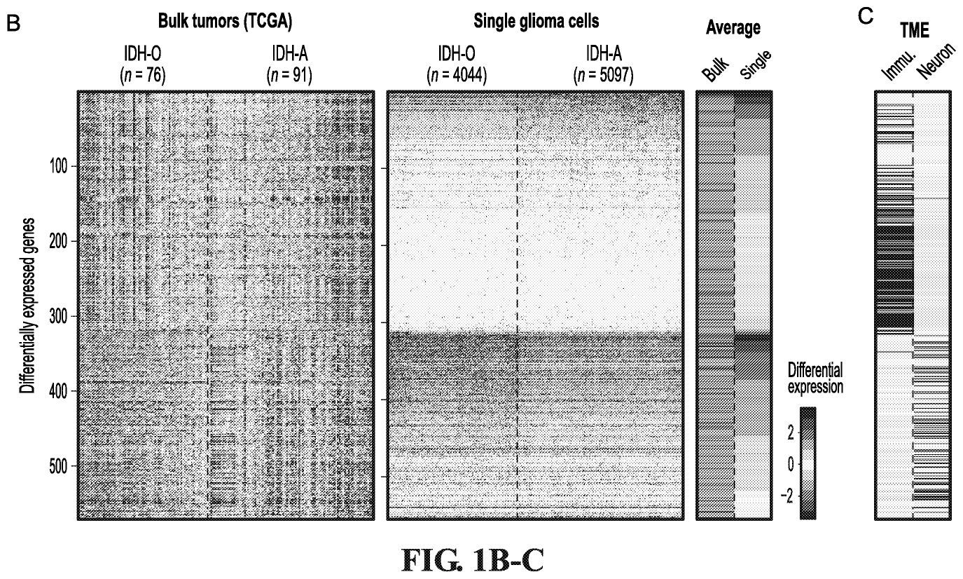

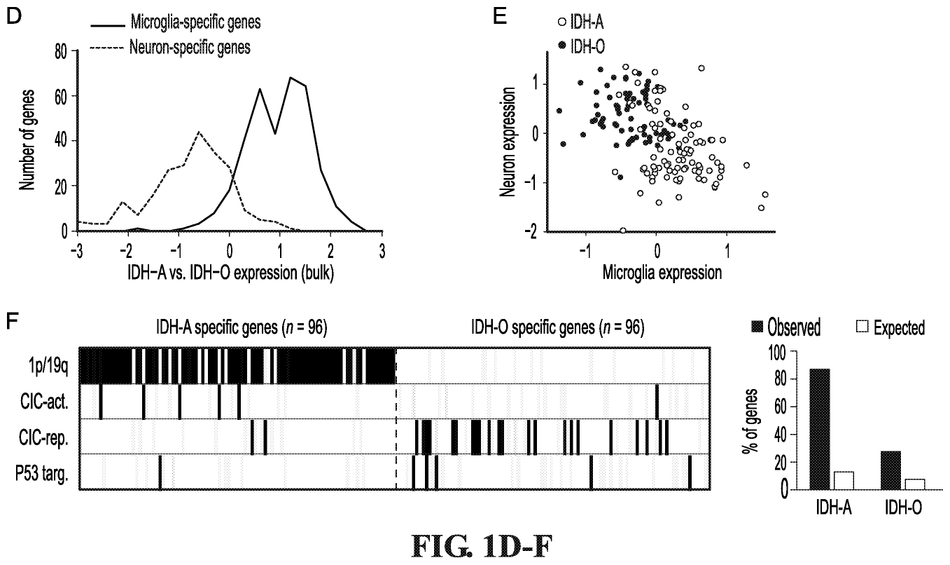

TCGA data was downloaded from the Broad Firehose website (gdac.broadinstitute.org/), including RNA-seq (rnaseqv2-RSEM_genes_normalized), mutation and copy number files from the GBMLGG dataset. Applicants used integrated molecular and histological classification to define 76 IDH-O tumors (oligodendroglioma histology plus IDH1/2 mutation and co-deletion of chromosome arms 1p and 19q), and 91 IDH-A tumors (astrocytoma histology plus IDH1/2 mutation, without co-deletion of chromosome arms 1p and 19q, and with mutations in P53 or ATRX). Applicants log 2-transformed the expression data of all tumors, restricted our analysis to 10,375 genes with an average expression above 4 (after log transformation), and then identified differentially expressed genes between IDH-A and IDH-O by a combination of fold-change and P-value criteria (based on t-test); the strict definition was based on fold-change of 2 and a P value of 10⁻⁵ (before correcting for multiple hypothesis testing), while the lenient definition was based on fold-change of 1.5 and a P-value of 10-. The strict definition was used to identify differentially expressed genes based on bulk analysis alone (and subsequently examine the genes in single cells, as shown in FIG. 1B), while the lenient definition was used as additional support for genes first detected as differentially expressed in single cell comparison of IDH-A and IDHO malignant cells. To define signature scores for bulk samples Applicants centered the expression log transformed values of each gene and calculated the average expression of the respective genesets.

### Classification of Single Cells to Malignant and Non-Malignant Cell Types

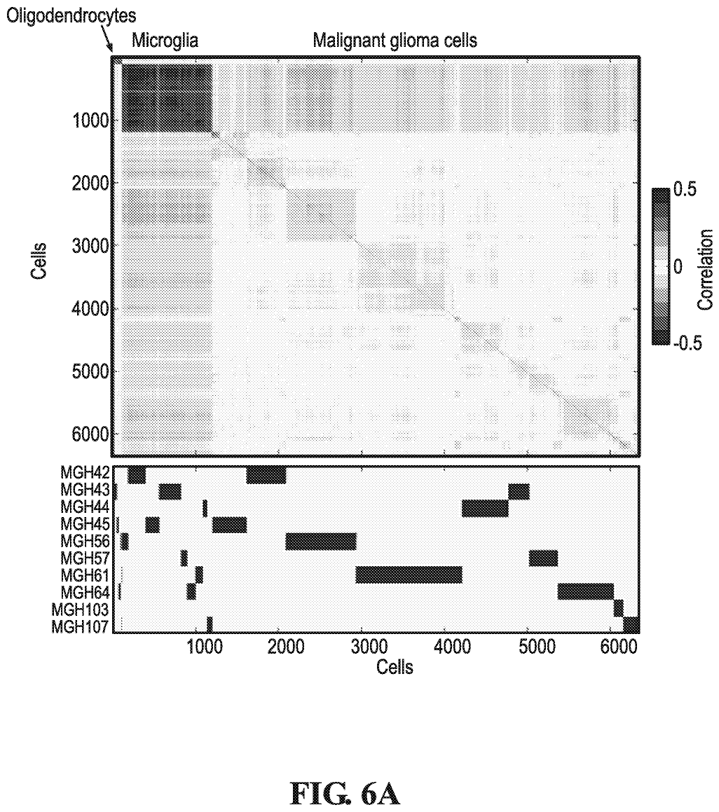

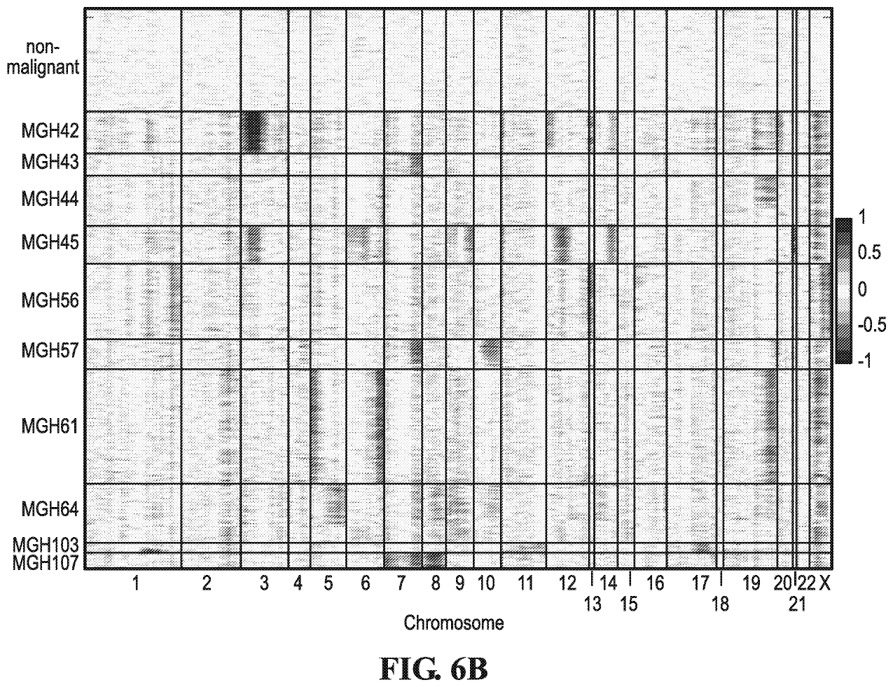

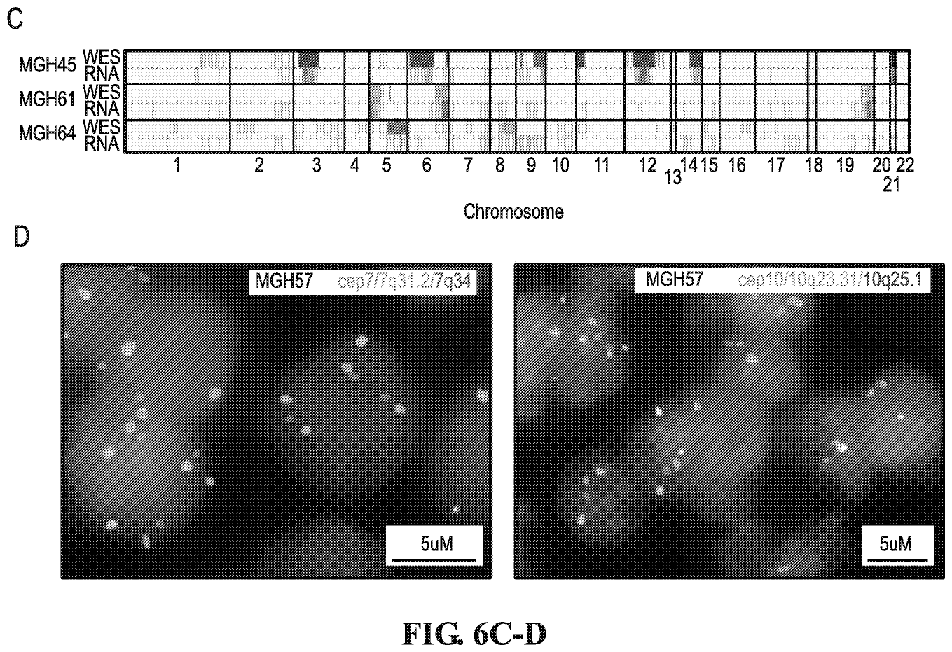

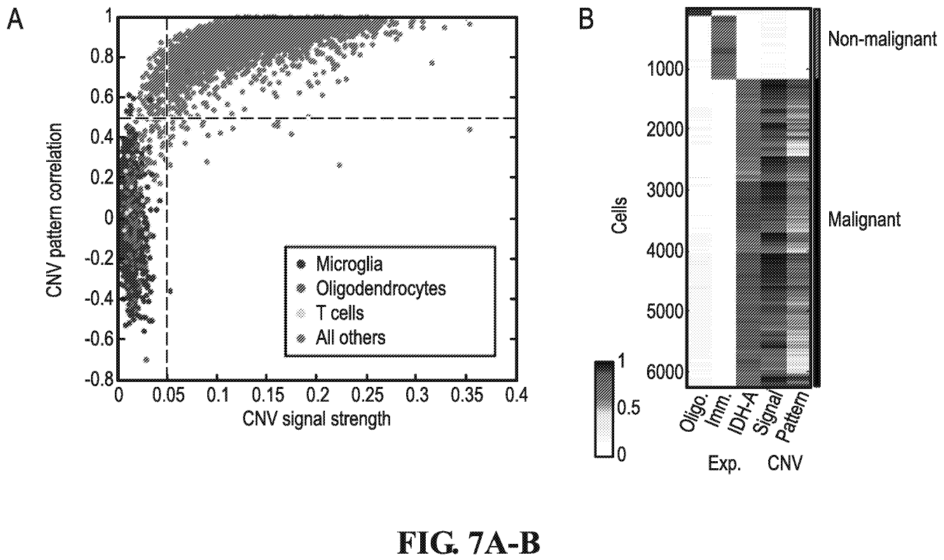

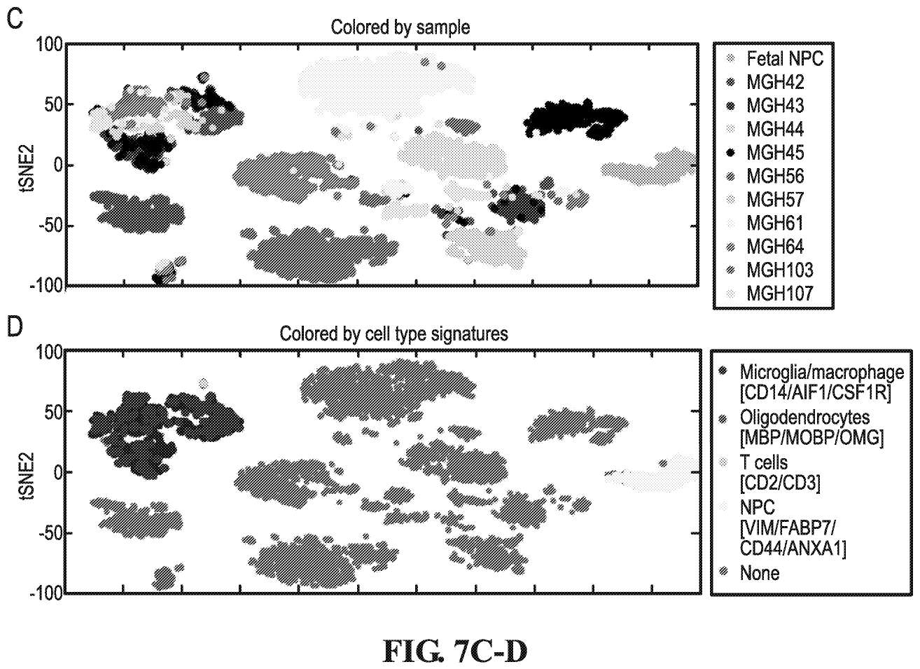

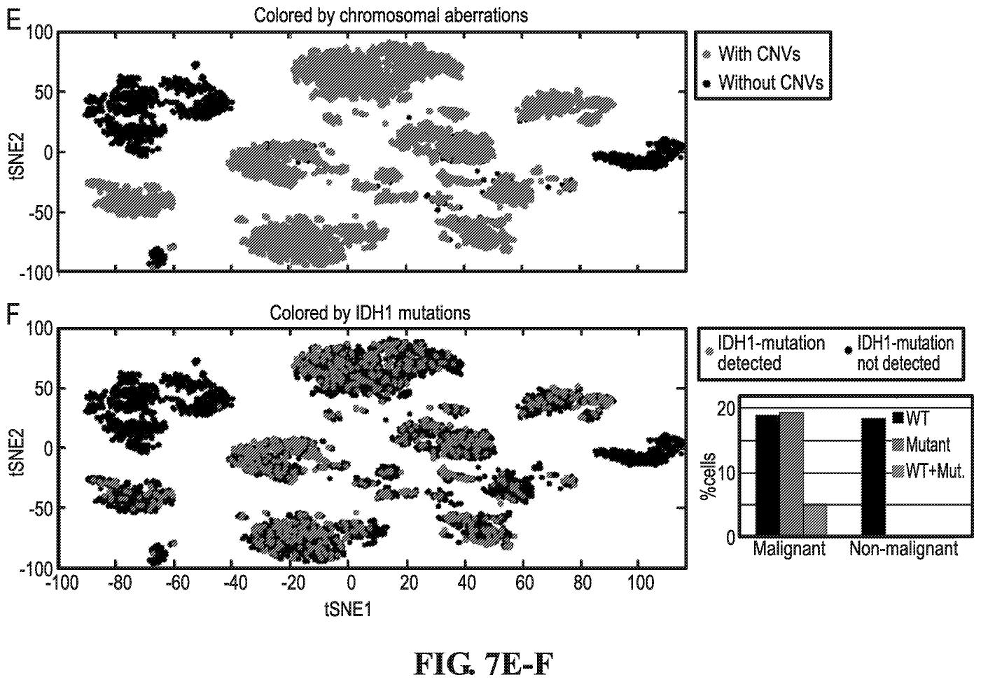

Hierarchal clustering of all IDH-A single cells revealed three main clusters (FIG. 6A), including cluster #1 that preferentially expressed oligodendrocytic markers (MBP, MOBP, PLLP, CLDN11) and cluster #2 that preferentially expressed markers of microglia or macrophages (CD14, CD163, CX3CR1, IFNGR1) and primarily included cells from plates which were sorted as CD45+ cells. Applicants thus hypothesized that the first two clusters reflect non-malignant oligodendrocytes and microglia/macrophages, while the third cluster corresponds to malignant cells. To further verify this, Applicants inferred chromosomal copy numbers, as described below (FIG. 6B). Applicants then defined two initial classifications based on gene expression and CNVs. First, Applicants scored cells by their correlation with the average expression profile of each cluster to derive expression based scores for oligodendrocytes, microglia/macrophages and malignant cells, and classified cells to the highest scoring cluster, if the correlation for that cluster was higher than that of the other clusters by at least 0.3; cells with a lower difference in correlation scores were defined as borderline. Second, Applicants classified cells as malignant, non-malignant and borderline based on the extent and profile of CNVs. Applicants scored each cell for the extent of CNV signal, defined as the sum of squares of CNV values across the genome, and for the correlation between the CNV profile of each cell with the average CNV profile of all cells from the corresponding tumor that are classified by expression as malignant. Applicants defined malignant cells as those with CNV signal above 0.05 and CNV correlation above 0.5 (FIG. 7A); non-malignant as those that satisfy neither of these thresholds; and borderline as those that satisfy only one threshold. Finally, Applicants classified cells as oligodendrocytes or microglia/macrophages if they were defined as non-malignant by CNV and as the corresponding expression cluster; and Applicants classified cells as malignant if they were classified as such either in both expression and CNV analysis or in one of those analyses but as borderline in the other analysis.

### CNV Estimation

Initial CNVs (CNV₀) were estimated by sorting the analyzed genes by their chromosomal location and applying a moving average to the relative expression values, with a sliding window of 100 genes within each chromosome, as previously described herein. To avoid considerable impact of any particular gene on the moving average, Applicants limited the relative expression values to [−3,3] by replacing all values above 3 by a ceiling of 3, and replacing values below −3 by a floor of −3. This was performed only in the context of CNV estimation. For visualization purposes, in order to include the two chromosomes with fewest analyzed genes (chromosome 18 and 21 with 105 and 75 genes, respectively) Applicants extended the moving average to include up to 50 genes from the flanking chromosomes (e.g. the first window in chromosome 18 consisted of the last 50 genes of chromosome 17 and the first 50 genes of chromosome 18, while the 51 through 56 windows in that chromosome consisted only of chromosome 18 genes). This initial analysis is based on the average expression of genes in each cell compared to the other cells and therefore does not have a proper reference to define the baseline. However, Applicants detected a cluster of cells that have higher values at chromosome 1p and 19q, which Applicants know are deleted in three oligodendroglioma tumors, and that have consistent “CNV patterns” across the genome despite the fact that they originate from all three tumors. Applicants thus defined the gene expression clusters annotated as oligodendrocytes and microglia/macrophages by gene expression as the nonmalignant cells, and used the average CNV estimate at each gene across those cells as the baseline. As the non-malignant cells include both microglia/macrophages and oligodendrocytes, which differ in gene expression patterns and therefore also in expression-based CNV estimates, Applicants defined two baselines, as the average of all microglia and the average of all oligodendrocytes, and based on these the maximal (BaseMax) and minimal (BaseMin) baseline at each window. The final CNV estimate of cell i at position j was defined as:

$${CNV}_{f} ( i , j ) = \{ \begin{matrix}{CNV}_{0} ( i , j ) - BaseMax ( j ) , & if {CNV}_{0} ( i , j ) > BaseMax ( j ) + 0.2 \\ {CNV}_{0} ( i , j ) - BaseMin ( j ) , & if {CNV}_{0} ( i , j ) < BaseMax ( j ) - 0.2 \\ 0 , & if BaseMin ( j ) - 0.2 < {CNV}_{0} ( i , j ) < BaseMin ( j ) + 0.2\end{matrix}$$

Single Cell Comparison of IDH-A and IDH-O Malignant Cells

Applicants compared the average relative expression of each gene between all malignant IDH-A and IDH-O cells and defined a fold-change difference. To assign a P-value, Applicants shuffled the assignments of cells to tumor types 10,000 times and counted the fraction of times where an equal or larger difference is obtained for subsets of cells of the same size as the IDH-O and IDHO cells. Applicants then defined differentially expressed genes as those with fold-change of 2 and P<0.01. The extent to which differential expression in single cell analysis recapitulates the differences observed in bulk analysis depends on the choice of specific thresholds, and therefore Applicants examined these fractions with a range of thresholds (FIG. 8A).

### Principal Component Analysis

Applicants performed principal component analysis (PCA) for the relative expression values of all malignant cells (as defined by CNV analysis). The covariance matrix used for PCA was generated using an approach previously outlined (61) to decrease the weight of less reliable “missing” values in the data. Due to the limited sensitivity of single cell RNA-seq, many genes are not detected in individual cells despite being expressed. This is particularly pronounced for genes that are more lowly expressed, and for cells that have lower library complexity (i.e., for which relatively fewer genes are detected), and results in non-random patterns in the data, whereby cells may cluster based on their complexity and genes may cluster based on their expression levels, rather than “true” co-variation. To mitigate this effect, Applicants assigned weights to missing values, such that the weight of E_{i,j} is proportional to the expectation that gene i will be detected in cell j given the average expression of gene i and the total complexity (number of detected genes) of cell j.

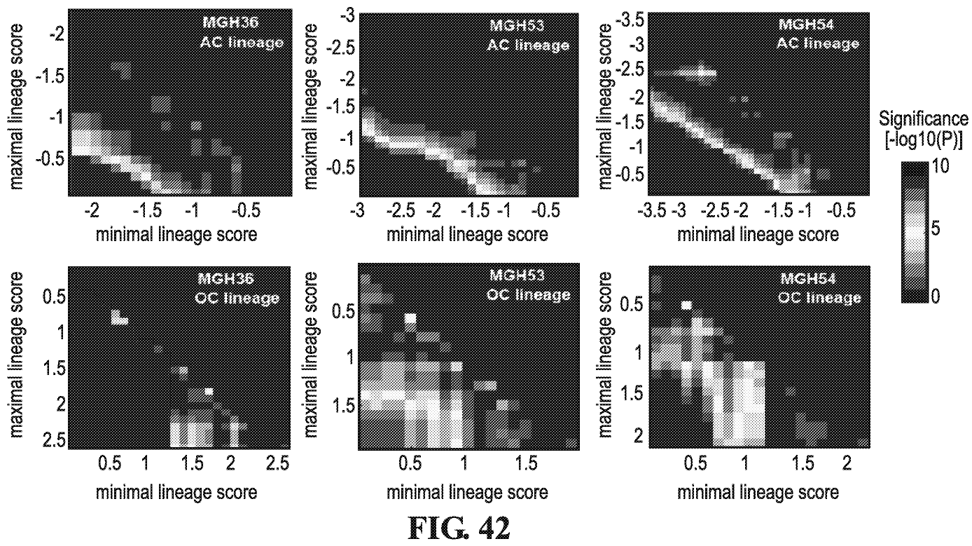

To further verify that the PCA results are not driven by library complexity Applicants compared the PCA results to those of shuffled data. Applicants iteratively swapped the expression of individual genes between pairs of cells with similar complexities, swapping each gene in each cell at least once. In that way Applicants shuffled the data and removed the biological clustering, but maintained the distribution of complexities across cells, as well as the distribution of expression levels for each gene. PCA over the shuffled data defined the complexity-based effect, as evident by a Pearson correlation of 0.96 between the PC1 cell scores and their complexities (in the original data this correlation is only 0.41). Applicants then compared PC1 gene scores between the original and the shuffled data (FIG. 42D). While PC1 gene scores of most genes are comparable between the two analyses, the loadings of the oligo and astro gene-sets were highly affected. Oligo genes were originally associated with highly positive PC1 scores, and their scores are significantly decreased upon shuffling (97% of the oligodendroglial genes were among the 5% genes with the most decreased loadings, P<10⁻³²); similarly, astrocytic genes were originally associated with negative PC1 scores, and their scores are significantly increased upon shuffling (all astrocytic genes were among the 5% genes with most increased loadings, P<10⁻³²). As a result, none of the genes with highest and lowest PC1 scores (after shuffling) overlap with our oligodendroglial and astrocytic gene-sets. Thus, complexity does not account for the association of PC1 with the differentiation programs. Similarly, complexity clearly does not account for the PC2/3 stemness program, as PC2 cell scores are positively correlated with complexity (R=0.27), while PC3 cell scores are negatively correlated with complexity (R=−0.24) and stemness genes were defined as those associated with both PC2 and PC3.

PC1-Associated Genes and Lineage Scores

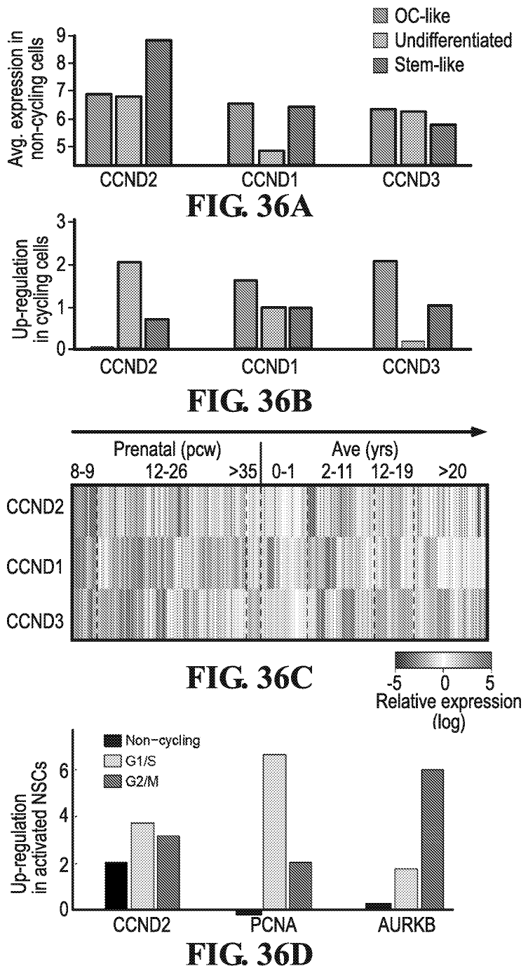

The top correlated genes with PC1 scores (across all tumor cells) were defined as PC1-associated genes. Applicants focused on the genes with an absolute correlation value above 0.35, but note that other thresholds gave similar results (not shown). Of those genes, the subset that was differentially expressed by at least 3-fold between OC and AC mouse cells (97), and for which the two comparisons were consistent (i.e., PC1-positively correlated genes with higher OC expression, and PC1-negatively correlated genes with higher AC expression) were defined as the OC and AC lineage gene-sets. Lineage scores were then calculated as the average relative expression of the lineage gene-set minus the average relative expression of a control gene-set, i.e. Lin_{i,j}=average[Er(G_{j,i})]−average[Er(Gⱼ^{cont},i)], where Lin_{i,j} is the score of cell i to lineage j, Gⱼ is the gene-set for lineage j and Gⱼ^{cont} is a control gene-set for lineage j. The control gene-set was defined by first binning all 8008 analyzed genes into 25 bins of aggregate expression levels and then, for each gene in the lineage gene-set, randomly select 100 genes from the same expression bin. In this way, the control gene-set has a comparable distribution of expression levels to that of the lineage gene-set and the control gene set is 100-fold larger, such that its average expression is analogous to averaging over 100 randomly-selected gene-sets of the same size as the lineage gene-set. The final lineage score of each cell was defined as the maximal score over the two lineages, LINᵢ=max(Linᵢ OC, Linᵢ AC). For visualization purposes in FIG. 36, 37, 38 and in FIGS. 48, 49 and 55 where the two lineage scores are shown in a single axis, Applicants first assigned random scores within [0-0.15] to all cells with LIN<0, to avoid having many overlapping cells at X=0. Second, Applicants assigned negative scores to the cells with higher AC than OC scores (i.e. a cell with AC and OC scores of 0.1 and 1, respectively would be assigned a lineage score of −1 while a cell with AC and OC scores of 1 and 0.1 would be assigned a lineage score of 1).

PC2 3-Associated Genes and Stemness Scores

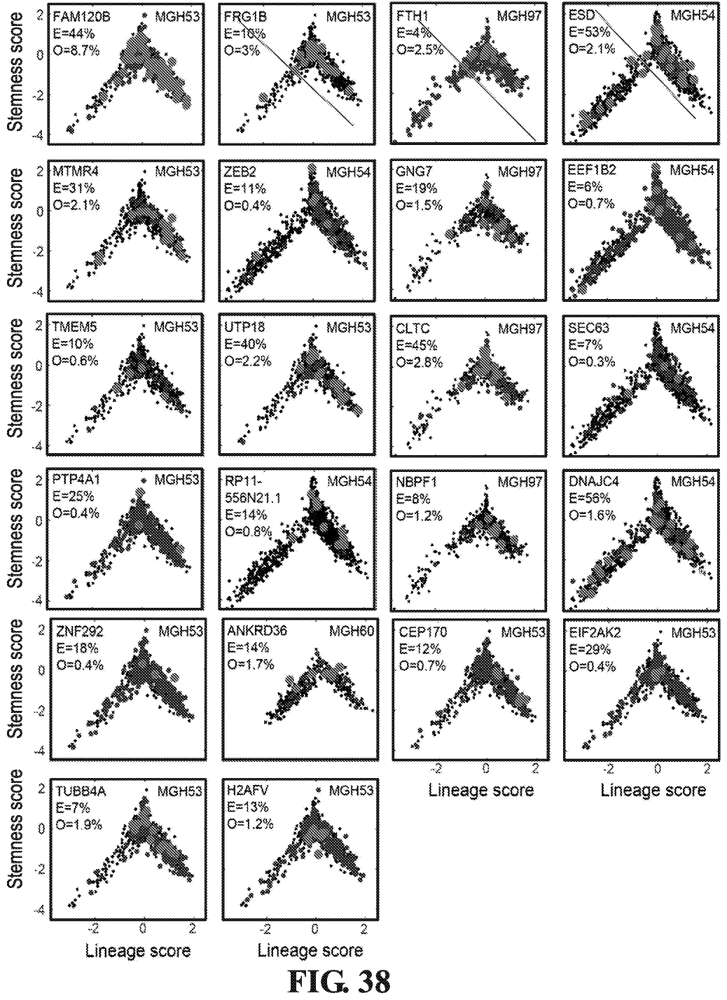

Both PC2 and PC3 were associated with intermediate values of PC1 (FIG. 38) and therefore with presumably less differentiated cells, and Applicants considered their sum as a potential stemness program. To detect potential stem-related genes Applicants chose the top 100 most positively correlated genes with PC2+PC3 scores across all cancer cells from the three tumors. The 100 candidate genes were then restricted to (1) genes that are positively correlated with both PC2 and PC3, which primarily excluded ribosomal protein genes that were only correlated with PC2; (2) genes for which the average relative expression among the stem-like cells (top third of cells by PC2+PC3 scores with a zero lineage score) was above average. Stemness scores for each cell, stem(i), were then defined as the average relative expression of the stemness gene-set (Gₛₜₑₘ) minus the average of a control gene set (Gₛₜₑₘ^{cont}) and minus the lineage score of cell i.

Stem(i)=average[Er(Gₛₜₑₘ)]−average[Er(Gₛₜₑₘ^{cont})]−LIN(i)

Assignment of Cells to Four Subpopulations: Stem/Progenitor-Like, Undifferentiated, OC-Like and AC-Like

Cells were scored for the three programs defined above (two lineage scores and a stemness score) and assigned to the subpopulation that corresponds to their highest scoring program, if the maximal score was above 0.5 and was higher by 0.5 than the score for the other programs. Cells in which the maximal score did not pass these thresholds were assigned to the undifferentiated subpopulation, for which Applicants did not detect a specific expression program. Applicants note that the expression programs are continuous and thus it is difficult to assign all cells to discrete subpopulations. Nevertheless, most cells are highly biased towards one of the three states, and the overall estimates are consistent between analysis of single cell RNA-seq data and tissue staining experiments (FIG. 36F, Table 4). Furthermore, very few cells (˜1% on average, and 5% at most) scored for two programs simultaneously (with the same threshold of 0.5 and no additional criteria, Table 4), with an average frequency of ˜1% of and a maximal frequency of ˜5% cells across the different combinations of programs and different tumors.

### Definition of Cell Type-Specific Gene-Sets

Applicants defined astrocytic-specific, oligodendrocytic-specific, neuron-specific and endothelial specific gene-sets using RNA-seq data from sorted cell types from mouse brain (97). For each cell type, Applicants identified genes with a higher expression in the respective cell type than in all other brain cell types (astrocytes, oligodendrocytes, neurons, endothelial cells and microglia) by at least 4 fold. As a more lenient definition (FIG. 8), Applicants reduced this threshold to 2-fold. Microglia/macrophage-specific genes were defined based on the IDH-A and IDH-O single cell data, comparing the average expression of all microglia/macrophage cells to that of malignant cells and to that of oligodendrocytes with a 8-fold threshold (in both comparisons); a 2-fold threshold was used for the lenient definition in FIG. 8.

### Defining Cell and Sample Scores

Given a set of genes (Gⱼ) reflecting a specific cell type or biological function, Applicants define a score, SCⱼ(i), for each cell i, quantifying the relative expression of Gⱼ in cell i, as the average relative expression (Er) of the genes in Gⱼ, compared to the average relative expression of a control gene set (Gⱼ^{cont}): SCⱼ(i)=average[Er(Gⱼ,i)]−average[Er(Gⱼ^{cont},i)]. The control gene-set is defined by first binning all analyzed genes into 25 bins of aggregate expression levels and then, for each gene in the considered gene-set, randomly selecting 100 genes from the same expression bin. In this way, the control gene-set has a comparable distribution of expression levels to that of the considered gene-set and the control gene set is 100-fold larger, such that its average expression is analogous to averaging over 100 randomly-selected gene-sets of the same size as the considered gene-set. A similar approach was used to define bulk sample scores.

### Genetic Causes of Expression Differences Between IDH-A and IDH-O Malignant Cells

To test the degree to which expression differences between TDH-A and IDH-O could be explained by known genetic differences, Applicants focused on genetic events specific to IDH-O (codeletion of chromosome arms 1p and 19q, decreased or loss of function of the transcriptional repressor CIC) and those specific to IDH-A (mutations in P53 and ATRX). The immediate impact of the co-deletion is reduction in the expression of all genes on the corresponding chromosome arms. Additional effects could reflect trans-effects, e.g. due to reduced expression of regulators on these chromosomes; while these effects are generally difficult to infer, one of the regulators on these chromosomes is CIC, which is further mutated (i.e. causing loss-of-function of the second allele) in most IDH-O tumors, and thus reduced CIC activity is a universal feature of IDH-O that is driven by both co-deletion and additional loss of function mutations. To infer the effects of reduced CIC activity, Applicants combined the results of two analyses. First, Applicants identified a subclonal CIC mutation in the oligodendroglioma MGH53, as described herein, and defined subsets of mutant cells and wild-type cells by single cell analysis, thus enabling a direct comparison and identification of differentially expressed genes within the same tumor. Second, Applicants compared the expression of all IDH-O TCGA tumors with a CIC mutation to those without CIC mutations and identified differentially expressed genes that are either activated or repressed by CIC, using a fold-change threshold of 2 and a t-test p-value of 0.01. Applicants combined the results of these two analyses to define putative sets of CIC repressed and activated genes. P53 targets were defined based on chromatin-immunoprecipitation and presence of a binding motif (134).

### Lineage and Differentiation Scores

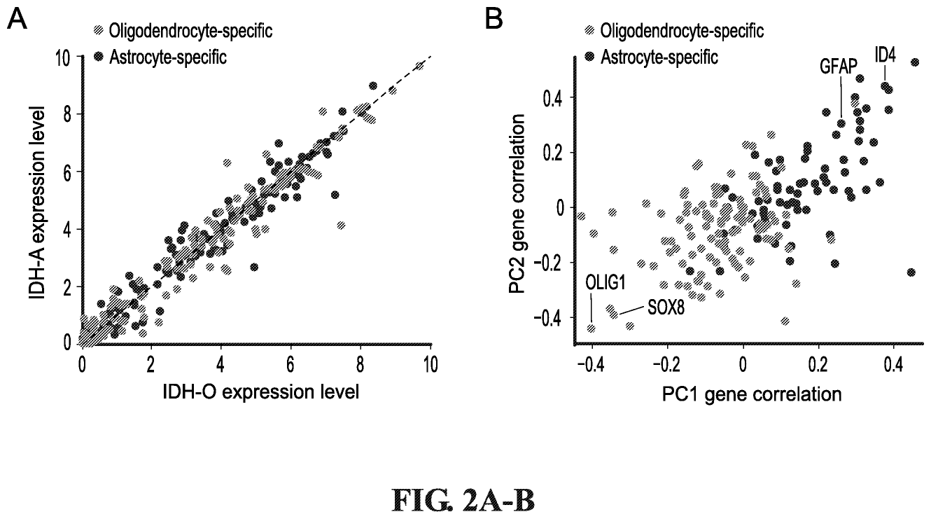

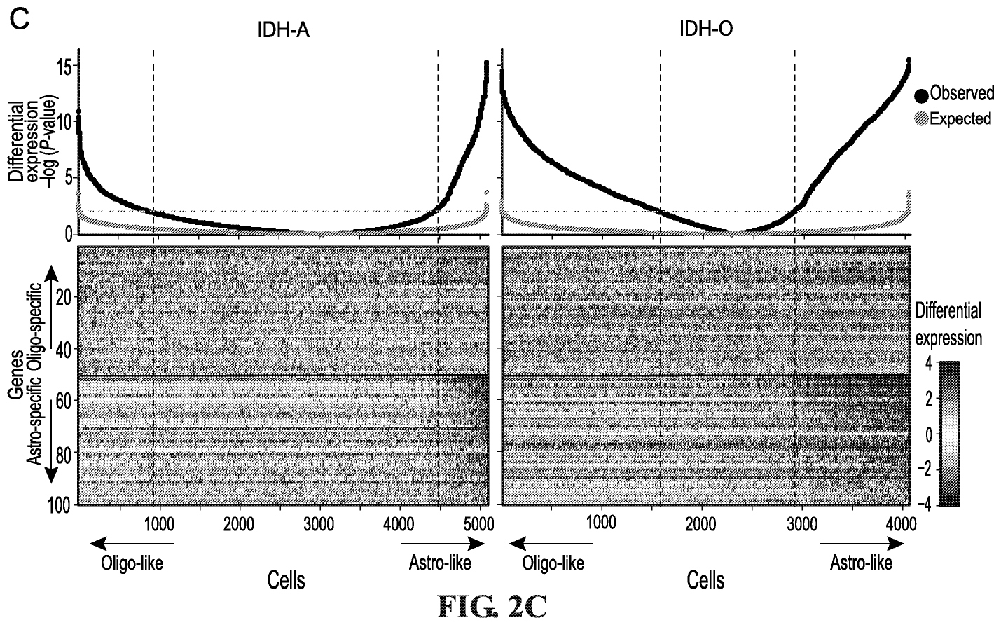

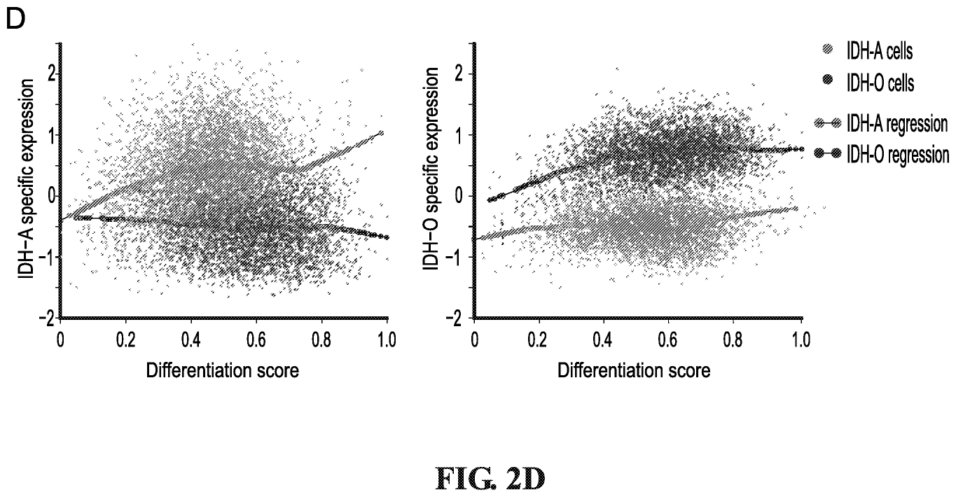

Variability among malignant IDH-A cells, as reflected by the first principal component (PC1), is consistent with astrocyte-specific (PC1-low genes) and oligodendrocyte-specific (PC1-high) genes (FIG. 2B; Table 2). However, this consistency is partial, reflecting the differences between differentiation programs as measured in mice (97) and as Applicants observe in IDH-A and IDH-O tumors. To refine the definition of these expression programs in the context of IDH-A, Applicants first scored each cell based on the expression of the above gene-sets to define initial astrocytic and oligodendrocytic scores (SCₐₛₜᵣₒ and SC_{oligo}). Applicants then calculated the correlation of each gene with SCₐₛₜᵣₒ-SC_{oligo} across all malignant IDH-A cells. The 50 genes with highest and 50 genes with lowest correlations were then used to define the refined astrocytic and oligodendrocytic scores (SC^{ref}ₐₛₜᵣₒ and SC^{ref}_{oligo}), which were used in all subsequent analyses. Thus, genes associated with glial differentiation that do not correlate with the program in the tumor cells were removed, while other genes which are not known to be involved in glial differentiation, but are co-expressed with the glial programs, are added, resulting in gene-sets which are coherently expressed across tumor cells but maintain high similarity to developmental glial expression programs. Applicants then scaled these scores to the range [0 1], by subtracting the minimal score and dividing by the range of scores. Finally, Applicants defined a differentiation score for each cell (regardless of lineage) as max(SC^{ref}ₐₛₜᵣₒ, SC^{ref}_{oligo}).

### Cell Cycle Analysis

Gene-sets reflecting the expression program of the G1/S and G2/M phases of the cell cycle were defined as the overlap between gene-sets identified in several previous studies, as described previously (11). Applicants used the average relative expression of these gene-set to derive G1/S and G2/M scores. Cycling cells were defined as those in which one of the scores was above 1.5 and where the P-value from one sample t-test over the corresponding gene-set was below 10′.

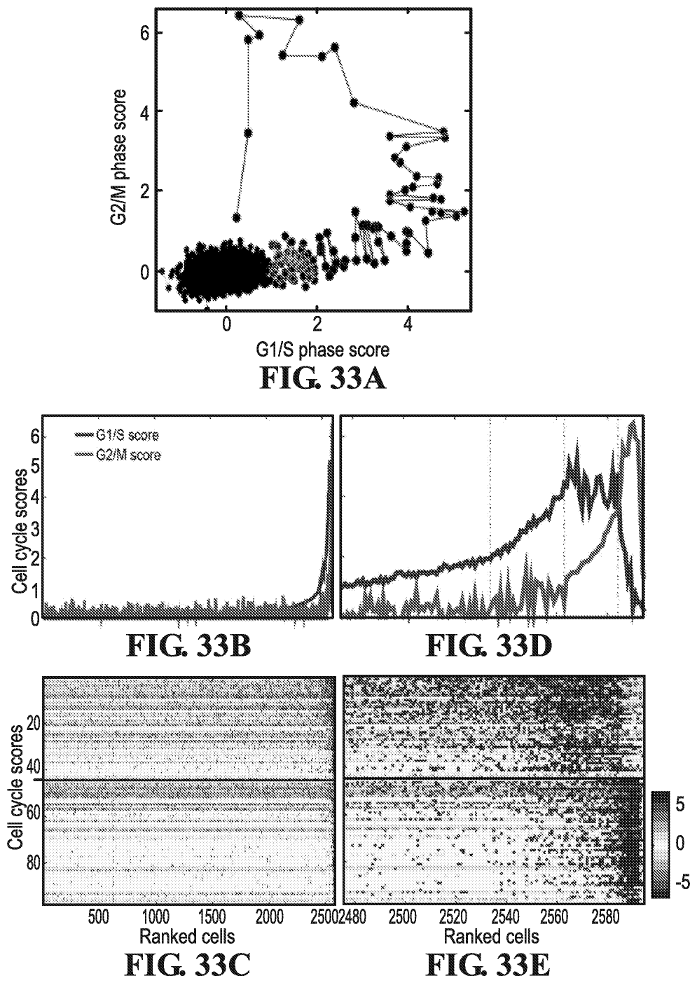

Analysis of single-cell RNA-seq in human (293T) and mouse (3T3) cell lines (16), and in mouse hematopoietic stem cells (124) revealed in each case two prominent cell cycle expression programs that overlap considerably with genes that are known to function in replication and mitosis, respectively, and that have also been found to be expressed at G1/S phases and G2/M phases, respectively, in bulk samples of synchronized HeLa cells (62). Applicants thus defined a core set of 43 G1/S and 55 G2/M genes that included those genes that were detected in the corresponding expression clusters in all four datasets from the three studies described above (Table 2). As expected, the genes in each of those expression programs were highly co-regulated in a small fraction of the oligodendroglioma cells, such that some cells expressed only the G1/S or the G2/M programs and other cells expressed both programs (FIG. 33). Plotting the average expression of these programs revealed an approximate circle (FIG. 33A), which Applicants speculate describes the progression along the cell cycle. While Applicants cannot confidently define the regions that correspond to each phase of the cell cycle in an automatic way, Applicants manually defined four regions in the apparent circle and assigned them to approximate cell cycle phases.

### Identification of a Putative Stemness Program

Applicants searched for genes that are preferentially expressed in undifferentiated cells, after excluding cycling cells, in order to avoid cell-cycle related effects. In each tumor, Applicants compared the average relative expression of each gene between undifferentiated cells (differentiation score below 0.25) and differentiated cells (differentiation score above 0.4), separated into those with a higher astrocytic or a higher oligodendrocytic score. This resulted in two values of fold-change (undif vs. astro-like and vs. oligo-like) and two corresponding P-values, which were calculated by shuffling cell identities 10,000 times. Significant genes were defined in each tumor as those with a fold-change above 1.5 and a P-value below 0.05; Applicants used these lenient criteria within each tumor due to the limited number of undifferentiated cells, but then focused on genes that were significant across multiple tumors. A control analysis after shuffling cell identities within each tumor led to genes that were significant in one or at most two tumors, and thus Applicants used a threshold of significance in three tumors. Ninety genes satisfied this criterion. To restrict those genes to a subset of coherently regulated genes that may reflect a stemness program, Applicants hierarchically clustered the genes in IDH-A and in IDH-O using 1-R, where R is a Pearson correlation coefficient across all undifferentiated cells in the corresponding tumor type. In both IDH-A and IDH-O Applicants observed one dominant cluster; Applicants defined that cluster as the largest cluster when cutting the hierarchical clustering tree at a correlation of R=0.4. Applicants then ranked the genes by their association with that cluster, defined as the average correlation with the genes in that cluster.

### Analysis of Microglia Macrophages

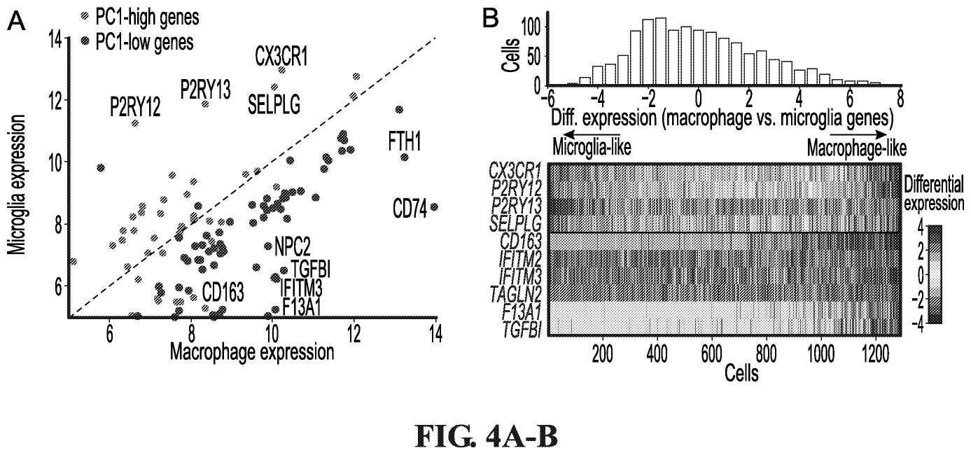

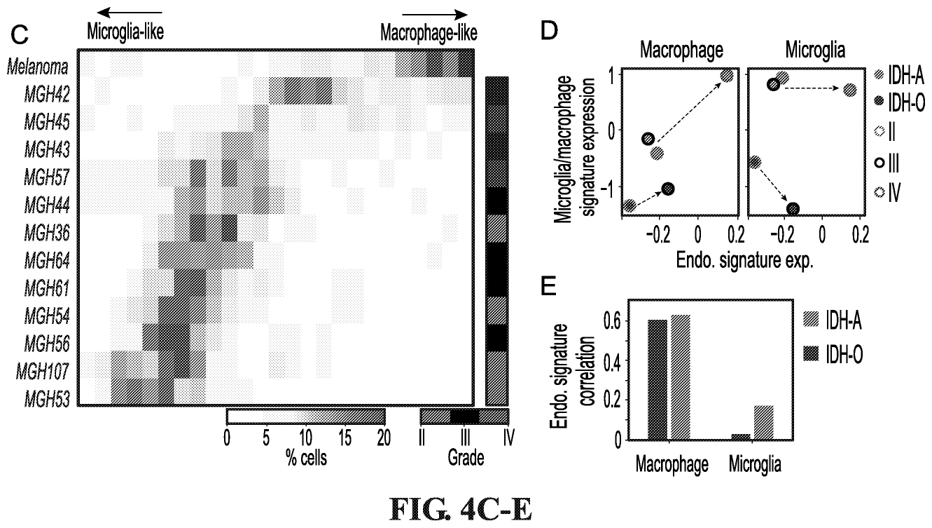

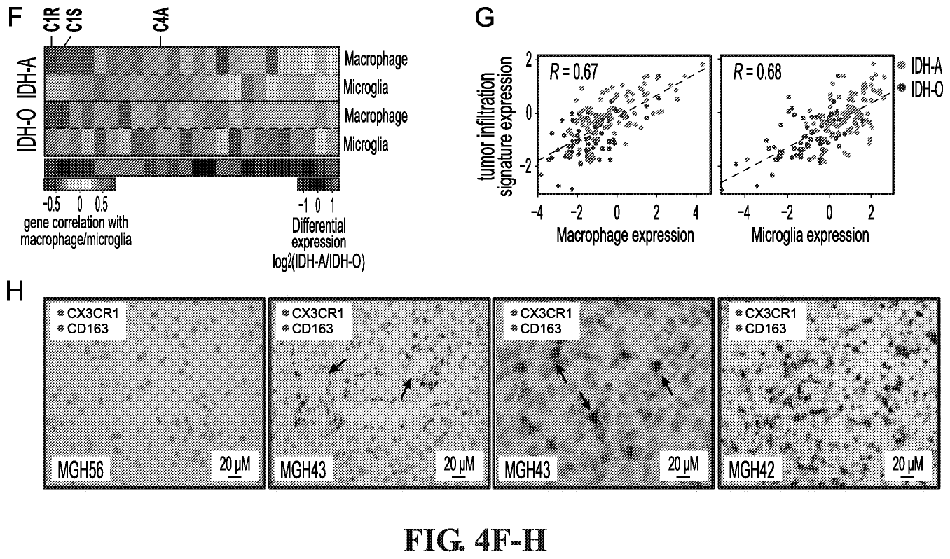

PCA was performed over the relative expression of all microglia/macrophages from IDH-A and IDH-O, including all genes with Ea>4 (defined only based on microglia/macrophages cells). PC1 genes were defined as those with a Pearson correlation above 0.3 (PC1-high genes) or below −0.3 (PC1-low genes). Applicants then examined the expression of the mouse orthologs of those genes in mouse microglia and macrophages (130); since multiple types of macrophages were previously profiled Applicants considered the maximal expression and the average expression of each gene across those macrophage subtypes. Applicants then defined microglia-specific genes as those with at least a 5- fold higher expression in microglia than the maximal macrophage expression, and macrophage specific genes as those with at least a 5-fold higher maximal macrophage expression than microglia expression, as well as at least a 2-fold higher average macrophage expression than microglia expression. Applicants focused on the genes that were defined as both microglia-specific and PC1-high (CX3CR1, P2RY12, P2RY13 and SELPLG), and on genes defined as both macrophage-specific and PC1-low (e.g., CD163, CD74, TGFBI, IFITM2, IFITM3, F13A1, NPC2, TAGLN2 and FTH1); the average relative expression of those genes defined the microglia-specific and macrophage-specific scores, and their difference defined the macrophage vs. microglia score, which is shown in FIG. 4B.

### Analysis of Whole-Exome DNA Sequencing Data

Output from Illumina software was processed by the Picard processing pipeline to yield BAM files containing aligned reads (bwa version 0.5.9, to the NCBI Human Reference Genome Build hg19) with well-calibrated quality scores (52, 53). Sample contamination by DNA originating from a different individual was assessed using ContEst57 (121). Somatic single nucleotide variations (sSNVs) were then detected using MuTect (55). Following this standard procedure, Applicants filter sSNVs by (1) removing potential DNA oxidation artifacts (122); (2) removing events seen in sequencing data of a large panel of ˜8,000 TCGA normal samples; (3) realigning identified sSNVs with NovoAlign (www.novocraft.com) and performing an additional iteration of MuTect with the newly aligned BAM files. sSNVs were finally annotated using Oncotator⁶⁰. Sample purity and ploidy, as well as Cancer Cell Fraction (CCF) of identified sSNVs were determined by ABSOLUTE (35). Genome-wide copy-ratio profiles were inferred using CapSeg. Read depth at capture targets in tumor samples was calibrated to estimate copy ratio using the depths observed in a panel of normal genomes. Next, Applicants performed allelic copy analysis using reference and alternate counts at germline heterozygous SNP sites.

### Mutation Calling in Single Cells

sSNVs that were identified by WES were examined in single-cell RNA-seq data by the mpileup command of SAMtools (Li, H. et al. Bioinformatics 25; 2078-2079 (2009)). The fraction of cells in which Applicants identified these mutations was, on average, only 1.3% of the expected fraction estimated by ABSOLUTE. This low sensitivity primarily reflects the low coverage of the RNA-seq reads over the transcriptome of single cells. Accordingly, sensitivity was correlated with the expression levels of the genes that harbor the mutations, and reached 20.4% for the top 10% most highly expressed genes. Sensitivity was also affected by heterozygosity and allele-specific expression, since in some heterozygote mutant cells Applicants might only sequence the wild-type allele.

Applicants used a targeted sequencing approach to increase our sensitivity for three specific mutations in MGH54 which were identified by WES but detected in very few cells by single cell RNA-seq. Applicants designed primers flanking these three mutations (in ZEB2, EEF1B2 and DNAJC4), PCR-amplified single cell cDNAs (frozen stocks of product from the pre-amplification reaction of the Smart-seq2 protocol) and sequenced the amplified material. This approach was applied for 1056 cells from MGH54. Mutant cells were defined as those with at least 50 reads that mapped to the mutant allele as defined by WES, and for which the fraction of mutant reads was at least 20% of all reads and 5-fold higher than the overall rate of mutant reads (in order to exclude a low rate of mutant reads due to PCR or sequencing errors). The mutations detected by this criteria were highly consistent with those identified from single cell RNA-seq (P<10⁻⁵, hypergeometric test) and uncovered 19 additional mutant calls (three for ZEB2, three for EEF1B2 and 13 for DNAJC4).

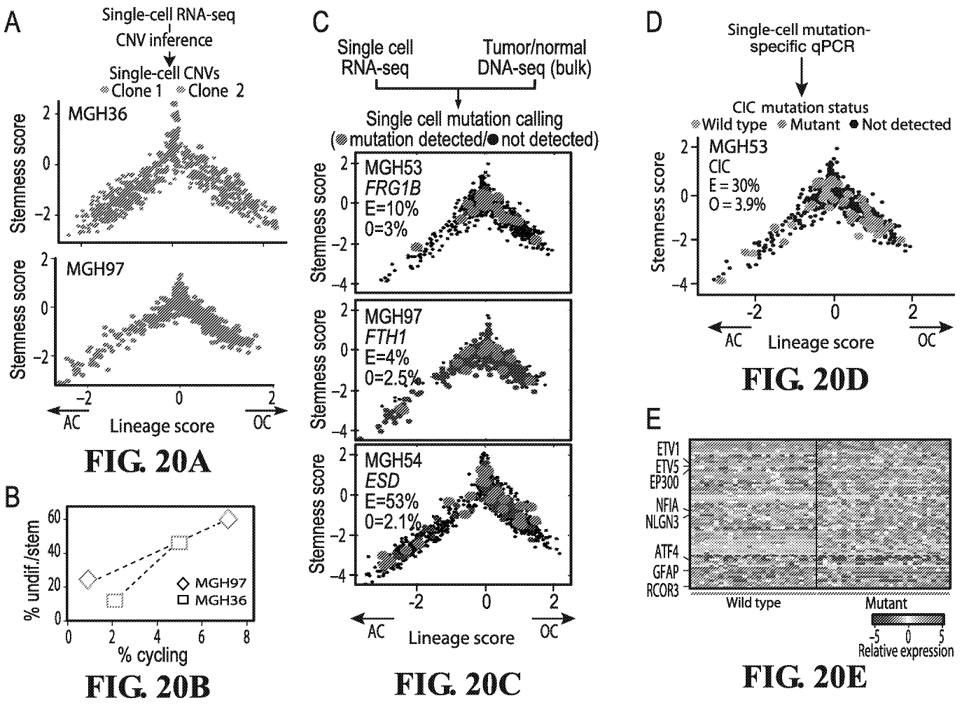

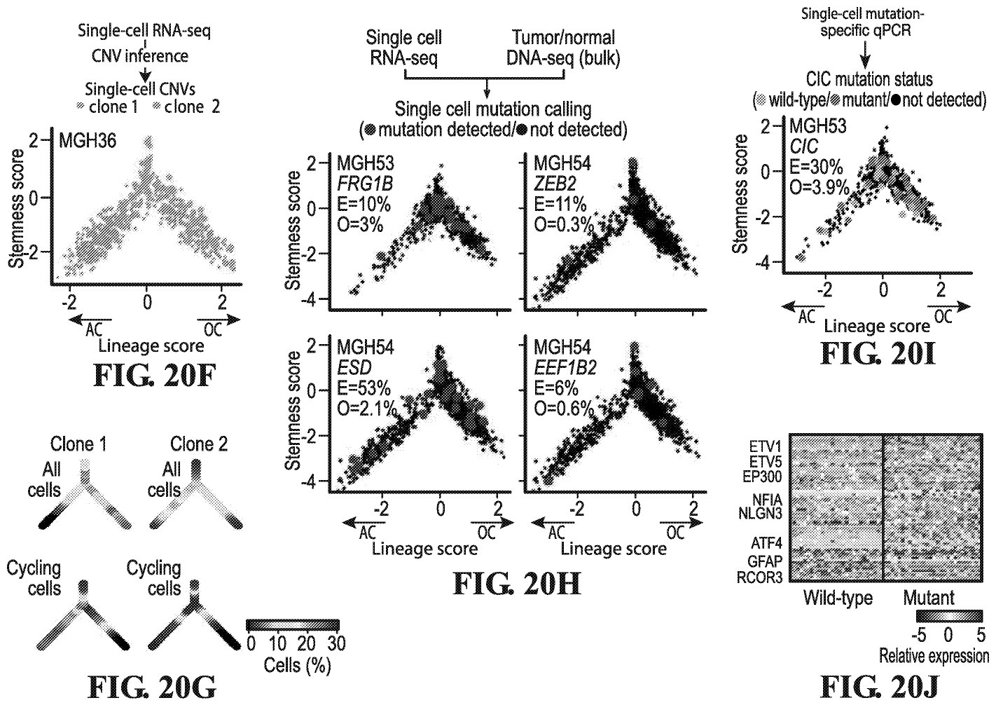

Applicants next focused on the 23 subclonal mutations for which (1) the estimated clonal fraction by ABSOLUTE was at most 60%; (2) at least three cells were identified as harboring the mutation; and (3) at least one cell was identified as having a wild-type allele of the mutant gene. For each of those 19 mutations Applicants plotted the lineage and stemness scores of all mutant cells to examine their distribution of expression states (FIG. 20 and FIG. 38). Note that for these 19 mutations Applicants detected on average 9.4% of the expected fraction by ABSOLUTE.

To estimate the frequency of false-positive errors Applicants defined, for each mutation that is detected by WES and analyzed by RNA-seq mutation calling, (i) “expected mutations”: the number of events in which Applicants find the exact mutation reported by WES, and (ii) “false mutations”: the number of events in which Applicants find a mismatch in the same exact site but to a different base than expected by WES (there are 2 such possible bases). This approach focuses on the exact genomic context of the real mutations to obtain a reliable estimate of the false positive rate. This estimate is half the number of false mutations divided by the number of expected mutations (given 4 bases, one of which is the WT, there are two type of “false mutations” but only one type of “expected mutations”). The result of this analysis was an estimated false positive rate of 0.85%, suggesting that the confidence of each detected mutation is higher than 99%. Accordingly, even in the most extreme case (e.g. ZEB2) where only a single mutant cell is detected in one of the compartments of the hierarchy, Applicants still have a 99% confidence that the mutation is represented in that compartment.

Mutation-Detecting qPCR and Analysis of CIC Mutations

To detect CIC mutations in single cells from MGH53, Applicants performed qPCR using SuperSelective PCR primers, which are highly specific to single base changes due to a loop-out sequence adjacent to the mutant base (legacy.labroots.com/user/webinars/details/id/95). The following qPCR primers were designed to target the c.4543 C>T, p.1515 R>C mutation on CIC cDNA which had been identified as subclonal in MGH53 via whole exome sequencing analysis:

Wild-type-specific forward:
(SEQ ID NO: 19)
5′-CCCTCCAAGGTTTGTCTGCAGccattcGAGGTGC-3′
Mutant-specific forward:
(SEQ ID NO: 20)
5′-CCCTCCAAGGTTTGTCTGCAGccattcGAGGTGT-3′
Universal reverse:
(SEQ ID NO: 21)
5′-tcgGGCAGCCTGCATGATCTT-3′

The specificity of the single cell qPCR primers was validated by two approaches. First, by qPCR on artificial templates differing by only the mutant base. Second, by qPCR on cDNA of single MGH53 tumor cells for which RNA-seq already detected mutant or wild-type reads. These positive control reactions were highly consistent between duplicates and with the mutation status as inferred from RNA-seq: qPCR identified 7 out of 7 mutant cells and 12 out of 15 wild-type cells while the remaining three cells had no qPCR signal, and therefore all qPCR signal was consistent with RNA-seq data. Applicants also took advantage of the fact that CIC is located on chr19q which is deleted in MGH53 cancer cells and therefore each cell only contains one CIC allele (loss-of-heterozygosity, LOH). Thus, in a single MGH53 cancer cell, Applicants expect evidence of either mutant or wild-type CIC, but not both. Indeed, all cells with a signal in the positive control assay showed difference in Ct of at least 5 between mutant and wild-type reactions, consistent with LOH.

cDNA was taken from frozen stocks of product from the preamplification reaction of the Smartseq2 protocol. 1 μl from each well of cDNA was used as template for a second round of Smartseq2 preamplification and bead purification in order to increase overall signal downstream. qPCR was performed with the Fast Plus EvaGreen qPCR Master Mix Low Rox (Biotium 31014-1) according to the manufacturer's instructions with the sole modification of adding EDTA to a final reaction concentration of 1.6 mM to enhance primer selectivity. Cp≥33 were considered negative signal; Cp<33 was considered positive signal.

Applicants performed SuperSelective qPCR on cDNA from 467 single MGH53 tumor cells. Of these, 61 cells had signal in both replicates for either mutant or wild type primers, but never for both. These were used to define 28 CIC mutant cells and 27 CIC wild-type cells, after excluding 6 cells which did not pass the single cell RNA-seq QC filters.

To identify genes regulated by the CIC mutation, Applicants compared the 28 CIC mutant and 27 CIC wild-type cells and identified genes with at least 2-fold average expression difference and P<0.01 (before correction for multiple hypothesis testing) based both on a permutation test and a t-test. To further filter the list of differentially expressed genes Applicants also compared the CIC mutant cells to the 671 unresolved cells (in which Applicants did not detect signal for either mutant or wild-type alleles by qPCR and by RNA-seq). Since the fraction of CIC mutants was estimated as 30% by ABSOLUTE Applicants expect the unresolved cells to be a mixture of ˜third CIC-mutants and ˜2/3 CIC-wild type cells, and thus CIC-regulated genes should also differ between this mixture and the CIC mutants but to a lower extent; Applicants used a threshold of 1.5-fold difference between the average expression in CIC mutants and in unresolved cells. The resulting set of differentially expressed genes is given in Table 6. Applicants simulated this analysis with 1,000 randomly selected sets of cells (to replace the CIC mutant and CIC wild-type cells) and found an average of only five upregulated genes by the same criteria, suggesting FDR<0.1 for the genes upregulated by CIC mutation.

### Example 2: Decoupling Genetic, Developmental and Micro-Environmental Programs in IDH-Mutant Gliomas Through Single-Cell RNA-Seq

Applicants reasoned that scRNA-seq of a limited number of representative tumors could be combined with existing bulk data from large cohorts to decouple these distinct effects, and sought to apply this approach to understand the differences between two types of diffuse gliomas. In adults, diffuse gliomas are classified into three main categories based on integrated genetic and histologic criteria: IDH-wildtype glioblastoma (GBM) is the most prevalent and aggressive form of the disease, while mutations in IDH1/2 define two major classes of gliomas: astrocytoma (IDH-A) and oligodendroglioma (IDH-O) (98). IDH-A and IDH-O are two distinct tumor types that differ in their genetics, histopathology and prognosis. Genetically, IDH-A are characterized by TP53 and ATRX mutations, while IDH-O are characterized by mutations in TERT promoter and loss of chromosome arms 1p and 19q, defining a robust genetic separation into two disease entities (112). In histopathology, IDH-A and IDH-O are distinct and thought to predominantly recapitulate astrocytic and oligodendrocytic lineage differentiation, respectively. The notion that lineages differ between astrocytoma and oligodendroglioma, as implied by their names, originates from distinct morphology and tissue staining. However, expression of both oligodendroglial (e.g., OLIG2) and astrocytic (e.g., GFAP) markers can be readily identified in both diseases (98), mixtures of cells with histological features of neoplastic astrocytic and oligodendroglial cells are frequently observed within individual tumors, and cellular morphologies are only partially reminiscent of distinct glial cells, thus questioning the hypothesis of distinct lineages. Two models may explain morphological differences in IDH-mutant gliomas: in one model, distinct glial cells or glial progenitor cells give rise to different types of gliomas; in another model, all IDH-mutant gliomas originate from the same progenitors, but distinct signature genetic events give rise to two different classes of tumors of different morphology (127).

Applicants first sought to classify single cells into malignant and non-malignant. While genetic mutations may be used for such classification, mutation calling from scRNA-seq has limited sensitivity and specificity and combined single-cell DNA and RNA profiling is not yet scalable to thousands of cells (135, 136). Applicants thus combined two complementary approaches. First, gene expression clustering separated cells into three groups, consistent with programs of glioma cells, immune cells and oligodendrocytes (FIG. 6). Second, since glioma cells frequently harbor large-scale chromosomal aberrations (112), Applicants estimated copy number variations (CNVs) from the average expression of genes in large chromosomal regions within each cell (69), and validated some of our predictions by whole exome sequencing and DNA FISH (FIG. 6; SOM). Expression-based and CNV-based classifications were highly consistent with one another, and Applicants used both criteria to identify 5,097 malignant cells (FIG. 7). The classification scheme was further validated by IDH mutations whose detection, while technically limited in scRNA-seq data, was highly specific to cells classified as malignant (FIG. 7; P<10⁻¹⁶, hypergeometric test). Applicants then directly compared the IDH-A malignant cells to 4,044 malignant cells that Applicants recently profiled from six IDH-O tumors (137) (FIG. 1B).

Surprisingly, only approximately half of the genes that were differentially expressed based on bulk TCGA samples were also differentially expressed between the single malignant cells of the two tumor types (FIG. 1B, FIG. 8), suggesting that the remaining differentially expressed genes may reflect differences in the TME rather than differences in the expression programs of malignant cells. Indeed, most of the remaining expression differences between bulk samples involved either microglia/macrophage-specific genes or neuron-specific genes (SOM), which were preferentially expressed in bulk IDH-A or IDH-O samples, respectively (FIG. 1C-E, FIG. 8). Differential expression between IDH-A and IDH-O was highly consistent both when considering microglia/macrophage-specific genes and when considering neuron-specific genes (FIG. 1D), allowing for the estimation of the relative abundance of microglia/macrophages and neuronal cells in each of the bulk tumors, based on the average expression of these two signatures (FIG. 1E). Thus, IDH-A tumors are associated with more microglia/macrophages and less neuronal cells than IDH-O tumors, with only few exceptions (FIG. 1E). Importantly, these differences are observed also between IDH-A and IDH-O tumors of the same clinical grade (FIG. 8).

Next, Applicants focused on the expression differences between IDH-A and IDH-O that are significant both when comparing bulk samples and between single malignant cells of the two tumor types (SOM). Applicants reasoned that genetic differences might determine at least some of these differences and indeed observed that most genes with higher expression in single malignant cells in IDH-A are located on chromosomes 1p and 19q, which are co-deleted in IDH-O (FIG. 1F). Loss-of-function of the transcriptional repressor CIC, another genetic event specific to IDH-O, accounted for an additional ˜10% of the expression differences (FIG. 1F), as inferred from a CIC expression signature Applicants defined from analysis of single cells and bulk samples (SOM) (137, 116). Applicants also found a limited, yet significant, enrichment (P=0.018, hypergeometric test) of p53 targets among genes more highly expressed in IDH-O tumors, consistent with a mutated TP53 in IDH-A. Since the effects of TP53 mutations are context-specific, and since Applicants were unable to define the expression signature for ATRX mutations, Applicants expect that genetic mutations further contribute to the remaining differences. Taken together, these results suggest that differences between bulk TCGA expression signatures of IDH-A and IDH-O primarily reflect genetic influences (in the malignant cells) and TME composition.

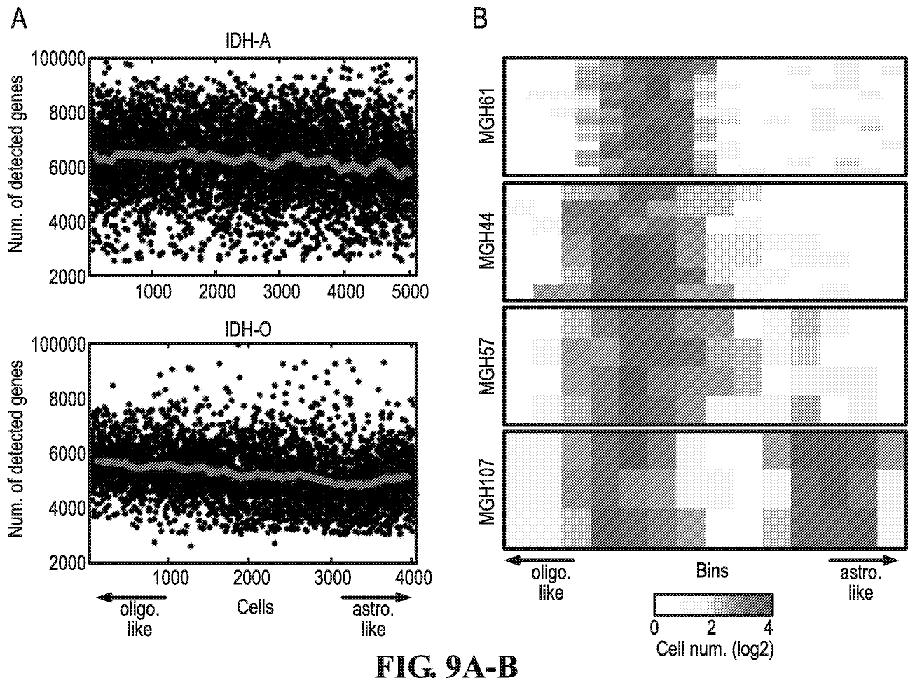

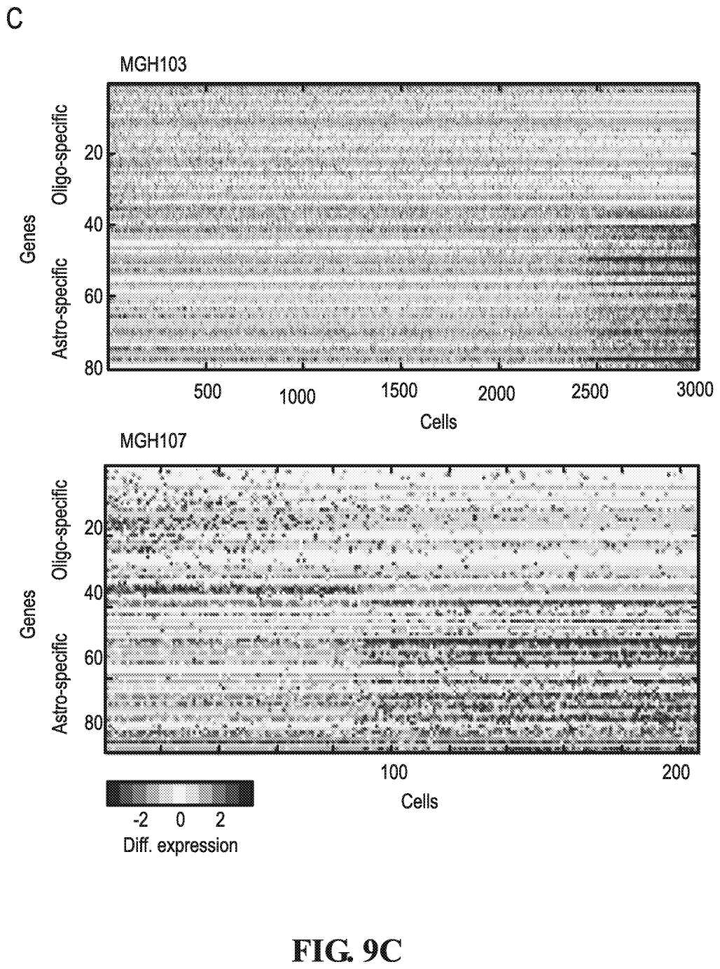

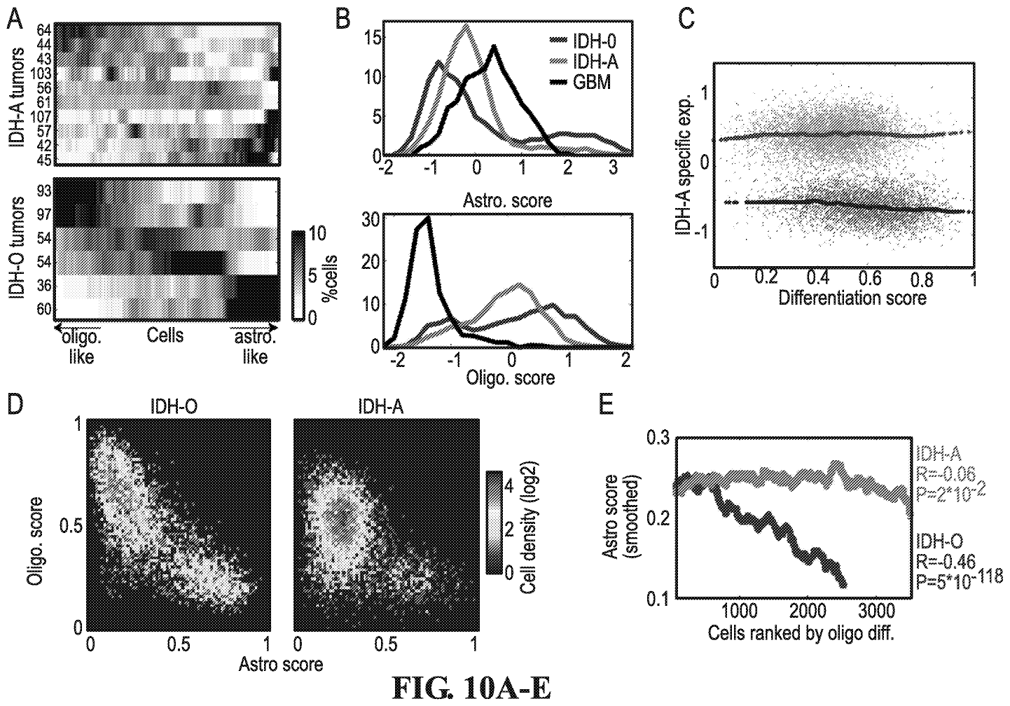

IDH-A and IDH-O are thought to primarily recapitulate the astrocytic and oligodendrocytic glial lineages, respectively (98). However, the results above demonstrate that most differences between IDH-A and IDH-O may be accounted by genetics and TME, and question the hypothesis of distinct lineages. Indeed, Applicants found only very limited differences in the expression of astrocyte-specific and oligodendrocyte-specific genes between IDH-A and IDH-O, either in bulk or in single cells profiles (FIG. 2A). Instead, the expression of these glial lineage genes varied substantially across the cells within each of the IDH-A tumors. After subtracting inter-tumor differences (SOM), principal component analysis (PCA) across all IDH-A cells demonstrated that PC1 and PC2 are associated with astrocyte-specific (PC1/2-high) and oligodendrocyte-specific (PC1/2-low) genes (FIG. 2B; P<10⁻⁹, hypergeometric test). In a recent study (137), Applicants identified astrocyte specific and oligodendrocyte specific signatures (Table 2). Applicants refined the sets of glial lineage genes using the scRNA-seq data to define astrocyte-like and oligodendrocyte-like expression programs which co-vary across IDH-A cells (FIG. 2C; SOM). These expression programs were not accounted for by inter-tumor differences, or by technical and batch effects (FIG. 9A,B and FIG. 10A), were reproduced in analysis of an additional 3,538 cells from two IDH-A tumors using a different protocol of single cell RNA-seq (FIG. 9C), and were co-expressed also among IDH-O cells (FIG. 2C). Applicants scored individual cells in each tumor type for expression of these programs, and classified cells into those with preferential expression of each program, as well as into intermediate cellular states (FIG. 2C). All tumors had a wide distribution of cellular states, yet there were more IDH-A cells in intermediate states (FIG. 2C and FIG. 10A). Interestingly, the distribution of single cell profiles from IDH-wildtype GBMs was drastically different, with a bias towards the astrocytic program, supporting the notion that the cellular architecture Applicants uncover in IDH-A and IDH-O is specific to IDH-mutant tumors and is not shared across all diffuse gliomas (FIG. 10B).

Since IDH-A and IDH-O contain diverse subpopulations with respect to glial differentiation programs, Applicants next investigated whether the 192 genes differentially expressed between the malignant compartments of IDH-A and IDH-O (FIG. 1F) are shared across all malignant cells or whether they are specific to certain subpopulations. As expected, expression differences that could be accounted for by genetic influences (FIG. 1F) were shared across all cells (FIG. 10C). However, differences between IDH-A and IDH-O in the expression of the remaining (“non-genetic”) 83 differentially expressed genes were most pronounced when comparing the more differentiated cells. In particular, when Applicants scored each malignant cell for its degree of differentiation (i.e., maximal expression of oligodendrocyte-like and astrocyte-like programs), “non-genetic” expression differences between IDH-A and IDH-O are almost completely abolished among the most undifferentiated cells (FIG. 2D). These results suggest that while the glial lineages are shared between IDH-A and IDH-O, some differences between these tumor types may be acquired during differentiation. Accordingly, undifferentiated cells from these tumor types might be functionally highly similar.

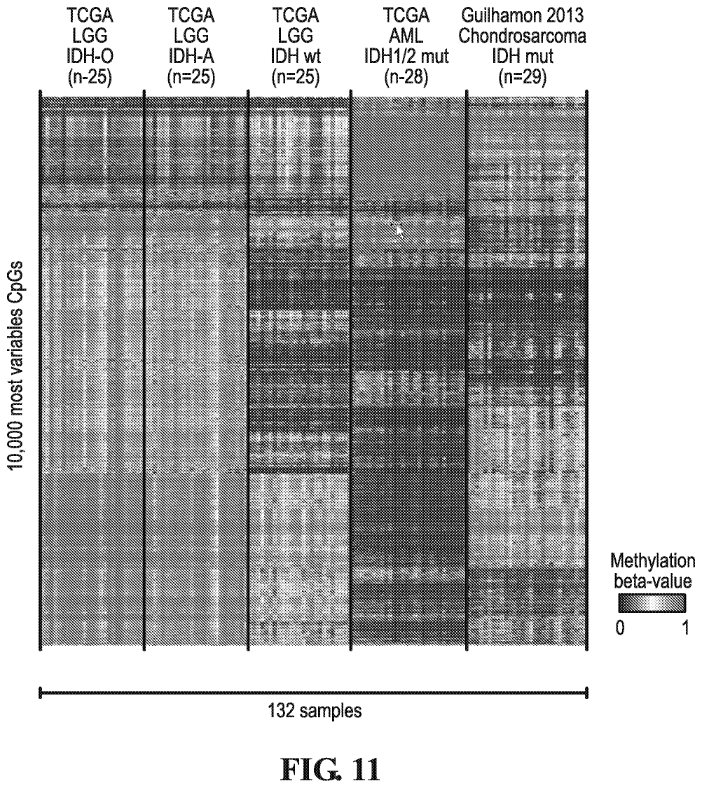

Taken together, the data supports a model in which malignant cells in IDH-A and IDH-O (but not in IDH-wild-type tumors) share similar cellular lineages, but differ primarily by genetics. To further test this hypothesis, Applicants analyzed DNA bulk methylation patterns, as DNA methylation may preserve epigenetic signatures of the cell-of-origin that are not evident by gene expression analysis. Applicants found high similarity in DNA methylation between IDH-A and IDH-O compared to both IDH-wildtype gliomas and to IDH-mutant non-glioma tumors (FIG. 11). While DNA methylation is highly influenced by the IDH mutation, this high similarity is consistent with a shared histogenesis of IDH-A and IDH-O.

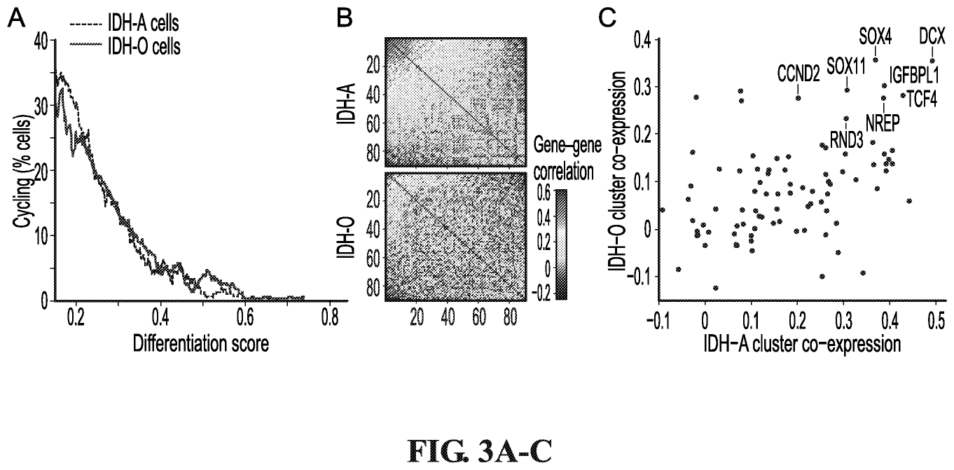

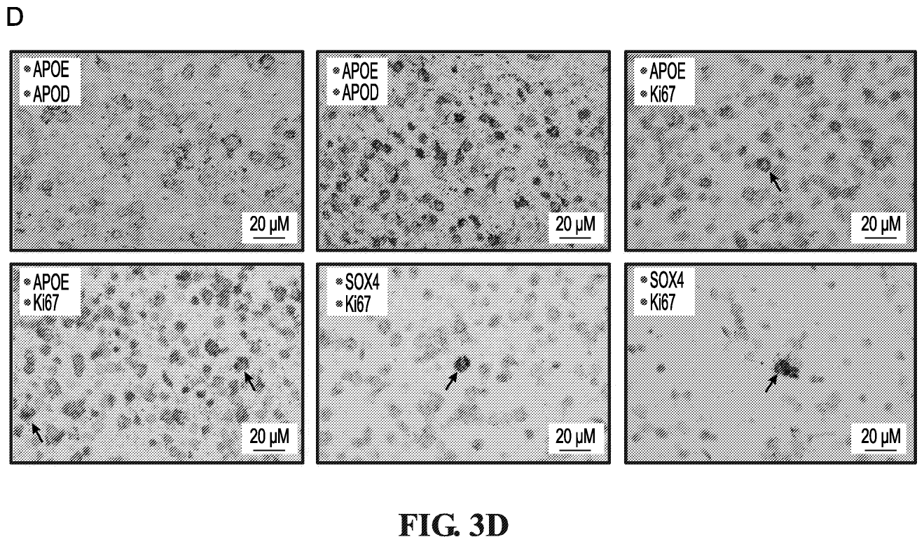

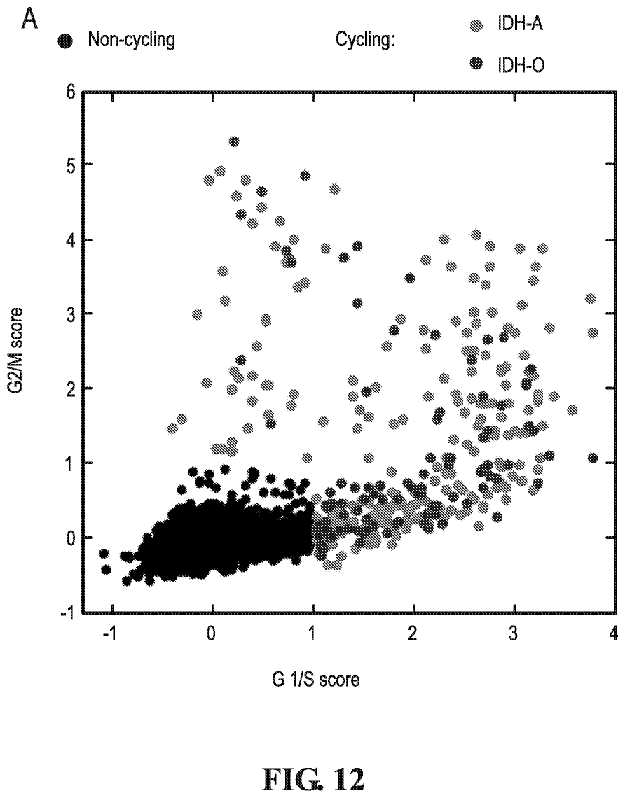

The high degree of expression similarity between undifferentiated cells in IDH-A and IDH-O and the possibility that these might reflect stem/progenitor cells prompted the Applicants to further investigate their programs. In a recent study (137), Applicants identified cancer stem-like cells in IDH-O that display neural stem/progenitor programs and are highly enriched in cell cycle programs (Table 1). Generalizing this finding across all IDH-mutant gliomas classes, Applicants identified cycling cells based on expression of consensus cell cycle signatures (FIG. 12A, SOM) (137, 124), and found that in both IDH-A and IDH-O only a small proportion of cells are proliferating (˜4% on average in this cohort), and that there is an inverse correlation between proliferation and differentiation (FIG. 3A). Remarkably, the fraction of cycling cells for a given state of differentiation is highly similar between IDH-A and IDH-O (FIG. 3A). This strongly supports a model in which proliferation and cell identity are tightly coupled in IDH-mutant tumors.

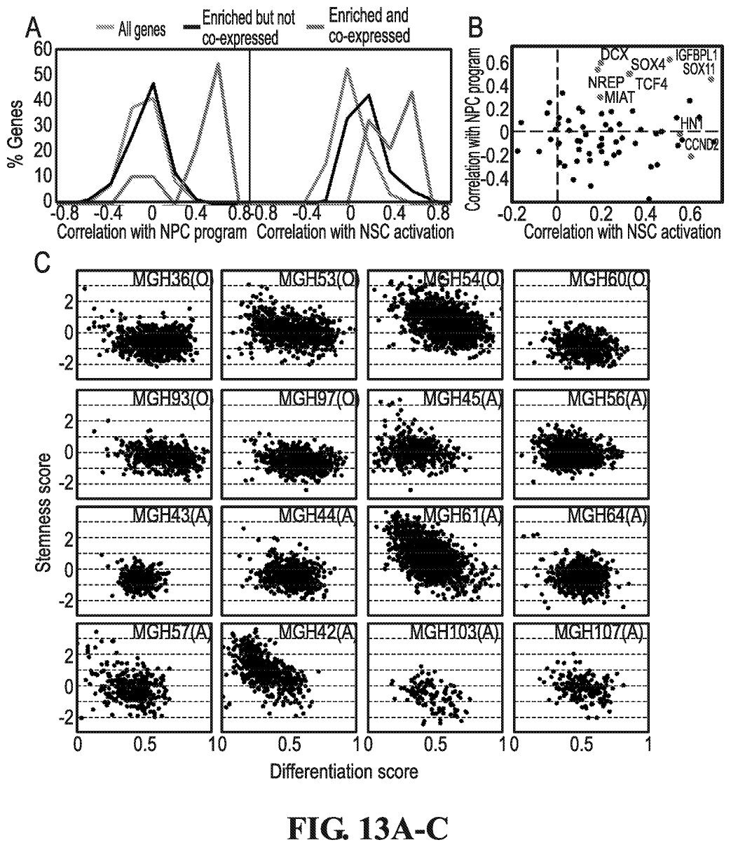

Applicants derived a gene signature of the undifferentiated cells (excluding cycling cells) across the IDH-A and IDH-O tumors. Ninety genes were enriched within undifferentiated cells of at least three distinct tumors and were examined further for their co-expression among undifferentiated IDH-A and IDH-O cells (FIG. 3B). Applicants defined the subset of genes (FIG. 3C) that are both enriched and co-expressed in undifferentiated cells of both IDH-A and IDH-O as a putative glioma stemness program. Indeed, this program includes neurodevelopmental transcription factors (e.g., SOX4, SOX11 and TCF4) and is highly consistent with the expression program of human neural stem cells (NSCs) and neural progenitor cells (NPCs) and with a program Applicants highlighted in IDH-O (FIG. 13). Applicants validated this tumor architecture in IDH-A tissues in a validation group of fourteen additional cases, showing in each tumor: (i) two lineages of glial differentiation, (ii) mutually exclusive expression of cycling (by Ki-67 staining) and differentiation (by ApoE expression) markers, and (iii) co-expression of cycling (Ki-67) and putative stem cell (SOX4) markers (FIG. 3D). This architecture has also been validated in a cohort of sixteen IDH-O (137).

While IDH-A and IDH-O share the same lineage programs, these analyses reveal three inter-related differences: (1) the overall fraction of cycling cells (FIG. 12), and (2) of undifferentiated cells (FIG. 2D) are higher in our IDH-A cases; and (3) the two lineage scores are inversely related in IDH-O, consistent with a differentiation process in which one lineage represses the other, while such a relationship is not observed in IDH-A (FIG. 10D-E).

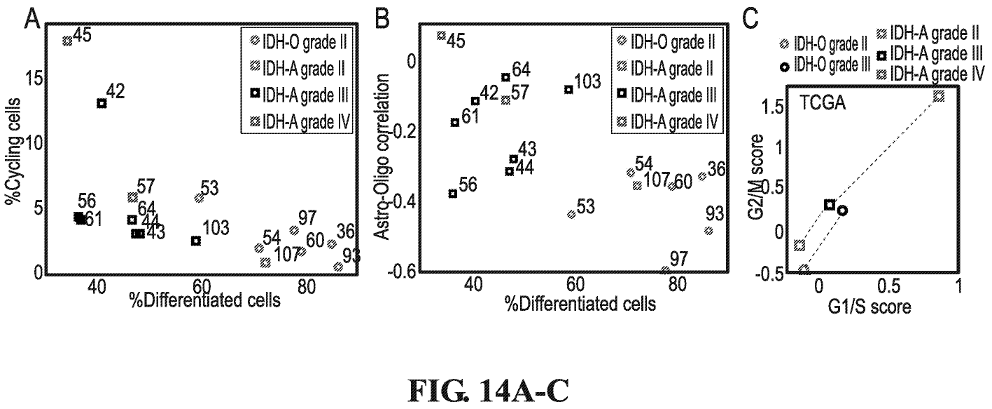

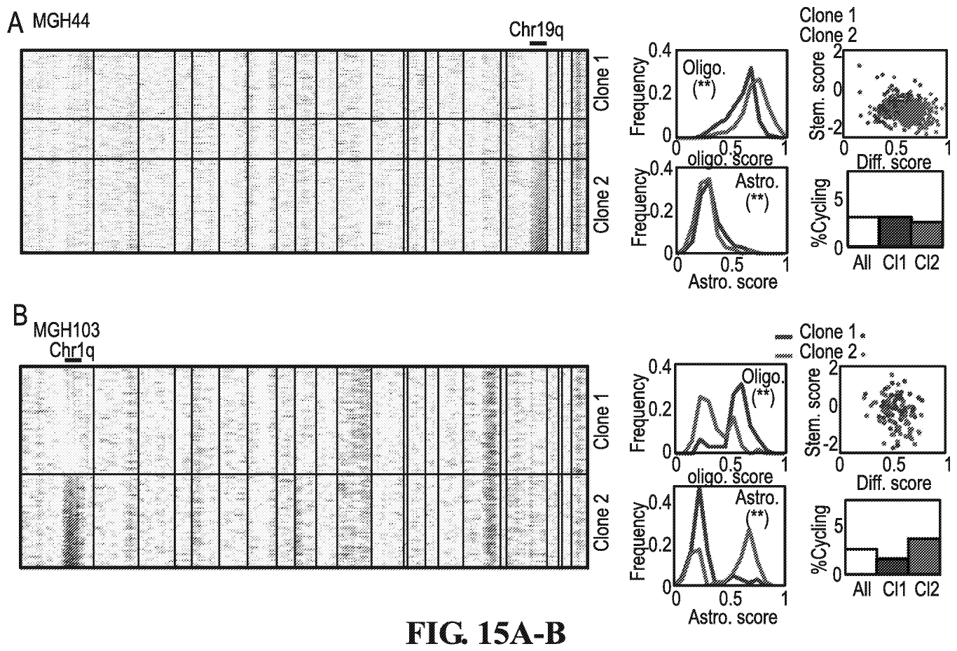

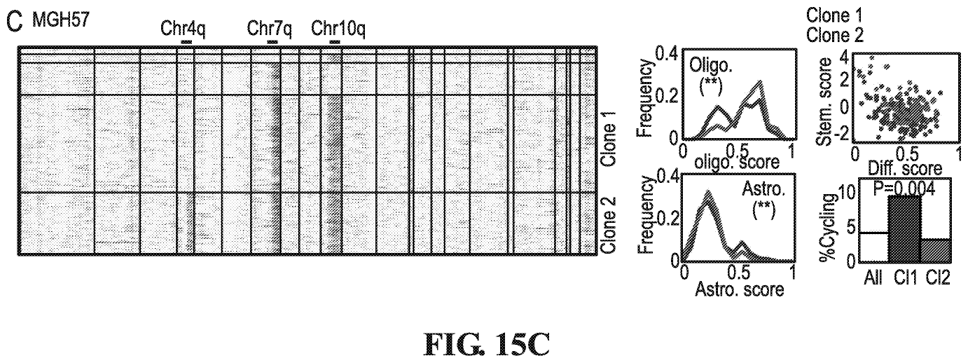

Notably, all three aspects also vary significantly within the IDH-A tumors and partially correlate with tumor grade, such that higher grade tumors tend to have more cycling and undifferentiated cells and a more limited association between lineage programs (FIG. 14A-B). This provides a molecular fingerprint for tumor progression, as IDH-A tumors often begin as grade II lesions and gradually progress to grade III and IV. Applicants validated the correlation between the frequency of cycling cells (as reflected by the cell cycle program) with grade in analysis of TCGA bulk samples (FIG. 14C).

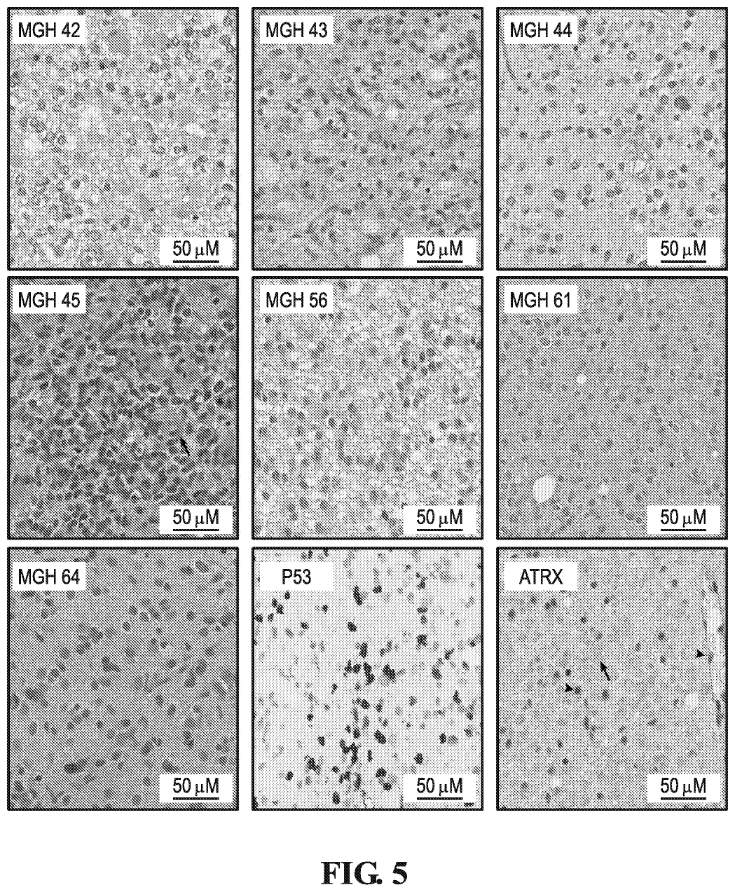

Next, Applicants hypothesized that the observed fingerprint of tumor grade-associated changes might also be reflected in clonal evolution, whereby genetically distinct subclones within the same tumor vary in their frequency of cycling and undifferentiated cells, and that selection favors the more aggressive subclones which tends to be enriched for proliferation and depleted for differentiation. To study genetic intra-tumoral heterogeneity, Applicants examined the CNVs inferred from single cell expression profiles (FIG. 5B), and predicted subclones in three of our tumors, MGH44, MGH57, and MGH103 (FIG. 6, 15). In each of these cases, while the overall tumor architecture was preserved, Applicants also observed variability either in the fraction of cycling cells or in differentiation patterns. Overall, these cases together with two IDH-O cases (137), demonstrate that patterns of differentiation and proliferation can be partially modulated by genetics and be subjected to selection.

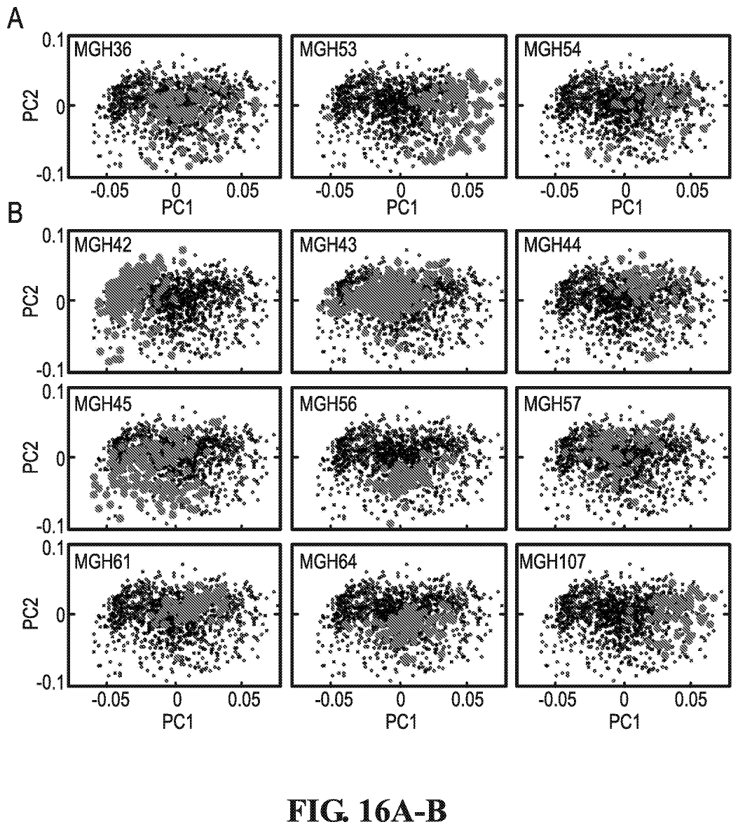

Finally, Applicants analyzed the diversity of microglia/macrophage cells, the predominant subset of non-malignant cells in the TME (n=1,043 in IDH-A and 246 in IDH-O) using PCA (FIG. 16). The second PC (PC2) reflected an inflammatory program consisting of cytokines (IL1, IL8, TNF), chemokines (CCL3, CCL4), NF_{K}B-related genes (REL, NFKBIA, NFKBIZ) and immediate early genes (JUNB, FOSB, EGR3, IER3, ATF3). The program was active in most non-tumoral cells across IDH-A and IDH-O tumors and is highly similar to our previously reported program in IDH-O (137). PC1 highlighted two mutually opposing programs, which were highly consistent with microglia (PC1-high) and macrophage (PC1-low) expression programs (FIG. 4A). Top PC1-high genes included well-known microglia markers, such as CX3CR1, P2RY12 and P2RY13 (129), whereas CD163, TGFBI and F13A1 were among the PC1-low genes and are more highly expressed in diverse macrophage populations than in microglia (130) (FIG. 4A). Thus, PC1 may correspond to the differences between brain-resident microglia, and infiltrating macrophages that reach the tumor through the circulation and must pass through the blood-brain barrier.

However, scoring cells by the relative expression of microglia-specific to macrophage-specific genes revealed a continuum, rather than a bimodal distribution (FIG. 4B), which is difficult to account for by a simple model of two populations (microglia and macrophages) and suggests additional influences on these expression programs. Accordingly, when Applicants performed RNA in situ hybridization (ISH) for CX3CR1 and CD163, Applicants observed some cells that co-express microglia and macrophage programs in tumors (FIG. 4H). Furthermore, even the top macrophage-like cells in gliomas have lower macrophage scores compared to macrophages from melanoma tumors (FIG. 4C) (126). Thus, the glioma microenvironment might have altered the expression profiles of macrophages, thereby decreasing their difference from microglia. Moreover, microglia/macrophages from each individual tumor had a limited range of scores, with some tumors biased towards macrophage-like (e.g. MGH42) and other towards microglia-like cells (e.g. MGH56) (FIG. 4C). This indicates that specific properties of the microenvironment of each tumor may be dominant over the immune cell-of-origin with respect to macrophage-like and microglia-like expression states, consistent with recent studies (129).

This observed inter-tumor variability in macrophage/microglia states correlated with grade, such that cells from higher-grade tumors were preferentially associated with macrophage-like expression states. Applicants validated this association by comparing the expression of macrophage-specific and microglia-specific genes across grades in bulk TCGA IDH-A and IDH-O tumors (FIG. 4D) and by RNA in situ hybridization (ISH) for CX3CR1 and CD163 in the cohort described herein (FIG. 4H). These results suggest that early in their development, gliomas primarily contain brain-resident microglia-like cells, while macrophage-like programs are associated with higher grades, possibly coinciding with other grade-associated changes, such as increased angiogenesis and alterations of the blood brain barrier.

Accordingly, this effect may parallel changes in tumor vascularity. Applicants derived a signature of endothelial-specific genes (SOM) and used their average expression to estimate the abundance of endothelial cells in each bulk tumor. This endothelial signature is correlated with the macrophage-specific, but not with microglia-specific, programs across IDH-O and IDH-A tumors (FIG. 4E). Moreover, the endothelial signature increases with tumor grade, paralleling changes in the macrophage-specific, but not microglia-specific, expression programs (FIG. 4D). While the endothelial program correlates with variability in the macrophage-like expression program between cells it does not account for the variability in the overall proportion of microglia and macrophages. IDH-A tumors have a considerably higher proportion of microglia+macrophage cells than IDH-O tumors, as noted above (FIG. 1C), and this difference is not accounted for by endothelial cells or by grade (FIG. 4D).

To search for additional mechanisms that might regulate infiltration of macrophage/microglia cells into the tumor Applicants searched for genes that are not expressed by macrophage/microglia, but are correlated with the inferred abundance of macrophage/microglia cells across bulk tumor samples. Applicants found 24 genes which are correlated both with microglia and with macrophage expression across IDH-A tumors, and separately, across IDH-O tumors (FIG. 4F, left). Although these analyses were performed within a tumor type and thus were not directly influenced by differences between IDH-A and IDH-O, these genes were preferentially expressed in IDH-A (FIG. 4F, right), consistent with the increased macrophage/microglia signatures in IDH-A. While Applicants cannot determine if these association are causal (i.e., Applicants cannot distinguish whether these genes influence, or are influenced by, immune infiltration, or whether both are affected by a third hidden factor), the ability of this expression program to predict the extent of macrophage/microglia infiltration across tumors and tumor types (FIG. 4G) suggests interactions between immune infiltration and other cells in the tumor. Interestingly, three of those genes were components of the complement system, as Applicants recently observed in melanoma (126). Taken together, our observations (i) define for the first time microglia and macrophage programs in gliomas at single-cell resolution, (ii) associate the macrophage, but not the microglia program, with clinical grade and increased vascularity, (iii) highlight a continuity in transcriptional programs of microglia/macrophage in tumors (rather than a bimodal distribution), suggesting plasticity of cellular states, (iv) reveal an overall increase in microglia/macrophage infiltration in IDH-A compared to IDH-O, and (v) define a tumor expression signature associated with increased microglia/macrophage infiltration.

In conclusion, the results described herein provide a general framework to decouple genetic, TME and lineage influences in cancer, combining single-cell analysis of a limited set of representative tumors with bulk samples collected for larger cohorts, such as those from TCGA. In IDH-mutant gliomas, this approach uncovers shared developmental lineages in IDH-A and IDH-O, suggesting that IDH-mutant gliomas are primarily composed of three subpopulations of cells including non-proliferating differentiated cells of two glial lineages, and proliferative undifferentiated cells that resemble neural stem/progenitor cells. The shared lineages and developmental hierarchies suggest a common progenitor for all IDH-mutant gliomas with NSC/NPC-like programs, shedding light on a longstanding debate in gliomagenesis (131).

This study, as described herein, represents a shift in our understanding of the histogenesis of glial tumors and supports a model where, from a glial lineage perspective, IDH-mutant gliomas subclasses share lineages and differ primarily by genetic mutations and TME composition; all IDH-mutant glioma Applicants examined at single cell resolution, including 10 IDH-A and 6 IDH-O tumors by genetics and histopathology, contained mixed glial lineages and shared a developmental architecture. While the cohort is fairly limited, the cases have had little selection bias (consecutive cases operated at MGH), and the observations have been validated in larger cohorts by tissue staining and by analysis of the TCGA datasets.

Given the similar developmental architecture of IDH-A and IDH-O, the morphological differences between these two entities might be linked to genetic differences between IDH-A and IDH-O and to TME composition. Accordingly, at least two genes involved in cytoskeleton and cell shape are downregulated by IDH-O-specific mutations. (I) glial fibrillary acidic protein (GFAP), a marker commonly used to assess lineages in histopathology, is regulated by CIC (137) and thus more highly expressed in IDH-A than IDH-O. (II) RHOC, encoding RhoC GTPase, a well-known regulator of cell shape and motility (138, 139) is located on chromosome arm 1p and therefore more highly expressed in IDH-A. Thus, signature genetic events might influence the morphology of cancer cells and underlie at least some of the histopathologic differences.

Interestingly, Applicants also found a considerable difference in the TME composition of IDH-mutant gliomas, whereby IDH-A is enriched with microglia/macrophages signatures. These differences in TME composition may also at least in part be driven by genetic influences. For example, TP53 (mutated only in IDH-A) has been implicated with effects on inflammation and immune infiltration (140).

While the data supports a shared architecture for all IDH-mutant gliomas, the cellular composition in other diffuse gliomas might differ; indeed, Applicants were not able to clearly identify a similar architecture in IDH-wildtype GBM; as much of the literature on cellular lineages in gliomas preceded the discovery of the IDH1/2 mutations, IDH-wildtype GBM might have confounded lineages in those studies. By analyzing for the first time IDH-mutant gliomas of different clinical grades (spanning II-IV) at single cell resolution, Applicants identified a potential molecular fingerprint of tumor progression, with support in TCGA datasets; these analyses suggest that high-grade lesions show increased proliferation, larger pools of undifferentiated cells, partially aberrant differentiation programs and increased infiltration by macrophages over resident microglia. Finally, from a therapeutic standpoint, the data shows for the first time that triggering cellular differentiation or targeting a specific stem cell phenotype with immunotherapies can be used for the treatment of these currently incurable malignancies.

### Example 3

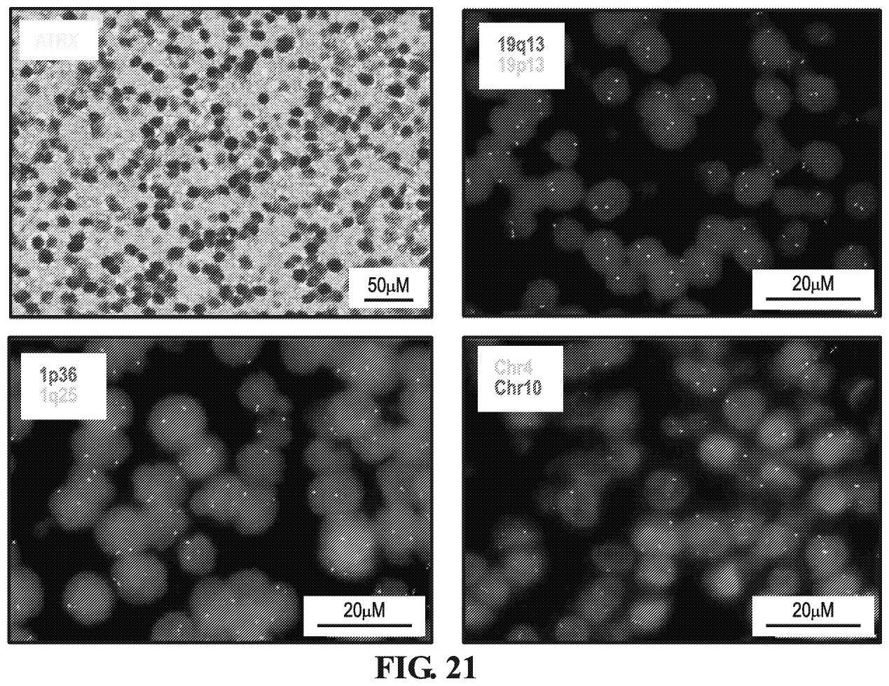

The data described herein characterizing oligodendrogliomas is described in further detail below. Using human oligodendrogliomas as a model, Applicants profiled 4,347 single cells from six patient tumors by RNA-seq, reconstructed their transcriptional architecture and related it to genetic mutations. Application of larger scale single-cell profiling in grade II lesions may more definitively unmask developmental hierarchies in brain tumors, because low-grade gliomas are typically well differentiated and driven by a limited number of genetic events. To further limit inter-tumoral heterogeneity, Applicants focused on oligodendroglioma, a major glioma class that remains incurable (91) and is characterized by signature mutations in IDH1/2 and co-deletion of chromosome arms 1p and 19q. Applicants studied six grade II oligodendrogliomas where IDH1 R132H mutation (or IDH2 R172K mutation) and chromosome 1p/19q co-deletion were confirmed and that had not received pre-operative chemotherapy or radiation (Table 1; FIG. 21) (92).

TABLE 1

| | | | | | Clinical IDH1 | Clinical FISH | Integrated clinical |
|---|---|---|---|---|---|---|---|
| Designation | Age | Gender | Location | Grade | result | result | diagnosis |
| MGH36 | 67 | male | Right fronttotemporoinsular | WHO II/III | R132H mutation | 1p19q loss | oligodendroglioma, 1p/19q codelted |
| MGH53 | 37 | male | Left frontal | WHO II | R132H mutation | 1p19q loss | oligodendroglioma, 1p/19q codelted |
| MGH54 | 35 | male | Right parietal | WHO II | R132H mutation | 19q loss, | oligodendroglioma, 1p/19q codelted |
| | | | | | | borderline | |
| | | | | | | 1p loss | |
| MGH60 | 51 | male | Left frontotemporoinsular | WHO II | R132H mutation | 1p19q loss | oligodendroglioma, 1p/19q codelted |

VALIDATION COHORT

| | | | | | | | |
|---|---|---|---|---|---|---|---|
| Oligo 1 | 30 | male | Right frontal | WHO II | R132H mutation | 1p19q loss | Recurrent oligodendroglima, 1p/19q |
| | | | | | | | codeleted |
| Oligo 2 | 51 | male | Right occipital | WHO II | R132H mutation | 1p19q loss | oligodendroglioma, 1p/19q codelted |
| Oligo 3 | 60 | female | Left temporal | WHO III | R132H mutation | 1p19q loss | anaplastic oligodendroglioma, 1p/19q |
| | | | | | | | codelted |
| Oligo 4 | 63 | male | Left frontal | WHO III | R132H mutation | 1p19q loss | recurrent anaplastic oligodendroglioma, |
| | | | | | | | 1p/19q codeleted |
| Oligo 5 | 65 | female | Left frontal | WHO II | R132H mutation | 1p19q loss | oligodendroglioma, 1p/19q codeleted |
| Oligo 6 | 13 | female | Left frontal | WHO II | R132H mutation | 1p19q loss | oligodendroglioma, 1p/19q codeleted |
| Oligo 7 | 65 | female | Left parietal | WHO III | R132H mutation | 1p19q loss | recurrent anaplastic oligodendroglioma, |
| | | | | | | | 1p/19q codeleted |
| Oligo 8 | 59 | female | Cerebellar vermis | WHO III | R132H mutation | 1p19q loss | recurrent anaplastic oligodendroglioma, |
| | | | | | | | 1p/19q codeleted |
| Oligo 9 | 50 | male | Left frontal | WHO II | R132H mutation | 1p19q loss | oligodendroglioma, 1p/19q codeleted |
| Oligo 10 | 77 | male | Right frontotemporoinsular | WHO II | R132H mutation | 1p19q loss | oligodendroglioma, 1p/19q codeleted |

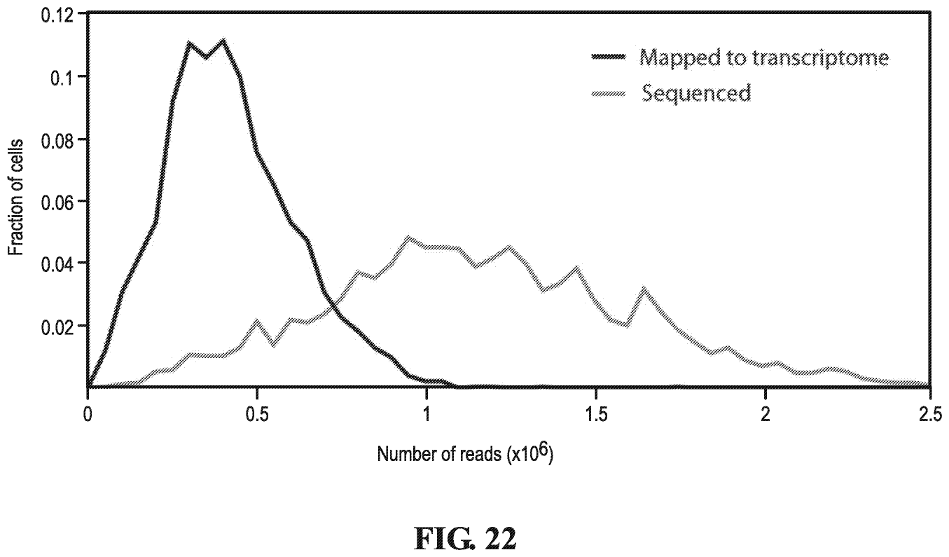

Overall, Applicants performed single cell RNA-seq (93) on 5,172 cells at an average depth of ˜1.2 million reads per cell (FIG. 22), resulting in 4,347 cells that passed the quality controls. Three tumors were analyzed more deeply (MGH36, 53, 54; 791-1,229 cells per tumor that passed our quality controls) and three tumors (MGH60, 93 and 97) were profiled at medium depth (430-598 cells).

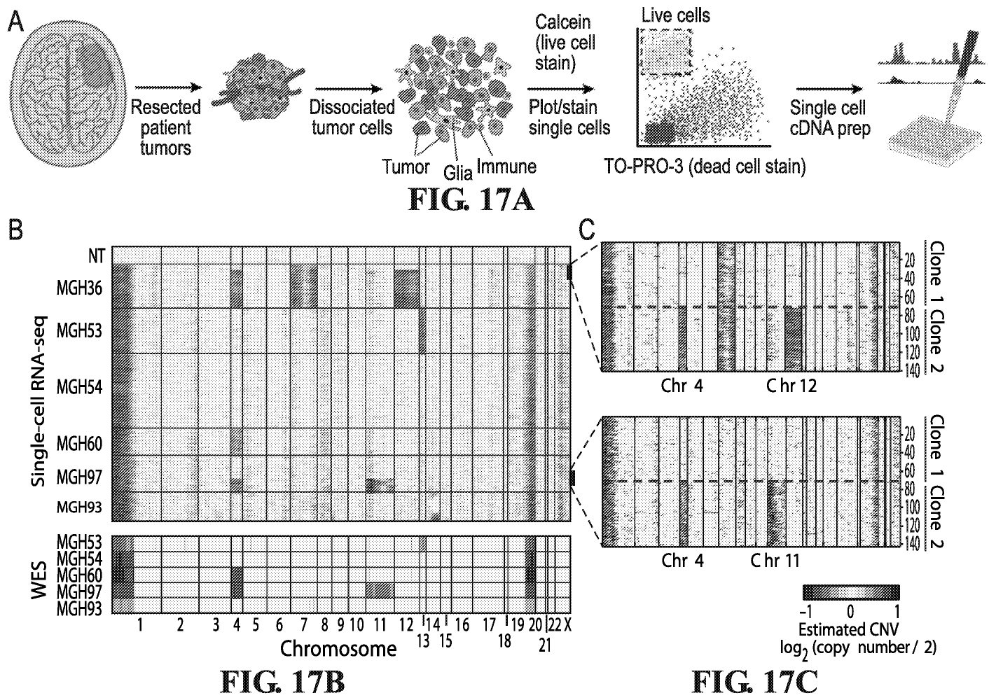

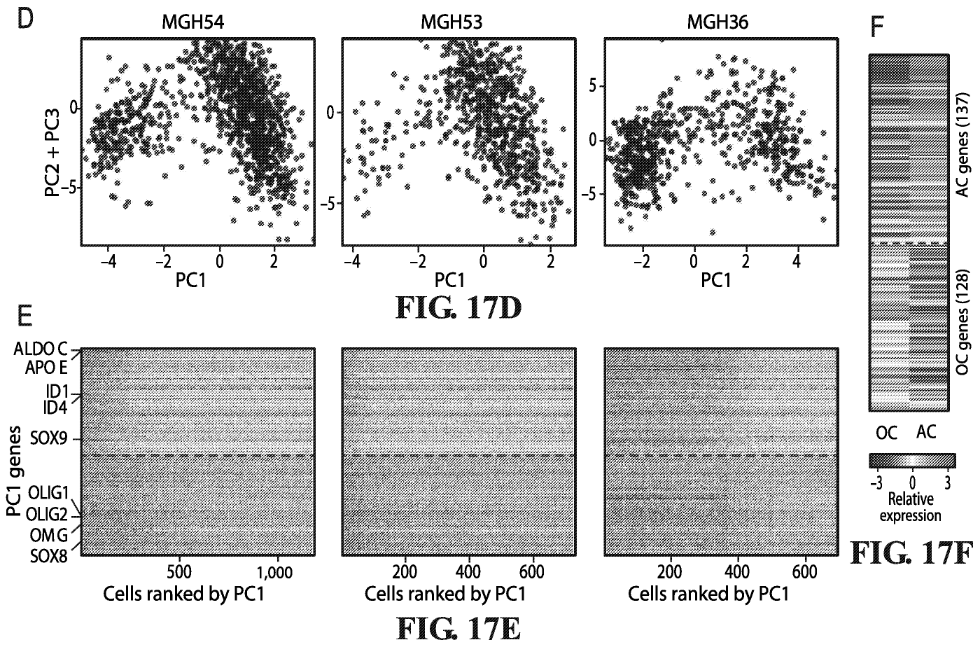

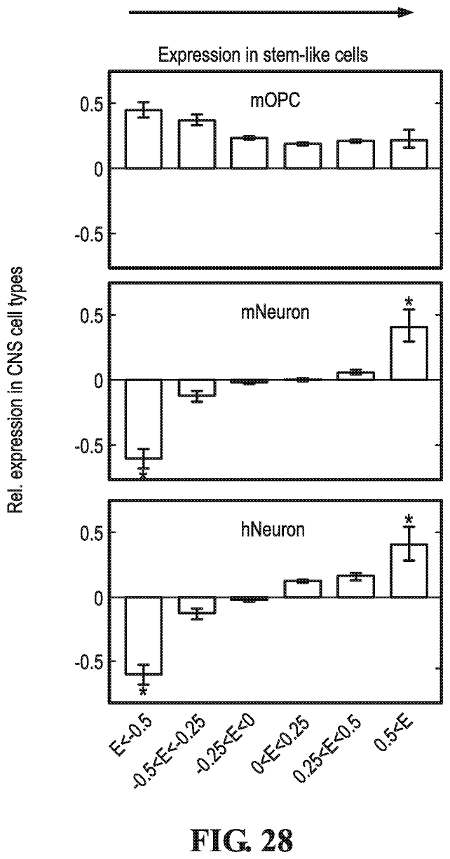

Applicants distinguished malignant from possible non-malignant cells in the tumor microenvironment, by estimating chromosomal copy number variations (CNVs) from the average expression of genes in large chromosomal regions within each cell (FIG. 17B and FIG. 28; Methods) (15). Each tumor contained a large majority of malignant cells with deletions of chromosomes 1p and 19q, the hallmarks of oligodendroglioma, as well as in some cases additional tumor-specific CNVs, which were validated by FISH and by DNA whole-exome sequencing (WES) (FIG. 17B, FIGS. 21 and 28). In two tumors (MGH36, MGH97), CNV analysis pointed to the existence of two clones (FIG. 17B,C) whereby Clone 2 harbored all the CNVs present in Clone 1, as well as additional CNVs, suggesting that Clone 2 was in each case derived through subsequent tumor evolution.

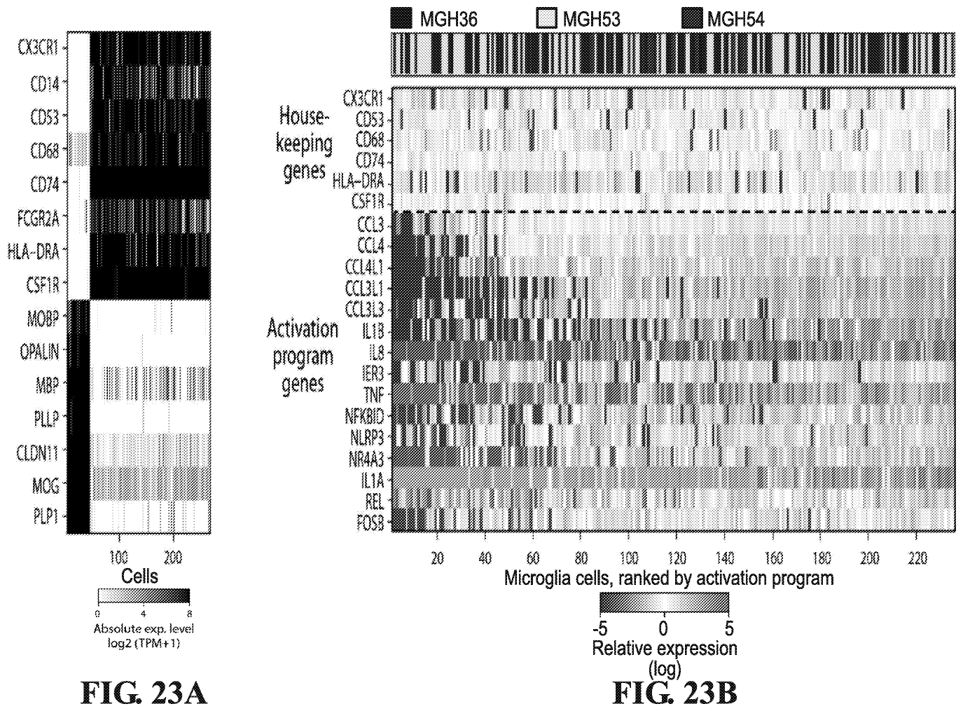

Another 304 cells across the six tumors lacked any detectable CNVs, and clustered by gene expression into two subsets, which differed markedly from the malignant cells and expressed microglia and mature oligodendrocyte markers, respectively, consistent with being non-malignant cell types (FIG. 23A). Applicants detected significant variability between the microglia cells, in which a set of pro-inflammatory cytokines (IL1A/B, IL8 and TNF), chemokines (CCL3/4) and early response genes were coordinately expressed by ˜80% of the microglia (FIG. 23B). This expression program differs from canonical macrophage M1/M2 responses (94) and therefore suggests an unknown microglia expression program that appears to be glioma-specific.

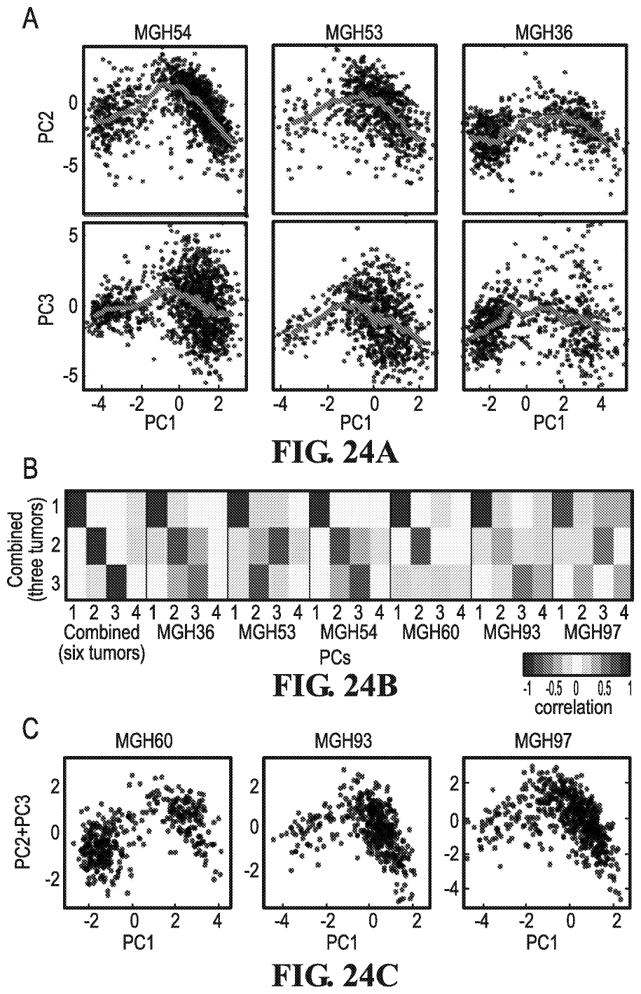

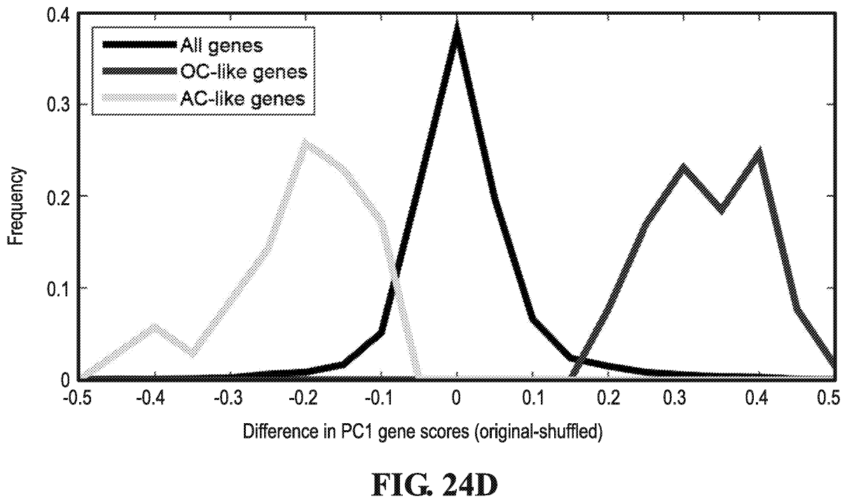

Applicants examined the heterogeneity of the cancer cells from the three tumors for which Applicants analyzed the largest cell numbers by a combined principal component analysis (PCA), while controlling for data quality per transcript and per cell and inter-tumor heterogeneity (Methods). Applicants identified two prominent groups of cells, corresponding to low and high PC1 scores (FIG. 17D) and expressing distinct lineage markers of astrocytes and oligodendrocytes, respectively. These results were highly consistent across all six tumors, and were not simply accounted for by technical and batch effects (FIG. 24 and Note 1). Specifically, in each tumor, cells with high PC1 scores were strongly associated with high expression of 137 genes, including markers of oligodendroglial lineage (e.g., OLIG1/2, OMG), and with low expression of 128 genes, including markers of astrocytic lineage (e.g., APOE, ALDOC, SOX9) (FIG. 17E, Table 2) (95). Cells with low PC1 scores had the opposite pattern of expression. Consistent with these specific markers, the orthologs of most PC1-associated genes were preferentially expressed in mice oligodendrocytes (OC) and astrocytes (AC), respectively (FIG. 17F) (97). This indicates that oligodendrogliomas are primarily composed of two subpopulations of cells with transcriptional states of distinct glial lineages; this mirrors histopathology, where cancer cells of astrocytic lineage within oligodendrogliomas are known as “microgemistocytes” (98).

### Table 2. Ranked Gene-Sets Used to Define Cell Cycle, Stemness and Lineage Scores.

Each gene-set is ranked from most significant (top) to least significant gene (bottom).
Significance was determined by average fold-change of upregulation in G1/S, G2/M and stem-like cells (first three columns) or by the correlation with PC1 (positive correlation for OC genes and negative for AC genes).
Two gene-sets are given for each of the lineages:
“PCA−only” denote genes that were identified from PCA analysis of oligodendroglioma cells and are presented in FIG. 17.
“PCA+mice” denote genes that were both identified in the PCA analysis of oligodendroglioma cells and are preferentially expressed in the resective lineage in mice (Methods), and these were used to estimate lineage scores.

| G1/S | G2/M | stemness | AC (PCA-only) | AC (PCA + mice) | OC (PCA-only) | OC (PCA + mice) |
|---|---|---|---|---|---|---|
| MCM5 | HMGB2 | SOX4 | APOE | APOE | LMF1 | OLIG1 |
| PCNA | CDK1 | CCND2 | SPARCL1 | SPARCL1 | OLIG1 | SNX22 |
| TYMS | NUSAP1 | SOX11 | SPOCK1 | ALDOC | SNX22 | GPR17 |
| FEN1 | UBE2C | RBM6 | CRYAB | CLU | POLR2F | DLL3 |
| MCM2 | BIRC5 | HNRNPH1 | ALDOC | EZR | LPPR1 | SOX8 |
| MCM4 | TPX2 | HNRNPL | CLU | SORL1 | GPR17 | NEU4 |
| RRM1 | TOP2A | PTMA | EZR | MLC1 | DLL3 | SLC1A1 |
| UNG | NDC80 | TRA2A | SORL1 | ABCA1 | ANGPTL2 | LIMA1 |
| GINS2 | CKS2 | SET | MLC1 | ATP1B2 | SOX8 | ATCAY |
| MCM6 | NUF2 | C6orf62 | ABCA1 | RGMA | RPS2 | SERINC5 |
| CDCA7 | CKS1B | PTPRS | ATP1B2 | AGT | FERMT1 | LHFPL3 |
| DTL | MKI67 | CHD7 | PAPLN | EEPD1 | PHLDA1 | SIRT2 |
| PRIM1 | TMPO | CD24 | CA12 | CST3 | RPS23 | OMG |
| UHRF1 | CENPF | H3F3B | BBOX1 | SOX9 | NEU4 | APOD |
| MLF1IP | TACC3 | C14orf23 | RGMA | EDNRB | SLC1A1 | MYT1 |
| HELLS | FAM64A | NFIB | AGT | GABRB1 | LIMA1 | OLIG2 |
| RFC2 | SMC4 | SRGAP2C | EEPD1 | PLTP | ATCAY | RTKN |
| RPA2 | CCNB2 | STMN2 | CST3 | JUNB | SERINC5 | FA2H |
| NASP | CKAP2L | SOX2 | SSTR2 | DKK3 | CDH13 | MARCKSL1 |
| RAD51AP1 | CKAP2 | TFDP2 | SOX9 | ID4 | CXADR | LIMS2 |
| GMNN | AURKB | CORO1C | RND3 | ADCYAP1R1 | LHFPL3 | PHLDB1 |
| WDR76 | BUB1 | EIF4B | EDNRB | GLUL | ARL4A | RAB33A |
| SLBP | KIF11 | FBLIM1 | GABRB1 | PFKFB3 | SHD | OPCML |
| CCNE2 | ANP32E | SPDYE7P | PLTP | CPE | RPL31 | SHISA4 |
| UBR7 | TUBB4B | TCF4 | JUNB | ZFP36L1 | GAP43 | TMEFF2 |
| POLD3 | GTSE1 | ORC6 | DKK3 | JUN | IFITM10 | NME1 |
| MSH2 | KIF20B | SPDYE1 | ID4 | SLC1A3 | SIRT2 | NXPH1 |
| ATAD2 | HJURP | NCRUPAR | ADCYAP1R1 | CDC42EP4 | OMG | GRIA4 |
| RAD51 | HJURP | BAZ2B | GLUL | NTRK2 | RGMB | SGK1 |
| RRM2 | CDCA3 | NELL2 | EPAS1 | CBS | HIPK2 | ZDHHC9 |
| CDC45 | HN1 | OPHN1 | PFKFB3 | DOK5 | APOD | CSPG4 |
| CDC6 | CDC20 | SPHKAP | ANLN | FOS | NPPA | LRRN1 |
| EXO1 | TTK | RAB42 | HEPN1 | TRIL | EEF1B2 | BIN1 |
| TIPIN | CDC25C | LOH12CR2 | CPE | SLC1A2 | RPS17L | EBP |
| DSCC1 | KIF2C | ASCL1 | RASL10A | ATP13A4 | FXYD6 | CNP |
| BLM | RANGAP1 | BOC | SEMA6A | ID1 | MYT1 | |
| CASP8AP2 | NCAPD2 | ZBTB8A | ZFP36L1 | TPCN1 | RGR | |
| USP1 | DLGAP5 | ZNF793 | HEY1 | FOSB | OLIG2 | |
| CLSPN | CDCA2 | TOX3 | PRLHR | LIX1 | ZCCHC24 | |
| POLA1 | CDCA8 | EGFR | TACR1 | IL33 | MTSS1 | |
| CHAF1B | ECT2 | PGM5P2 | JUN | TIMP3 | GNB2L1 | |
| BRIP1 | KIF23 | EEF1A1 | GADD45B | NHSL1 | C17orf76-AS1 | |
| E2F8 | HMMR | MALAT1 | SLC1A3 | ZFP36L2 | ACTG1 | |
| | AURKA | TATDN3 | CDC42EP4 | DTNA | EPN2 | |
| | PSRC1 | CCL5 | MMD2 | ARHGEF26 | PGRMC1 | |
| | ANLN | EVI2A | CPNE5 | TBC1D10A | TMSB10 | |
| | LBR | LYZ | CPVL | LHFP | NAP1L1 | |
| | CKAP5 | POU5F1 | RHOB | NOG | EEF2 | |
| | CENPE | FBXO27 | NTRK2 | LCAT | MIAT | |
| | CTCF | CAMK2N1 | CBS | LRIG1 | CDHR1 | |
| | NEK2 | NEK5 | DOK5 | GATSL3 | TRAF4 | |
| | G2E3 | PABPC1 | TOB2 | ACSL6 | TMEM97 | |
| | GAS2L3 | AFMID | FOS | HEPACAM | NACA | |
| | CBX5 | QPCTL | TRIL | SCG3 | RPSAP58 | |
| | CENPA | MBOAT1 | NFKBIA | RFX4 | SCD | |
| | | HAPLN1 | SLC1A2 | NDRG2 | TNK2 | |
| | | LOC90834 | MTHFD2 | HSPB8 | RTKN | |
| | | LRTOMT | IER2 | ATF3 | UQCRB | |
| | | GATM-AS1 | EFEMP1 | PON2 | FA2H | |
| | | AZGP1 | ATP13A4 | ZFP36 | MIF | |
| | | RAMP2-AS1 | KCNIP2 | PER1 | TUBB3 | |
| | | SPDYE5 | ID1 | BTG2 | COX7C | |
| | | TNFAIP8L1 | TPCN1 | NRP1 | AMOTL2 | |
| | | | LRRC8A | PRRT2 | THY1 | |
| | | | MT2A | F3 | NPM1 | |
| | | | FOSB | | MARCKSL1 | |
| | | | L1CAM | | LIMS2 | |
| | | | LIX1 | | PHLDB1 | |
| | | | HLA-E | | RAB33A | |
| | | | PEA15 | | GRIA2 | |
| | | | MT1X | | OPCML | |
| | | | IL33 | | SHISA4 | |
| | | | LPL | | TMEFF2 | |
| | | | IGFBP7 | | ACAT2 | |
| | | | C1orf61 | | HIP1 | |
| | | | FXYD7 | | NME1 | |
| | | | TIMP3 | | NXPH1 | |
| | | | RASSF4 | | FDPS | |
| | | | HNMT | | MAP1A | |
| | | | JUND | | DLL1 | |
| | | | NHSL1 | | TAGLN3 | |
| | | | ZFP36L2 | | PID1 | |
| | | | SRPX | | KLRC2 | |
| | | | DTNA | | AFAP1L2 | |
| | | | ARHGEF26 | | LDHB | |
| | | | SPON1 | | TUBB4A | |
| | | | TBC1D10A | | ASIC1 | |
| | | | DGKG | | TM7SF2 | |
| | | | LHFP | | GRIA4 | |
| | | | FTH1 | | SGK1 | |
| | | | NOG | | P2RX7 | |
| | | | LCAT | | WSCD1 | |
| | | | LRIG1 | | ATP5E | |
| | | | GATSL3 | | ZDHHC9 | |
| | | | EGLN3 | | MAML2 | |
| | | | ACSL6 | | UGT8 | |
| | | | HEPACAM | | C2orf27A | |
| | | | ST6GAL2 | | VIPR2 | |
| | | | KIF21A | | DHCR24 | |
| | | | SCG3 | | NME2 | |
| | | | METTL7A | | TCF12 | |
| | | | CHST9 | | MEST | |
| | | | RFX4 | | CSPG4 | |
| | | | P2RY1 | | GAS5 | |
| | | | ZFAND5 | | MAP2 | |
| | | | TSPAN12 | | LRRN1 | |
| | | | SLC39A11 | | GRIK2 | |
| | | | NDRG2 | | FABP7 | |
| | | | HSPB8 | | EIF3E | |
| | | | IL11RA | | RPL13A | |
| | | | SERPINA3 | | ZEB2 | |
| | | | LYPD1 | | EIF3L | |
| | | | KCNH7 | | BIN1 | |
| | | | ATF3 | | FGFBP3 | |
| | | | TMEM151B | | RAB2A | |
| | | | PSAP | | SNX1 | |
| | | | HIF1A | | KCNIP3 | |
| | | | PON2 | | EBP | |
| | | | HIF3A | | CRB1 | |
| | | | MAFB | | RPS10-NUDT3 | |
| | | | SCG2 | | GPR37L1 | |
| | | | GRIA1 | | CNP | |
| | | | ZFP36 | | DHCR7 | |
| | | | GRAMD3 | | MICAL1 | |
| | | | PER1 | | TUBB | |
| | | | TNS1 | | FAU | |
| | | | BTG2 | | TMSB4X | |
| | | | CASQ1 | | PHACTR3 | |
| | | | GPR75 | | | |
| | | | TSC22D4 | | | |
| | | | NRP1 | | | |
| | | | DNASE2 | | | |
| | | | DAND5 | | | |
| | | | SF3A1 | | | |
| | | | PRRT2 | | | |
| | | | DNAJB1 | | | |
| | | | F3 | | | |

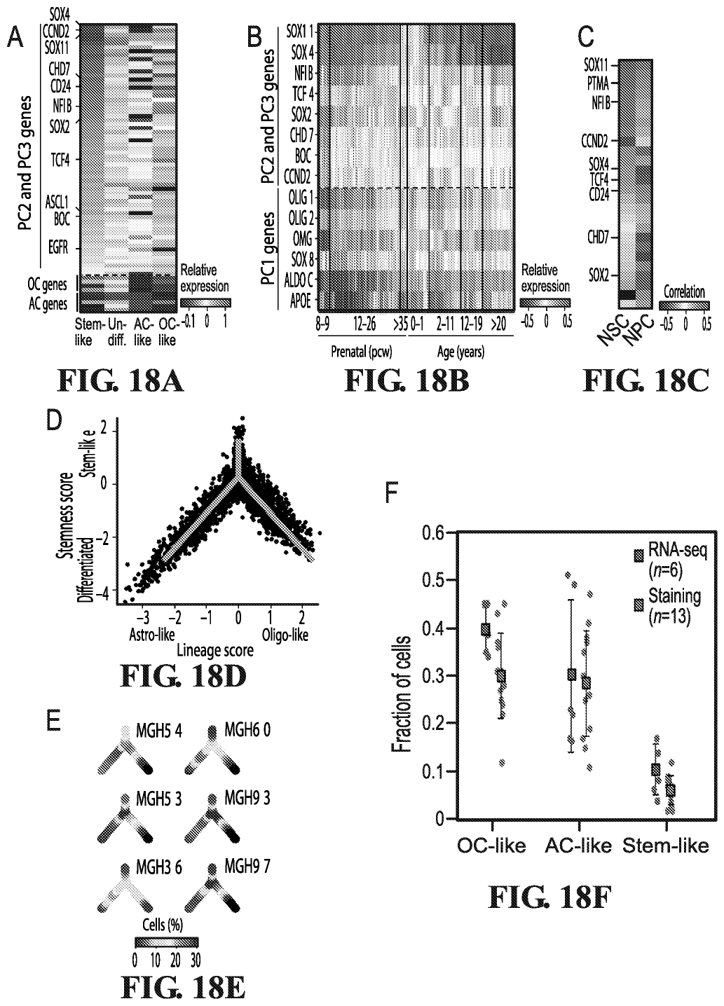

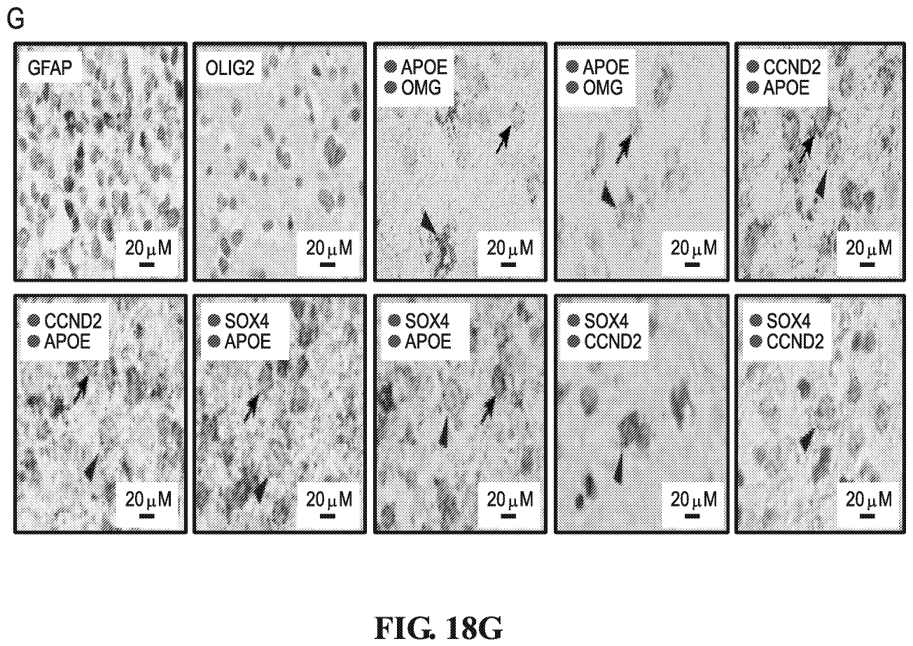

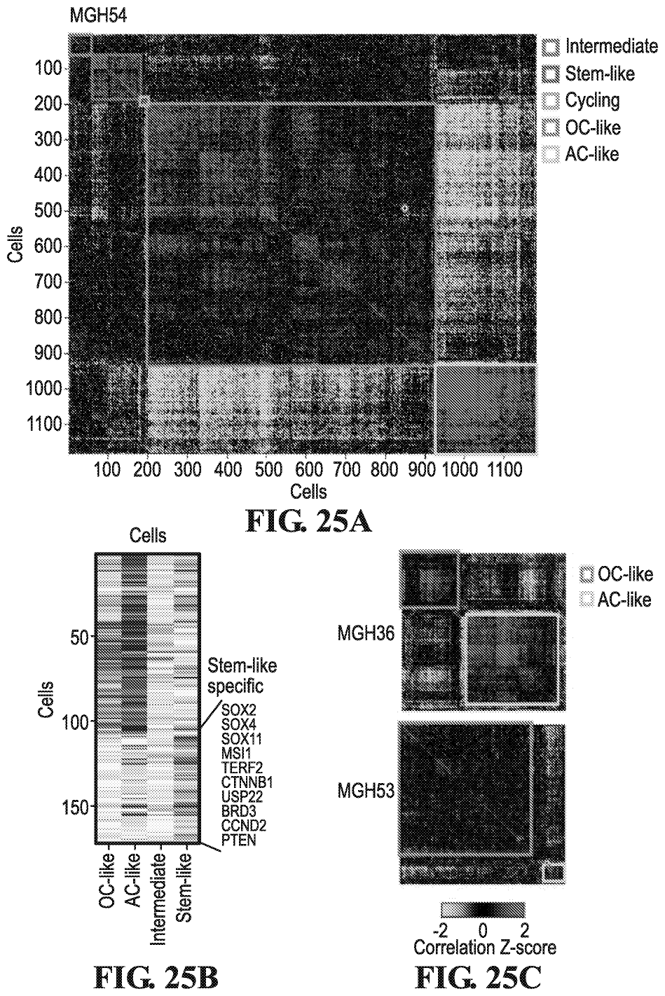

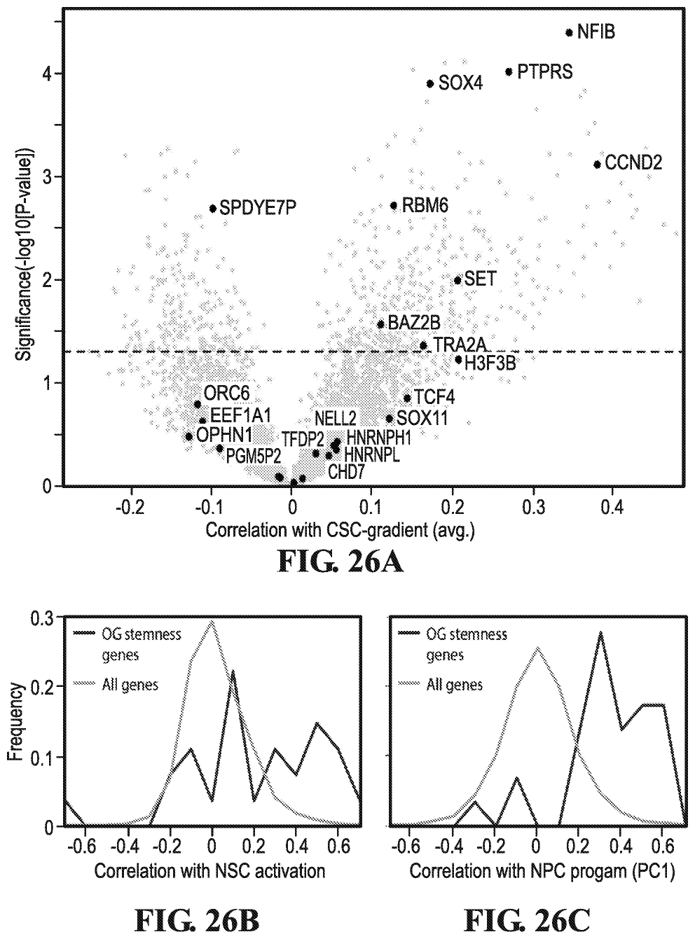

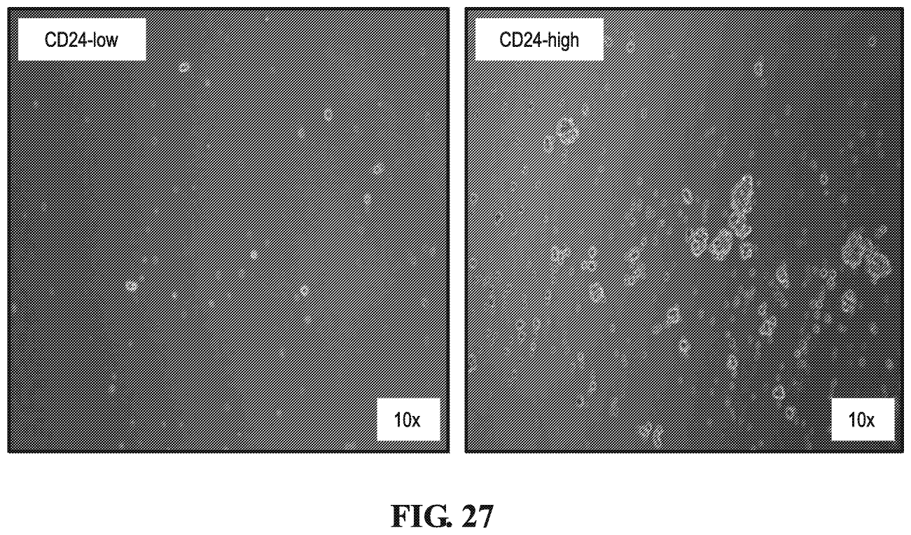

Cells with high PC2 and PC3 scores showed an association with intermediate values of PC1 (shown both for PC2+PC3 (FIG. 17D), (FIG. 24C) and separately for PC2 and for PC3 (FIG. 24A)), indicating a lack of differentiation and prompting us to explore additional programs. (As for PC11, these patterns were not the result of technical or batch effects; Note 1). 63 genes were associated with both PC2 and PC3 (Table 2). Several lines of evidence indicate that this represents a “stemnness” program. First, among the 20 highest-ranking genes associated with PC2/3 (FIG. 18A) were SOX4, SOX11 and SOX2, neurodevelopmental transcription factors critical to neural stem cells and self-renewal of glioma stem cells (99-101). Additional genes with important roles in neurogenesis and in the CSC program of gliomas included the transcription factors NFIB and ASCL1, the chromatin remodeler CHD7, the cell surface protein CD24, and BOC and TCF4, which function in signaling pathways central to stem cell maintenance (74, 15, 99-104). Similar results were obtained by hierarchical clustering, showing a distinct cluster of cells that preferentially express these PC2/3-associated stemness regulators (FIG. 25). Second, several genes of this oligodendroglioma “stemness” program were previously identified by our study on single cell RNA-seq in primary human glioblastoma CSC (FIG. 26A, P=1.5*10⁻⁴ for the overlap between the two stemness programs, hypergeometric test), albeit each program also contains specific regulators, such as CD24 which emerged as the top cell surface marker in the oligodendroglioma program. Third, analysis of the human brain transcriptome dataset from the Allen Brain Atlas showed that the expression of PC2/3-associated regulators was highest in early prenatal human brain samples and dropped significantly after birth, in childhood and adult samples, further indicating a role in neural development (FIG. 18B, P=8*10¹⁸ for the enrichment of PC2/3-associated genes in prenatal vs. adult samples, t-test) (105). This pattern was particularly pronounced for SOX4 and for SOX11, which was the gene most significantly enriched in prenatal samples across the human genome (P=4*10⁻⁵⁰, t-test), while an opposite pattern was found for AC and OC lineage genes (FIG. 18B). Similarly, interrogating a recently published study of single-cell RNA-seq analysis of the human brain, Applicants identified several PC2/3-associated genes as preferentially expressed in single-cells in fetal human brain, while Applicants did not identify any adult human brain cell type expressing this signature (P=0.006 for enrichment of PC2/3-associated genes in the fetal vs. adult programs, hypergeometric test) (106). Based on these four lines of evidence, cells with intermediate PC1 values were thus separated into “undifferentiated” (low PC2/3) and “stem/progenitors” (high PC2/3) cells (FIG. 18A).

Oligodendrogliomas are often thought to arise from transformation of oligodendrocyte progenitor cells (OPCs) (108), raising the possibility that the “stem/progenitors” PC2/3 genes may reflect an OPC-like program. However, the PC2/3-associated genes were not preferentially expressed in OPCs; instead, these genes were preferentially expressed in cells of neuronal lineage (FIG. 28) (97, 123). Thus, although oligodendroglioma display only glial differentiation (both molecularly and histologically) and are thought to be derived from glial precursors, they may harbor rare cells that resemble primitive neural stem/progenitor cells that are normally tri-potent, capable of producing both glial lineages as well as neurons; genetic mutations may skew these tri-potent cancer cells towards generating glia (109,110). Consistent with this possibility, most PC2/3-associated genes, including SOX4 and SOX11, were upregulated upon activation of tri-potent mice neural stem cell (111) (NSCs) (FIG. 18C, FIG. 26B; P=3*10⁻⁶, t-test).

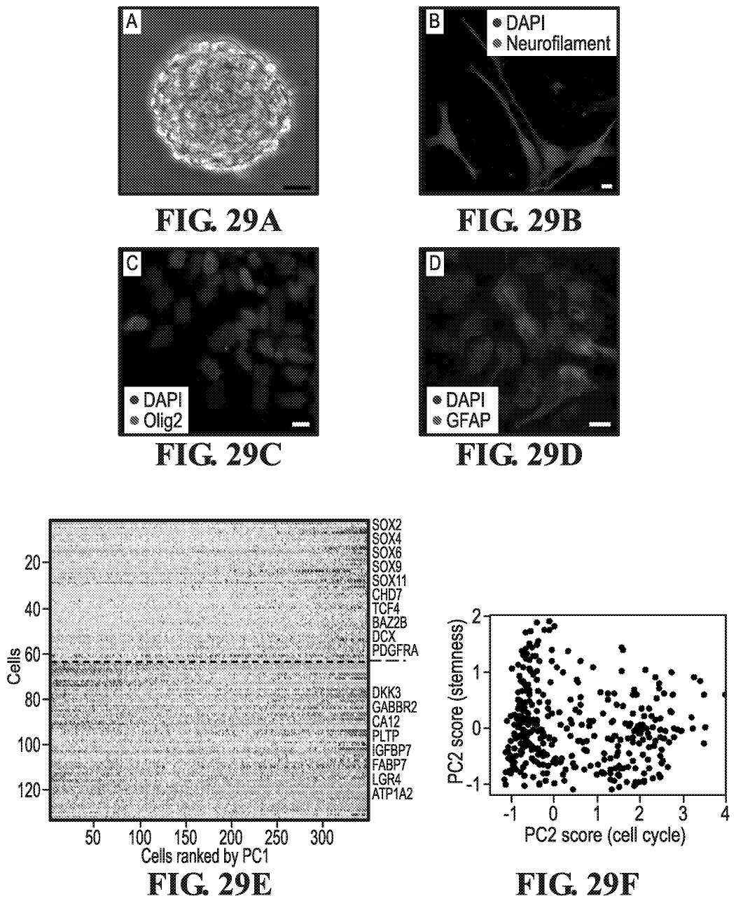

To further test the hypothesis that the stemness program is closely associated with tri-potent stem/progenitor cells, Applicants profiled by single-cell RNA-seq human neural progenitor cells (NPCs) isolated from fetal brain at 19 weeks of gestation and that can be differentiated into astrocytic, oligodendrocytic and neuronal lineages (FIG. 29A-D). While Applicants observed variation in the expression programs of these NPCs (FIG. 29E-F), unbiased PCA of the single cell NPC profiles identified a program highly similar to the PC2/3-associated program of tumor cells (FIG. 18C, FIG. 26C, Table 3; P=2*10⁻³⁵, t-test). Thus, a common program is shared by subsets of our putative oligodendroglioma stem cells and normal NPCs and NSCs. Taken together, the analysis revealed three main expression patterns that recapitulate oligodendrocytic and astrocytic differentiation (PC1 high and low, respectively) and stem/progenitor programs of early neural development (PC2/3 high).

TABLE 3
Top-correlated genes (R > 0.3) for PC1 and PC2 from
analysis of single cell RNA-seq of human NPCs.

| PC1 genes | PC1 correlation | PC2 genes | PC2 correlation |
|---|---|---|---|
| NEDD4L | 0,6929 | MAD2L1 | 0,8389 |
| KCNQ1OT1 | 0,6906 | ZWINT | 0,8234 |
| UGDH-ASI | 0,6732 | MLF1IP | 0,8209 |
| ORC4 | 0,6701 | RRM2 | 0,8182 |
| IGFBPL1 | 0,6615 | CCNA2 | 0,8173 |
| SHISA9 | 0,6593 | TPX2 | 0,8106 |
| ASTN2 | 0,6347 | UBE2T | 0,7881 |
| DCX | 0,633 | KIF11 | 0,7872 |
| METTL21A | 0,6096 | MELK | 0,7859 |
| TMEM212 | 0,5971 | NCAPG | 0,7816 |
| OPHN1 | 0,5828 | MKI67 | 0,7789 |
| NRXN3 | 0,5804 | NUSAP1 | 0,7758 |
| NREP | 0,5709 | CDK1 | 0,7745 |
| ARHGEF26-AS1 | 0,557 | HMGB2 | 0,7734 |
| ODF2L | 0,551 | NCAPH | 0,7724 |
| ABCC9 | 0,5483 | KIAA0101 | 0,7716 |
| PEG10 | 0,5471 | FANCI | 0,7657 |
| SOX9 | 0,5449 | NUF2 | 0,7582 |
| SOX4 | 0,5391 | TACC3 | 0,7570 |
| TCF4 | 0,535 | PRC1 | 0,7545 |
| CHD7 | 0,5242 | CDCA5 | 0,7544 |
| UGT8 | 0,516 | FOXMI | 0,7482 |
| DLX5 | 0,513 | CENPF | 0,7444 |
| XKR9 | 0,5036 | KIFC1 | 0,7441 |
| DLX6-AS1 | 0,4987 | TOP2A | 0,7434 |
| SOX11 | 0,4904 | KIF2C | 0,7431 |
| PDGFRA | 0,4865 | SMC2 | 0,7428 |
| DLX1 | 0,4783 | AURKB | 0,7409 |
| NPY | 0,4771 | FAM64A | 0,7375 |
| L2HGDH | 0,4728 | ASPM | 0,7325 |
| PTP RS | 0,4582 | DIAPH3 | 0,7292 |
| GLIPR1L2 | 0,4582 | UBE2C | 0,7285 |
| REX01L1 | 0,4549 | BUB1B | 0,7279 |
| CCL5 | 0,45 | NDC80 | 0,7234 |
| CTDSP2 | 0,4476 | ASF1B | 0,7224 |
| SOX2 | 0,4444 | KIF22 | 0,7214 |
| MAB21L3 | 0,4385 | TK1 | 0,7205 |
| TP53I11 | 0,4377 | FANCD2 | 0,7182 |
| GATS | 0,437 | CASC5 | 0,7177 |
| ZFHX4 | 0,4348 | GTS El | 0,7144 |
| BAZ2B | 0,4323 | RRM1 | 0,7133 |
| DCLK2 | 0,4313 | RACGAP1 | 0,7126 |
| GRIA2 | 0,4286 | TYMS | 0,7095 |
| LPAL2 | 0,4274 | BIRC5 | 0,7083 |
| CREBBP | 0,42 | PBK | 0,7048 |
| MARCH6 | 0,4198 | SPAG5 | 0,7004 |
| PGM5P2 | 0,4198 | KIF23 | 0,6977 |
| RERE | 0,4163 | TMPO | 0,6977 |
| SPC25 | 0,4143 | KIF15 | 0,6920 |
| GRIK3 | 0,4078 | DHFR | 0,6903 |
| CCDC88A | 0,4056 | H2AFZ | 0,6896 |
| PVRIG | 0,4038 | ANLN | 0,6871 |
| BRD3 | 0,4011 | ORC6 | 0,6857 |
| GRIA3 | 0,3996 | ARHGAP11A | 0,6809 |
| MOXD1 | 0,399 | ESCO2 | 0,6808 |
| SNTG1 | 0,3988 | KIF4A | 0,6806 |
| TAGLN3 | 0,3973 | RNASEH2A | 0,6802 |
| GSG1 | 0,3969 | RAD51AP1 | 0,6734 |
| DLX2 | 0,3946 | KIAA1524 | 0,6727 |
| ATCAY | 0,3877 | SMC4 | 0,6716 |
| NUMA1 | 0,3868 | CENPN | 0,6654 |
| LMO1 | 0,3861 | KIF18B | 0,6650 |
| POGZ | 0,3851 | VRK1 | 0,6636 |
| BPTF | 0,3849 | CCNB2 | 0,6609 |
| CHRM3 | 0,3848 | CKS1B | 0,6608 |
| RUFY3 | 0,3846 | CKAP2L | 0,6608 |
| SOX6 | 0,3833 | SHCBP1 | 0,6575 |
| RPS11 | 0,3833 | HIST1H1B | 0,6566 |
| TNFAIP8L1 | 0,3798 | SGOL1 | 0,6519 |
| FOXN3 | 0,3784 | HIST1H3B | 0,6452 |
| DAPKI | 0,3781 | CENPM | 0,6443 |
| DLL3 | 0,373 | CCNB1 | 0,6435 |
| HERC2P4 | 0,3728 | BUBI | 0,6434 |
| TFDP2 | 0,3724 | CENPK | 0,6433 |
| GTF2IP1 | 0,3704 | HMGN2 | 0,6427 |
| DLX6 | 0,37 | ECT2 | 0,6408 |
| IGFIR | 0,3698 | HMGB1 | 0,6399 |
| MLL3 | 0,3692 | UHRF1 | 0,6385 |
| NCAMI | 0,368 | NCAPD2 | 0,6370 |
| CHL1 | 0,3632 | HJURP | 0,6359 |
| GNRHR2 | 0,3553 | PKMYT1 | 0,6347 |
| CLIP3 | 0,3542 | MYBL2 | 0,6333 |
| FBLIM1 | 0,3508 | CDC45 | 0,6324 |
| MATR3 | 0,3505 | CDCA2 | 0,6322 |
| CCNG2 | 0,3498 | DLGAP5 | 0,6308 |
| NEK5 | 0,3469 | TUBB | 0,6302 |
| ETV1 | 0,3454 | MCM10 | 0,6259 |
| KAT6B | 0,3448 | ATAD2 | 0,6230 |
| SRRM2 | 0,3434 | MXD3 | 0,6226 |
| FOXP1 | 0,3423 | TUBAIB | 0,6192 |
| DDX17 | 0,3408 | SGOL2 | 0,6187 |
| GOSRI | 0,3391 | DTYMK | 0,6166 |
| GATAD2B | 0,3381 | CDC25C | 0,6162 |
| MAP4K4 | 0,3375 | TROAP | 0,6145 |
| MIAT | 0,3364 | DTL | 0,6134 |
| CD24 | 0,3327 | CDCA3 | 0,6120 |
| ZNF638 | 0,3317 | H2AFX | 0,6118 |
| HNRNPH1 | 0,3314 | LIG1 | 0,6110 |
| BRD8 | 0,3312 | TRIP13 | 0,6089 |
| MLL | 0,3285 | HAUS8 | 0,6087 |
| PCMTD1 | 0,328 | KIF20B | 0,6083 |
| AGPAT4 | 0,3251 | NCAPG2 | 0,6064 |
| YPEL1 | 0,3246 | CDKN3 | 0,6048 |
| TNIK | 0,3234 | MIS18BP1 | 0,6028 |
| PUMI | 0,3232 | BRCA1 | 0,5958 |
| RFTN2 | 0,3231 | PLK4 | 0,5924 |
| NNAT | 0,3188 | CENPW | 0,5910 |
| MALATI | 0,3185 | CDC20 | 0,5845 |
| GADI | 0,318 | SKA3 | 0,5837 |
| ZNF37BP | 0,3172 | HIST1H4C | 0,5834 |
| IRGQ | 0,3172 | LMNB1 | 0,5828 |
| FXYD6 | 0,3165 | CDCA8 | 0,5820 |
| PRRC2B | 0,3165 | PLK1 | 0,5796 |
| FAM110B | 0,3162 | RFC3 | 0,5795 |
| YPEL3 | 0,3151 | CENPO | 0,5778 |
| ZMIZ1 | 0,3148 | DNMT1 | 0,5764 |
| CLASP1 | 0,3142 | EX01 | 0,5741 |
| SYNE2 | 0,3134 | OIP5 | 0,5740 |
| BASP1 | 0,3134 | CHAF1A | 0,5738 |
| LYZ | 0,3133 | CENPE | 0,5713 |
| ROCK1P1 | 0,3117 | POC1A | 0,5705 |
| DPY19L2P2 | 0,3108 | DEK | 0,5663 |
| RSF1 | 0,3096 | NUCKSI | 0,5655 |
| HIP1 | 0,3083 | MCM7 | 0,5646 |
| KANSL1 | 0,3082 | MIS18A | 0,5645 |
| ELAVL4 | 0,3079 | DEPDCIB | 0,5641 |
| TET3 | 0,3058 | CHEK1 | 0,5632 |
| ZEB2 | 0,3054 | SPC24 | 0,5623 |
| ZBTB8A | 0,3052 | GMNN | 0,5586 |
| MTSS1 | 0,3051 | PTTG1 | 0,5583 |
| TNRC6B | 0,3036 | EZH2 | 0,5565 |
| FOXO3 | 0,3032 | MCM4 | 0,5552 |
| ANKRD12 | 0,3031 | FEN1 | 0,5549 |
| MEIS3 | 0,302 | GINS1 | 0,5543 |
| JMJDIC | 0,3018 | TTK | 0,5497 |
| RICTOR | 0,3004 | CDC6 | 0,5497 |
| MEST | 0,3003 | RAD51 | 0,5495 |
| | | C19orf48 | 0,5488 |
| | | KIF20A | 0,5461 |
| | | CKAP2 | 0,5453 |
| | | CDCA4 | 0,5442 |
| | | RFC5 | 0,5441 |
| | | SKA1 | 0,5440 |
| | | CENPQ | 0,5426 |
| | | FANCA | 0,5407 |
| | | PCNA | 0,5398 |
| | | RFC4 | 0,5395 |
| | | PARP2 | 0,5390 |
| | | TMEM194A | 0,5383 |
| | | FBXO5 | 0,5360 |
| | | TIMELESS | 0,5355 |
| | | PSMC3IP | 0,5348 |
| | | HIRIP3 | 0,5316 |
| | | POLAI | 0,5297 |
| | | RANBP1 | 0,5293 |
| | | KIF18A | 0,5291 |
| | | TCF19 | 0,5285 |
| | | USP1 | 0,5284 |
| | | LRR1 | 0,5277 |
| | | GGH | 0,5210 |
| | | HMMR | 0,5188 |
| | | CKS2 | 0,5186 |
| | | DNAJC9 | 0,5163 |
| | | SAE1 | 0,5142 |
| | | ITGB3BP | 0,5138 |
| | | TMEM106C | 0,5112 |
| | | FANCG | 0,5101 |
| | | KPNA2 | 0,5096 |
| | | NCAPD3 | 0,5078 |
| | | HELLS | 0,5071 |
| | | TMEM48 | 0,5069 |
| | | CBX5 | 0,5044 |
| | | SNRPB | 0,5011 |
| | | KNTC1 | 0,4975 |
| | | NASP | 0,4960 |
| | | MCM3 | 0,4946 |
| | | ZWILCH | 0,4933 |
| | | RPA3 | 0,4908 |
| | | CHTF18 | 0,4907 |
| | | ANP32E | 0,4903 |
| | | HIST1H3I | 0,4857 |
| | | POLA2 | 0,4854 |
| | | MZT1 | 0,4842 |
| | | MCM2 | 0,4839 |
| | | DEPDC1 | 0,4836 |
| | | DUT | 0,4835 |
| | | POLE | 0,4824 |
| | | PHIP | 0,4817 |
| | | PTMA | 0,4805 |
| | | CSEIL | 0,4786 |
| | | DSCC1 | 0,4780 |
| | | CDC7 | 0,4764 |
| | | HMGB3 | 0,4756 |
| | | TUBB4B | 0,4748 |
| | | STMN1 | 0,4747 |
| | | RPA2 | 0,4739 |
| | | RCC1 | 0,4726 |
| | | CENPH | 0,4719 |
| | | GINS2 | 0,4712 |
| | | EXOSC9 | 0,4710 |
| | | NCAPH2 | 0,4708 |
| | | NUDT15 | 0,4697 |
| | | SPC25 | 0,4674 |
| | | HNRNPA2B1 | 0,4674 |
| | | MND1 | 0,4643 |
| | | DSN1 | 0,4631 |
| | | MASTL | 0,4607 |
| | | RAD21 | 0,4604 |
| | | PHGDH | 0,4603 |
| | | ZNF331 | 0,4594 |
| | | RANGAP1 | 0,4588 |
| | | SAPCD2 | 0,4582 |
| | | PARPBP | 0,4579 |
| | | ANP32B | 0,4562 |
| | | SMCIA | 0,4554 |
| | | NEK2 | 0,4527 |
| | | BARD1 | 0,4526 |
| | | NIF3L1 | 0,4520 |
| | | PRR11 | 0,4506 |
| | | HNRNPD | 0,4500 |
| | | MCM5 | 0,4480 |
| | | SMC3 | 0,4479 |
| | | FAM111A | 0,4473 |
| | | POLDI | 0,4460 |
| | | CDK2 | 0,4458 |
| | | FUS | 0,4426 |
| | | PHF19 | 0,4399 |
| | | ARHGAP33 | 0,4345 |
| | | NUP205 | 0,4344 |
| | | CDC25B | 0,4335 |
| | | PA2G4 | 0,4323 |
| | | NUDT1 | 0,4311 |
| | | CHEK2 | 0,4307 |
| | | WDR34 | 0,4305 |
| | | H2AFY | 0,4271 |
| | | HAUS1 | 0,4239 |
| | | BUB3 | 0,4236 |
| | | CHAF1B | 0,4206 |
| | | PRIM2 | 0,4190 |
| | | CCDC34 | 0,4176 |
| | | POLE2 | 0,4175 |
| | | PRPS2 | 0,4174 |
| | | RFWD3 | 0,4171 |
| | | UBR7 | 0,4155 |
| | | CCNE2 | 0,4145 |
| | | RAN | 0,4144 |
| | | DDX11 | 0,4142 |
| | | NUP50 | 0,4131 |
| | | CACYBP | 0,4128 |
| | | HNRNPAB | 0,4123 |
| | | DBF4 | 0,4120 |
| | | TMSB15A | 0,4114 |
| | | AURKA | 0,4106 |
| | | MAD2L2 | 0,4095 |
| | | GINS3 | 0,4095 |
| | | ASRGL1 | 0,4086 |
| | | PPIF | 0,4084 |
| | | CKAP5 | 0,4060 |
| | | UBE2S | 0,4053 |
| | | LMNB2 | 0,4040 |
| | | POLD3 | 0,4039 |
| | | TEX30 | 0,4002 |
| | | SUV39H1 | 0,3999 |
| | | CCP110 | 0,3997 |
| | | WHSCI | 0,3988 |
| | | MCM6 | 0,3986 |
| | | ACYP1 | 0,3983 |
| | | GNG4 | 0,3957 |
| | | PRIMI | 0,3933 |
| | | NSMCE4A | 0,3920 |
| | | EXOSC8 | 0,3916 |
| | | COMMD4 | 0,3910 |
| | | SNRPD1 | 0,3887 |
| | | HAT1 | 0,3885 |
| | | H2AFV | 0,3870 |
| | | CMC2 | 0,3868 |
| | | SSRP1 | 0,3858 |
| | | HIST1H1E | 0,3852 |
| | | RBMX | 0,3844 |
| | | LBR | 0,3842 |
| | | RPL39L | 0,3818 |
| | | EMP2 | 0,3818 |
| | | CENPL | 0,3813 |
| | | CEP78 | 0,3809 |
| | | TRAIP | 0,3807 |
| | | COPS3 | 0,3781 |
| | | LSM4 | 0,3779 |
| | | RBBP8 | 0,3774 |
| | | HIST1H1C | 0,3743 |
| | | RPAl | 0,3733 |
| | | RADI | 0,3714 |
| | | NUP210 | 0,3712 |
| | | HSPB11 | 0,3701 |
| | | RFC2 | 0,3684 |
| | | ACTL6A | 0,3671 |
| | | SRRT | 0,3663 |
| | | NUP107 | 0,3655 |
| | | GPN3 | 0,3614 |
| | | LSM3 | 0,3606 |
| | | SUV39H2 | 0,3602 |
| | | POLR2D | 0,3597 |
| | | HAUS5 | 0,3594 |
| | | WDR76 | 0,3588 |
| | | LSM5 | 0,3575 |
| | | NXT1 | 0,3563 |
| | | TUBG1 | 0,3557 |
| | | C16orf59 | 0,3554 |
| | | REEP4 | 0,3539 |
| | | BTG3 | 0,3538 |
| | | RNASEH2B | 0,3538 |
| | | TUBB6 | 0,3534 |
| | | PPIA | 0,3524 |
| | | RBL1 | 0,3522 |
| | | ARL6IP6 | 0,3504 |
| | | COX17 | 0,3501 |
| | | SYNE2 | 0,3500 |
| | | GUSB | 0,3499 |
| | | MSH5 | 0,3479 |
| | | CRNDE | 0,3472 |
| | | DDX39A | 0,3467 |
| | | SUPT16H | 0,3467 |
| | | HNRNPULl | 0,3455 |
| | | POLE3 | 0,3454 |
| | | HAUS4 | 0,3449 |
| | | IDH2 | 0,3448 |
| | | H1FX | 0,3439 |
| | | DCP2 | 0,3427 |
| | | NUP188 | 0,3417 |
| | | MPHOSPH9 | 0,3415 |
| | | PPIG | 0,3407 |
| | | MAGOHB | 0,3400 |
| | | RIF1 | 0,3393 |
| | | MLH1 | 0,3386 |
| | | MSH2 | 0,3367 |
| | | SNRNP40 | 0,3363 |
| | | HADH | 0,3346 |
| | | GABPB1 | 0,3341 |
| | | NUDC | 0,3332 |
| | | PHTF2 | 0,3328 |
| | | NUP85 | 0,3325 |
| | | NUP35 | 0,3316 |
| | | SKP2 | 0,3310 |
| | | THOC3 | 0,3292 |
| | | ANAPC11 | 0,3283 |
| | | TFAM | 0,3283 |
| | | AKR1B1 | 0,3281 |
| | | ILF2 | 0,3276 |
| | | TMEM237 | 0,3268 |
| | | RAD54B | 0,3258 |
| | | SMPD4 | 0,3258 |
| | | HMGN1 | 0,3255 |
| | | CBX3 | 0,3253 |
| | | TPRKB | 0,3250 |
| | | GGCT | 0,3249 |
| | | FBL | 0,3249 |
| | | RFC1 | 0,3247 |
| | | CCT5 | 0,3231 |
| | | PRKDC | 0,3222 |
| | | CDK5RAP2 | 0,3221 |
| | | SRSF2 | 0,3204 |
| | | CEP112 | 0,3191 |
| | | LDHA | 0,3189 |
| | | SRSF3 | 0,3183 |
| | | HSP90AA1 | 0,3179 |
| | | SRSF7 | 0,3175 |
| | | HAUS6 | 0,3150 |
| | | CCHCR1 | 0,3143 |
| | | CEP57 | 0,3135 |
| | | HMGA1 | 0,3129 |
| | | UCHL5 | 0,3122 |
| | | Clorfl74 | 0,3120 |
| | | CTPS1 | 0,3120 |
| | | ACOT7 | 0,3119 |
| | | SNHG1 | 0,3119 |
| | | PSMC3 | 0,3116 |
| | | ZNF93 | 0,3106 |
| | | 10/sep | 0,3100 |
| | | PCM1 | 0,3091 |
| | | SFPQ | 0,3089 |
| | | RMI1 | 0,3084 |
| | | NUP37 | 0,3057 |
| | | DCK | 0,3056 |
| | | AHI1 | 0,3052 |
| | | SVIP | 0,3051 |
| | | CHCHD2 | 0,3049 |
| | | ZNF714 | 0,3049 |
| | | XRCC5 | 0,3048 |
| | | NFATC2IP | 0,3040 |
| | | SLC25A5 | 0,3036 |
| | | WRAP53 | 0,3034 |
| | | PSIP1 | 0,3029 |
| | | MRPS6 | 0,3021 |
| | | NT5DC2 | 0,3015 |
| | | NOP58 | 0,3003 |

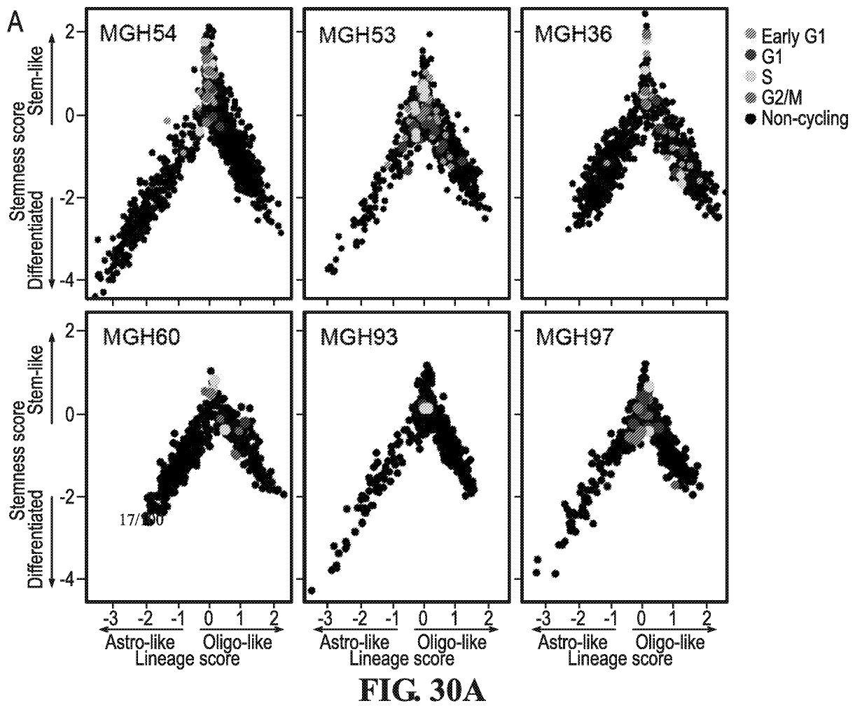

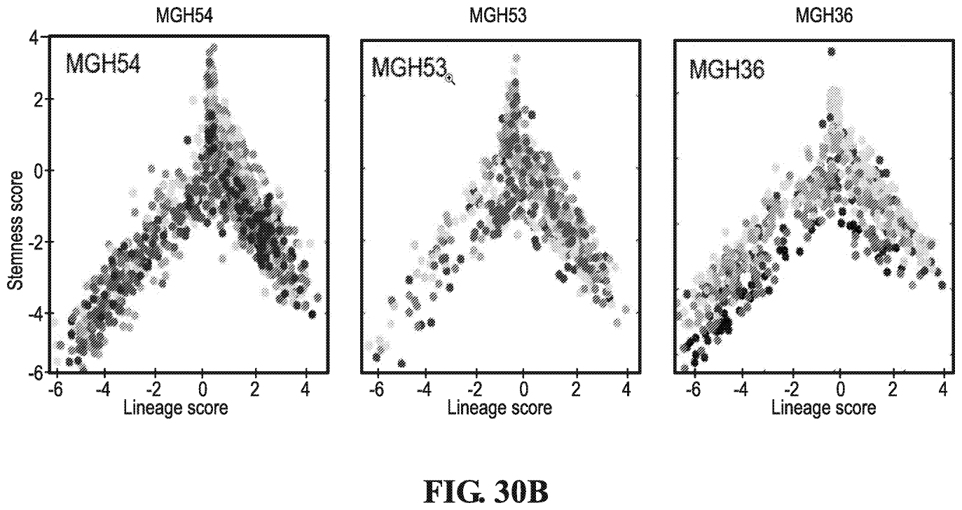

To precisely assign a cellular state to each individual tumor cell, Applicants defined an OC vs. AC lineage score and a sternness vs. differentiation score (Methods). Plotting these two scores across the cells of all three tumors together revealed a striking similarity to normal cellular hierarchies (FIG. 18D), with a transition from a stem/progenitor program branching into differentiation along two glial lineages. Importantly, the same architecture was observed in each of the six tumors (FIG. 18E, FIG. 29). Statistical analysis of the variation in lineage score compared to expected technical noise suggests that the transition involves intermediate states for each lineage (FIG. 30), but the exact number of states and whether they are discrete or form a more continuous trajectory is difficult to determine due to technical limitations associated with noise in single cell RNA-seq data (Note 2).

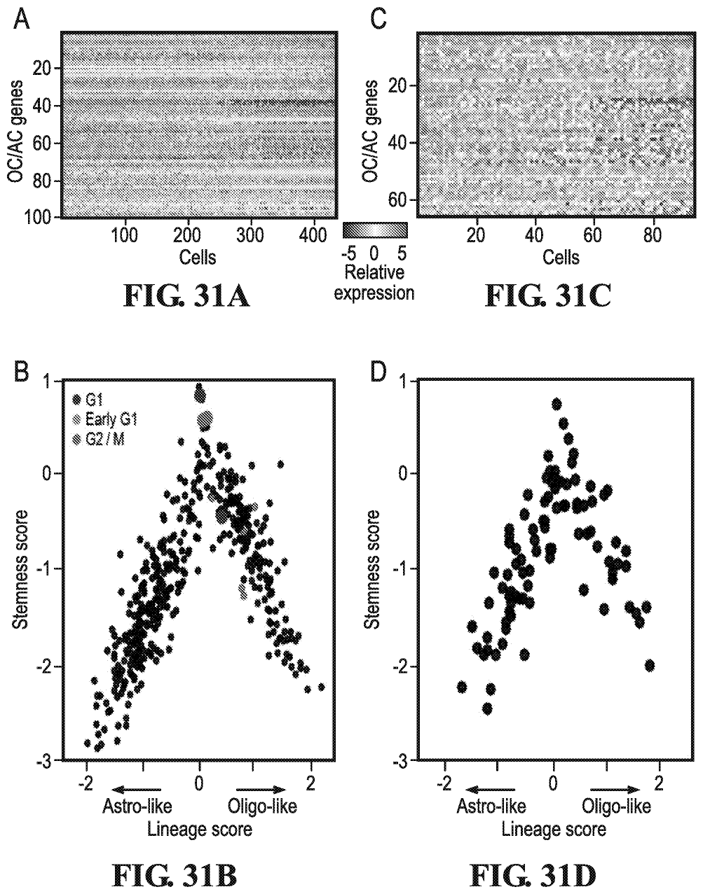

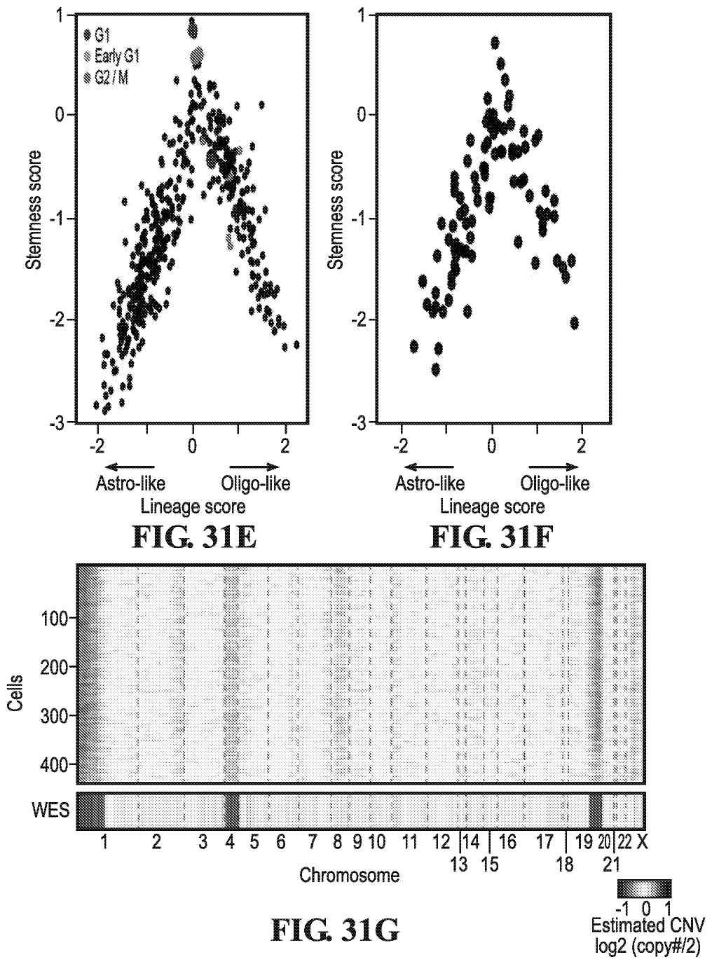

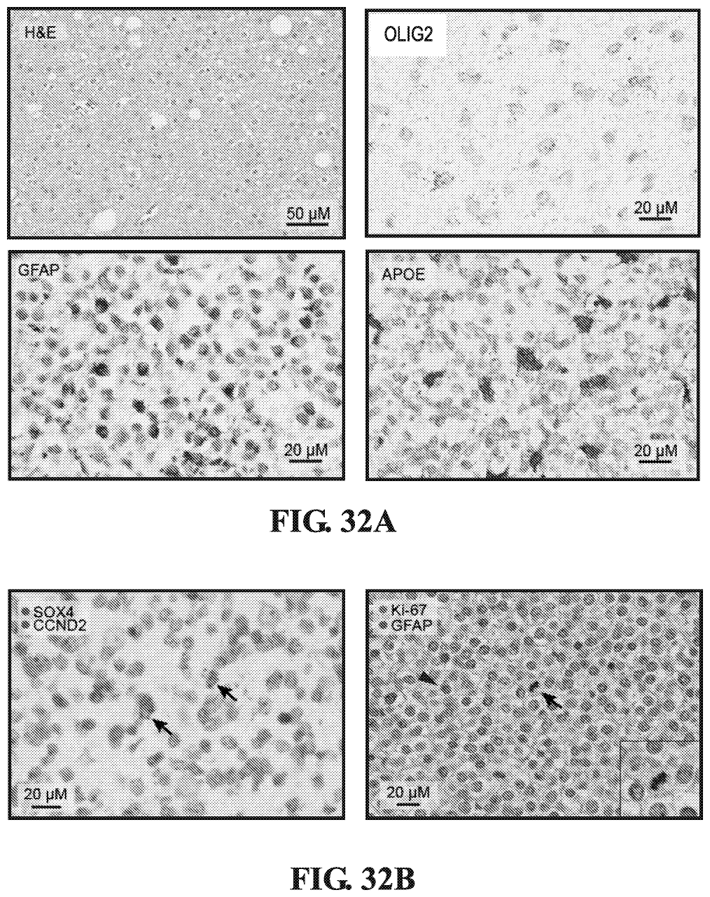

Applicants validated the generality of these findings in two ways. First, Applicants observed the same architecture when Applicants independently profiled one of the tumors (MGH60) with a different method for single cell RNA-seq (Methods; FIG. 31). Second, Applicants confirmed these patterns in tumors by both RNA in situ hybridization and immunohistochemistry with markers of AC (GFAP and APOE), OC (OLIG2, OMG) and stem/progenitor cells (SOX4, CCND2) performed in each of the original 6 tumors and in a validation cohort of ten additional tumors (FIG. 18F,G, FIG. 32 and Table 4).

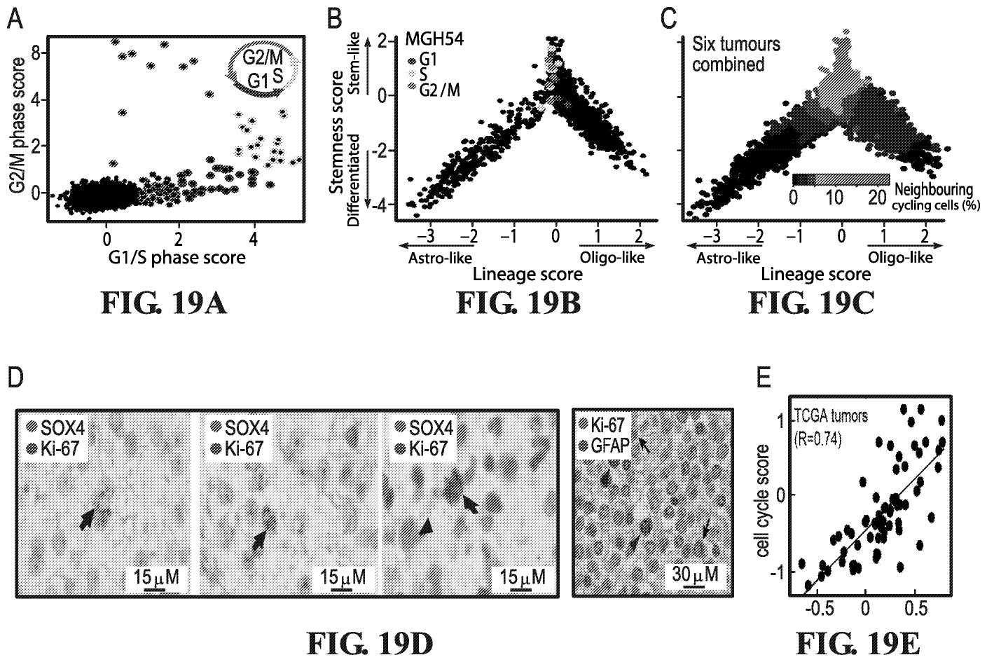

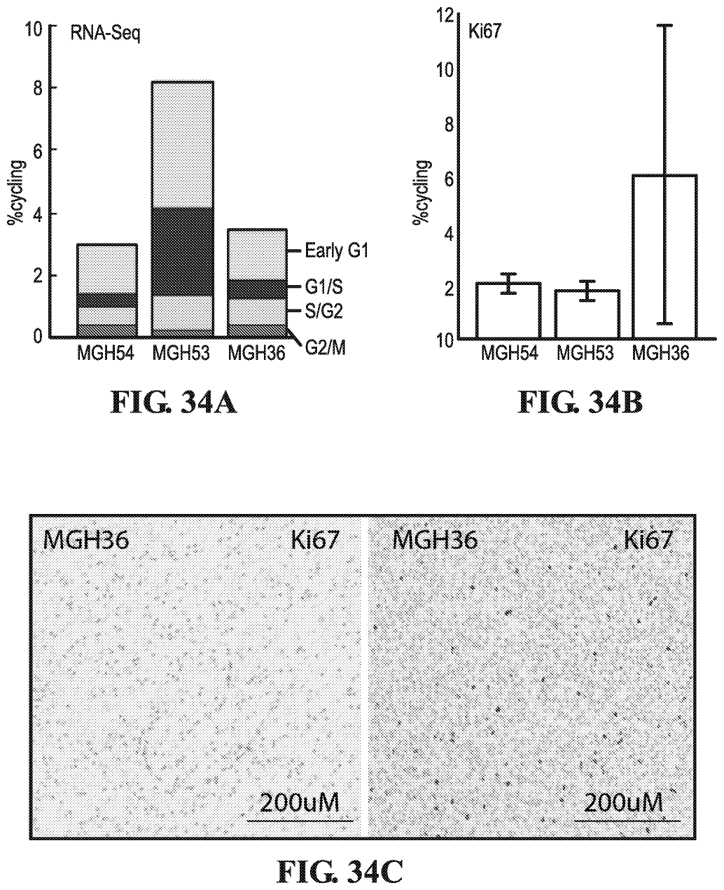

This architecture suggests a developmental hierarchy in which tumor stem/progenitor cells give rise to differentiated progeny. To assess how patterns of tumor proliferation and self-renewal may relate to the developmental hierarchy, Applicants next scored each cell for the expression of consensus gene sets for the G1/S phases and the G2/M phases, which Applicants defined based on consistent association with those phases across multiple datasets (Methods) (16, 124) Applicants found that only a small proportion of cells in each tumor (1.5-8%) are proliferating (FIG. 19A, FIG. 33-34). The fraction of proliferating cells Applicants identified by expression program is within the expected range for oligodendrogliomas and comparable to the percentage of cycling cells identified by Ki-67 staining in these tumors, with the caveat that proliferation can vary substantially between different regions of the same tumor (FIG. 34). Applicants further distinguished cycling cells by their G1/S and G2/M scores, to identify four distinct cell cycle phases (FIG. 19A).

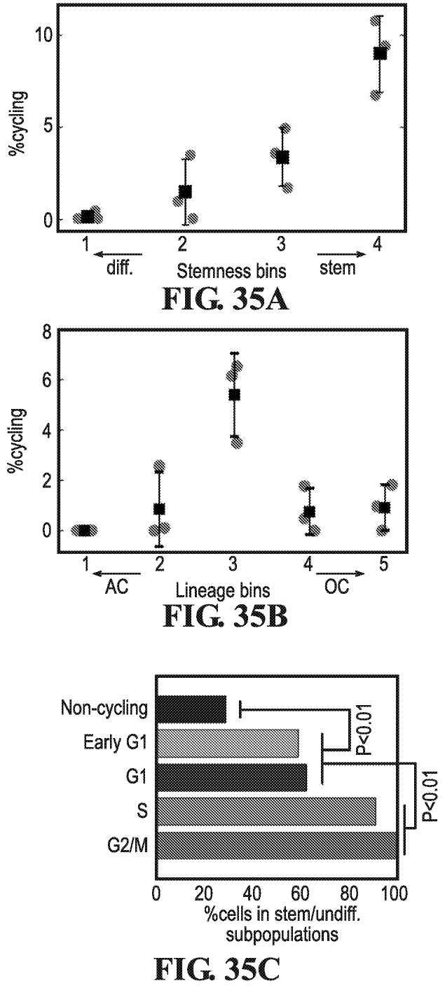

Strikingly, almost all cycling cancer cells were confined to the stem/progenitor and undifferentiated compartment of the tumor (FIG. 19B,C, FIG. 35A,B), suggesting that this represents the compartment responsible for the growth of oligodendrogliomas in humans. Several lines of evidence support the finding that stem/progenitor and undifferentiated cells account for tumor proliferation. First, Applicants validated the co-expression of a stem/progenitor marker (SOX4) and the cell proliferation marker (Ki67) in tissue staining across 14 patients, as well as a negative correlation for cycling and glial differentiation markers (FIG. 19D and FIG. 32 and Table 4). Second, there is a strong correlation between our cell-cycle signature and our stem/progenitor signature across 69 bulk oligodendroglioma samples in the TCGA dataset (FIG. 19E) (112). Finally, the enrichment of cell cycle among stem/progenitor and undifferentiated cells was even more striking for cells inferred to be in G2/M phases compared to those in the G1 phase (FIG. 35C), possibly reflecting the short G1 phase observed in tissue and embryonic stem cells (113).

TABLE 4
Fraction of cells in each subpopulation as estimated by single cell RNA-seq (top)
and tissue staining (bottom)

| | | | | | Cycling stem- | Cycling stem- | Cycling OC- | Cycling AC- | | | |
|---|---|---|---|---|---|---|---|---|---|---|---|
| | OC- | AC- | Stem- | | like (with | like + undif. | like (with | like (with | OC + | OC + | AC + |
| | like | like | like | Undif. | early G1) | (with early G1) | early G1) | early G1) | AC | stem | stem |
| MGH36 | 34.21% | 49.20% | 10.04% | 6.55% | 0.72% (1.01%) | 1.15% (1.44)% | 0.43% (1.01%) | 0% (0.14%) | 0.15% | 4.22% | 1.60% |
| MGH53 | 38.64% | 17.33% | 14.35% | 29.69% | 0.55% (1.65%) | 2.62% (4.96)% | 0.14% (0.14%) | 0% (0.14%) | 0.14% | 0.43% | 0.99% |
| MGH54 | 44.57% | 23.10% | 16.90% | 15.43% | 0.77% (1.53%) | 1.28% (2.56)% | 0% (0%) | 0% (0.09%) | 0.17% | 1.29% | 0.78% |
| MGH60 | 34.66% | 50.82% | 4.22% | 10.30% | 0.47% (0.93%) | 0.7% (2.09)% | 0.23% (0.7%) | 0% (0.7%) | 0.00% | 3.28% | 0.23% |
| Average | 38.02% | 35.11% | 11.38% | 15.49% | 0.63% (1.28%) | 1.44% (2.76)% | 0.2% (0.46%) | 0% (0.27%) | 0.12% | 2.31% | 0.90% |

| | | | | SOX4 + | CCND2 + | CCND2 + | CCND2 + |
|---|---|---|---|---|---|---|---|
| | OMG | APOE | SOX4 | Ki67 | SOX4 | OMG | APOE |
| MGH36 | 31.00% | 41.00% | 8.00% | 2.10% | 1.90% | 0.20% | 0% |
| MGH53 | 30.00% | 15.00% | 12.00% | 1.30% | 1.00% | 0% | 0% |
| MGH54 | 37.00% | 25.00% | 9.00% | 0.90% | 1.10% | 0.20% | 0% |
| Oligo 1 | 28.00% | 26.00% | 7.00% | 0.90% | 1.00% | 0% | 0% |
| Oligo 2 | 31.00% | 17.00% | 2.00% | 0.90% | 1.00% | 0% | 0.10% |
| Oligo 3 | 43.00% | 19.00% | 6.00% | 1.60% | 1.30% | 0% | 0% |
| Oligo 4 | 45.00% | 11.00% | 8.00% | 1.90% | 2.00% | 0.30% | 0.10% |
| Oligo 5 | 24.00% | 30.00% | 3.00% | 0.90% | 1.00% | 0% | 0% |
| Oligo 6 | 12.00% | 47.00% | 5.00% | 0.30% | 0.90% | 0% | 0% |
| Oligo 7 | 22.00% | 35.00% | 4.00% | 3.00% | 4.00% | 0.50% | 0.50% |
| Oligo 8 | 25.00% | 37.00% | 2.00% | 1.30% | 1.50% | 0% | 0.20% |
| Oligo 9 | 27.00% | 38.00% | 7.00% | 0.50% | 1.00% | 0.10% | 0% |
| Oligo 10 | 36.00% | 29.00% | 9.00% | 0.70% | 0.90% | 0% | 0% |
| Average | 30.00% | 28.50% | 6.30% | 1.25% | 1.43% | 0.10% | 0.07% |

Although cycling cells were highly enriched among stem/progenitors, the frequency of cycling cells was low (˜10%) even among stem/progenitors. Because cycling cells are a minority even among stem/progenitor cells, the PC2/3 stem/progenitor program did not include a signature for cell cycle. The notable exception is CCND2 (FIG. 18A), a gene which plays a major role in controlling the cell cycle and was previously associated with self-renewal of glioma CSC (114). Interestingly, CCND2 was highly expressed both in cycling cells as well in non-cycling stem/progenitor cells (FIG. 36A,B), consistent with previous work that implicated it in priming cells to enter the cell cycle (113). Stem/progenitor tumor cells preferentially express CCND2, whereas differentiated tumor cells express CCND1 and CCND3, mirroring the high expression of CCND2 in early neurodevelopment, which is later replaced by CCND1 and CCND3 (FIG. 36C). CCND2 was also upregulated in activated mouse NSCs prior to entering the cell cycle (FIG. 36D). Taken together, these results indicate a role of CCND2 in both normal and malignant neural stem cell programs.

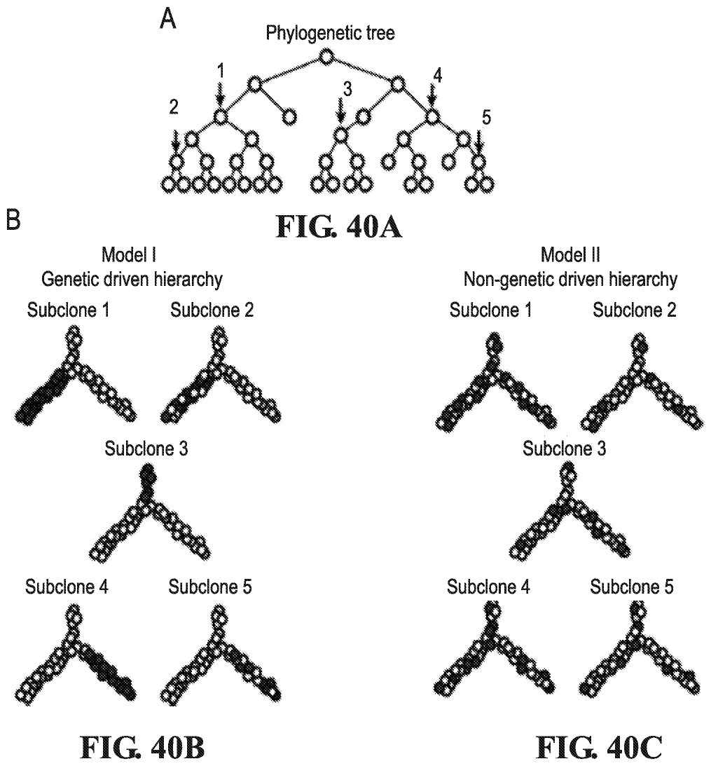

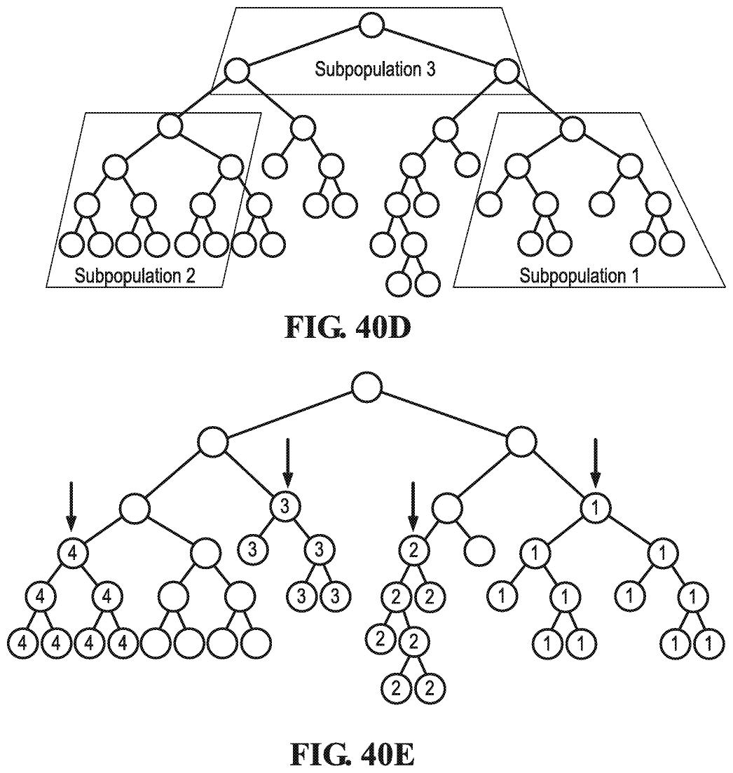

Finally, Applicants explored the role of genetic events in shaping the cellular identity, devising two approaches to obtain genetic information from single cell RNA-seq and classify cells into tumor subclones. In the first approach, Applicants used the CNV inference (FIG. 17B,C) of each cell to relate its genetic state with its transcriptional profile. In this approach, Applicants can ascertain the CNV features for every cell, but the number of genetic features is small (few CNVs). In the second approach, Applicants identified subclonal point mutations from bulk DNA whole-exome sequencing, using the ABSOLUTE method (35), and then searched for these mutations in the RNA-seq reads of individual cells (Methods). This approach assesses a larger number of mutations, but its sensitivity is limited by RNA-seq coverage, heterozygosity and allele-specific expression, such that Applicants could only ascertain (observe) mutations in a small fraction of cells compared to the expected subclonal fraction (Methods). Applicants performed whole-exome sequencing from bulk tumors and matched blood, identified tumor-specific single-point mutations (Table 5) and mapped them to our single profiled cells based on RNA-seq reads that harbored these exact mutations (FIG. 20C). However, the confidence of the ascertained mutations is illustrated by a low estimated false positive rate (<1%) (Methods) and by validation of a subset of mutations by qPCR (below) and targeted sequencing (Methods). The genetic information obtained with these two approaches is partial and is not sufficient to reconstruct a full phylogenetic tree. However, Applicants reasoned that it is sufficient to test if each subclonal genetic feature is restricted to a certain developmental state or if alternatively, according to the model of non-genetically-driven hierarchy, subclones span distinct developmental states (FIG. 40).

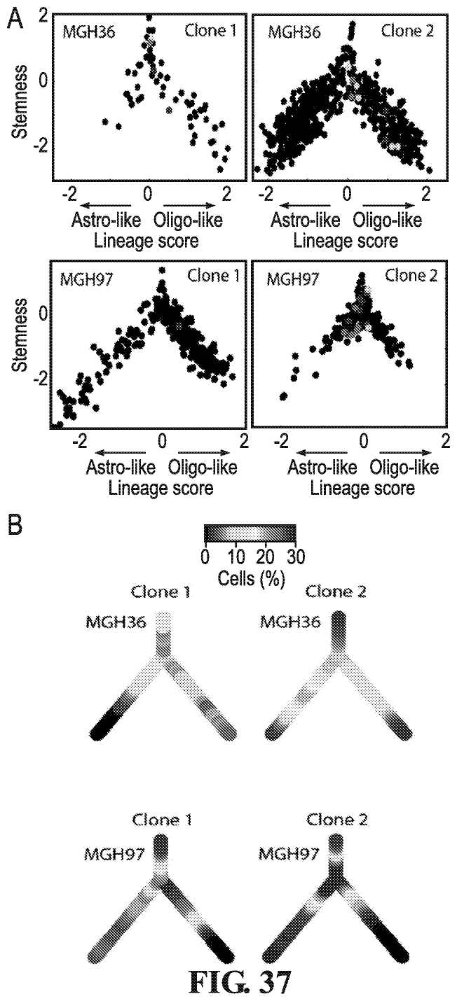

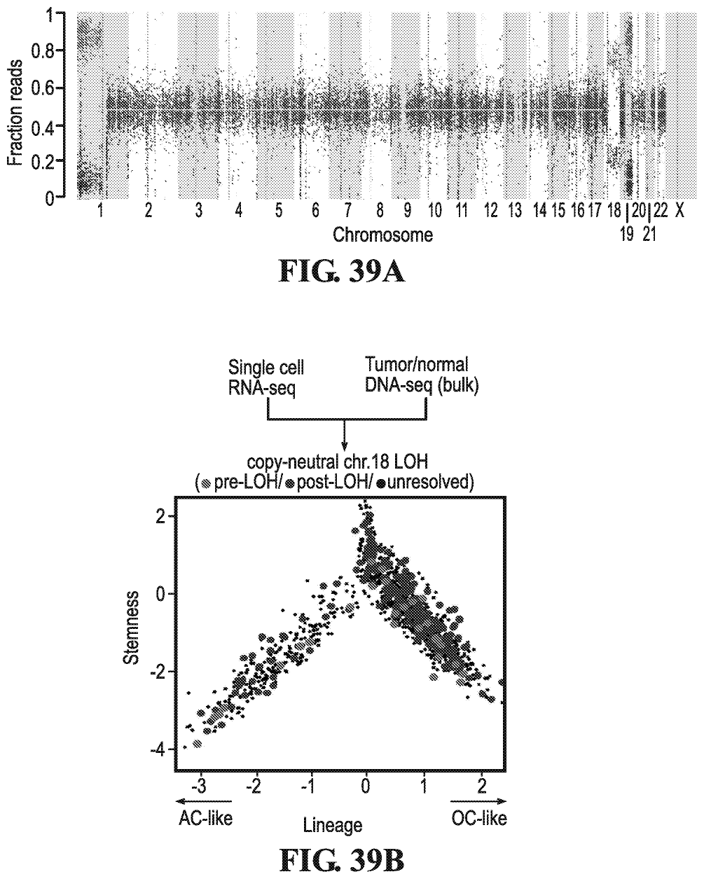

Applicants observed the same 3 sub-population architecture within distinct CNV sub-clones in MGH36 and in MGH97 (FIG. 17C), with cycling stem/progenitor cells and two lineages of differentiated non-cycling cells (FIG. 20A,B, FIG. 37). This suggests that distinct CNV profiles do not dictate a specific cellular state, and rather that developmental programs are over-imposed over CNV clones. Similarly, examining the distribution of transcriptional states for cells that harbor subclonal point mutations, Applicants found that 23 subclonal point mutations (FIG. 20C,D and FIG. 38) and a subclonal loss-of-heterozygosity event (FIG. 39) are not significantly restricted to particular developmental states and often span all three states. In particular, these include multiple cases with low subclonal fraction (<12% based on ABSOLUTE) that nevertheless span all three compartments in the transcriptional hierarchy (e.g., point mutations in ZEB2, EEF1B2, FTH1, FRG1B, and CNV clone 1 in MGH36). Regardless of whether a mutation has low fraction because it arose early (and did not rise in frequency) or late (and is thus a minor deep branch), the fact that it spans all compartments strongly argues against a genetic explanation.

Thus, our approach, applied across CNVs and multiple point mutations provides many examples of distinct genetic subclones that span the developmental hierarchy. This indicates that oligodendroglioma's developmental hierarchy is largely maintained during genetic evolution. The presence of a similar hierarchy in each of the tumors examined and across multiple subclones within each tumor, together with the lack of shared subclonal mutations across these oligodendrogliomas, strongly argues that the hierarchy is not driven by genetics.

TABLE 5
Mutations identified by DNA whole exome sequencing of tumor tissue and matched
blood, their ABSOLUTE-estimated clonal fraction

| | | | | | | | | |
|---|---|---|---|---|---|---|---|---|
| | | | cancer cell | | | | | |
| | | | fraction | Variant_ | Reference_ | Alternative_ | cDNA_ | Protein_ |
| Hugo_Symbol | Chromosome | position | (ABSOLUTE) | Classification | Allele | Allele | Change | Change |

MGH53

| | | | | | | | | |
|---|---|---|---|---|---|---|---|---|
| DDX11L1 | 1 | 15906 | 0.28 | RNA | A | G | | |
| DDX11L1 | 1 | 15922 | 0.21 | RNA | A | G | | |
| PLCH2 | 1 | 2435349 | 1 | Intron | A | C | | |
| PLCH2 | 1 | 2435352 | 0.89 | Intron | T | C | | |
| PLCH2 | 1 | 2435357 | 1 | Intron | A | C | | |
| NBPF1 | 1 | 16892724 | 0.04 | Intron | A | T | | |
| Unknown | 1 | 16974745 | 0.08 | IGR | G | A | | |
| ZNF362 | 1 | 33747370 | 0.96 | Missense_Mutation | A | G | c.866A > G | p.D289G |
| OSBPL9 | 1 | 52226257 | 0.64 | Intron | T | G | | |
| IGSF3 | 1 | 117158772 | 0.13 | Silent | C | T | c.351G > A | p.E117E |
| LCE1A | 1 | 152799987 | 0.5 | Silent | T | C | c.39T > C | p.P13P |
| PMVK | 1 | 154897570 | 1 | 3′UTR | T | C | | |
| THBS3 | 1 | 155167452 | 0.6 | Splice_Site | T | G | | |
| KIAA0907 | 1 | 155887387 | 0.76 | Missense_Mutation | T | G | c.1343A > C | p.Q448P |
| KIAA0907 | 1 | 155887393 | 0.58 | Missense_Mutation | T | G | c.1337A > C | p.Q446P |
| SH2D2A | 1 | 156777070 | 0.61 | Missense_Mutation | T | G | c.1070A > C | p.Q357P |
| SH2D2A | 1 | 156777073 | 0.79 | Missense_Mutation | T | G | c.1067A > C | p.H356P |
| DARS2 | 1 | 173795839 | 0.2 | Missense_Mutation | G | T | c.142G > T | p.V48F |
| CR1 | 1 | 207787753 | 0.1 | Nonsense_Mutation | C | T | c.6580C > T | p.R2194* |
| LYST | 1 | 235938295 | 0.11 | Missense_Mutation | T | G | c.5552A > C | p.E1851A |
| FMN2 | 1 | 240371436 | 0.35 | Silent | T | C | c.3324T > C | p.P1108P |
| CEP170 | 1 | 243319558 | 0.25 | Silent | G | T | c.3876C > A | p.I1292I |
| CEP170 | 1 | 243333027 | 0.12 | Silent | A | G | c.1746T > C | p.R582R |
| KIF26B | 1 | 245765965 | 0.11 | Missense_Mutation | G | T | c.1437G > T | p.K479N |
| C2orf71 | 2 | 29293879 | 0.31 | Silent | A | G | c.3249T > C | p.P1083P |
| ALK | 2 | 29455195 | 0.55 | Silent | C | A | c.2607G > T | p.G869G |
| EIF2AK2 | 2 | 37374837 | 0.29 | Missense_Mutation | T | G | c.113A > C | p.D38A |
| CTNNA2 | 2 | 80136918 | 0.59 | Missense_Mutation | A | C | c.1051A > C | p.N351H |
| IL1RL2 | 2 | 102835512 | 0.21 | Missense_Mutation | A | C | c.824A > C | p.D275A |
| RGPD3 | 2 | 107049681 | 0.04 | Missense_Mutation | T | C | c.2266A > G | p.N756D |
| FOXD4L1 | 2 | 114256759 | 0.21 | 5′UTR | A | G | | |
| KIF5C | 2 | 149633151 | 1 | 5′UTR | A | C | | |
| KIF5C | 2 | 149633155 | 0.98 | 5′UTR | A | C | | |
| KIF5C | 2 | 149633161 | 0.68 | 5′UTR | G | C | | |
| RAPH1 | 2 | 204322299 | 0.09 | Missense_Mutation | T | C | c.1112A > G | p.K371R |
| ADAM23 | 2 | 207452868 | 0.09 | Silent | C | A | c.1842C > A | p.I614I |
| CPO | 2 | 207833951 | 0.34 | Missense_Mutation | T | G | c.916T > G | p.S306A |
| IDH1 | 2 | 209113112 | 0.95 | Missense_Mutation | C | T | c.395G > A | p.R132H |
| IRS1 | 2 | 227660628 | 0.14 | Missense_Mutation | T | G | c.2827A > C | p.K943Q |
| UBE2F-SCLY | 2 | 238965872 | 0.28 | 3′UTR | T | A | | |
| TPRXL | 3 | 14106174 | 0.28 | Silent | T | C | c.498T > C | p.S166S |
| NR2C2 | 3 | 15084335 | 0.77 | Intron | TT | GG | | |
| NGLY1 | 3 | 25770654 | 0.42 | Silent | T | G | c.1581A > C | p.I527I |
| PLXNB1 | 3 | 48461609 | 0.5 | Missense_Mutation | T | G | c.2086A > C | p.T696P |
| PLXNB1 | 3 | 48461613 | 0.49 | Silent | T | G | c.2082A > C | p.P694P |
| BTLA | 3 | 112198364 | 0.14 | Missense_Mutation | C | T | c.341G > A | p.R114H |
| PIK3CB | 3 | 138433351 | 0.77 | Missense_Mutation | T | G | c.1261A > C | p.N421H |
| CLRN1 | 3 | 150645448 | 0.15 | 3′UTR | T | C | | |
| P2RY12 | 3 | 151055868 | 0.34 | Nonsense_Mutation | G | A | c.766C > T | p.R256* |
| EGFEM1P | 3 | 168530083 | 0.81 | RNA | A | T | | |
| MUC4 | 3 | 195507144 | 0.07 | Silent | C | T | c.11307G > A | p.V3769V |
| MUC4 | 3 | 195513285 | 0.05 | Silent | G | T | c.5166C > A | p.S1722S |
| MFI2 | 3 | 196736499 | 0.21 | Silent | G | A | c.1515C > T | p.D505D |
| ATP5I | 4 | 667819 | 0.35 | Intron | A | G | | |
| CLOCK | 4 | 56304585 | 0.2 | Missense_Mutation | G | A | c.2225C > T | p.A742V |
| PDCL2 | 4 | 56435894 | 0.43 | Missense_Mutation | T | G | c.353A > C | p.Y118S |
| GYPE | 4 | 144797983 | 0.91 | Silent | C | T | c.162G > A | p.A54A |
| PDE4D | 5 | 58295396 | 0.18 | Intron | G | A | | |
| KIF2A | 5 | 61602215 | 1 | 5′UTR | T | C | | |
| NBPF22P | 5 | 85589141 | 0.07 | RNA | T | G | | |
| SYCP2L | 6 | 10942975 | 0.21 | Missense_Mutation | C | A | c.1950C > A | p.D650E |
| ACOT13 | 6 | 24701717 | 0.32 | Missense_Mutation | T | G | c.297T > G | p.D99E |
| BTN2A3P | 6 | 26422353 | 0.13 | RNA | C | T | | |
| ZNF165 | 6 | 28053590 | 0.34 | Missense_Mutation | A | C | c.332A > C | p.E111A |
| Unknown | 6 | 29856906 | 0.17 | IGR | G | A | | |
| NRM | 6 | 30658769 | 0.46 | 5′UTR | T | G | | |
| BAG6 | 6 | 31610160 | 0.78 | Silent | T | G | c.1974A > C | p.P658P |
| GPR116 | 6 | 46856205 | 0.12 | Silent | A | G | c.195T > C | p.V65V |
| PTP4A1 | 6 | 64289971 | 0.25 | Silent | T | G | c.414T > G | p.R138R |
| ZNF292 | 6 | 87965630 | 0.18 | Missense_Mutation | T | G | c.2283T > G | p.F761L |
| ORC3 | 6 | 88318940 | 1 | Missense_Mutation | A | C | c.706A > C | p.I236L |
| CDC40 | 6 | 110534309 | 0.86 | Missense_Mutation | G | T | c.888G > T | p.L296F |
| LAMA2 | 6 | 129371133 | 0.03 | Silent | A | G | c.183A > G | p.K61K |
| VNN1 | 6 | 133014444 | 1 | Missense_Mutation | A | C | c.545T > G | p.F182C |
| MAP7 | 6 | 136699003 | 0.34 | Missense_Mutation | C | T | c.641G > A | p.R214H |
| UNC93A | 6 | 167728954 | 0.16 | 3′UTR | C | T | | |
| FAM120B | 6 | 170627052 | 0.44 | Missense_Mutation | T | G | c.574T > G | p.S192A |
| PHF14 | 7 | 11013807 | 1 | 5′UTR | G | A | | |
| H2AFV | 7 | 44874056 | 0.13 | 3′UTR | A | C | | |
| ABCA13 | 7 | 48232645 | 0.18 | Silent | C | T | c.159C > T | p.D53D |
| TMEM248 | 7 | 66413644 | 0.26 | Missense_Mutation | A | C | c.559A > C | p.T187P |
| POM121 | 7 | 72398976 | 0.06 | Missense_Mutation | A | G | c.1076A > G | p.N359S |
| POM121 | 7 | 72413896 | 0.06 | Missense_Mutation | A | G | c.3364A > G | p.T1122A |
| COL1A2 | 7 | 94052281 | 0.62 | Missense_Mutation | C | T | c.2416C > T | p.P806S |
| LRRC17 | 7 | 102585014 | 0.19 | Missense_Mutation | C | G | c.1286C > G | p.T429S |
| LRRN3 | 7 | 110763972 | 0.16 | Missense_Mutation | A | C | c.1144A > C | p.N382H |
| KMT2C | 7 | 151970855 | 0.02 | Missense_Mutation | G | C | c.947C > G | p.T316S |
| Unknown | 8 | 12517307 | 0.14 | IGR | C | T | | |
| PDLIM2 | 8 | 22447026 | 0.87 | Intron | A | C | | |
| LRRCC1 | 8 | 86019547 | 0.2 | Missense_Mutation | C | T | c.17C > T | p.A6V |
| TG | 8 | 134147138 | 0.83 | 3′UTR | G | A | | |
| COL22A1 | 8 | 139824118 | 0.58 | Missense_Mutation | T | G | c.1373A > C | p.Q458P |
| COL22A1 | 8 | 139824129 | 1 | Silent | T | G | c.1362A > C | p.P454P |
| TSTA3 | 8 | 144697039 | 0.54 | Missense_Mutation | T | G | c.308A > C | p.E103A |
| CPSF1 | 8 | 145620768 | 0.57 | Splice_Site | T | G | | |
| KIFC2 | 8 | 145694024 | 0.78 | Missense_Mutation | C | A | c.994C > A | p.Q332K |
| SMU1 | 9 | 33068870 | 0.08 | Silent | G | A | c.453C > T | p.G151G |
| FAM205B | 9 | 34835480 | 0.06 | RNA | C | T | | |
| GLIPR2 | 9 | 36147796 | 0.25 | Missense_Mutation | T | G | c.27T > G | p.F9L |
| MIR4477B | 9 | 68414704 | 0.41 | RNA | A | C | | |
| MIR4477B | 9 | 68414853 | 0.48 | RNA | C | T | | |
| Unknown | 9 | 69067873 | 0.5 | IGR | A | C | | |
| Unknown | 9 | 69067929 | 0.58 | IGR | G | A | | |
| CCDC180 | 9 | 100105896 | 0.52 | Intron | C | A | | |
| CDK5RAP2 | 9 | 123151373 | 0.29 | 3′UTR | A | G | | |
| LCN1 | 9 | 138413373 | 0.11 | Silent | T | C | c.30T > C | p.L10L |
| TSPAN15 | 10 | 71267418 | 0.23 | 3′UTR | T | G | | |
| BTBD10 | 11 | 13435092 | 0.36 | Missense_Mutation | T | G | c.793A > C | p.K265Q |
| OR4C6 | 11 | 55433000 | 0.9 | Missense_Mutation | C | T | c.358C > T | p.R120C |
| FOSL1 | 11 | 65664326 | 0.95 | Missense_Mutation | C | T | c.251G > A | p.R84Q |
| UNC93B1 | 11 | 67759316 | 0.13 | Missense_Mutation | C | T | c.1492G > A | p.V498M |
| GRAMD1B | 11 | 123431287 | 0.58 | Intron | A | C | | |
| TIRAP | 11 | 126162750 | 0.15 | Missense_Mutation | C | T | c.446C > T | p.P149L |
| IQSEC3 | 12 | 250285 | 0.69 | Intron | T | C | | |
| WNK1 | 12 | 1018024 | 0.52 | 3′UTR | T | G | | |
| PRMT8 | 12 | 3649787 | 1 | Missense_Mutation | T | C | c.91T > C | p.S31P |
| PTMS | 12 | 6879650 | 0.61 | 3′UTR | T | G | | |
| PTMS | 12 | 6879662 | 0.98 | 3′UTR | T | G | | |
| LAG3 | 12 | 6881952 | 0.68 | 5′UTR | A | C | | |
| C12orf60 | 12 | 14975932 | 0.66 | Missense_Mutation | T | G | c.63T > G | p.F21L |
| KIF21A | 12 | 39705411 | 0.21 | Intron | A | C | | |
| PCED1B | 12 | 47629658 | 0.17 | Missense_Mutation | C | A | c.812C > A | p.P271H |
| RAB5B | 12 | 56380682 | 0.87 | 5′UTR | T | C | | |
| RDH16 | 12 | 57345813 | 0.54 | Nonstop_Mutation | T | G | c.954A > C | p.*318C |
| TMEM5 | 12 | 64196045 | 0.1 | Silent | C | T | c.603C > T | p.L201L |
| NAV3 | 12 | 78571071 | 0.64 | Missense_Mutation | A | C | c.5275A > C | p.K1759Q |
| PPFIA2 | 12 | 81671191 | 0.46 | Missense_Mutation | G | T | c.3215C > A | p.T1072K |
| PPFIA2 | 12 | 81671194 | 0.42 | Splice_Site | C | T | | |
| RASSF9 | 12 | 86199652 | 0.14 | Missense_Mutation | G | A | c.136C > T | p.R46C |
| POLR3B | 12 | 106820982 | 0.32 | Missense_Mutation | C | T | c.1109C > T | p.S370F |
| RP11-556N21.1 | 13 | 25144833 | 0.43 | RNA | A | G | | |
| TDRD3 | 13 | 60971461 | 0.61 | Intron | A | C | | |
| TFDP1 | 13 | 114240102 | 0.3 | 5′UTR | C | T | | |
| HSPA2 | 14 | 65008372 | 1 | Missense_Mutation | G | A | c.805G > A | p.A269T |
| ELMSAN1 | 14 | 74185939 | 0.92 | 3′UTR | A | C | | |
| SPTLC2 | 14 | 78036825 | 0.22 | Nonsense_Mutation | C | A | c.658G > T | p.E220* |
| RP11-96O20.2 | 15 | 45848224 | 0.55 | lincRNA | G | T | | |
| DUT | 15 | 48634301 | 0.41 | 3′UTR | G | A | | |
| MNS1 | 15 | 56736654 | 0.53 | Missense_Mutation | T | G | c.674A > C | p.E225A |
| SIN3A | 15 | 75706577 | 0.99 | Missense_Mutation | G | C | c.442C > G | p.L148V |
| CREBBP | 16 | 3779204 | 0.48 | Silent | C | G | c.5844G > C | p.P1948P |
| COG7 | 16 | 23457283 | 0.21 | Splice_Site | C | T | | |
| NPIPB9 | 16 | 28763851 | 0.06 | 5′UTR | T | C | | |
| CORO1A | 16 | 30199933 | 1 | Intron | A | G | | |
| CORO1A | 16 | 30199937 | 1 | Intron | T | G | | |
| CORO1A | 16 | 30199942 | 1 | Intron | T | G | | |
| SETD1A | 16 | 30990536 | 0.69 | Silent | T | C | c.3429T > C | p.P1143P |
| BCL6B | 17 | 6927768 | 0.31 | Silent | A | C | c.450A > C | p.P150P |
| BCL6B | 17 | 6927777 | 0.45 | Silent | A | C | c.459A > C | p.P153P |
| PFAS | 17 | 8151409 | 1 | 5′Flank | T | G | | |
| PFAS | 17 | 8172087 | 0.08 | Missense_Mutation | G | T | c.3619G > T | p.A1207S |
| RP11-219A15.4 | 17 | 16722846 | 0.66 | RNA | G | A | | |
| RP11-744K17.9 | 17 | 21904125 | 0.11 | lincRNA | G | A | | |
| NF1 | 17 | 29422162 | 1 | 5′UTR | T | C | | |
| HNF1B | 17 | 36104902 | 0.69 | 5′UTR | T | G | | |
| HNF1B | 17 | 36104904 | 1 | 5′UTR | A | G | | |
| HNF1B | 17 | 36104910 | 1 | 5′UTR | T | G | | |
| HNF1B | 17 | 36104914 | 1 | 5′UTR | T | G | | |
| MSL1 | 17 | 38289899 | 0.23 | Nonsense_Mutation | G | T | c.1669G > T | p.E557* |
| SP6 | 17 | 45924796 | 0.2 | Missense_Mutation | T | G | c.1000A > C | p.K334Q |
| HOXB2 | 17 | 46622286 | 0.64 | 5′UTR | T | G | | |
| UTP18 | 17 | 49340654 | 0.4 | Missense_Mutation | C | G | c.362C > G | p.S121W |
| MTMR4 | 17 | 56584217 | 0.31 | Missense_Mutation | G | A | c.878C > T | p.A293V |
| ENTHD2 | 17 | 79203046 | 0.87 | Silent | T | G | c.1260A > C | p.P420P |
| HRH4 | 18 | 22057482 | 0.51 | Missense_Mutation | A | C | c.1129A > C | p.K377Q |
| REXO1 | 19 | 1827048 | 0.38 | Silent | T | G | c.1740A > C | p.P580P |
| AES | 19 | 3056403 | 1 | Intron | T | G | | |
| TUBB4A | 19 | 6495887 | 0.07 | Missense_Mutation | T | C | c.623A > G | p.Y208C |
| ZNF627 | 19 | 11728631 | 0.74 | Missense_Mutation | A | C | c.1313A > C | p.E438A |
| ZNF791 | 19 | 12739215 | 0.37 | Missense_Mutation | A | C | c.872A > C | p.E291A |
| CPAMD8 | 19 | 17006740 | 0.11 | Intron | G | A | | |
| NXNL1 | 19 | 17566477 | 1 | Silent | G | C | c.618C > G | p.G206G |
| NXNL1 | 19 | 17566484 | 1 | Missense_Mutation | T | C | c.611A > G | p.E204G |
| SLC5A5 | 19 | 17983031 | 1 | 5′UTR | A | C | | |
| KMT2B | 19 | 36224209 | 0.74 | Silent | G | C | c.6759G > C | p.P2253P |
| KMT2B | 19 | 36224215 | 0.5 | Silent | G | C | c.6765G > C | p.P2255P |
| ZNF850 | 19 | 37253563 | 0.32 | 5′UTR | A | C | | |
| CYP2A13 | 19 | 41601920 | 0.71 | 3′UTR | A | G | | |
| CIC | 19 | 42799059 | 0.3 | Missense_Mutation | C | T | c.4543C > T | p.R1515C |
| PHLDB3 | 19 | 43983726 | 0.63 | Missense_Mutation | T | G | c.1505A > C | p.H502P |
| PHLDB3 | 19 | 43983731 | 0.89 | Silent | T | G | c.1500A > C | p.P500P |
| PHLDB3 | 19 | 43983736 | 0.93 | Missense_Mutation | T | G | c.1495A > C | p.T499P |
| ZNF525 | 19 | 53887191 | 0.15 | IGR | T | A | | |
| PLCB4 | 20 | 9319601 | 0.62 | Missense_Mutation | C | T | c.286C > T | p.R96W |
| FAM182B | 20 | 25755527 | 0.27 | Silent | G | A | c.429C > T | p.S143S |
| FRG1B | 20 | 29614275 | 0.41 | 5′UTR | G | A | | |
| FRG1B | 20 | 29633900 | 0.1 | Missense_Mutation | A | G | c.539A > G | p.E180G |
| B4GALT5 | 20 | 48257072 | 0.29 | Missense_Mutation | T | G | c.737A > C | p.Y246S |
| VAPB | 20 | 56964368 | 0.39 | 5′UTR | A | C | | |
| TPTE | 21 | 11029682 | 0.11 | 5′UTR | G | A | | |
| BAGE2 | 21 | 11038748 | 0.17 | RNA | C | T | | |
| BAGE2 | 21 | 11058353 | 0.2 | RNA | T | C | | |
| BAGE2 | 21 | 11098764 | 0.04 | RNA | G | A | | |
| SMIM11 | 21 | 35751748 | 0.34 | 5′UTR | T | G | | |
| TMPRSS3 | 21 | 43815505 | 0.12 | Missense_Mutation | C | T | c.22G > A | p.A8T |
| AIRE | 21 | 45709677 | 0.07 | Missense_Mutation | G | T | c.790G > T | p.A264S |
| KRTAP10-11 | 21 | 46066486 | 0.5 | Silent | C | T | c.111C > T | p.C37C |
| AC008132.13 | 22 | 18844763 | 0.15 | 3′UTR | T | C | | |
| POM121L4P | 22 | 21044816 | 0.05 | RNA | G | A | | |
| CHCHD10 | 22 | 24108456 | 0.58 | Missense_Mutation | T | G | c.268A > C | p.T90P |
| SMARCB1 | 22 | 24176559 | 0.59 | 3′UTR | A | C | | |
| CSNK1E | 22 | 38757479 | 0.11 | 5′UTR | A | G | | |
| EFCAB6 | 22 | 44083353 | 0.42 | Missense_Mutation | A | T | c.1140T > A | p.N380K |
| PHF21B | 22 | 45309895 | 0.58 | Missense_Mutation | A | G | c.638T > C | p.L213P |
| TLR7 | X | 12906275 | ND | Missense_Mutation | G | A | c.2648G > A | p.R883H |
| BCOR | X | 39921456 | ND | Missense_Mutation | C | T | c.4364G > A | p.R1455K |
| Unknown | X | 47658044 | ND | IGR | T | G | | |
| TGIF2LX | X | 89177570 | ND | Missense_Mutation | G | T | c.486G > T | p.L162F |
| DCAF12L1 | X | 125686202 | ND | Silent | G | A | c.390C > T | p.I130I |
| L1CAM | X | 153141379 | ND | 5′UTR | C | G | | |
| L1CAM | X | 153141386 | ND | 5′UTR | T | G | | |
| L1CAM | X | 153141401 | ND | Splice_Site | T | G | | |

MGH54

| | | | | | | | | |
|---|---|---|---|---|---|---|---|---|
| PLCH2 | 1 | 2435352 | 0.69 | Intron | T | C | | |
| PLCH2 | 1 | 2435357 | 0.69 | Intron | A | C | | |
| CEP85 | 1 | 26566306 | 0.7 | Missense_Mutation | G | A | c.32G > A | p.G11E |
| OSBPL9 | 1 | 52226257 | 0.34 | Intron | T | G | | |
| LRP8 | 1 | 53793514 | 0.08 | Missense_Mutation | A | T | c.71T > A | p.L24Q |
| DOCK7 | 1 | 62941517 | 0.06 | Missense_Mutation | A | C | c.5729T > G | p.F1910C |
| RP11-417J8.6 | 1 | 142635475 | 0.09 | lincRNA | T | G | | |
| Unknown | 1 | 144619403 | 0.08 | IGR | A | G | | |
| PMVK | 1 | 154897570 | 0.37 | 3′UTR | T | C | | |
| THBS3 | 1 | 155167452 | 0.22 | Splice_Site | T | G | | |
| KIAA0907 | 1 | 155887387 | 0.37 | Missense_Mutation | T | G | c.1343A > C | p.Q448P |
| KIAA0907 | 1 | 155887393 | 0.51 | Missense_Mutation | T | G | c.1337A > C | p.Q446P |
| SH2D2A | 1 | 156777059 | 0.37 | Missense_Mutation | C | G | c.1081G > C | p.A361P |
| SH2D2A | 1 | 156777070 | 0.38 | Missense_Mutation | T | G | c.1070A > C | p.Q357P |
| LRRC71 | 1 | 156893843 | 0.23 | Missense_Mutation | A | C | c.263A > C | p.H88P |
| VANGL2 | 1 | 160395211 | 1 | 3′UTR | A | G | | |
| VANGL2 | 1 | 160395221 | 1 | 3′UTR | A | G | | |
| CPSF3 | 2 | 9599742 | 0.27 | Missense_Mutation | G | A | c.1781G > A | p.R594K |
| CTNNA2 | 2 | 80136918 | 0.37 | Missense_Mutation | A | C | c.1051A > C | p.N351H |
| ZEB2 | 2 | 145146471 | 0.11 | 3′UTR | T | A | | |
| GTF3C3 | 2 | 197657782 | 0.06 | Silent | C | T | c.309G > A | p.E103E |
| EEF1B2 | 2 | 207025358 | 0.06 | Missense_Mutation | A | G | c.127A > G | p.S43G |
| EEF1B2 | 2 | 207025366 | 0.06 | Silent | G | A | c.135G > A | p.P45P |
| CPO | 2 | 207833951 | 0.19 | Missense_Mutation | T | G | c.916T > G | p.S306A |
| IDH1 | 2 | 209113112 | 1 | Missense_Mutation | C | T | c.395G > A | p.R132H |
| AC131097.3 | 2 | 242946237 | 0.03 | RNA | G | C | | |
| NR2C2 | 3 | 15084335 | 0.67 | Intron | T | G | | |
| ZBTB47 | 3 | 42700699 | 0.21 | Missense_Mutation | G | C | c.852G > C | p.E284D |
| PLXNB1 | 3 | 48461613 | 0.25 | Silent | T | G | c.2082A > C | p.P694P |
| FAM86DP | 3 | 75475709 | 0.06 | RNA | T | C | | |
| EFCAB12 | 3 | 129120540 | 0.06 | Missense_Mutation | C | G | c.1615G > C | p.V539L |
| PIK3CB | 3 | 138433351 | 0.31 | Missense_Mutation | T | G | c.1261A > C | p.N421H |
| IQCJ-SCHIP1 | 3 | 159482850 | 0.09 | Missense_Mutation | G | A | c.601G > A | p.E201K |
| OTOP1 | 4 | 4228226 | 0.18 | Silent | G | A | c.366C > T | p.R122R |
| LGI2 | 4 | 25005792 | 0.94 | Missense_Mutation | C | T | c.919G > A | p.E307K |
| USP46 | 4 | 53522601 | 0.55 | Intron | C | G | | |
| PDGFRA | 4 | 55131029 | 0.16 | Intron | A | T | | |
| PDLIM5 | 4 | 95508331 | 0.95 | Intron | A | C | | |
| ZNF827 | 4 | 146744679 | 0.19 | Splice_Site | T | G | | |
| KLHL2 | 4 | 166199030 | 0.38 | Intron | A | G | | |
| SDHA | 5 | 228257 | 0.08 | Intron | T | G | | |
| CCT5 | 5 | 10250663 | 0.67 | Intron | A | G | | |
| C5orf51 | 5 | 41909846 | 0.37 | Splice_Site | A | T | | |
| KIF2A | 5 | 61602215 | 0.65 | 5′UTR | T | C | | |
| KIF2A | 5 | 61602219 | 1 | 5′UTR | A | C | | |
| SNRNP48 | 6 | 7609118 | 0.69 | 3′UTR | G | T | | |
| BMP6 | 6 | 7727541 | 0.08 | Missense_Mutation | A | T | c.353A > T | p.Q118L |
| TFAP2A | 6 | 10402545 | 0.24 | Intron | T | G | | |
| CASC14 | 6 | 22136876 | 0.72 | lincRNA | T | G | | |
| LRRC16A | 6 | 25551276 | 0.58 | Silent | T | C | c.2467T > C | p.L823L |
| SCAND3 | 6 | 28543205 | 1 | Missense_Mutation | G | A | c.1277C > T | p.T426I |
| ZNRD1-AS1 | 6 | 29977327 | 0.07 | RNA | T | C | | |
| NRM | 6 | 30658764 | 0.34 | 5′UTR | A | G | | |
| NRM | 6 | 30658769 | 0.32 | 5′UTR | T | G | | |
| RNF5 | 6 | 32147865 | 0.07 | Missense_Mutation | C | T | c.407C > T | p.T136I |
| RGL2 | 6 | 33269389 | 0.73 | 5′Flank | T | G | | |
| TTK | 6 | 80717709 | 0.13 | Missense_Mutation | G | T | c.323G > T | p.S108I |
| ORC3 | 6 | 88318940 | 1 | Missense_Mutation | A | C | c.706A > C | p.I236L |
| COQ3 | 6 | 99819447 | 0.31 | Missense_Mutation | A | C | c.746T > G | p.F249C |
| SOBP | 6 | 107955437 | 0.23 | Silent | G | C | c.1389G > C | p.P463P |
| SEC63 | 6 | 108214765 | 0.07 | Nonsense_Mutation | A | T | c.1595T > A | p.L532* |
| VNN1 | 6 | 133014444 | 0.57 | Missense_Mutation | A | C | c.545T > G | p.F182C |
| INTS1 | 7 | 1526685 | 0.06 | Missense_Mutation | C | T | c.2699G > A | p.G900D |
| SP4 | 7 | 21467806 | 0.64 | 5′UTR | G | C | | |
| WIPF3 | 7 | 29874364 | 0.68 | Silent | A | C | c.24A > C | p.P8P |
| WIPF3 | 7 | 29874367 | 0.84 | Silent | T | C | c.27T > C | p.P9P |
| PTPRZ1 | 7 | 121651723 | 0.9 | Nonsense_Mutation | C | T | c.2623C > T | p.Q875* |
| TRIM24 | 7 | 138145895 | 0.06 | Intron | C | T | | |
| PRSS1 | 7 | 142459042 | 0.22 | Intron | C | T | | |
| RP11-481A20.11 | 8 | 11872530 | 0.09 | Missense_Mutation | G | A | c.29C > T | p.A10V |
| RP11-481A20.11 | 8 | 11872550 | 0.09 | Missense_Mutation | G | C | c.9C > G | p.S3R |
| PDLIM2 | 8 | 22447026 | 0.49 | Intron | A | C | | |
| ZNF395 | 8 | 28210808 | 0.34 | Missense_Mutation | T | G | c.701A > C | p.H234P |
| ASPH | 8 | 62491435 | 0.07 | Intron | C | T | | |
| CHMP4C | 8 | 82665470 | 0.31 | Missense_Mutation | A | C | c.362A > C | p.E121A |
| SUFU | 10 | 104263946 | 0.29 | Missense_Mutation | A | C | c.37A > C | p.T13P |
| SUFU | 10 | 104263957 | 0.29 | Silent | G | C | c.48G > C | p.P16P |
| CALHM2 | 10 | 105209523 | 0.04 | Missense_Mutation | G | A | c.176C > T | p.A59V |
| CALY | 10 | 135137975 | 0.33 | IGR | T | G | | |
| CALY | 10 | 135137979 | 0.38 | IGR | C | G | | |
| TSSC2 | 11 | 3424149 | 0.06 | RNA | C | T | | |
| BTBD10 | 11 | 13435092 | 0.19 | Missense_Mutation | T | G | c.793A > C | p.K265Q |
| TRIM48 | 11 | 55035844 | 0.08 | Missense_Mutation | T | C | c.574T > C | p.Y192H |
| RPLP0P2 | 11 | 61405030 | 0.15 | RNA | T | A | | |
| DNAJC4 | 11 | 64000291 | 0.56 | Missense_Mutation | C | T | c.481C > T | p.L161F |
| FOLH1B | 11 | 89395322 | 0.15 | RNA | C | T | | |
| STT3A | 11 | 125476327 | 0.29 | Silent | A | C | c.747A > C | p.I249I |
| PTMS | 12 | 6879650 | 0.37 | 3′UTR | T | G | | |
| PTMS | 12 | 6879653 | 0.68 | 3′UTR | A | G | | |
| PTMS | 12 | 6879656 | 0.58 | 3′UTR | T | G | | |
| FAM90A1 | 12 | 8380196 | 0.17 | 5′UTR | A | G | | |
| RDH16 | 12 | 57345813 | 0.43 | Nonstop_Mutation | T | G | c.954A > C | p.*318C |
| DTX3 | 12 | 58001051 | 0.4 | Silent | T | C | c.405T > C | p.A135A |
| NAV3 | 12 | 78571071 | 0.33 | Missense_Mutation | A | C | c.5275A > C | p.K1759Q |
| APAF1 | 12 | 99117444 | 0.18 | Missense_Mutation | G | A | c.3232G > A | p.E1078K |
| SETD1B | 12 | 122261027 | 0.26 | Silent | A | C | c.4542A > C | p.P1514P |
| RP11-556N21.1 | 13 | 25168489 | 0.14 | RNA | G | A | | |
| ESD | 13 | 47345484 | 0.53 | 3′UTR | G | T | | |
| TDRD3 | 13 | 60971461 | 0.61 | Intron | A | C | | |
| TDRD3 | 13 | 60971466 | 0.61 | Intron | A | C | | |
| COL4A1 | 13 | 110833688 | 0.06 | Missense_Mutation | C | T | c.2144G > A | p.R715H |
| OR4Q3 | 14 | 20216484 | 0.25 | Missense_Mutation | A | C | c.898A > C | p.K300Q |
| TM9SF1 | 14 | 24661303 | 0.86 | Intron | C | G | | |
| GPX2 | 14 | 65406817 | 0.42 | Intron | G | T | | |
| CALM1 | 14 | 90870229 | 0.66 | Missense_Mutation | G | A | c.202G > A | p.E68K |
| Unknown | 14 | 106134738 | 0.05 | IGR | T | C | | |
| HERC2 | 15 | 28459392 | 0.06 | Missense_Mutation | G | A | c.6385C > T | p.R2129C |
| LPCAT4 | 15 | 34659245 | 0.25 | Silent | T | G | c.57A > C | p.P19P |
| WDR72 | 15 | 53994476 | 0.69 | Missense_Mutation | G | A | c.1424C > T | p.S475L |
| MNS1 | 15 | 56736654 | 0.24 | Missense_Mutation | T | G | c.674A > C | p.E225A |
| CLN6 | 15 | 68500436 | 0.52 | 3′UTR | A | C | | |
| CYP1A2 | 15 | 75045612 | 0.81 | Splice_Site | G | A | | |
| TSC2 | 16 | 2121833 | 0.12 | Silent | T | C | c.1995T > C | p.P665P |
| CREBBP | 16 | 3779210 | 0.38 | Silent | T | G | c.5838A > C | p.P1946P |
| GRIN2A | 16 | 10273739 | 0.98 | Intron | A | C | | |
| PFAS | 17 | 8151415 | 0.9 | 5′Flank | T | G | | |
| RP11-744K17.9 | 17 | 21904093 | 0.19 | lincRNA | A | G | | |
| TLCD1 | 17 | 27051858 | 0.29 | Silent | A | G | c.414T > C | p.G138G |
| HNF1B | 17 | 36104904 | 0.85 | 5′UTR | A | G | | |
| HNF1B | 17 | 36104910 | 0.62 | 5′UTR | T | G | | |
| HNF1B | 17 | 36104914 | 0.69 | 5′UTR | T | G | | |
| WNK4 | 17 | 40946930 | 0.18 | Missense_Mutation | A | C | c.2491A > C | p.I831L |
| WNK4 | 17 | 40946954 | 0.27 | Missense_Mutation | A | C | c.2515A > C | p.S839R |
| WNK4 | 17 | 40946965 | 0.29 | Silent | A | C | c.2526A > C | p.P842P |
| ITGA2B | 17 | 42452325 | 0.21 | Intron | G | C | | |
| SP6 | 17 | 45924796 | 0.12 | Missense_Mutation | T | G | c.1000A > C | p.K334Q |
| HOXB2 | 17 | 46622302 | 1 | 5′UTR | T | G | | |
| WBP2 | 17 | 73851262 | 0.59 | Intron | G | C | | |
| USP36 | 17 | 76799999 | 0.42 | Missense_Mutation | T | G | c.2278A > C | p.T760P |
| C1QTNF1 | 17 | 77021988 | 0.1 | 5′UTR | T | C | | |
| AATK | 17 | 79093349 | 0.62 | Silent | C | T | c.3915G > A | p.P1305P |
| ENTHD2 | 17 | 79203046 | 0.57 | Silent | T | G | c.1260A > C | p.P420P |
| EPG5 | 18 | 43534623 | 1 | Nonsense_Mutation | G | A | c.745C > T | p.Q249* |
| SMARCA4 | 19 | 11132437 | 0.78 | Missense_Mutation | C | T | c.2653C > T | p.R885C |
| SMARCA4 | 19 | 11132513 | 0.04 | Missense_Mutation | C | T | c.2729C > T | p.T910M |
| ZNF627 | 19 | 11728631 | 0.63 | Missense_Mutation | A | C | c.1313A > C | p.E438A |
| BRD4 | 19 | 15353841 | 1 | Silent | T | G | c.3039A > C | p.P1013P |
| CPAMD8 | 19 | 17006740 | 0.06 | Intron | G | A | | |
| NXNL1 | 19 | 17566481 | 0.89 | Missense_Mutation | T | C | c.614A > G | p.E205G |
| NXNL1 | 19 | 17566484 | 0.52 | Missense_Mutation | T | C | c.611A > G | p.E204G |
| C19orf60 | 19 | 18702255 | 0.81 | Intron | C | T | | |
| Unknown | 19 | 34583535 | 0.53 | IGR | T | C | | |
| CYP2A13 | 19 | 41601925 | 0.34 | 3′UTR | C | G | | |
| CIC | 19 | 42796236 | 0.69 | Splice_Site | A | G | | |
| ARHGAP35 | 19 | 47440657 | 0.32 | Missense_Mutation | A | C | c.3818A > C | p.E1273A |
| FUZ | 19 | 50310295 | 0.11 | 3′UTR | T | C | | |
| SIRPB1 | 20 | 1585397 | 0.18 | Intron | T | C | | |
| OCSTAMP | 20 | 45170141 | 0.04 | Silent | G | A | c.1473C > T | p.T491T |
| B4GALT5 | 20 | 48257072 | 0.2 | Missense_Mutation | T | G | c.737A > C | p.Y246S |
| VAPB | 20 | 56964377 | 0.33 | 5′UTR | A | C | | |
| MIS18A | 21 | 33641263 | 0.4 | 3′UTR | G | T | | |
| PI4KA | 22 | 21064203 | 0.04 | Missense_Mutation | G | A | c.5992C > T | p.L1998F |
| CHCHD10 | 22 | 24108440 | 0.22 | Missense_Mutation | T | G | c.284A > C | p.Q95P |
| Unknown | 22 | 25053920 | 0.04 | IGR | C | T | | |
| TTC28 | 22 | 28692203 | 0.08 | Missense_Mutation | T | G | c.916A > C | p.K306Q |
| BIK | 22 | 43524599 | ND | Silent | A | C | c.358A > C | p.R120R |
| IQSEC2 | X | 53296215 | ND | Intron | C | A | | |
| MSN | X | 64956699 | ND | Silent | G | A | c.1002G > A | p.E334E |
| LONRF3 | X | 118143186 | ND | Missense_Mutation | A | C | c.1628A > C | p.E543A |
| MAGEA4 | X | 151091946 | ND | 5′UTR | C | T | | |
| GABRQ | X | 151815566 | ND | Missense_Mutation | A | C | c.464A > C | p.D155A |
| ARHGAP4 | X | 153175924 | ND | Intron | T | C | | |

MGH60

| | | Start_ | | Variant_ | Tumor_Seq_ | Tumor_Seq_ | cDNA_ | Protein_ |
|---|---|---|---|---|---|---|---|---|
| Hugo_Symbol | Chromosome | position | ccf_hat | Classification | Allele1 | Allele2 | Change | Change |
| MST1L | 1 | 17084569 | NA | RNA | G | A | | |
| PADI3 | 1 | 17596854 | 1 | Missense_Mutation | G | A | c.779G > A | p.G260D |
| LCE1A | 1 | 152799991 | 0.18 | Missense_Mutation | A | C | c.43A > C | p.K15Q |
| LCE1A | 1 | 152800003 | 0.17 | Missense_Mutation | A | C | c.55A > C | p.K19Q |
| PMVK | 1 | 154897570 | 0.56 | 3′UTR | T | C | | |
| THBS3 | 1 | 155167452 | 0.43 | Splice_Site | T | G | | |
| SH2D2A | 1 | 156777070 | 0.26 | Missense_Mutation | T | G | c.1100A > C | p.Q367P |
| APCS | 1 | 159558233 | 0.04 | Missense_Mutation | A | G | c.407A > G | p.K136R |
| PPP1R12B | 1 | 202407176 | 0.05 | Silent | G | A | c.1482G > A | p.G494G |
| LAMB3 | 1 | 209797025 | 0.02 | Missense_Mutation | G | C | c.2183C > G | p.A728G |
| SMYD3 | 1 | 246093457 | 0.24 | Intron | T | C | | |
| CAD | 2 | 27456266 | 0.96 | Silent | G | T | c.3078G > T | p.A1026A |
| GGCX | 2 | 85776973 | 0.21 | 3′UTR | G | A | | |
| ANKRD36 | 2 | 97869931 | 0.14 | Missense_Mutation | A | T | c.2992A > T | p.T998S |
| TMEM182 | 2 | 103378601 | 0.53 | 5′UTR | G | T | | |
| KIF5C | 2 | 149633155 | 0.49 | 5′UTR | A | C | | |
| XIRP2 | 2 | 168103475 | 0.37 | Missense_Mutation | C | T | c.5573C > T | p.T1858M |
| PGAP1 | 2 | 197791356 | 0.1 | 5′UTR | G | A | | |
| FASTKD2 | 2 | 207632128 | 1 | Silent | C | T | c.711C > T | p.H237H |
| IDH1 | 2 | 209113112 | 0.84 | Missense_Mutation | C | T | c.395G > A | p.R132H |
| NGLY1 | 3 | 25770654 | 0.16 | Silent | T | G | c.1527A > C | p.I509I |
| SUCLG2 | 3 | 67559234 | 0.26 | Missense_Mutation | G | T | c.754C > A | p.Q252K |
| CHMP2B | 3 | 87303046 | 0.24 | 3′UTR | C | A | | |
| GPR31 | 6 | 167571126 | 0.16 | Missense_Mutation | G | A | c.194C > T | p.A65V |
| ZNF395 | 8 | 28210802 | 0.26 | Missense_Mutation | T | G | c.707A > C | p.Q236P |
| COL22A1 | 8 | 139824118 | 0.53 | Missense_Mutation | T | G | c.1373A > C | p.Q458P |
| SEMA4D | 9 | 92003803 | 0.99 | Missense_Mutation | G | C | c.934C > G | p.L312V |
| C10orf112 | 10 | 19981478 | 1 | Silent | A | G | c.4260A > G | p.P1420P |
| SVILP1 | 10 | 30986357 | 0.06 | RNA | T | C | | |
| ANKRD30A | 10 | 37431050 | 0.06 | Missense_Mutation | G | C | c.1057G > C | p.A353P |
| PTEN | 10 | 89720659 | 0.23 | Missense_Mutation | G | T | c.810G > T | p.M270I |
| RRP12 | 10 | 99118376 | 0.84 | Splice_Site | T | C | c.3708_splice | p.K1237_splice |
| AFAP1L2 | 10 | 116059958 | 0.94 | Missense_Mutation | C | T | c.1952G > A | p.S651N |
| ZNF511 | 10 | 135137975 | 0.36 | Intron | T | G | | |
| MRVI1 | 11 | 10647847 | 0.07 | Missense_Mutation | G | A | c.761C > T | p.P254L |
| BTBD10 | 11 | 13435092 | 0.18 | Missense_Mutation | T | G | c.793A > C | p.K265Q |
| OR5AK2 | 11 | 56757259 | 0.53 | Missense_Mutation | A | C | c.871A > C | p.S291R |
| DLG2 | 11 | 83252723 | 0.87 | Splice_Site | A | C | | |
| CCDC81 | 11 | 86133688 | 0.09 | Silent | C | T | c.1095C > T | p.T365T |
| NPAT | 11 | 108031631 | 0.88 | Missense_Mutation | T | C | c.4182A > G | p.I1394M |
| PTS | 11 | 112099324 | 0.29 | Silent | C | T | c.91C > T | p.L31L |
| ESAM | 11 | 124623472 | 1 | 3′UTR | C | T | | |
| STT3A | 11 | 125476327 | 0.23 | Silent | A | C | c.747A > C | p.I249I |
| WNK1 | 12 | 1018024 | 0.36 | 3′UTR | T | G | | |
| PTMS | 12 | 6879662 | 0.39 | 3′UTR | T | G | | |
| LINC00937 | 12 | 8549081 | 0.14 | lincRNA | C | G | | |
| BICD1 | 12 | 32481354 | 0.82 | Silent | G | A | c.1965G > A | p.A655A |
| RPAP3 | 12 | 48096569 | 0.81 | Nonsense_Mutation | C | A | c.55G > T | p.E19* |
| TIMELESS | 12 | 56818562 | 0.89 | Missense_Mutation | G | A | c.1849C > T | p.L617F |
| RDH16 | 12 | 57345813 | 0.16 | Nonstop_Mutation | T | G | c.954A > C | p.*318C |
| NAV3 | 12 | 78571071 | 0.34 | Missense_Mutation | A | C | c.5275A > C | p.K1759Q |
| SLC8B1 | 12 | 113756885 | 1 | Intron | G | A | | |
| PDS5B | 13 | 33332227 | 0.48 | Missense_Mutation | G | T | c.3059G > T | p.C1020F |
| PDS5B | 13 | 33332229 | 0.47 | Missense_Mutation | C | T | c.3061C > T | p.L1021F |
| RP11-483E23.2 | 15 | 28599954 | 0.02 | RNA | A | G | | |
| CHRNE | 17 | 4802379 | 1 | Missense_Mutation | C | T | c.1243G > A | p.A415T |
| BCL6B | 17 | 6927768 | 0.3 | Silent | A | C | c.450A > C | p.P150P |
| CYP2A13 | 19 | 41601907 | 0.31 | 3′UTR | C | G | | |
| CYP2A13 | 19 | 41601920 | 0.23 | 3′UTR | A | G | | |
| CYP2A13 | 19 | 41601925 | 0.28 | 3′UTR | C | G | | |
| CIC | 19 | 42791757 | 1 | Missense_Mutation | C | T | c.3370C > T | p.R1124W |
| VAPB | 20 | 56964377 | 0.18 | 5′UTR | A | C | | |
| POM121L4P | 22 | 21044374 | 0.17 | RNA | G | C | | |
| PPM1F | 22 | 22277819 | 0.93 | Silent | C | T | c.507G > A | p.V169V |
| AR | X | 66765161 | | Missense_Mutation | A | T | c.173A > T | p.Q58L |
| IGBP1 | X | 69354420 | | Missense_Mutation | T | G | c.236T > G | p.L79R |
| SAGE1 | X | 134989127 | | Missense_Mutation | A | G | c.779A > G | p.K260R |
| MECP2 | X | 153296115 | | Silent | T | G | c.1164A > C | p.P388P |

Finally, to explore point mutations with an additional strategy, independent of single cell RNA-seq, Applicants also tested specific mutations in single cells by mutation-sensitive qPCR (Methods). While most subclonal mutations were of unknown functional relevance, Applicants were intrigued by the identification of a subclonal CIC mutation in MGH53 (˜30% frequency by ABSOLUTE). CIC is a known tumor suppressor in oligodendroglioma (115), and this missense p.R1515C mutation, also observed in four patients in the TCGA cohort (112) (the second most common across 66 patients with any CIC mutation). CIC is haploid (as it is coded on chromosome 19q) and thus allows us to ascertain both mutant and WT status. Because RNA-seq reads detected the CIC mutation in only 7 of MGH53 cells, Applicants tested its presence in additional cells using a mutation-sensitive qPCR approach and were able to ascertain 28 CIC mutant cells (including validation of all 7 cells detected by RNA-seq reads) and 27 CIC wild-type MGH53 cells (FIG. 20D). Importantly, Applicants identified a signature of expression changes between the CIC mutant and WT cells (FIG. 20E, Table 6), including increased expression of the transcription factors ETV1 and ETV5, which were recently shown to be regulated by CIC (116). Despite these specific transcriptional changes that accompany tumor progression, both CIC mutant and CIC wild-type cells spanned all the tumors' subpopulations (FIG. 20D), indicating that the tumor hierarchy is maintained during clonal evolution.

TABLE 6
Genes upregulated (top) or downregulated (bottom)
in CIC-mutant cells of MGH53.
Genes in CIC-mutant

| | | |
|---|---|---|
| | CIC mutant vs. CIC | CIC mutant vs. |
| Gene | WT (log2-ratio) | unresolved (log2-ratio) |

upnregulated in CIC-mutants

| | | |
|---|---|---|
| ALG9 | 1,227 | 0,8928 |
| AP3S1 | 1,5968 | 0,7338 |
| ARRDC3 | 1,9209 | 1,4759 |
| BRAT1 | 1,4686 | 0,7514 |
| CLN3 | 1,5573 | 1,0239 |
| CNTNAP2 | 1,0757 | 0,7058 |
| COL16A1 | 1,3021 | 0,6934 |
| CTTN | 1,8597 | 1,461 |
| DLD | 1,7493 | 1,278 |
| DOCK10 | 1,1863 | 0,8959 |
| DSEL | 1,3431 | 0,9541 |
| ECI2 | 1,4268 | 0,6268 |
| EP300 | 1,05 | 0,8556 |
| ETVI | 1,7266 | 1,3677 |
| ETV5 | 1,4806 | 1,2395 |
| FAR1 | 1,1284 | 0,6152 |
| FOXRED1 | 1,3849 | 0,6961 |
| FYTTD1 | 1,3993 | 0,7856 |
| GATS | 1,2712 | 0,7535 |
| GFRA1 | 1,1055 | 0,6877 |
| GLT25D2 | 1,8813 | 1,4116 |
| GPR56 | 1,2726 | 1,1663 |
| IGSF8 | 1,6315 | 1,2388 |
| KANKI | 1,8026 | 1,4367 |
| KIAA1467 | 1,3175 | 0,9784 |
| KIF22 | 1,7248 | 1,1386 |
| LNX1 | 1,2214 | 0,7705 |
| LPCAT1 | 1,4064 | 0,9667 |
| ME3 | 1,3976 | 0,9663 |
| MEGF11 | 1,4456 | 0,6222 |
| MRPS16 | 1,3175 | 0,6551 |
| NAVI | 1,3141 | 0,796 |
| NFIA | 1,2509 | 0,931 |
| NIN | 1,4232 | 0,8497 |
| NLGN3 | 1,47 | 0,8141 |
| NUP188 | 1,3793 | 0,8259 |
| PCDH15 | 1,3156 | 0,9597 |
| PCDHB9 | 1,5753 | 0,7125 |
| PPP2R2B | 1,7528 | 0,9681 |
| PPWD1 | 1,5658 | 0,7861 |
| PTN | 1,7714 | 0,8994 |
| RASD1 | 2,0831 | 0,9614 |
| RNF214 | 1,4118 | 0,9173 |
| SDC3 | 1,3395 | 0,884 |
| SEC24B | 1,2845 | 0,6596 |
| SLC38A10 | 1,3295 | 1,4766 |
| STI Ml | 1,268 | 0,9125 |
| TMEM181 | 1,3799 | 0,9492 |
| TTLL5 | 1,1704 | 0,7158 |
| VARS | 1,2929 | 0,7738 |
| YJEFN3 | 1,5865 | 0,7356 |
| ZNF451 | 1,0488 | 0,6191 |
| ZNF564 | 1,3004 | 0,9083 |

downregulated in CIC-mutants

| | | |
|---|---|---|
| ANKMY2 | −1,579 | −0,6162 |
| ATF4 | −1,9523 | −1,3151 |
| BRK1 | −1,837 | −1,9774 |
| BTF3L4 | −1,3483 | −1,0247 |
| EIF3C | −2,0108 | −0,8491 |
| EVI2A | −1,3452 | −0,8935 |
| GFAP | −2,281 | −0,82 |
| MAD2L2 | −1,5275 | −1,1485 |
| MPV17 | −1,761 | −1,2259 |
| MRPL46 | −1,6656 | −0,5991 |
| NDUFVI | −1,8719 | −1,4593 |
| NFE2L2 | −2,1095 | −0,634 |
| RAB1A | −1,5867 | −0,9021 |
| RCOR3 | −1,261 | −0,8461 |
| RSL1D1 | −1,2432 | −0,8095 |
| TTC14 | −1,3767 | −0,727 |

Taken together, the CNV and point-mutation analyses demonstrate that various subclonal mutations span the cellular hierarchy defined by expression profiles and strongly argue that this hierarchy reflects non-genetic states. Similar results were also obtained for analysis of a loss-of-heterozygosity event in MGH54 (FIG. 39). While our genetic analysis does not cover all possible mutations due to technical limitations, Applicants note that the alternative model of genetically-driven hierarchy would predict that all subclonal mutations should conform to a global phylogenetic structure that distinguishes between tumor compartments, and is thus highly inconsistent with our results (FIG. 40). Interestingly, Applicants also identified down-regulation of GFAP in CIC mutant cells, possibly contributing to the weaker GFAP expression in oligodendrogliomas than astrocytomas (95). Despite these specific transcriptional changes, both CIC mutant and CIC wild-type cells spanned all the tumors' subpopulations (FIG. 20D), further indicating that the tumor hierarchy is maintained during clonal evolution.

While genetic events do not appear to define the hierarchy, they may nevertheless influence it. The two clones detected in MGH36 and MGH97 each included cells from all three compartments of the cellular hierarchy, yet they differed in their relative distributions (FIG. 20A,B, FIG. 37). Clone 1 of MGH36 displayed higher frequency of stem/progenitors (P=4*10⁻¹⁰, Fisher's exact test) while clone 2 displayed higher frequency of AC-like cells (P=2*10⁻¹⁰). Similarly, clone 2 of MGH97 contained higher frequency of stem/progenitors (P<10⁻¹⁶) suggesting that genetic evolution may have modulated the patterns of self-renewal and differentiation in these tumors. Furthermore, the frequencies of cycling cells were higher in clone 1 of MGH36 and in clone 2 of MGH97, consistent with their increased frequencies of stem/progenitors. In MGH36 Applicants also observed rare OC-like cells in the G1/S phases exclusively in clone 2 (FIG. 37). Thus, the coupling between cell cycle and stemness may also be partially affected by genetic events.

In conclusion, this large-scale analysis of single-cell composition in grade II gliomas uncovers a developmental hierarchy shared across multiple oligodendrogliomas and multiple genetic subclones, indicating a model of tumorigenesis where a subpopulation of stem/progenitor cells propagates these tumors in humans, while accruing new mutations, as well as giving rise to differentiated and non-cycling cells of two distinct glial lineages with similar genotypes. Indeed, this hierarchy is recapitulated in clones that are genetically distinguishable in our data, such as in CIC wild-type vs. mutant cells. Interestingly, our single-cell data indicate that oligodendroglioma stem/progenitor cells resemble a primitive tri-potent neural cell type, such as NSC or NPC, more so than a more committed glial progenitor like an OPC(108, 117).

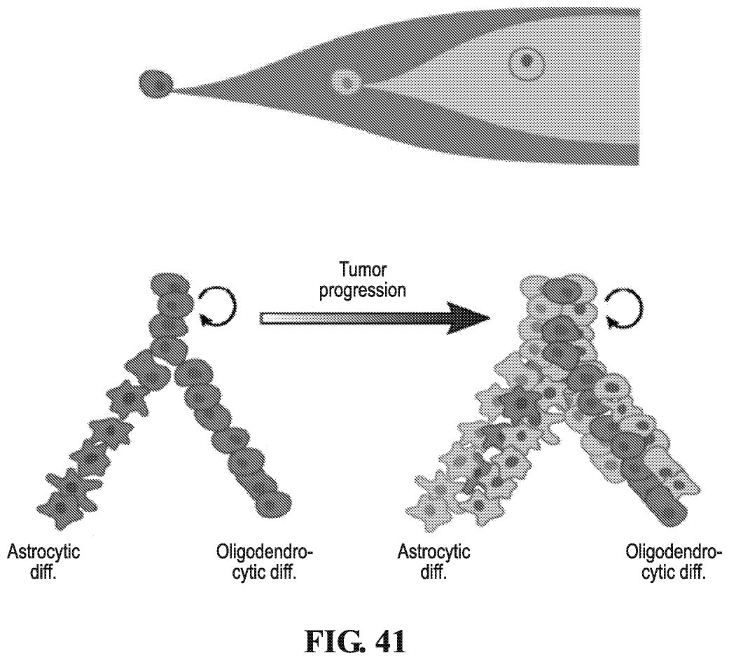

One limitation of studying low-grade oligodendrogliomas is that Applicants could neither perform functional validation of tumoral lineages nor test the capacity of different populations to initiate tumors in animals, since human grade II oligodendrogliomas do not grow in mouse xenograft assays, and even in-vitro models are sparse and maintain only limited similarity to cancer cells in situ. Yet our approach and analyses highlight the key role of single cell genomics as a tool for unbiased analysis of single-cell states directly in patient tumors, without confounding factors such as xenogeneic milieu and conditions that are drastically different from the native environment (72). Outlining genetic from non-genetic influences—albeit with limitations in sensitivity due to single cell RNA-Seq—allows us to present an integrated model of how diverse genetic clones, each with their own developmental hierarchy, coordinate tumor maintenance and evolution in humans, unifying the cancer stem cell and the genetic models of cancer in this clinical context (72) (FIG. 41).

The results described herein highlight a subpopulation of undifferentiated cells that possess stem cell transcriptional signatures and also show enriched proliferative potential. Thus, the most primitive and undifferentiated population of cancer cells are the main source of proliferating cells in patients with oligodendroglioma. This might explain the relative clinical sensitivity of these tumors to treatments that selectively kill proliferating cells such as radiochemotherapies (118). At least early in their pathogenesis these tumors may maintain hierarchies from normal development with stem cells that robustly follow differentiation programs, leaving oligodendroglioma stem cells as the only cycling populations. This architecture might differ in other brain tumors and in higher-grade lesions where differentiation might be compromised. By providing the genome-wide transcriptional signature of cancer stem/progenitor cells in oligodendroglioma, this work delineates cellular programs that represent valuable targets to impact tumor growth. The verticality of the observed hierarchy indicates that, in this clinical context, triggering cells to differentiate along one of two glial axes may yield therapeutic benefit. It is postulated that further studies, deploying large-scale single-cell profiling technologies in genetically defined human malignancies will demonstrate the generality of our findings and investigate opportunities for clinical translation.

Note 1. Accounting for the impact of technical and batch effects. Applicants used several approaches to ascertain that our transcriptional signatures are observed independently of technical effects. First, different batches are indistinguishable with respect to the expression hierarchy, as shown in FIG. 24B. Second, to minimize the impact of technical effects, namely the differences in complexity (e.g. the number of genes detected per cell), Applicants use a weighted version of principal component analysis as described in Methods. Third, the biological clusters Applicants describe are not driven by complexity. As described in Methods, Applicants performed control PCA on shuffled data. Comparison of the PCA on the original and shuffled data (FIG. 24D) shows that the OC-like and AC-like genes used in our analysis lose their association with PC1 in the shuffled data, indicating that their patterns are not driven by complexity. Similarly, complexity does not account for the PC2/3 stemness program, as PC2 cell scores are positively correlated with complexity (R=0.27), while PC3 cell scores are negatively correlated with complexity (R=−0.24) and stemness genes were defined as those correlated with both PC2 and PC3.

Note 2. Assessing the presence of intermediate differentiation states. Technical noise is not expected to distinguish functionally-related from functionally-unrelated sets of genes. Within a given cell, the level of each gene can be over-estimated or under-estimated due to the capture of only a subset of transcripts and their potentially biased amplification; but there is no reason to expect that two functionally related genes will have the same pattern, i.e., commonly over-estimated or commonly under-estimated, except as correlated to their global expression levels. That is, the exception is if the two genes are both highly expressed or both lowly expressed and thus could be commonly affected by the “complexity” of single cell libraries, such that two lowly expressed genes tend to be undetected in cells with a lower overall number of detected genes. However, this does not affect our lineage scores, both because the set of AC and OC genes are not associated with very different overall expression levels, and because Applicants use “control” gene-sets with comparable expression levels when defining lineage scores. In each of the three tumors that Applicants profiled at high depth, and within each of the two lineages Applicants find significant co-expression patterns that suggest distinct differentiation states (FIG. 42). For example, within the AC lineage, Applicants find significant co-expression patterns in the range of 0.5 to 1, as well as within the range of 1 to 2. However, in more limited ranges Applicants typically do not detect significant co-expression patterns (e.g., in the range 1.5 to 2, Applicants detect significant co-expression only in one of the three tumors). Applicants conclude that cells likely exist in distinct stages of differentiation although the number of distinct states may be limited.

### REFERENCES

1. D. Hanahan, R. A. Weinberg, Hallmarks of cancer: the next generation. Cell. 144, 646-674 (2011).
2. C. E. Meacham, S. J. Morrison, Tumour heterogeneity and cancer cell plasticity. Nature. 501, 328-337 (2013).
3. F. S. Hodi et al., Improved Survival with Ipilimumab in Patients with Metastatic Melanoma. N. Engl. J. Med. 363, 711-723 (2010).
4. J. R. Brahmer et al., Phase I study of single-agent anti-programmed death-1 (MDX-1106) in refractory solid tumors: safety, clinical activity, pharmacodynamics, and immunologic correlates. J. Clin. Oncol. Off. J. Am. Soc. Clin. Oncol. 28, 3167-3175 (2010).
5. J. R. Brahmer et al., Safety and Activity of Anti-PD-L1 Antibody in Patients with Advanced Cancer. N. Engl. J. Med 366, 2455-2465 (2012).
6. S. L. Topalian et al., Safety, activity, and immune correlates of anti-PD-1 antibody in cancer. N. Engl. J. Med 366, 2443-2454 (2012).
7. O. Hamid et al., Safety and tumor responses with lambrolizumab (anti-PD-1) in melanoma. N. Engl. J. Med 369, 134-144 (2013).
8. J. S. Weber et al., Safety, efficacy, and biomarkers of nivolumab with vaccine in ipilimumabrefractory or -naive melanoma. J Clin. Oncol. Off J. Am. Soc. Clin. Oncol. 31, 4311-4318 (2013).
9. K. M. Mahoney, M. B. Atkins, Prognostic and predictive markers for the new immunotherapies. Oncol. Williston Park N. 28 Suppl 3, 39-48 (2014).
10. J. Larkin et al., Combined Nivolumab and Ipilimumab or Monotherapy in Untreated Melanoma. N. Engl. J. Med 373, 23-34 (2015).
11. A. Snyder et al., Genetic basis for clinical response to CTLA-4 blockade in melanoma. N. Engl. J. Med 371, 2189-2199 (2014).
12. N. Wagle et al., Dissecting Therapeutic Resistance to RAF Inhibition in Melanoma by Tumor Genomic Profiling. J. Clin. Oncol. (2011), doi:10.1200/JCO.2010.33.2312.
13. E. M. Van Allen et al., The genetic landscape of clinical resistance to RAF inhibition in metastatic melanoma. Cancer Discov. 4, 94-109 (2014).
14. A. K. Shalek et al., Single-cell transcriptomics reveals bimodality in expression and splicing in immune cells. Nature. 498, 236-240 (2013).
15. A. P. Patel et al., Single-cell RNA-seq highlights intratumoral heterogeneity in primary glioblastoma. Science. 344, 1396-1401 (2014).
16. E. Z. Macosko et al., Highly Parallel Genome-wide Expression Profiling of Individual Cells Using Nanoliter Droplets. Cell. 161, 1202-1214 (2015).
17. L. van der Maaten, G. Hinton, Visualizing Data using t-SNE. 9, 2579-2605 (2008).
18. M. Ester, H. Kriegel, J. Sander, and X. Xu, “A density-based algorithm for discovering clusters in large spatial databases with noise,” in Proc. 2nd Int. Conf. Knowledge Discovery and Data Mining (KDD '96), 1996, pp. 226-231.
19. M. L. Whitfield, L. K. George, G. D. Grant, C. M. Perou, Common markers of proliferation. Nat. Rev. Cancer. 6, 99-106 (2006).
20. A. Roesch et al., A temporarily distinct subpopulation of slow-cycling melanoma cells is required for continuous tumor growth. Cell. 141, 583-594 (2010).
21. A first-in-human phase I study of the CDK4/6 inhibitor, LY2835219, for patients with advanced cancer. J. Clin. Oncol. (available at meetinglibrary.asco.org/content/111069-132).
22. C. M. Johannessen et al., A melanocyte lineage program confers resistance to MAP kinase pathway inhibition. Nature. 504, 138-142 (2013).
23. D. J. Konieczkowski et al., A melanoma cell state distinction influences sensitivity to MAPK pathway inhibitors. Cancer Discov. 4, 816-827 (2014).
24. L. A. Garraway et al., Integrative genomic analyses identify MITF as a lineage survival oncogene amplified in malignant melanoma. Nature. 436, 117-122 (2005).
25. Z. Zhang et al., Activation of the AXL kinase causes resistance to EGFR-targeted therapy in lung cancer. Nat. Genet. 44, 852-860 (2012).
26. X. Wu et al., AXL kinase as a novel target for cancer therapy. Oncotarget. 5, 9546-9563 (2014).
27. A. D. Boiko et al., Human melanoma-initiating cells express neural crest nerve growth factor receptor CD271. Nature. 466, 133-137 (2010).
28. K. S. Hoek et al., In vivo Switching of Human Melanoma Cells between Proliferative and Invasive States. Cancer Res. 68, 650-656 (2008).
29. J. Müller et al., Low MITF/AXL ratio predicts early resistance to multiple targeted drugs in melanoma. Nat. Commun. 5, 5712 (2014).
30. F. Z. Li, A. S. Dhillon, R. L. Anderson, G. McArthur, P. T. Ferrao, Phenotype switching in melanoma. implications for progression and therapy. Mol. Cell. Oncol. 5, 31 (2015).
31. W. Hugo et al., Non-genomic and Immune Evolution of Melanoma Acquiring MAPKi Resistance. Cell. 162, 1271-1285 (2015).
32. R. Nazarian et al., Melanomas acquire resistance to B-RAF(V600E) inhibition by RTK or N-RAS upregulation. Nature. 468, 973-977 (2010).
33. J. Barretina et al., The Cancer Cell Line Encyclopedia enables predictive modelling of anticancer drug sensitivity. Nature. 483, 603-607 (2012).
34. W. H. Fridman, F. Pages, C. Sautes-Fridman, J. Galon, The immune contexture in human tumours: impact on clinical outcome. Nat. Rev. Cancer. 12, 298-306 (2012).
35. S. L. Carter et al., Absolute quantification of somatic DNA alterations in human cancer. Nat. Biotechnol. 30, 413-421 (2012).
36. Roadmap Epigenomics Consortium et al., Integrative analysis of 111 reference human epigenomes. Nature. 518, 317-330 (2015).
37. R. Akbani et al., Genomic Classification of Cutaneous Melanoma. Cell. 161, 1681-1696 (2015).
38. M. M. Markiewski et al., Modulation of the antitumor immune response by complement. Nat. Immunol. 9, 1225-1235 (2008).
39. E. J. Wherry, T cell exhaustion. Nat. Immunol. 12, 492-499 (2011).
40. L. Chen, D. B. Flies, Molecular mechanisms of T cell co-stimulation and co-inhibition. Nat. Rev. Immunol. 13, 227-242 (2013).
41. H. Borghaei et al., Nivolumab versus Docetaxel in Advanced Nonsquamous Non-Small-Cell Lung Cancer. N. Engl. J. Med 373, 1627-1639 (2015).
42. R. J. Motzer et al., Nivolumab versus Everolimus in Advanced Renal-Cell Carcinoma. N. Engl. J.Med 373, 1803-1813 (2015).
43. N. A. Rizvi et al., Cancer immunology. Mutational landscape determines sensitivity to PD-1 blockade in non-small cell lung cancer. Science. 348, 124-128 (2015).
44. E. M. Van Allen et al., Genomic correlates of response to CTLA-4 blockade in metastatic melanoma. Science. 350, 207-211 (2015).
45. E. J. Wherry et al., Molecular signature of CD8+ T cell exhaustion during chronic viral infection. Immunity. 27, 670-684 (2007).
46. L. Baitsch et al., Exhaustion of tumor-specific CD8+ T cells in metastases from melanoma patients. J. Clin. Invest. 121, 2350-2360 (2011).
47. G. J. Martinez et al., The transcription factor NFAT promotes exhaustion of activated CD8+ T cells. Immunity. 42, 265-278 (2015).
48. S. D. Blackburn, H. Shin, G. J. Freeman, E. J. Wherry, Selective expansion of a subset of exhausted CD8 T cells by αPD-L1 blockade. Proc. Natl. Acad Sci. U.S.A (2008) (available at agris.fao.org/agris-search/search.do?recordID=US201301547699).
49. L. Baitsch et al., Extended Co-Expression of Inhibitory Receptors by Human CD8 T-Cells Depending on Differentiation, Antigen-Specificity and Anatomical Localization. PLoS ONE. 7, e30852 (2012).
50. S. Picelli et al., Smart-seq2 for sensitive full-length transcriptome profiling in single cells. Nat. Methods. 10, 1096-1098 (2013).
51. J. J. Trombetta et al., Preparation of Single-Cell RNA-Seq Libraries for Next Generation Sequencing. Curr. Protoc. Mol. Biol. Ed Frederick M Ausubel Al. 107, 4.22.1-4.22.17 (2014).
52. H. Li, R. Durbin, Fast and accurate short read alignment with Burrows-Wheeler transform. Bioinforma. Oxf. Engl. 25, 1754-1760 (2009).
53. A. McKenna et al., The Genome Analysis Toolkit: a MapReduce framework for analyzing nextgeneration DNA sequencing data. Genome Res. 20, 1297-1303 (2010).
54. M. F. Berger et al., The genomic complexity of primary human prostate cancer. Nature. 470, 214-20 (2011).
55. K. Cibulskis et al., Sensitive detection of somatic point mutations in impure and heterogeneous cancer samples. Nat. Biotechnol. 31, 213-9 (2013).
56. C. T. Saunders et al., Strelka: accurate somatic small-variant calling from sequenced tumornormal sample pairs. Bioinforma. Oxf Engl. 28, 1811-7 (2012).
57. A. H. Ramos et al., Oncotator: cancer variant annotation tool. Hum. Mutat. 36, E2423-9 (2015).
58. E. S. Venkatraman, A. B. Olshen, A faster circular binary segmentation algorithm for the analysis of array CGH data. Bioinforma. Oxf Engl. 23, 657-63 (2007).
59. B. Langmead, C. Trapnell, M. Pop, S. L. Salzberg, Ultrafast and memory-efficient alignment of short DNA sequences to the human genome. Genome Biol. 10, R25 (2009).
60. B. Li, C. N. Dewey, RSEM: accurate transcript quantification from RNA-Seq data with or without a reference genome. BMC Bioinformatics. 12, 323 (2011).
61. A. K. Shalek et al., Single-cell RNA-seq reveals dynamic paracrine control of cellular variation. Nature. 510, 363-369 (2014).
62. M. L. Whitfield et al., Identification of genes periodically expressed in the human cell cycle and their expression in tumors. Mol. Biol. Cell. 13, 1977-2000 (2002).
63. D. E. Campton et al., High-recovery visual identification and single-cell retrieval of circulating tumor cells for genomic analysis using a dual-technology platform integrated with automated immunofluorescence staining. BMC Cancer. 15, 360 (2015).
64. I. Skaland et al., Comparing subjective and digital image analysis HER2/neu expression scores with conventional and modified FISH scores in breast cancer. J. Clin. Pathol. 61, 68-71 (2008).
65. J. Konsti et al., Development and evaluation of a virtual microscopy application for automated assessment of Ki-67 expression in breast cancer. BMC Clin. Pathol. 11, 3 (2011).
66. W. Hugo et al., Non-genomic and Immune Evolution of Melanoma Acquiring MAPKi Resistance. Cell. 162, 1271-1285 (2015).
67. L. Baitsch et al., Extended Co-Expression of Inhibitory Receptors by Human CD8 T-Cells Depending on Differentiation, Antigen-Specificity and Anatomical Localization. PLoS ONE. 7, e30852 (2012).
68. E. J. Wherry et al., Molecular signature of CD8+ T cell exhaustion during chronic viral infection. Immunity. 27, 670-684 (2007).
69. G. J. Martinez et al., The transcription factor NFAT promotes exhaustion of activated CD8+ T cells. Immunity. 42, 265-278 (2015).
70. E. A. Eisenhauer et al., New response evaluation criteria in solid tumours: revised RECIST guideline (version 1.1). Eur. J. Cancer Oxf. Engl. 1990. 45, 228-247 (2009).
71. J. Barretina et al., The Cancer Cell Line Encyclopedia enables predictive modelling of anticancer drug sensitivity. Nature. 483, 603-607 (2012).
72. Kreso, A. & Dick, J. E. Evolution of the cancer stem cell model. Cell stem cell 14, 275-291, (2014).
73. Baylin, S. B. & Jones, P. A. A decade of exploring the cancer epigenome—biological and translational implications. Nature reviews. Cancer 11, 726-734, (2011).
74. Suva, M. L., Riggi, N. & Bernstein, B. E. Epigenetic reprogramming in cancer. Science 339, 1567-1570, (2013).
75. Bao, S., Wu, Q., McLendon, R. E., Hao, Y., Shi, Q., Hjelmeland, A. B. et al. Glioma stem cells promote radioresistance by preferential activation of the DNA damage response. Nature 444, 756-760, (2006).
76. Chen, J., Li, Y., Yu, T. S., McKay, R. M., Burns, D. K., Kernie, S. G. et al. A restricted cell population propagates glioblastoma growth after chemotherapy. Nature 488, 522-526, (2012).
77. Ito, K., Bernardi, R., Morotti, A., Matsuoka, S., Saglio, G., Ikeda, Y. et al. PML targeting eradicates quiescent leukaemia-initiating cells. Nature 453, 1072-1078, (2008).
78. Lathia, J. D., Gallagher, J., Heddleston, J. M., Wang, J., Eyler, C. E., Macswords, J. et al. Integrin alpha 6 regulates glioblastoma stem cells. Cell stem cell 6, 421-432, (2010).
79. Piccirillo, S. G., Reynolds, B. A., Zanetti, N., Lamorte, G., Binda, E., Broggi, G. et al. Bone morphogenetic proteins inhibit the tumorigenic potential of human brain tumour-initiating cells. Nature 444, 761-765, (2006).
80. Singh, S. K., Hawkins, C., Clarke, I. D., Squire, J. A., Bayani, J., Hide, T. et al. Identification of human brain tumour initiating cells. Nature 432, 396-401, (2004).
81. Anido, J., Saez-Borderias, A., Gonzalez-Junca, A., Rodon, L., Folch, G., Carmona, M. A. et al. TGF-beta Receptor Inhibitors Target the CD44(high)/Id1(high) Glioma-Initiating Cell Population in Human Glioblastoma. Cancer cell 18, 655-668, (2010).
82. Son, M. J., Woolard, K., Nam, D. H., Lee, J. & Fine, H. A. SSEA-1 is an enrichment marker for tumor-initiating cells in human glioblastoma. Cell stem cell 4, 440-452, (2009).
83. Srikanth, M., Kim, J., Das, S. & Kessler, J. A. BMP signaling induces astrocytic differentiation of clinically derived oligodendroglioma propagating cells. Mol Cancer Res 12 283-294 (2014).
84. Friedmann-Morvinski, D., Bushong, E. A., Ke, E., Soda, Y., Marumoto, T., Singer, O. et al. Dedifferentiation of neurons and astrocytes by oncogenes can induce gliomas in mice. Science 338, 1080-1084, (2012).
85. Dalerba, P., Kalisky, T., Sahoo, D., Rajendran, P. S., Rothenberg, M. E., Leyrat, A. A. et al. Single-cell dissection of transcriptional heterogeneity in human colon tumors. Nature biotechnology 29, 1120-1127 (2011).
86. Lawson, D. A., Bhakta, N. R., Kessenbrock, K., Prummel, K. D., Yu, Y., Takai, K. et al. Single-cell analysis reveals a stem-cell program in human metastatic breast cancer cells. Nature 526 131-135 (2015).
87. Jaitin, D. A., Kenigsberg, E., Keren-Shaul, H., Elefant, N., Paul, F., Zaretsky, I. et al. Massively parallel single-cell RNA-seq for marker-free decomposition of tissues into cell types. Science 343 776-779 (2014).
88. Pollen, A. A., Nowakowski, T. J., Shuga, J., Wang, X., Leyrat, A. A., Lui, J. H. et al. Low-coverage single-cell mRNA sequencing reveals cellular heterogeneity and activated signaling pathways in developing cerebral cortex. Nature biotechnology 32 1053-1058 (2014).
89. Treutlein, B., Brownfield, D. G., Wu, A. R., Neff, N. F., Mantalas, G. L., Espinoza, F. H. et al. Reconstructing lineage hierarchies of the distal lung epithelium using single-cell RNA-seq. Nature 509 371-375 (2014).
90. Zeisel, A., Munoz-Manchado, A. B., Codeluppi, S., Lonnerberg, P., La Manno, G., Jureus, A. et al. Brain structure. Cell types in the mouse cortex and hippocampus revealed by single-cell RNA-seq. Science 347 1138-1142 (2015).
91. Suva, M. L. & Louis, D. N. Next-generation molecular genetics of brain tumours. Current opinion in neurology 26, 681-687, (2013).
92. Louis, D. N., Perry, A., Burger, P., Ellison, D. W., Reifenberger, G., von Deimling, A. et al. International Society Of Neuropathology--Haarlem consensus guidelines for nervous system tumor classification and grading. Brain pathology 24, 429-435, (2014).
93. Picelli, S., Faridani, O. R., Bjorklund, A. K., Winberg, G., Sagasser, S. & Sandberg, R. Full-length RNA-seq from single cells using Smart-seq2. Nat Protoc 9 171-181 (2014).
94. Butovsky, O., Jedrychowski, M. P., Moore, C. S., Cialic, R., Lanser, A. J., Gabriely, G. et al. Identification of a unique TGF-beta-dependent molecular and functional signature in microglia. Nat Neurosci 17 131-143 (2014).
95. Rousseau, A., Nutt, C. L., Betensky, R. A., Iafrate, A. J., Han, M., Ligon, K. L. et al. Expression of oligodendroglial and astrocytic lineage markers in diffuse gliomas: use of YKL-96. ApoE, ASCL1, and NKX2-2. Journal of neuropathology and experimental neurology 65 1149-1156 (2006).
97. Zhang, Y., Chen, K., Sloan, S. A., Bennett, M. L., Scholze, A. R., O'Keeffe, S. et al. An RNA-sequencing transcriptome and splicing database of glia, neurons, and vascular cells of the cerebral cortex. J Neurosci 34 11929-11947 (2014).
98. Louis, D. N., Ohgaki, H., Wiestler, O. D., Cavenee, WHO classification of tumours of the central nervous system. (Revised 4^{th} edition). IARC: Lyon., (2016).
99. Feng, W., Khan, M. A., Bellvis, P., Zhu, Z., Bernhardt, O., Herold-Mende, C. et al. The chromatin remodeler CHD7 regulates adult neurogenesis via activation of SoxC transcription factors. Cell stem cell 13, 62-72, (2013).
100. Ikushima, H., Todo, T., Ino, Y., Takahashi, M., Miyazawa, K. & Miyazono, K. Autocrine TGF-beta signaling maintains tumorigenicity of glioma-initiating cells through Sry-related HMG-box factors. Cell stem cell 5, 504-514, (2009).
101. Suva, M. L., Rheinbay, E., Gillespie, S. M., Patel, A. P., Wakimoto, H., Rabkin, S. D. et al. Reconstructing and reprogramming the tumor-propagating potential of glioblastoma stem-like cells. Cell 157, 580-594, (2014).
102. Mille, F., Tamayo-Orrego, L., Levesque, M., Remke, M., Korshunov, A., Cardin, J. et al. The Shh receptor Boc promotes progression of early medulloblastoma to advanced tumors. Developmental cell 31, 34-47, (2014).
103. Panchision, D. M., Chen, H. L., Pistollato, F., Papini, D., Ni, H. T. & Hawley, T. S. Optimized flow cytometric analysis of central nervous system tissue reveals novel functional relationships among cells expressing CD133, CD15, and CD24. Stem cells 25 1560-1570 (2007).
104. Rheinbay, E., Suva, M. L., Gillespie, S. M., Wakimoto, H., Patel, A. P., Shahid, M. et al. An Aberrant Transcription Factor Network Essential for Wnt Signaling and Stem Cell Maintenance in Glioblastoma. Cell reports 3, 1567-1579, (2013).
105. Miller, J. A., Ding, S. L., Sunkin, S. M., Smith, K. A., Ng, L., Szafer, A. et al. Transcriptional landscape of the prenatal human brain. Nature 508, 199-206, (2014).
106. Darmanis, S., Sloan, S. A., Zhang, Y., Enge, M., Caneda, C., Shuer, L. M. et al. A survey of human brain transcriptome diversity at the single cell level. Proceedings of the National Academy of Sciences of the United States of America, (2015).
107. Kelly, J. J., Blough, M. D., Stechishin, O. D., Chan, J. A., Beauchamp, D., Perizzolo, M. et al. Oligodendroglioma cell lines containing t(1;19)(q10;p10). Neuro-oncology 12 745-755 (2010).
108. Sugiarto, S., Persson, A. I., Munoz, E. G., Waldhuber, M., Lamagna, C., Andor, N. et al. Asymmetry-defective oligodendrocyte progenitors are glioma precursors. Cancer cell 20 328-340 (2011).
109. Aguirre, A., Dupree, J. L., Mangin, J. M. & Gallo, V. A functional role for EGFR signaling in myelination and remyelination. Nat Neurosci 10 990-1002 (2007).
110. Shah, N. M., Marchionni, M. A., Isaacs, I., Stroobant, P. & Anderson, D. J. Glial growth factor restricts mammalian neural crest stem cells to a glial fate. Cell 77 349-360 (1994).
111. Shin, J., Berg, D. A., Zhu, Y., Shin, J. Y., Song, J., Bonaguidi, M. A. et al. Single-Cell RNA-Seq with Waterfall Reveals Molecular Cascades underlying Adult Neurogenesis. Cell stem cell 17, 360-372, (2015).
112. Cancer Genome Atlas Research, N., Brat, D. J., Verhaak, R. G., Aldape, K. D., Yung, W. K., Salama, S. R. et al. Comprehensive, Integrative Genomic Analysis of Diffuse Lower-Grade Gliomas. The New England journal of medicine 372, 2481-2498, (2015).
113. Lange, C. & Calegari, F. Cdks and cyclins link G1 length and differentiation of embryonic, neural and hematopoietic stem cells. Cell Cycle 9 1893-1900 (2010).
114. Koyama-Nasu, R., Nasu-Nishimura, Y., Todo, T., Ino, Y., Saito, N., Aburatani, H. et al. The critical role of cyclin D2 in cell cycle progression and tumorigenicity of glioblastoma stem cells. Oncogene 32 3840-3845 (2013).
115. Bettegowda, C., Agrawal, N., Jiao, Y., Sausen, M., Wood, L. D., Hruban, R. H. et al. Mutations in CIC and FUBP1 contribute to human oligodendroglioma. Science 333 1453-1455 (2011).
116. Padul, V., Epari, S., Moiyadi, A., Shetty, P. & Shirsat, N. V. ETV/Pea3 family transcription factor-encoding genes are overexpressed in CIC-mutant oligodendrogliomas. Genes, chromosomes & cancer 54, 725-733, (2015).
117. Liu, C., Sage, J. C., Miller, M. R., Verhaak, R. G., Hippenmeyer, S., Vogel, H. et al. Mosaic analysis with double markers reveals tumor cell of origin in glioma. Cell 146 209-221 (2011).
118. Ducray, F. & Idbaih, A. Neuro-oncology: anaplastic oligodendrogliomas-value of early chemotherapy. Nat Rev Neurol 9 7-8 (2013).
119. Satija, R., Farrell, J. A., Gennert, D., Schier, A. F. & Regev, A. Spatial reconstruction of single-cell gene expression data. Nature biotechnology 33 495-502 (2015).
120. Mohapatra, G., Betensky, R. A., Miller, E. R., Carey, B., Gaumont, L. D., Engler, D. A. et al. Glioma test array for use with formalin-fixed, paraffin-embedded tissue: array comparative genomic hybridization correlates with loss of heterozygosity and fluorescence in situ hybridization. J Mol Diagn 8 268-276 (2006).
121. Cibulskis, K., McKenna, A., Fennell, T., Banks, E., DePristo, M. & Getz, G. ContEst: estimating cross-contamination of human samples in next-generation sequencing data. Bioinformatics 27 2601-2602 (2011).
122. Costello, M., Pugh, T. J., Fennell, T. J., Stewart, C., Lichtenstein, L., Meldrim, J. C. et al. Discovery and characterization of artifactual mutations in deep coverage targeted capture sequencing data due to oxidative DNA damage during sample preparation. Nucleic Acids Res 41 e67 (2013).
123. Zhang, Y., Sloan, S. A., Clarke, L. E., Caneda, C., Plaza, C. A., Blumenthal, P. D. et al. Purification and Characterization of Progenitor and Mature Human Astrocytes Reveals Transcriptional and Functional Differences with Mouse. Neuron 89, 37-53, (2016).
124. Kowalczyk, M. S., Tirosh, I., Heckl, D., Rao, T. N., Dixit, A., Haas, B. J. et al. Single-cell RNA-seq reveals changes in cell cycle and differentiation programs upon aging of hematopoietic stem cells. Genome Res 25; 1860-1872 (2015).
125. Lawrence M. S., Stojanov P., Mermel C. H., Robinson J. T., Garraway L. A., Golub T. R. et al. Discovery and saturation analysis of cancer genes across 21 tumour types. Nature 505, 495-501 (2014).
126. Tirosh I., Izar B., Prakadan S. M., Wadsworth M. H. 2nd, Treacy D., Trombetta J. J. et al. Dissecting the multicellular ecosystem of metastatic melanoma by single-cell RNA-seq. Science, 352, 189-96 (2016).
127. Filbin, M. G. and Suva, M. L. Gliomas Genomics and Epigenomics: Arriving at the Start and Knowing It for the First Time. Annual review of pathology, 11: 497-521 (2016).
128. Dai C, Celestino J C, Okada Y, Louis D N, Fuller G N, Holland E C, PDGF autocrine stimulation dedifferentiates cultured astrocytes and induces oligodendrogliomas and oligoastrocytomas from neural progenitors and astrocytes in vivo. Genes & development 15, 1913 (2001).
129. Bennett M. L., Bennett F. C., Liddelow S. A., Ajami B., Zamanian J. L., Fernhoff N. B. et al., New tools for studying microglia in the mouse and human CNS. Proceedings of the National Academy of Sciences of the United States of America. 113, E1738-46 (2016).
130. Lavin Y., Winter D., Blecher-Gonen R., David E., Keren-Shaul H., Merad M. et al., Tissue-resident macrophage enhancer landscapes are shaped by the local microenvironment. Cell 159, 1312 (2014).
131. H. Zong, L. F. Parada, S. J. Baker, Cell of origin for malignant gliomas and its implication in therapeutic development. Cold Spring Harbor perspectives in biology 7(5) (2015).
132. Sahm F., Reuss D., Koelsche C., Capper D., Schittenhelm J., Heim S. et al., Farewell to oligoastrocytoma: in situ molecular genetics favor classification as either oligodendroglioma or astrocytoma. Acta neuropathologica 128, 551 (2014).
133. Matcovitch-Natan O., Winter D. R., Giladi A., Vargas Aguilar S., Spinrad A., Sarrazin S. et al. Microglia development follows a stepwise program to regulate brain homeostasis. Science, 19; 353, 6301 (2016).
134. Wei C. L., Wu Q., Vega V. B., Chiu K. P., Ng P., Zhang T. et al., A global map of p53 transcription-factor binding sites in the human genome. Cell. 124, 207-19 (2006).
135. I. C. Macaulay et al., in Nat Methods. (United States, 2015), vol. 12, pp. 519-522.
136. I. C. Macaulay et al., in Nat Protoc. (England, 2016), vol. 11, pp. 2081-2103.
137. Tirosh et al., in Nature. (England, 2016), vol. 539, pp. 309-313.
138. L. Sequeira, C. W. Dubyk, T. A. Riesenberger, C. R. Cooper, K. L. van Golen, Rho GTPases in PC-3 prostate cancer cell morphology, invasion and tumor cell diapedesis. Clin Exp Metastasis 25, 569-579 (2008).
139. M. Tseliou et al., in Cell Physiol Biochem. (Switzerland, 2016), vol. 38, pp. 94-109.
140. T. Cooks, C. C. Harris, M. Oren, in Carcinogenesis. (England, 2014), vol. 35, pp. 1680-1690.
141. N. Cancer Genome Atlas Research, Genomic and epigenomic landscapes of adult de novo acute myeloid leukemia. The New England journal of medicine 368, 2059-2074 (2013).
142. P. Guilhamon et al., in Nat Commun. (England, 2013), vol. 4, pp. 2166.
143. M. J. Aryee et al., in Bioinformatics. (England, 2014), vol. 30, pp. 1363-1369.
144. J. Ihmels et al., in Nature genetics. (United States, 2002), vol. 31, pp. 370-377.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of treating IDH-mutant glioma, comprising administering to a subject in need thereof having an IDH-mutant glioma a therapeutically effective amount of an agent:
capable of reducing the expression or inhibiting the activity of one or more stem cell or progenitor cell signature genes or polypeptides; or
capable of targeting or binding to one or more cell surface exposed stem cell or progenitor cell signature polypeptides, preferably
wherein said one or more stem cell or progenitor cell signature genes or polypeptides is selected from the group consisting of SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, COROIC, EIF4B, FBLIM1, SPDYE7P, TCF4, ORC6, SPDYEl, NCRUPAR, BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5 and TNFAIP8L1; or SOX4, SOX11, SOX2, NFIB, ASCL1, CDH7, CD24, BOC, and TCF4; or SOX4, CCND2, SOX11, CDH7, CD24, NFIB, SOX2, TCF4, ASCL1, BOC, and EGFR: or SOX11, SOX4, NFIB TCF4, SOX2, CDH7, BOC, and CCND2; or SOX11, PTMA, NFIB, CCND2, SOX4, TCF4, CD24, CHD7, and SOX2; or SOX2, SOX4, SOX11, MSI1, TERF2, CTNNB1, USP22, BRD3, CCND2, and PTEN: or SOX4, PTPRS, NFIB, CCND2, RBM6, SET, BAZ2B and TRA2A: or SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2.

2. The method according to claim 1, wherein said agent capable of targeting or binding to one or more cell surface exposed stem cell or progenitor cell signature polypeptides comprises a CAR T-cell capable of targeting or binding to one or more cell surface exposed stem cell or progenitor cell signature polypeptides, and/or
wherein the method comprises reducing the viability of or rendering non-viable stem cells or progenitor cells comprised by the glioma: or
wherein the method comprises inducing differentiation of stem cells or progenitor cells comprised by the glioma; and/or
wherein said stem cell or progenitor cell is a neural stem cell or progenitor cell; and/or
wherein said glioma is oligodendroglioma, astrocytoma, or oligoastrocytoma; and/or
wherein said glioma is low grade glioma or grade II glioma; and/or
wherein said glioma is characterized by IDH1 and/or IDH2 mutations; and/or
wherein said glioma is characterized by CIC mutations; and/or
wherein said glioma is characterized by mutations in one or more genes selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36; and/or
wherein said glioma is characterized by deletion of chromosome arms 1p and/or 19q, and/or
wherein said subject has not previously received chemotherapy and/or radiotherapy.

3. A method of treating IDH-mutant glioma, comprising administering to a subject having an IDH-mutant glioma a therapeutically effective amount of an agent capable of inducing the expression or increasing the activity of one or more astrocyte and/or oligodendrocyte cell signature genes or polypeptides,
preferably, wherein said one or more astrocyte signature gene or polypeptide is selected from the group consisting of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1 and F3; or selected from the group consisting of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2 and F3: or selected from the group consisting of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1 and DNAJB1,
preferably, wherein said one or more oligodendrocyte signature gene or polypeptide is selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X and PHACTR3; or selected from the group consisting of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP and CNP; or selected from the group consisting of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X and PHACTR3.

4. The method according to claim 3, wherein said subject has not previously received chemotherapy and/or radiotherapy, and/or
wherein the method comprises inducing differentiation of stem cells or progenitor cells comprised by the glioma, preferably, wherein said differentiation comprises induction of expression or activity of one or more astrocyte and/or oligodendrocyte signature genes or polypeptides in the stem cells or progenitor cells, and/or
wherein said stem cell or progenitor cell is a neural stem cell or progenitor cell; and/or
wherein said glioma is oligodendroglioma, astrocytoma, or oligoastrocytoma; and/or
wherein said glioma is low grade glioma or grade II glioma; and/or
wherein said glioma is characterized by IDH1 and/or IDH2 mutations; and/or
wherein said glioma is characterized by CIC mutations; and/or
wherein said glioma is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36; and/or
wherein said glioma is characterized by deletion of chromosome arms 1p and/or 19q.

5. (canceled)

6. (canceled)

7. (canceled)

8. A method of diagnosing, prognosing, or stratifying IDH-mutant glioma, comprising determining expression or activity of one or more stem cell or progenitor cell signature genes or polypeptides in cells comprised by the glioma,
preferably, wherein said one or more stem cell or progenitor cell signature genes or polypeptides is selected from the group consisting of SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, COROIC, EIF4B, FBLIM1, SPDYE7P, TCF4, ORC6, SPDYEl, NCRUPAR, BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5 and TNFAIP8L1; or SOX4, SOX11, SOX2, NFIB, ASCL1, CDH7, CD24, BOC, and TCF4; or SOX4, CCND2, SOX11, CDH7, CD24, NFIB, SOX2, TCF4, ASCL1, BOC, and EGFR: or SOX11, SOX4, NFIB TCF4, SOX2, CDH7, BOC, and CCND2; or SOX11, PTMA, NFIB, CCND2, SOX4, TCF4, CD24, CHD7, and SOX2; or SOX2, SOX4, SOX11, MSI1, TERF2, CTNNB1, USP22, BRD3, CCND2, and PTEN: or SOX4, PTPRS, NFIB, CCND2, RBM6, SET, BAZ2B and TRA2A: or SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2, preferably,
wherein said glioma is oligodendroglioma, astrocytoma, or oligoastrocytoma; and/or
wherein said stem cell or progenitor cell is a neural stem cell or progenitor cell; and/or
wherein said glioma is low grade glioma or grade II glioma; and/or
wherein said glioma is characterized by IDH1 and/or IDH2 mutations; and/or
wherein said glioma is characterized by CIC mutations; and/or
wherein said glioma is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36; and/or
wherein said glioma is characterized by deletion of chromosome arms 1p and/or 19q, and/or
wherein said subject has not previously received chemotherapy and/or radiotherapy.

9. The method according to claim 8, comprising determining the relative expression level of one or more stem cell or progenitor cell signature genes or polypeptides compared to one or more astrocyte and/or oligodendrocyte signature genes or polypeptides in the cells comprised by the glioma,
preferably, wherein said one or more astrocyte signature gene or polypeptide is selected from the group consisting of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, IDI, TPCN1, LRRC8A, MT2A, FOSB, LICAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1 and F3; or selected from the group consisting of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2 and F3: or selected from the group consisting of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1 and DNAJB1, and
preferably, wherein said one or more oligodendrocyte signature gene or polypeptide is selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X and PHACTR3; or selected from the group consisting of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP and CNP; or selected from the group consisting of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X and PHACTR3.

10. The method according to claim 8, comprising determining the fraction of the cells comprised by the glioma, which express one or more stem cell or progenitor cell signature genes or polypeptides.

11. A method of identifying a therapeutic for IDH-mutant glioma, comprising administering to a IDH-mutant glioma cell in vitro a candidate therapeutic and monitoring expression or activity of one or more stem cell or progenitor cell signature genes or polypeptides, preferably wherein reduction in expression or activity of said one or more stem cell or progenitor cell signature genes or polypeptides is indicative of a therapeutic effect.

12. (canceled)

13. A method of monitoring IDH-mutant glioma treatment or evaluating IDH-mutant glioma treatment efficacy, comprising determining expression or activity of one or more stem cell or progenitor cell signature genes or polypeptides in cells comprised by the IDH-mutant glioma, preferably,
comprising determining the relative expression level of one or more stem cell or progenitor cell signature genes or polypeptides compared to one or more astrocyte and/or oligodendrocyte signature genes or polypeptides in the cells comprised by the glioma; and/or
comprising determining the fraction of the cells comprised by the glioma, which express one or more stem cell or progenitor cell signature genes or polypeptides.

14. (canceled)

15. (canceled)

16. A method of diagnosing, prognosing, or stratifying IDH-mutant glioma, comprising identifying cells comprised by the IDH-mutant glioma, which express one or more of CX3CR1, CD14, CD53, CD68, CD74, FCGR2A, HLA-DRA, or CSF1R, or one or more of MOBP, OPALIN, MBP, PLLP, CLDN11, MOG, or PLP1, preferably,
wherein said cells comprise a stem cell or progenitor cell, more preferably, wherein said stem cell or progenitor cell is a neural stem cell or progenitor cell, more preferably wherein said stem cell or progenitor cell signature genes or polypeptides are not oligodendrocyte precursor cell signature genes or polypeptides; and/or
wherein said glioma is oligodendroglioma, astrocytoma, or oligoastrocytoma; and/or
wherein said glioma is low grade glioma or grade II glioma; and/or
wherein said glioma is characterized by IDH1 and/or IDH2 mutations; and/or
wherein said glioma is characterized by CIC mutations; and/or
wherein said glioma is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36; and/or
wherein said glioma is characterized by deletion of chromosome arms 1p and/or 19q, and/or
wherein said subject has not previously received chemotherapy and/or radiotherapy.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The method according to claim 8, further comprising detecting one or more signature genes that is an indicator of a low-cycling or a high-cycling tumor, preferably, wherein the one or more signature genes comprises cyclin D3 (CCND3) or KDM5B (JAR1D1B), wherein CCND3 indicates high-cycling tumors and KDM5B indicates non-cycling cells.

37. (canceled)

38. An isolated cell characterized by the expression of one or more signature genes or polypeptides,
wherein said cell expresses one or more stem cell or progenitor cell signature genes or polypeptides selected from the group consisting of SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, COROIC, EIF4B, FBLIM1, SPDYE7P, TCF4, ORC6, SPDYEl, NCRUPAR, BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5 and TNFAIP8L1; or SOX4, SOX11, SOX2, NFIB, ASCL1, CDH7, CD24, BOC, and TCF4; or SOX4, CCND2, SOX11, CDH7, CD24, NFIB, SOX2, TCF4, ASCL1, BOC, and EGFR: or SOX11, SOX4, NFIB TCF4, SOX2, CDH7, BOC, and CCND2; or SOX11, PTMA, NFIB, CCND2, SOX4, TCF4, CD24, CHD7, and SOX2; or SOX2, SOX4, SOX11, MSI1, TERF2, CTNNB1, USP22, BRD3, CCND2, and PTEN: or SOX4, PTPRS, NFIB, CCND2, RBM6, SET, BAZ2B and TRA2A: or SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2, or
wherein said cell expresses one or more astrocyte signature genes or polypeptides selected from the group consisting of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1 and F3; or APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2 and F3; or SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1 and DNAJB1, or
wherein said cell expresses one or more oligodendrocyte signature genes or polypeptides selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X and PHACTR3; or OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP and CNP, or LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X and PHACTR3.

39. (canceled)

40. A method of monitoring an IDH-mutant glioma, comprising determining expression or activity of one or more macrophage and microglia signature genes or polypeptides in cells comprised by the IDH-mutant glioma, whereby an increase in macrophage over microglia programs in the tumor microenvironment indicates an increase in tumor grade and an increase in proliferation of malignant cells.

41. The method according to claim 40, wherein the microglia signature genes comprise CX3CR1, P2RY12, P2RY13 and SELPLG, and wherein the macrophage signature genes comprise CD163, CD74, TGFBI, IFITM2, IFITM3, F13A1, NPC2, TAGLN2 and FTH1.
